(19) United States
(12) Patent Application Publication
(10) Pub. No.: US 2009/0042726 A1
(43) Pub. Date: Feb. 12, 2009

(54) Novel Herbicides

(75) Inventors: Janice Black, Berkshire (GB); Jutta Elisabeth Boehmer, Berkshire (GB); Ewan James Turner Chrystal, Berkshire (GB); Anthony Marian Kozakiewicz, Berkshire (GB); Andrew Plant, Berkshire (GB)

Correspondence Address: SYNGENTA CROP PROTECTION , INC.;PATENT AND TRADEMARK DEPARTMENT, 410 SWING ROAD, GREENSBORO, NC, 27409, US

(73) Assignee: SYNGENTA LIMITED, Guildford, Surrey (GB)

(21) Appl. No.: 12/097,930

(22) PCT Filed: Nov. 21, 2006
(86) PCT No.: PCT/GB06/04334

§ 371 Date: Jun. 18, 2008

(30) Foreign Application Priority Data
Dec. 21, 2005 (GB) 0526044.3

(51) Int. Cl. A01N 25/32; C07D 261/04; A01N 43/80; A01N 43/40; A01P 21/00; C07D 413/12; A01N 43/54

(52) U.S. Cl. 504/103; 548/243; 504/271; 504/252; 546/272.1; 544/333; 504/239

(57) ABSTRACT

Compounds of formula I: wherein R¹, R², R³, R⁴, m, R⁵, R⁶, n and Y are as defined in claim 1; or N-oxides, salts and optical isomers thereof. Furthermore, the present invention relates to processes for preparing compounds of formula (I), to herbicidal compositions comprising them and to methods of using them to control plants or to inhibit plant growth.

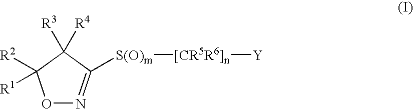

The present invention relates to novel, herbicidal isoxazoline compounds, to processes for their preparation, to compositions comprising those compounds, and to their use in controlling plants or in inhibiting plant growth.

Isoxazoline compounds which display a herbicidal action are described, for example, in WO 01/012613, WO 02/062770, WO 03/000686, WO 04/010165, JP 2005/035924, JP 2005/213168 and WO 06/024820. The preparation of these compounds is also described in WO 04/013106.

Novel isoxazoline compounds which display herbicidal and growth-inhibiting properties have now been found.

The present invention accordingly relates to compounds of formula I

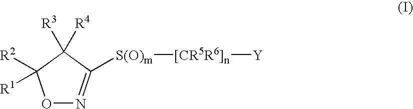

wherein
R¹ and R² are each independently of the other hydrogen, C₁-C₁₀alkyl, C₁-C₁₀haloalkyl, C₃-C₈cycloalkyl or C₃-C₈cycloalkyl-C₁-C₃alkyl, or
R¹ and R² together with the carbon atom to which they are bonded form a C₃-C₇ring,
R³ is halogen, azide, cyano, —SCN, C₂-C₁₀alkynyl, C₂-C₁₀alkenyl, formyl, C₁-C₁₀alkoxy, C₁-C₁₀alkylsulfanyl, C₁-C₁₀haloalkoxy, C₁-C₁₀haloalkylsulfanyl,
R⁴ is hydrogen, C₁-C₁₀alkyl, C₁-C₁₀haloalkyl, C₃-C₈cycloalkyl-C₁-C₁₀alkyl, C₁-C₆alkoxy-C₁-C₁₀alkyl or C₃-C₈cycloalkyl, halogen, azide, cyano, —SCN, C₂-C₁₀alkynyl, C₂-C₁₀alkenyl, formyl, C₁-C₁₀alkoxy, C₁-C₁₀alkylsulfanyl, C₁-C₁₀haloalkoxy, C₁-C₁₀halo-alkylsulfanyl, or
R² with R⁴ and together with the carbon atoms to which they are bonded form a C₃-C₈ring;
R⁵ and R⁶ are each independently of the other hydrogen, cyano, C₁-C₆alkyl, C₁-C₆alkoxycarbonyl, halogen or C₁-C₆haloalkyl;
m is 0, 1 or 2;
n is 1, 2 or 3;
Y is phenyl, naphthyl or tetrahydronaphthyl, which is optionally substituted by one to five substituents independently selected from C₁-C₆alkyl, C₃-C₆cycloalkyl, C₁-C₆halo-alkyl, C₁-C₆hydroxyalkyl, C₁-C₆alkoxy-C₁-C₆alkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl, nitro, cyano, formyl, carboxyl, halogen, azido, thiocyanato, tri(C₁-C₆alkyl)silyl, mercapto, phenylthio, phenylsulfinyl, —SF₅, C₁-C₆alkylthio, C₁-C₆haloalkyl-thio, C₁-C₆haloalkylsulfinyl, C₁-C₆haloalkylsulfonyl, C₁-C₆alkylsulfinyl, C₁-C₆alkyl-sulfonyl, benzylsulfonyl or benzylsulfonyl substituted by one to five R⁹, phenylsulfonyl or phenylsulfonyl substituted by one to five R⁹, hydroxyl, C₁-C₆alkoxy, C₃-C₆cyclo-alkyloxy wherein one of the CH₂ groups is optionally replaced by an oxygen atom, C₁-C₆haloalkoxy, C₂-C₆alkenyloxy, C₂-C₆alkynyloxy, C₁-C₆alkylsulfonyloxy, C₁-C₆halo-alkylsulfonyloxy, phenoxy or phenoxy substituted by one to five R⁹, benzyloxy or benzyloxy substituted by one to five R⁹, —CONH—SO₂—C₁-C₆alkyl, —CONH—SO₂—C₁-C₆haloalkyl, —NH—SO₂—C₁-C₆alkyl, —NH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —OCO—C₁-C₆alkyl, —OCO—C₁-C₆haloalkyl, —OCO-phenyl or —OCO-phenyl substituted by one to five R⁹, —OCONH—C₁-C₆alkyl, —OCONH—C₁-C₆haloalkyl, —OCONH-phenyl or —OCONH-phenyl substituted by one to five R⁹, or by one of the following groups Z, with Z

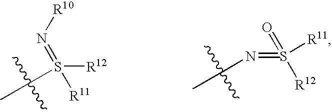

R¹⁰ is hydrogen, formyl, cyano, nitro, C₁-C₆alkylsulfonyl, C₁-C₁₀alkyl, C₁-C₁₀haloalkyl, C₁-C₁₀alkylcarbonyl, C₁-C₁₀haloalkylcarbonyl, C₁-C₁₀alkoxycarbonyl, and
R¹¹ and R¹² are independently of each other C₁-C₁₀alkyl, C₁-C₁₀haloalkyl, C₁-C₁₀cyclo-alkyl, C₁-C₁₀cycloalkylalkyl, C₁-C₁₀alkoxyalkyl, or by —CONR⁷R⁸ wherein R⁷ and R⁹ are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R⁷ and R⁸ form a C₃-C₈alkylene group which optionally contains one oxygen or sulfur atom or one to two amino or C₁-C₆alkylamino groups, or
Y is a 5- to 10-membered aromatic or non-aromatic heterocycle containing one to three nitrogen, oxygen or sulfur atoms, which is optionally benzo-fused, and which is optionally substituted by one to four substituents independently selected from C₁-C₆alkyl, C₃-C₆cycloalkyl, C₃-C₆cycloalkyl-C₁-C₆alkyl, C₁-C₆haloalkyl, C₁-C₆hydroxyalkyl, C₁-C₆alkoxy-C₁-C₆alkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, nitro, cyano, formyl, carboxyl, halogen, azido, thiocyanato, tri(C₁-C₆alkyl)silyl, mercapto, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to five R⁹, phenylsulfonyl or phenylsulfonyl substituted by one to five R⁹, hydroxyl, C₁-C₆alkoxy, C₁-C₆alkoxy-C₁-C₆alkoxy, C₃-C₆cycloalkyloxy wherein one of the CH₂ groups is optionally replaced by an oxygen atom, C₁-C₆haloalkoxy, C₂-C₆alkenyloxy, C₂-C₆alkynyloxy, C₁-C₆alkylsulfonyloxy, C₁-C₆haloalkylsulfonyloxy, phenoxy or phenoxy substituted by one to five R⁹, benzyloxy or benzyloxy substituted by one to five R⁹, —CONH—SO₂—C₁-C₆alkyl, —CONH—SO₂—C₁-C₆haloalkyl, —NH—SO₂—C₁-C₆alkyl, —NH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —OCO—C₁-C₆alkyl, —OCO—C₁-C₆haloalkyl, —OCO-phenyl or —OCO-phenyl substituted by one to five R⁹, —OCONH—C₁-C₆alkyl, —OCONH—C₁-C₆haloalkyl, —OCONH-phenyl or —OCONH-phenyl substituted by one to five R⁹, or by one of the following groups Z, with Z=

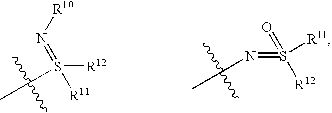

R¹⁰ is hydrogen, formyl, cyano, nitro, C₁-C₆alkylsulfonyl, C₁-C₁₀alkyl, C₁-C₁₀haloalkyl, C₁-C₁₀alkylcarbonyl, C₁-C₁₀haloalkylcarbonyl, C₁-C₁₀alkoxycarbonyl, and

R¹¹ and R¹² are independently of each other C₁-C₁₀alkyl, C₁-C₁₀haloalkyl, C₁-C₁₀cyclo-alkyl, C₁-C₁₀cycloalkylalkyl, C₁-C₁₀alkoxyalkyl, or by —CONR⁷R⁸ wherein R⁷ and R⁸ are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R⁷ and R⁸ form a C₃-C₈alkylene group which optionally contains one oxygen or sulfur atom or one to two amino or C₁-C₆alkylamino groups;

R⁹ are independently from each other C₁-C₆haloalkyl, C₁-C₆alkoxycarbonyl, nitro, cyano, formyl, carboxyl or halogen;
and to N-oxides, salts and optical isomers of compounds of formula I.

Preferably R¹ and R² are independently C₁-C₁₀alkyl or C₁-C₁₀haloalkyl, more preferably C₁-C₆alkyl or C₁-C₆haloalkyl, most preferably methyl.

Preferably R³ is halogen, azide or cyano, more preferably fluoro, chloro or bromo, even more preferably fluoro or chloro, most preferably fluoro.

Preferably R⁴ is hydrogen or halogen, more preferably hydrogen, fluoro or chloro, even more preferably hydrogen or fluoro, most preferably hydrogen.

Preferably R⁵ is hydrogen, C₁-C₆alkyl or halogen, more preferably hydrogen, methyl or halogen, even more preferably hydrogen, methyl, fluoro or chloro, most preferably hydrogen or fluoro.

Preferably R⁶ is hydrogen, methoxycarbonyl, C₁-C₆alkyl or halogen, more preferably hydrogen, methyl, fluoro or chloro.

Preferably m is 1 or 2.

Preferably n is 1.

A group of preferred compounds of formula I comprises those wherein

Y is phenyl, naphthyl or tetrahydronaphthyl, which is optionally substituted by one to five substituents independently selected from C₁-C₆alkyl, C₃-C₆cycloalkyl, C₁-C₆halo-alkyl, C₁-C₆hydroxyalkyl, C₁-C₆alkoxy-C₁-C₆alkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl, nitro, cyano, formyl, carboxyl, halogen, azido, thiocyanato, tri(C₁-C₆alkyl)silyl, mercapto, phenylthio, phenylsulfinyl, —SF₅, C₁-C₆alkylthio, C₁-C₆haloalkyl-thio, C₁-C₆haloalkylsulfinyl, C₁-C₆haloalkylsulfonyl, C₁-C₆alkylsulfinyl, C₁-C₆alkyl-sulfonyl, benzylsulfonyl or benzylsulfonyl substituted by one to five R⁹, phenylsulfonyl or phenylsulfonyl substituted by one to five R⁹, hydroxyl, C₁-C₆alkoxy, C₃-C₆cyclo-alkyloxy wherein one of the CH₂ groups is optionally replaced by an oxygen atom, C₁-C₆haloalkoxy, C₂-C₆alkenyloxy, C₂-C₆alkynyloxy, C₁-C₆alkylsulfonyloxy, C₁-C₆halo-alkylsulfonyloxy, phenoxy or phenoxy substituted by one to five R⁹, benzyloxy or benzyloxy substituted by one to five R⁹, —CONH—SO₂—C₁-C₆alkyl, —CONH—SO₂—C₁-C₆haloalkyl, —NH—SO₂—C₁-C₆alkyl, —NH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —OCO—C₁-C₆alkyl, —OCO—C₁-C₆haloalkyl, —OCO-phenyl or —OCO-phenyl substituted by one to five R⁹, —OCONH—C₁-C₆alkyl, —OCONH—C₁-C₆haloalkyl, —OCONH-phenyl or —OCONH-phenyl substituted by one to five R⁹, or by one of the following groups Z, with Z=

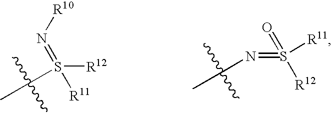

R¹⁰ is hydrogen, formyl, cyano, nitro, C₁-C₆alkylsulfonyl, C₁-C₁₀alkyl, C₁-C₁₀haloalkyl, C₁-C₁₀alkylcarbonyl, C₁-C₁₀haloalkylcarbonyl, C₁-C₁₀alkoxycarbonyl, and
R¹¹ and R¹² are independently of each other C₁-C₁₀alkyl, C₁-C₁₀haloalkyl, C₁-C₁₀cyclo-alkyl, C₁-C₁₀cycloalkylalkyl, C₁-C₁₀alkoxyalkyl, or by —CONR⁷R⁵ wherein R⁷ and R⁸ are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R⁷ and R⁸ form a C₃-C₈alkylene group which optionally contains one oxygen or sulfur atom or one to two amino or C₁-C₆alkylamino groups.

A group of preferred compounds of formula I comprises those wherein Y is phenyl which is optionally substituted by one to five substituents independently selected from C₁-C₆alkyl, C₁-C₆haloalkyl, C₁-C₆alkoxycarbonyl, halogen, cyano, nitro, C₁-C₆haloalkylsulfanyl, C₁-C₆haloalkylsulfinyl, C₁-C₆alkoxy, C₁-C₆haloalkoxy, C₂-C₆alkynyloxy, C₁-C₆alkylsulfonyloxy or phenyl, more preferably wherein Y is phenyl which is optionally substituted by one to five substituents independently selected from methyl, trifluoromethyl, methoxycarbonyl, ethoxycarbonyl, fluoro, chloro, cyano, nitro, trifluoromethylthio, trifluoromethylsulfinyl, methoxy, difluoromethoxy, trifluoro-methoxy, propargyloxy, methylsulfonyloxy or phenyl, most preferably wherein Y is phenyl which is optionally substituted by one to five substituents independently selected from fluoro or trifluoromethoxy. A group of particularly preferred compounds of formula I comprises those wherein Y is phenyl which is optionally substituted by one to three substituents independently selected from fluoro, chloro, cyano, difluoromethoxy, ethoxycarbonyl, methoxy, methoxycarbonyl, methyl, methylsulfonyloxy, nitro, phenyl, propargyloxy, trifluoromethoxy, trifluoromethyl, trifluoromethylthio or trifluoromethylsulfinyl.

A group of especially preferred compounds of formula I comprises those wherein Y is 2,6-difluorophenyl.

A group of especially preferred compounds of formula I comprises those wherein Y is 2-fluoro-6-chlorophenyl.

A group of especially preferred compounds of formula I comprises those wherein Y is 2-trifluoromethoxyphenyl.

A group of especially preferred compounds of formula I comprises those wherein Y is 2-difluoromethoxyphenyl.

A group of preferred compounds of formula I comprises those wherein Y is a 5- to 10-membered aromatic or non-aromatic heterocycle containing one to three nitrogen, oxygen or sulfur atoms, which is optionally benzo-fused, and which is optionally substituted by one to four substituents independently selected from C₁-C₆alkyl, C₃-C₆cycloalkyl, C₃-C₆cycloalkyl-C₁-C₆alkyl, C₁-C₆haloalkyl, C₁-C₆hydroxyalkyl, C₁-C₆alkoxy-C₁-C₆alkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, nitro, cyano, formyl, carboxyl, halogen, azido, thiocyanato, tri(C₁-C₆alkyl)silyl, mercapto, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to five R⁹, phenylsulfonyl or phenylsulfonyl substituted by one to five R⁹, hydroxyl, C₁-C₆alkoxy, C₁-C₆alkoxy-C₁-C₆alkoxy, C₃-C₆cycloalkyloxy wherein one of the CH₂ groups is optionally replaced by an oxygen atom, C₁-C₆haloalkoxy, C₂-C₆alkenyloxy, C₂-C₆alkynyloxy, C₁-C₆alkylsulfonyloxy, C₁-C₆haloalkylsulfonyloxy, phenoxy or phenoxy substituted by one to five R⁹, benzyloxy or benzyloxy substituted by one to five R⁹, —CONH—SO₂—C₁-C₆alkyl, —CONH—SO₂—C₁-C₆haloalkyl, —NH—SO₂—C₁-C₆alkyl, —NH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —OCO—C₁-C₆alkyl, —OCO—C₁-C₆haloalkyl, —OCO-phenyl or —OCO-phenyl substituted by one to five R⁹, —OCONH—C₁-C₆alkyl, —OCONH—C₁-C₆haloalkyl, —OCONH-phenyl or —OCONH-phenyl substituted by one to five R⁹, or by one of the following groups Z, with Z=

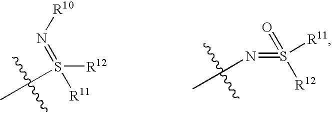

R¹⁰ is hydrogen, formyl, cyano, nitro, C₁-C₆alkylsulfonyl, C₁-C₁₀alkyl, C₁-C₁₀haloalkyl, C₁-C₁₀alkylcarbonyl, C₁-C₁₀haloalkylcarbonyl, C₁-C₁₀alkoxycarbonyl, and R¹¹ and R¹² are independently of each other C₁-C₁₀alkyl, C₁-C₁₀haloalkyl, C₁-C₁₀cyclo-alkyl, C₁-C₁₀cycloalkylalkyl, C₁-C₁₀alkoxyalkyl, or by —CONR⁷R⁸ wherein R⁷ and R⁸ are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R⁷ and R⁸ form a C₃-C₈alkylene group which optionally contains one oxygen or sulfur atom or one to two amino or C₁-C₆alkylamino groups.

A group of preferred compounds of formula I comprises those wherein Y is an optionally substituted pyridinyl or pyrimidinyl; a group of especially preferred compounds of formula I comprises those wherein Y is an optionally substituted pyridin-3-yl or pyrimidin-5-yl.

A group of preferred compounds of formula I comprises those wherein Y is pyridinyl which is optionally substituted by one to four substituents independently selected from C₁-C₆alkyl, C₁-C₆haloalkyl, halogen, cyano, C₁-C₆alkoxy or C₁-C₆halo-alkoxy, more preferably wherein Y is pyridyl which is optionally substituted by one to four substituents independently selected from methyl, trifluoromethyl, fluoro, chloro, cyano, methoxy, difluoromethoxy, trifluoromethoxy or 2,2,2-trifluoroethoxy, even more preferably wherein Y is pyridyl which is optionally substituted by one to four substituents independently selected from methyl, trifluoromethyl, chloro or methoxy, most preferably wherein Y is pyridyl which is optionally substituted by one to four substituents independently selected from methyl, trifluoromethyl or chloro. A group of especially preferred compounds of formula I comprises those wherein Y is pyridin-3-yl.

A group of especially preferred compounds of formula I comprises those wherein

Y is 2-methyl-6-trifluoromethyl-pyridin-3-yl.

A group of especially preferred compounds of formula I comprises those wherein

Y is 2-chloro-pyridin-3-yl.

A group of especially preferred compounds of formula I comprises those wherein

Y is 2-methoxy-pyridin-3-yl.

A group of preferred compounds of formula I comprises those wherein Y is pyrimidinyl which is optionally substituted by one to three substituents independently selected from C₁-C₆alkyl, C₁-C₆haloalkyl, halogen, cyano, C₁-C₆alkoxy or C₁-C₆halo-alkoxy, more preferably wherein Y is pyrimidinyl which is optionally substituted by one to three substituents independently selected from methyl, trifluoromethyl, fluoro, chloro, cyano, methoxy, difluoromethoxy, trifluoromethoxy or 2,2,2-trifluoroethoxy, most preferably wherein Y is pyrimidinyl which is optionally substituted by one to three substituents independently selected from trifluoromethyl or methoxy. A group of especially preferred compounds of formula I comprises those wherein Y is pyrimidin-5-yl.

A group of especially preferred compounds of formula I comprises those wherein Y is 4-methoxy-6-trifluoromethyl-pyrimidin-5-yl.

A group of preferred compounds of formula I comprises those wherein Y is an optionally substituted pyrazolyl, triazolyl, thiadiazolyl or triazolyl-N-oxide; a group of especially preferred compounds of formula I comprises those wherein Y is an optionally substituted pyrazol-3-yl, pyrazol-4-yl, pyrazol-5-yl, 1,2,3-triazol-4-yl, 1,2,3-triazol-5-yl, 1,2,4-triazol-3-yl, 1,2,4-triazol-5-yl, 1,2,3-thiadiazol-5-yl or 1,2,3-triazol-4-yl-1-N-oxide. A group of particularly preferred compounds of formula I comprises those wherein Y is an optionally substituted pyrazol-3-yl, pyrazol-4-yl, pyrazol-5-yl, 1,2,3-triazol-4-yl or 1,2,3-triazol-5-yl.

A group of preferred compounds of formula I comprises those wherein Y is pyrazolyl which is optionally substituted by one to three substituents independently selected from C₁-C₆alkyl, C₁-C₆haloalkyl, C₂-C₆alkenyl, C₂-C₆alkynyl, halogen, cyano, C₁-C₆alkoxy, C₁-C₆haloalkoxy or C₁-C₆alkoxy-C₁-C₆alkoxy, more preferably wherein Y is pyrazolyl which is optionally substituted by one to three substituents independently selected from methyl, ethyl, iso-propyl, monofluoromethyl, difluoromethyl, trifluoromethyl, allyl, propen-2-yl, propargyl, fluoro, chloro, cyano, methoxy, ethoxy, monofluoromethoxy, difluoromethoxy, trifluoromethoxy, 2,2-difluoroethoxy, 2,2,2-trifluoroethoxy, 2-fluoro-1-methyl-ethoxy, 2,2,2-trifluoro-1-methyl-ethoxy or 2-methoxy-ethoxy, even more preferably wherein Y is pyrazolyl which is optionally substituted by one to three substituents independently selected from methyl, ethyl, iso-propyl, difluoromethyl, trifluoromethyl, allyl, propen-2-yl, propargyl, fluoro, chloro, cyano, methoxy, difluoromethoxy, 2,2,2-trifluoroethoxy, 2-fluoro-1-methyl-ethoxy, 2,2,2-trifluoro-1-methyl-ethoxy or 2-methoxy-ethoxy, most preferably wherein Y is pyrazolyl which is optionally substituted by one to three substituents independently selected from methyl, ethyl, trifluoromethyl, allyl, propargyl, fluoro, chloro, cyano, methoxy, difluoromethoxy, 2,2,2-trifluoroethoxy, 2,2,2-trifluoro-1-methyl-ethoxy or 2-methoxy-ethoxy. A group of particularly preferred compounds of formula I comprises those wherein Y is pyrazolyl which is optionally substituted by one to three substituents independently selected from chloro, 2,2-difluoroethoxy, difluoromethoxy, ethoxy, methoxy, methyl, ethyl, 2,2,2-trifluoroethoxy, trifluoromethyl, difluoromethyl or monofluoromethyl.

A group of especially preferred compounds of formula I comprises those wherein Y is pyrazol-3-yl which is optionally substituted by one to three substituents independently selected from C₁-C₆alkyl, C₁-C₆haloalkyl, halogen, cyano, C₁-C₆alkoxy or C₁-C₆haloalkoxy, more preferably wherein Y is pyrazol-3-yl which is optionally substituted by one to three substituents independently selected from methyl, ethyl, monofluoromethyl, difluoromethyl, trifluoromethyl, fluoro, chloro, cyano, methoxy, ethoxy, monofluoromethoxy, difluoromethoxy, trifluoromethoxy, 2,2-difluoroethoxy or 2,2,2-trifluoroethoxy, most preferably wherein Y is pyrazol-3-yl which is optionally substituted by one to three substituents independently selected from methyl, trifluoro-methyl or cyano. A group of particularly preferred compounds of formula I comprises those wherein Y is pyrazol-3-yl which is optionally substituted by one to three substituents independently selected from chloro, 2,2-difluoroethoxy, difluoromethoxy, ethoxy, methoxy, methyl, ethyl, 2,2,2-trifluoroethoxy, trifluoromethyl, difluoromethyl or monofluoromethyl.

A group of especially preferred compounds of formula I comprises those wherein Y is 1-methyl-4-trifluoromethyl-pyrazol-3-yl.

A group of especially preferred compounds of formula I comprises those wherein Y is 5-cyano-1-methyl-4-trifluoromethyl-pyrazol-3-yl.

A group of especially preferred compounds of formula I comprises those wherein Y is pyrazol-4-yl which is optionally substituted by one to three substituents independently selected from C₁-C₆alkyl, C₁-C₆haloalkyl, C₂-C₆alkenyl, C₂-C₆alkynyl, halogen, cyano, C₁-C₆alkoxy, C₁-C₆haloalkoxy or C₁-C₆alkoxy-C₁-C₆alkoxy, more preferably wherein Y is pyrazol-4-yl which is optionally substituted by one to three substituents independently selected from methyl, ethyl, iso-propyl, monofluoromethyl, difluoro-methyl, trifluoromethyl, allyl, propen-2-yl, propargyl, fluoro, chloro, cyano, methoxy, ethoxy, monofluoromethoxy, difluoromethoxy, trifluoromethoxy, 2,2-difluoroethoxy, 2,2,2-trifluoroethoxy, 2-fluoro-1-methyl-ethoxy, 2,2,2-trifluoro-1-methyl-ethoxy or 2-methoxy-ethoxy, even more preferably wherein Y is pyrazol-4-yl which is optionally substituted by one to three substituents independently selected from methyl, ethyl, iso-propyl, difluoromethyl, trifluoromethyl, allyl, propen-2-yl, propargyl, fluoro, difluoro-methoxy, 2,2,2-trifluoroethoxy, 2-fluoro-1-methyl-ethoxy, 2,2,2-trifluoro-1-methyl-ethoxy or 2-methoxy-ethoxy, most preferably wherein Y is pyrazol-4-yl which is optionally substituted by one to three substituents independently selected from methyl, ethyl, trifluoromethyl, allyl, propargyl, fluoro, difluoromethoxy, 2,2,2-trifluoroethoxy, 2,2,2-trifluoro-1-methyl-ethoxy or 2-methoxy-ethoxy. A group of particularly preferred compounds of formula I comprises those wherein Y is pyrazol-4-yl which is optionally substituted by one to three substituents independently selected from chloro, 2,2-difluoro-ethoxy, difluoromethoxy, ethoxy, methoxy, methyl, ethyl, 2,2,2-trifluoroethoxy, trifluoromethyl, difluoromethyl or monofluoromethyl.

A group of especially preferred compounds of formula I comprises those wherein Y is 3-difluoromethoxy-1-methyl-5-trifluoromethyl-pyrazol-4-yl.

A group of especially preferred compounds of formula I comprises those wherein Y is 5-difluoromethoxy-1-methyl-3-trifluoromethyl-pyrazol-4-yl.

A group of especially preferred compounds of formula I comprises those wherein Y is 1-methyl-5-(2,2,2-trifluoroethoxy)-3-trifluoromethyl-pyrazol-4-yl.

A group of especially preferred compounds of formula I comprises those wherein Y is 1-methyl-3-trifluoromethyl-pyrazol-4-yl.

A group of especially preferred compounds of formula I comprises those wherein Y is 1-methyl-3-trifluoromethyl-5-(2,2,2-trifluoro-1-methyl-ethoxy)-pyrazol-4-yl.

A group of especially preferred compounds of formula I comprises those wherein Y is 1-methyl-3-trifluoromethyl-5-(2-fluoro-1-methyl-ethoxy)-pyrazol-4-yl.

A group of especially preferred compounds of formula I comprises those wherein Y is 1-methyl-3-(2,2,2-trifluoroethoxy)-5-trifluoromethyl-pyrazol-4-yl.

A group of especially preferred compounds of formula I comprises those wherein Y is 1-ethyl-3-trifluoromethyl-pyrazol-4-yl.

A group of especially preferred compounds of formula I comprises those wherein Y is 1-propargyl-3-trifluoromethyl-pyrazol-4-yl.

A group of especially preferred compounds of formula I comprises those wherein Y is 1-allyl-3-trifluoromethyl-pyrazol-4-yl.

A group of especially preferred compounds of formula I comprises those wherein Y is 5-fluoro-1-methyl-3-trifluoromethyl-pyrazol-4-yl.

A group of especially preferred compounds of formula I comprises those wherein Y is 5-(2-methoxy-ethoxy)-1-methyl-3-trifluoromethyl-pyrazol-4-yl.

A group of especially preferred compounds of formula I comprises those wherein Y is 1,3-dimethyl-5-(2,2,2-trifluoroethoxy)-pyrazol-4-yl.

A group of especially preferred compounds of formula I comprises those wherein Y is 1-methyl-5-(propen-2-yl)-3-trifluoromethyl-pyrazol-4-yl.

A group of especially preferred compounds of formula I comprises those wherein Y is 1-difluoromethyl-5-trifluoromethyl-pyrazol-4-yl.

A group of especially preferred compounds of formula I comprises those wherein Y is 1-difluoromethyl-3-trifluoromethyl-pyrazol-4-yl.

A group of especially preferred compounds of formula I comprises those wherein Y is 1-iso-propyl-3-trifluoromethyl-pyrazol-4-yl.

A group of especially preferred compounds of formula I comprises those wherein Y is pyrazol-5-yl which is optionally substituted by one to three substituents independently selected from C₁-C₆alkyl, C₁-C₆haloalkyl, halogen, cyano, nitro, C₁-C₆alkoxy or C₁-C₆haloalkoxy, more preferably wherein Y is pyrazol-5-yl which is optionally substituted by one to three substituents independently selected from methyl, ethyl, monofluoromethyl, difluoromethyl, trifluoromethyl, fluoro, chloro, cyano, methoxy, ethoxy, monofluoromethoxy, difluoromethoxy, trifluoromethoxy, 2,2-difluoroethoxy or 2,2,2-trifluoroethoxy, most preferably wherein Y is pyrazol-5-yl which is optionally substituted by one to three substituents independently selected from methyl, trifluoromethyl, chloro, methoxy or difluoromethoxy. A group of particularly preferred compounds of formula I comprises those wherein Y is pyrazol-5-yl which is optionally substituted by one to three substituents independently selected from chloro, 2,2-difluoroethoxy, difluoromethoxy, ethoxy, methoxy, methyl, ethyl, 2,2,2-trifluoroethoxy, trifluoromethyl, difluoromethyl or monofluoromethyl.

A group of especially preferred compounds of formula I comprises those wherein Y is 1-methyl-3-trifluoromethyl-pyrazol-5-yl.

A group of especially preferred compounds of formula I comprises those wherein Y is 4-chloro-1-methyl-3-trifluoromethyl-pyrazol-5-yl.

A group of especially preferred compounds of formula I comprises those wherein Y is 1-methyl-3-difluoromethoxy-pyrazol-5-yl.

A group of especially preferred compounds of formula I comprises those wherein Y is 3-methoxy-1-methyl-pyrazol-5-yl.

A group of preferred compounds of formula I comprises those wherein Y is triazolyl which is optionally substituted by one to two substituents independently selected from C₁-C₆alkyl, C₃-C₆cycloalkyl, C₃-C₆cycloalkyl-C₁-C₆-alkyl, C₁-C₆haloalkyl, C₁-C₆alkoxy-C₁-C₆alkyl, C₂-C₆alkenyl, halogen, cyano, C₁-C₆alkoxy or C₁-C₆haloalkoxy, more preferably wherein Y is triazolyl which is optionally substituted by one to two substituents independently selected from methyl, ethyl, iso-propyl, tert-butyl, cyclopentyl, cyclobutylmethyl, monofluoromethyl, difluoromethyl, trifluoromethyl, 2-methoxy-ethyl, allyl, fluoro, chloro, bromo, cyano, methoxy, ethoxy, monofluoro-methoxy, difluoromethoxy, trifluoromethoxy, 2,2-difluoroethoxy or 2,2,2-trifluoro-ethoxy, even more preferably wherein Y is triazolyl which is optionally substituted by one to two substituents independently selected from methyl, ethyl, iso-propyl, tert-butyl, cyclopentyl, cyclobutylmethyl, trifluoromethyl, 2-methoxy-ethyl, allyl, bromo, methoxy or difluoromethoxy, most preferably wherein Y is triazolyl which is optionally substituted by one to two substituents independently selected from methyl, ethyl, iso-propyl, cyclopentyl, cyclobutylmethyl, trifluoromethyl, 2-methoxy-ethyl, allyl or bromo. A group of particularly preferred compounds of formula I comprises those wherein Y is triazolyl which is optionally substituted by one to three substituents independently selected from chloro, 2,2-difluoroethoxy, difluoromethoxy, ethoxy, methoxy, methyl, ethyl, 2,2,2-trifluoroethoxy, trifluoromethyl, difluoromethyl or monofluoromethyl.

A group of especially preferred compounds of formula I comprises those wherein Y is 1,2,3-triazol-4-yl which is optionally substituted by one to two substituents independently selected from C₁-C₆alkyl, C₃-C₆cycloalkyl, C₃-C₆cycloalkyl-C₁-C₆-alkyl, C₁-C₆haloalkyl, C₁-C₆alkoxy-C₁-C₆alkyl, C₂-C₆alkenyl, halogen, cyano, C₁-C₆alkoxy or C₁-C₆haloalkoxy, more preferably wherein Y is 1,2,3-triazol-4-yl which is optionally substituted by one to two substituents independently selected from methyl, ethyl, iso-propyl, tert-butyl, cyclopentyl, cyclobutylmethyl, monofluoromethyl, difluoromethyl, trifluoromethyl, 2-methoxy-ethyl, allyl, fluoro, chloro, bromo, cyano, methoxy, ethoxy, monofluoromethoxy, difluoromethoxy, trifluoromethoxy, 2,2-difluoroethoxy or 2,2,2-trifluoroethoxy, even more preferably wherein Y is 1,2,3-triazol-4-yl which is optionally substituted by one to two substituents independently selected from methyl, ethyl, iso-propyl, tert-butyl, cyclopentyl, cyclobutylmethyl, trifluoromethyl, 2-methoxy-ethyl, allyl, bromo, methoxy or difluoromethoxy, most preferably wherein Y is 1,2,3-triazol-4-yl which is optionally substituted by one to two substituents independently selected from methyl, ethyl, iso-propyl, cyclopentyl, cyclobutylmethyl, trifluoromethyl, 2-methoxy-ethyl or allyl. A group of particularly preferred compounds of formula I comprises those wherein Y is 1,2,3-triazol-4-yl which is optionally, substituted by one to three substituents independently selected from chloro, 2,2-difluoroethoxy, difluoromethoxy, ethoxy, methoxy, methyl, ethyl, 2,2,2-trifluoroethoxy, trifluoromethyl, difluoromethyl or monofluoromethyl.

A group of especially preferred compounds of formula I comprises those wherein Y is 2,5-dimethyl-1,2,3-triazol-4-yl.

A group of especially preferred compounds of formula I comprises those wherein Y is 2-allyl-5-trifluoromethyl-1,2,3-triazol-4-yl.

A group of especially preferred compounds of formula I comprises those wherein Y is 2-cyclopentyl-5-trifluoromethyl-1,2,3-triazol-4-yl.

A group of especially preferred compounds of formula I comprises those wherein Y is 2-cyclobutylmethyl-5-trifluoromethyl-1,2,3-triazol-4-yl.

A group of especially preferred compounds of formula I comprises those wherein Y is 2-(2-methoxy-ethyl)-5-trifluoromethyl-1,2,3-triazol-4-yl.

A group of especially preferred compounds of formula I comprises those wherein Y is 2-methyl-1,2,3-triazol-4-yl.

A group of especially preferred compounds of formula I comprises those wherein Y is 2-iso-propyl-5-trifluoromethyl-1,2,3-triazol-4-yl.

A group of especially preferred compounds of formula I comprises those wherein Y is 2-ethyl-5-trifluoromethyl-1,2,3-triazol-4-yl.

A group of especially preferred compounds of formula I comprises those wherein Y is 5-ethyl-2-methyl-1,2,3-triazol-4-yl.

A group of especially preferred compounds of formula I comprises those wherein Y is 2-ethyl-1,2,3-triazol-4-yl.

A group of especially preferred compounds of formula I comprises those wherein Y is 2-iso-propyl-5-methyl-1,2,3-triazol-4-yl.

A group of especially preferred compounds of formula I comprises those wherein Y is 2-methyl-5-trifluoromethyl-1,2,3-triazol-4-yl.

A group of especially preferred compounds of formula I comprises those wherein Y is 5-methoxy-2-methyl-1,2,3-triazol-4-yl.

A group of especially preferred compounds of formula I comprises those wherein Y is 5-bromo-2-methyl-1,2,3-triazol-4-yl.

A group of especially preferred compounds of formula I comprises those wherein Y is 2-ethyl-5-methyl-1,2,3-triazol-4-yl.

A group of especially preferred compounds of formula I comprises those wherein Y is 5-difluoromethoxy-2-methyl-1,2,3-triazol-4-yl.

A group of especially preferred compounds of formula I comprises those wherein Y is 1-tert-butyl-1,2,3-triazol-4-yl.

A group of especially preferred compounds of formula I comprises those wherein Y is 1,5-dimethyl-1,2,3-triazol-4-yl.

A group of especially preferred compounds of formula I comprises those wherein Y is 1,2,3-triazol-5-yl which is optionally substituted by one to two substituents independently selected from C₁-C₆alkyl, C₁-C₆haloalkyl, C₂-C₆alkenyl, halogen, cyano, C₁-C₆alkoxy or C₁-C₆haloalkoxy, more preferably wherein Y is 1,2,3-triazol-5-yl which is optionally substituted by one to two substituents independently selected from methyl, ethyl, iso-propyl, monofluoromethyl, difluoromethyl, trifluoromethyl, allyl, fluoro, chloro, bromo, cyano, methoxy, ethoxy, monofluoromethoxy, difluoromethoxy, trifluoro-methoxy, 2,2-difluoroethoxy or 2,2,2-trifluoroethoxy, even more preferably wherein Y is 1,2,3-triazol-5-yl which is optionally substituted by one to two substituents independently selected from methyl, iso-propyl, allyl or bromo, most preferably wherein Y is 1,2,3-triazol-5-yl which is optionally substituted by one to two substituents independently selected from methyl or bromo. A group of particularly preferred compounds of formula I comprises those wherein Y is 1,2,3-triazol-5-yl which is optionally substituted by one to three substituents independently selected from chloro, 2,2-difluoroethoxy, difluoromethoxy, ethoxy, methoxy, methyl, ethyl, 2,2,2-trifluoro-ethoxy, trifluoromethyl, difluoromethyl or monofluoromethyl.

A group of especially preferred compounds of formula I comprises those wherein Y is 4-bromo-1-methyl-1,2,3-triazol-5-yl.

A group of especially preferred compounds of formula I comprises those wherein Y is 4-methyl-1-iso-propyl-1,2,3-triazol-5-yl.

A group of especially preferred compounds of formula I comprises those wherein Y is 1-allyl-4-methyl-1,2,3-triazol-5-yl.

A group of especially preferred compounds of formula I comprises those wherein Y is 1,2,4-triazol-3-yl which is optionally substituted by one to two substituents independently selected from C₁-C₆alkyl, C₁-C₆haloalkyl, halogen, cyano, C₁-C₆alkoxy or C₁-C₆haloalkoxy, more preferably wherein Y is 1,2,4-triazol-3-yl which is optionally substituted by one to two substituents independently selected from methyl, ethyl, monofluoromethyl, difluoromethyl, trifluoromethyl, fluoro, chloro, cyano, methoxy, ethoxy, monofluoromethoxy, difluoromethoxy, trifluoromethoxy, 2,2-difluoroethoxy or 2,2,2-trifluoroethoxy, most preferably wherein Y is 1,2,4-triazol-3-yl which is optionally substituted by one to two methyl groups.

A group of especially preferred compounds of formula I comprises those wherein Y is 1-methyl-1,2,4-triazol-3-yl.

A group of especially preferred compounds of formula I comprises those wherein Y is 4,5-dimethyl-1,2,4-triazol-3-yl.

A group of especially preferred compounds of formula I comprises those wherein Y is 1,2,4-triazol-5-yl which is optionally substituted by one to two substituents independently selected from C₁-C₆alkyl, C₁-C₆haloalkyl, halogen, cyano, C₁-C₆alkoxy or C₁-C₆haloalkoxy, more preferably wherein Y is 1,2,4-triazol-5-yl which is optionally substituted by one to two substituents independently selected from methyl, ethyl, monofluoromethyl, difluoromethyl, trifluoromethyl, fluoro, chloro, cyano, methoxy, ethoxy, monofluoromethoxy, difluoromethoxy, trifluoromethoxy, 2,2-difluoroethoxy or 2,2,2-trifluoroethoxy, most preferably wherein Y is 1,2,4-triazol-5-yl which is optionally substituted by one to two methyl groups.

A group of especially preferred compounds of formula I comprises those wherein Y is 1,3-dimethyl-1,2,4-triazol-5-yl.

A group of especially preferred compounds of formula I comprises those wherein Y is 1-methyl-1,2,4-triazol-5-yl.

A group of preferred compounds of formula I comprises those wherein Y is 1,2,3-thiadiazolyl which is optionally substituted by a substituent selected from C₁-C₆alkyl, C₁-C₆haloalkyl, halogen, cyano, C₁-C₆alkoxy or C₁-C₆haloalkoxy, more preferably wherein Y is 1,2,3-thiadiazolyl which is optionally substituted by a substituent selected from methyl, ethyl, monofluoromethyl, difluoromethyl, trifluoromethyl, fluoro, chloro, cyano, methoxy, ethoxy, monofluoromethoxy, difluoromethoxy, trifluoromethoxy, 2,2-difluoro-ethoxy or 2,2,2-trifluoroethoxy, most preferably wherein Y is 1,2,3-thiadiazolyl which is optionally substituted by a methyl group. A group of especially preferred compounds of formula I comprises those wherein Y is 1,2,3-thiadiazol-5-yl.

A group of especially preferred compounds of formula I comprises those wherein Y is 4-methyl-1,2,3-thiadiazol-5-yl.

A group of preferred compounds of formula I comprises those wherein Y is 1,2,3-triazolyl-N-oxide which is optionally substituted by one or two substituents selected from C₁-C₆alkyl, C₁-C₆haloalkyl, halogen, cyano, C₁-C₆alkoxy or C₁-C₆haloalkoxy, more preferably wherein Y is 1,2,3-triazolyl-N-oxide which is optionally substituted by one or two substituents selected from methyl, ethyl, monofluoromethyl, difluoromethyl, trifluoromethyl, fluoro, chloro, cyano, methoxy, ethoxy, monofluoromethoxy, difluoromethoxy, trifluoromethoxy, 2,2-difluoroethoxy or 2,2,2-trifluoroethoxy, most preferably wherein Y is 1,2,3-triazolyl-N-oxide which is optionally substituted by one or two methyl groups. A group of especially preferred compounds of formula I comprises those wherein Y is 1,2,3-triazol-4-yl-1-N-oxide.

A group of especially preferred compounds of formula I comprises those wherein Y is 2,5-dimethyl-1,2,3-triazol-4-yl-1-N-oxide.

Furthermore, the present invention accordingly relates to compounds of formula I wherein

R¹ and R² are each independently of the other hydrogen, C₁-C₁₀alkyl, C₁-C₁₀haloalkyl, C₃-C₈cycloalkyl or C₃-C₈cycloalkyl-C₁-C₃alkyl, or
R¹ and R² together with the carbon atom to which they are bonded form a C₃-C₇ring,

R³ is halogen, azide, cyano, —SCN, C₂-C₁₀alkynyl, C₂-C₁₀alkenyl, formyl, C₁-C₁₀alkoxy, C₁-C₁₀alkylsulfanyl, C₁-C₁₀haloalkoxy, C₁-C₁₀haloalkylsulfanyl,

R⁴ is hydrogen, C₁-C₁₀alkyl, C₁-C₁₀haloalkyl, C₃-C₈cycloalkyl-C₁-C₁₀alkyl, C₁-C₆alkoxy-C₁-C₁₀alkyl or C₃-C₈cycloalkyl, halogen, azide, cyano, —SCN, C₂-C₁₀alkynyl, C₂-C₁₀alkenyl, formyl, C₁-C₁₀alkoxy, C₁-C₁₀alkylsulfanyl, C₁-C₁₀haloalkoxy, C₁-C₁₀haloalkylsulfanyl,
or
R² with R⁴ and together with the carbon atoms to which they are bonded form a C₃-C₈ring;
R⁵ and R⁶ are each independently of the other hydrogen, cyano, C₁-C₆alkyl, C₁-C₆alkoxycarbonyl, halogen or C₁-C₆haloalkyl;
m is 0, 1 or 2;
n is 1, 2 or 3;
Y is phenyl, naphthyl or tetrahydronaphthyl, which is optionally substituted by one to three substituents independently selected from C₁-C₆alkyl, C₃-C₆cycloalkyl, C₁-C₆halo-alkyl, C₁-C₆hydroxyalkyl, C₁-C₆alkoxy-C₁-C₆alkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl, nitro, cyano, formyl, carboxyl, halogen, azido, thiocyanato, tri(C₁-C₆alkyl)silyl, mercapto, phenylthio, phenylsulfinyl, —SF₅, C₁-C₆alkylthio, C₁-C₆haloalkyl-thio, C₁-C₆haloalkylsulfinyl, C₁-C₆haloalkylsulfonyl, C₁-C₆alkylsulfinyl, C₁-C₆alkyl-sulfonyl, benzylsulfonyl or benzylsulfonyl substituted by one to three R⁹, phenylsulfonyl or phenylsulfonyl substituted by one to three R⁹, hydroxyl, C₁-C₆alkoxy, C₃-C₆cyclo-alkyloxy wherein one of the CH₂ groups is optionally replaced by an oxygen atom, C₁-C₆haloalkoxy, C₂-C₆alkenyloxy, C₂-C₆alkynyloxy, C₁-C₆alkylsulfonyloxy, C₁-C₆halo-alkylsulfonyloxy, phenoxy or phenoxy substituted by one to three R⁹, benzyloxy or benzyloxy substituted by one to three R⁹, —CONH—SO₂—C₁-C₆alkyl, —CONH—SO₂—C₁-C₆haloalkyl, —NH—SO₂—C₁-C₆alkyl, —NH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —OCO—C₁-C₆alkyl, —OCO—C₁-C₆haloalkyl, —OCO-phenyl or —OCO-phenyl substituted by one to three R⁹, —OCONH—C₁-C₆alkyl, —OCONH—C₁-C₆haloalkyl, —OCONH-phenyl or —OCONH-phenyl substituted by one to three R⁹, or by one of the following groups Z, with Z=

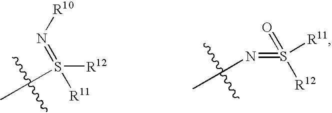

R¹⁰ is hydrogen, formyl, C₁-C₁₀alkyl, C₁-C₁₀haloalkyl, C₁-C₁₀alkylcarbonyl, C₁-C₁₀haloalkylcarbonyl, C₁-C₁₀alkoxycarbonyl, and
R¹¹ and R¹² are independently of each other C₁-C₁₀alkyl, C₁-C₁₀haloalkyl, C₁-C₁₀cycloalkyl, C₁-C₁₀cycloalkylalkyl, C₁-C₁₀alkoxyalkyl, or by —CONR⁷R⁸ wherein R⁷ and R⁸ are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R⁷ and R⁵ form a C₃-C₈alkylene group which optionally contains one oxygen or sulfur atom or one to two amino or C₁-C₆alkylamino groups, or
Y is a 5- to 10-membered aromatic or non-aromatic heterocycle containing one to three nitrogen, oxygen or sulfur atoms, which is optionally benzo-fused, and which is optionally substituted by one to three substituents independently selected from C₁-C₆alkyl, C₃-C₆cycloalkyl, C₁-C₆haloalkyl, C₁-C₆hydroxyalkyl, C₁-C₆alkoxy-C₁-C₆alkyl, C₂-C₆alkenyl, C₂-C₆alkynyl, C₂-C₆haloalkenyl, C₁-C₆alkylcarbonyl, C₁-C₆halo-alkylcarbonyl, C₁-C₆alkoxycarbonyl, nitro, cyano, formyl, carboxyl, halogen, azido, thiocyanato, tri(C₁-C₆alkyl)silyl, mercapto, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to three R⁹, phenylsulfonyl or phenylsulfonyl substituted by one to three R⁹, hydroxyl, C₁-C₆alkoxy, C₃-C₆cycloalkyloxy wherein one of the CH₂ groups is optionally replaced by an oxygen atom, C₁-C₆haloalkoxy, C₂-C₆alkenyloxy, C₂-C₆alkynyloxy, C₁-C₆alkylsulfonyloxy, C₁-C₆haloalkylsulfonyloxy, phenoxy or phenoxy substituted by one to three R⁹, benzyloxy or benzyloxy substituted by one to three R⁹, —CONH—SO₂—C₁-C₆alkyl, —CONH—SO₂—C₁-C₆haloalkyl, —NH—SO₂—C₁-C₆alkyl, —NH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —OCO—C₁-C₆alkyl, —OCO—C₁-C₆haloalkyl, —OCO-phenyl or —OCO-phenyl substituted by one to three R⁹, —OCONH—C₁-C₆alkyl, —OCONH—C₁-C₆haloalkyl, —OCONH-phenyl or —OCONH-phenyl substituted by one to three R⁹, or by one of the following groups Z, with Z=

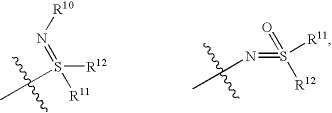

R¹⁰ is hydrogen, formyl, C₁-C₁₀alkyl, C₁-C₁₀haloalkyl, C₁-C₁₀alkylcarbonyl, C₁-C₁₀haloalkylcarbonyl, C₁-C₁₀alkoxycarbonyl, and
R¹¹ and R¹² are independently of each other C₁-C₁₀alkyl, C₁-C₁₀haloalkyl, C₁-C₁₀cycloalkyl, C₁-C₁₀cycloalkylalkyl, C₁-C₁₀alkoxyalkyl, or by —CONR⁷R⁸ wherein R⁷ and R⁸ are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R⁷ and R⁵ together form a C₃-C₈alkylene group which optionally contains one oxygen or sulfur atom or one to two amino or C₁-C₆alkylamino groups;
R⁹ are independently from each other C₁-C₆haloalkyl, C₁-C₆alkoxycarbonyl, nitro, cyano, formyl, carboxyl or halogen;
and to N-oxides, salts and optical isomers of compounds of formula I.

The compounds of the invention may contain one or more asymmetric carbon atoms, for example, in the —CR⁵R⁶-group or in the —CR³R⁴-group and may exist as enantiomers (or as pairs of diastereoisomers) or as mixtures of such. Further, when m is 1, the compounds of the invention are sulfoxides, which can exists in two enantiomeric forms, the adjacent carbon can also exists in two enantiomeric forms and the —CR³R⁴-group can also exist in two enantiomeric forms. Compounds of general formula I can therefore exist as racemates, diastereoisomers, or single enantiomers, and the invention includes all possible isomers or isomer mixtures in all proportions. It is to be expected that for any given compound, one isomer may be more herbicidal than another.

Alkyl groups, haloalkyl groups, hydroxyalkyl groups, alkoxy groups and alkylene groups can be straight or branched chain. Preferred alkyl groups, haloalkyl groups and hydroxyalkyl groups each independently contain 1 to 4 carbons. Examples of alkyl groups are methyl, ethyl, n- and iso-propyl and n-, sec-, iso- and tert-butyl, hexyl, nonyl and decyl. Examples of haloalkyl groups are difluoromethyl and 2,2,2-trifluoroethyl. Examples of hydroxyalkyl groups are 1,2-dihydroxyethyl and 3-hydroxypropyl. Examples of alkoxy groups are methoxy, ethoxy, propoxy, butoxy, hexyloxy, nonyloxy and decyloxy. Examples of alkylene groups are methylene, ethylene, n- and iso-propylene and n-, sec-, iso- and tert-butylene.

Examples of cycloalkyl groups are cyclopropyl, cyclobutyl, cyclopentyl, cyclohexyl, cycloheptyl and cyclooctyl. The cycloalkyl groups can be in bi- or tri-cyclic form.

Alkenyl and alkynyl groups and haloalkenyl groups and haloalkynyl groups can be straight or branched chain. Examples of alkenyl and alkynyl groups are allyl, but-2-enyl, 3-methylbut-2-enyl, ethynyl, propargyl and but-2-ynyl. Examples of haloalkenyl and haloalkynyl groups are trifluoroallyl and 1-chloroprop-1-yn-3-yl.

Halogen means fluoro, chloro, bromo and iodo, preferably fluoro, chloro or bromo, more preferably fluoro or chloro.

Referring to Y, examples of heterocycles are furyl, thienyl, pyrrolyl, pyrazolyl, imidazolyl, 1,2,3-triazolyl, 1,2,4-triazolyl, tetrazolyl, oxazolyl, isoxazolyl, thiazolyl, isothiazolyl, 1,2,3-oxadiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, 1,3,5-triazinyl, 1,2,3,4-tetrazinyl, 1,2,3,5-tetrazinyl, 1,2,4,5-tetrazinyl, benzofuryl, isobenzofuryl, benzothiophenyl, isobenzothiophenyl, indolyl, isoindolyl, indazolyl, benzimidazolyl, benztriazolyl, benzoxazolyl, 1,2-benzisoxazolyl, 2,1-benzisoxazolyl, benzothiazolyl, 1,2-benzisothiazolyl, 2,1-benzisothiazolyl, 1,2,3-benzoxadiazolyl, 2,1,3-benzoxadiazolyl, 1,2,3-benzothiadiazolyl, 2,1,3-benzothiadiazolyl, quinolyl, isoquinolyl, cinnolinyl, phthalazinyl, quinazolinyl, quinoxalinyl, naphthyridinyl, benzotriazinyl, purinyl, pteridinyl, indolizinyl, benzo-1,3-dioxolyl, 4H-benzo-1,3-dioxinyl, and 4H-benzo-1,4-dioxinyl groups, tetrahydrofuranyl, tetrahydropyranyl, 1,3-dioxolanyl, 1,3-dioxanyl, 1,4-dioxanyl, and morpholinyl groups, dihydro-1,3-dioxolyl, dihydroisoxazolyl, benziso-furyl, benzothienyl, benzisothienyl and indolyl group and, where appropriate, N-oxides and salts thereof.

The invention relates likewise to the salts which the compounds of formula I are able to form with amines, alkali metal and alkaline earth metal bases and quarternary ammonium bases.

Among the alkali metal and alkaline earth metal hydroxides as salt formers, special mention should be made of the hydroxides of lithium, sodium, potassium, magnesium and calcium, but especially the hydroxides of sodium and potassium. The compounds of formula I according to the invention also include hydrates which may be formed during the salt formation.

Examples of amines suitable for ammonium salt formation include ammonia as well as primary, secondary and tertiary C₁-C₁₈alkylamines, C₁-C₄hydroxyalkylamines and C₂-C₄alkoxyalkylamines, for example methylamine, ethylamine, n-propylamine, isopropylamine, the four butylamine isomers, n-amylamine, isoamylamine, hexylamine, heptylamine, octylamine, nonylamine, decylamine, pentadecylamine, hexadecylamine, heptadecylamine, octadecylamine, methylethylamine, methylisopropylamine, methylhexylamine, methylnonylamine, methylpentadecylamine, methyloctadecylamine, ethylbutylamine, ethylheptylamine, ethyloctylamine, hexylheptylamine, hexyloctylamine, dimethylamine, diethylamine, di-n-propylamine, diisopropylamine, di-n-butylamine, di-n-amylamine, diisoamylamine, dihexylamine, diheptylamine, dioctylamine, ethanolamine, n-propanolamine, isopropanolamine, N,N-diethanolamine, N-ethylpropanolamine, N-butylethanolamine, allylamine, n-butenyl-2-amine, n-pentenyl-2-amine, 2,3-dimethylbutenyl-2-amine, dibutenyl-2-amine, n-hexenyl-2-amine, propylenediamine, trimethylamine, triethylamine, tri-n-propylamine, triisopropylamine, tri-n-butylamine, triisobutylamine, tri-sec-butylamine, tri-n-amylamine, methoxyethylamine and ethoxyethylamine; heterocyclic amines such as, for example, pyridine, quinoline, isoquinoline, morpholine, piperidine, pyrrolidine, indoline, quinuclidine and azepine; primary arylamines such as, for example, anilines, methoxyanilines, ethoxyanilines, o-, m- and p-toluidines, phenylenediamines, benzidines, naphthylamines and o-, m- and p-chloroanilines; but especially triethylamine, isopropylamine and diisopropylamine.

Preferred quarternary ammonium bases suitable for salt formation correspond, for example, to the formula [N(RₐR_{b}R_{c}R_{d})]OH wherein Rₐ, R_{b}, R_{c} and R_{d} are each independently of the others C₁-C₄alkyl. Other suitable tetraalkylammonium bases with other anions can be obtained, for example, by anion exchange reactions.

The term “herbicide” as used herein means a compound that controls or modifies the growth of plants. The term “herbicidally effective amount” means the quantity of such a compound or combination of such compounds that is capable of producing a controlling or modifying effect on the growth of plants. Controlling or modifying effects include all deviation from natural development, for example: killing, retardation, leaf burn, albinism, dwarfing and the like. The term “plants” refers to all physical parts of a plant, including seeds, seedlings, saplings, roots, tubers, stems, stalks, foliage, and fruits. The term “locus” is intended to include soil, seeds, and seedlings, as well as established vegetation.

TABLE 1
Compounds of formula I.1.

I.1

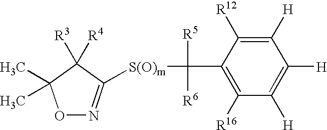

| R³ | R⁴ | m | R⁵ | R⁶ | R¹² | R¹⁶ |
|---|---|---|---|---|---|---|
| N₃ | H | 2 | H | CO₂Me | F | F |
| Cl | H | 1 | H | Cl | F | F |
| F | H | 0 | H | F | F | F |
| Cl | F | 0 | F | Cl | F | F |
| N₃ | H | 1 | H | F | F | F |
| CN | H | 0 | F | F | F | F |
| CN | H | 1 | H | H | F | F |
| Cl | F | 1 | H | Cl | F | F |
| F | F | 0 | H | CO₂Me | F | F |
| F | F | 2 | H | Cl | F | F |
| F | F | 2 | H | CO₂Me | F | F |
| F | H | 2 | H | CO₂Me | F | F |
| N₃ | H | 1 | F | F | F | F |
| Cl | H | 2 | H | Me | F | F |
| F | H | 2 | F | F | F | F |
| Cl | H | 0 | H | Me | F | F |
| Cl | H | 2 | H | F | F | F |
| Cl | F | 2 | H | CO₂Me | F | F |
| Cl | F | 1 | H | H | F | F |
| F | F | 2 | F | Cl | F | F |
| Cl | H | 0 | H | F | F | F |
| Cl | F | 2 | H | Me | F | F |
| F | F | 0 | H | H | F | F |
| N₃ | H | 1 | H | Me | F | F |
| CN | H | 1 | H | CO₂Me | F | F |
| Cl | F | 0 | H | CO₂Me | F | F |
| N₃ | H | 2 | F | Cl | F | F |
| CN | H | 2 | H | H | F | F |
| Cl | F | 2 | F | Cl | F | F |
| N₃ | H | 1 | F | Cl | F | F |
| Cl | F | 2 | H | Cl | F | F |
| Cl | H | 2 | F | F | F | F |
| F | H | 2 | H | Cl | F | F |
| Cl | H | 0 | H | H | F | F |
| Cl | H | 1 | H | Me | F | F |
| Cl | H | 0 | H | Cl | F | F |
| Cl | H | 1 | H | H | F | F |
| Cl | H | 1 | F | F | F | F |
| CN | H | 1 | H | Cl | F | F |
| Cl | F | 1 | H | CO₂Me | F | F |
| N₃ | H | 0 | H | H | F | F |
| F | F | 2 | H | H | F | F |
| F | F | 0 | H | Me | F | F |
| N₃ | H | 2 | H | F | F | F |
| Cl | H | 1 | H | F | F | F |
| F | F | 0 | F | F | F | F |
| Cl | F | 0 | H | H | F | F |
| Cl | H | 2 | H | H | F | F |
| F | H | 1 | F | F | F | F |
| F | H | 0 | H | Me | F | F |
| Cl | F | 2 | H | F | F | F |
| CN | H | 1 | F | Cl | F | F |
| Cl | F | 1 | H | Me | F | F |
| CN | H | 2 | F | F | F | F |
| F | F | 0 | H | F | F | F |
| Cl | F | 1 | F | F | F | F |
| F | F | 1 | F | F | F | F |
| N₃ | H | 0 | H | Cl | F | F |
| CN | H | 0 | H | F | F | F |
| CN | H | 1 | H | F | F | F |
| F | F | 1 | H | Cl | F | F |
| Cl | H | 1 | H | CO₂Me | F | F |
| F | H | 1 | F | Cl | F | F |
| Cl | H | 2 | F | Cl | F | F |
| CN | H | 0 | H | Cl | F | F |
| F | H | 2 | F | Cl | F | F |
| CN | H | 1 | H | Me | F | F |
| Cl | F | 2 | H | H | F | F |
| CN | H | 2 | F | Cl | F | F |
| CN | H | 2 | H | F | F | F |
| Cl | H | 0 | F | F | F | F |
| F | F | 1 | H | Me | F | F |
| CN | H | 2 | H | CO₂Me | F | F |
| CN | H | 1 | F | F | F | F |
| F | F | 2 | F | F | F | F |
| N₃ | H | 2 | F | F | F | F |
| N₃ | H | 1 | H | H | F | F |
| CN | H | 0 | H | Me | F | F |
| N₃ | H | 2 | H | Cl | F | F |
| Cl | F | 0 | H | Me | F | F |
| F | F | 1 | H | F | F | F |
| F | F | 1 | H | H | F | F |
| F | H | 0 | H | H | F | F |
| Cl | F | 0 | H | F | F | F |
| N₃ | H | 0 | H | Me | F | F |
| Cl | H | 0 | H | CO₂Me | F | F |
| N₃ | H | 2 | H | H | F | F |
| CN | H | 2 | H | Cl | F | F |
| Cl | H | 2 | H | Cl | F | F |
| F | H | 0 | H | Cl | F | F |
| F | F | 1 | F | Cl | F | F |
| F | H | 2 | H | F | F | F |
| F | F | 0 | F | Cl | F | F |
| Cl | F | 1 | F | Cl | F | F |
| Cl | H | 1 | F | Cl | F | F |
| CN | H | 0 | H | CO₂Me | F | F |
| F | H | 1 | H | Me | F | F |
| F | F | 2 | H | F | F | F |
| F | H | 1 | H | F | F | F |
| Cl | H | 0 | F | Cl | F | F |
| Cl | F | 0 | H | Cl | F | F |
| F | H | 2 | H | H | F | F |
| N₃ | H | 0 | H | F | F | F |
| N₃ | H | 1 | H | Cl | F | F |
| N₃ | H | 2 | H | Me | F | F |
| F | H | 1 | H | H | F | F |
| Cl | F | 0 | F | F | F | F |
| F | F | 0 | H | Cl | F | F |
| Cl | F | 1 | H | F | F | F |
| N₃ | H | 0 | F | F | F | F |
| F | H | 2 | H | Me | F | F |
| F | H | 0 | H | CO₂Me | F | F |
| CN | H | 0 | H | H | F | F |
| F | F | 1 | H | CO₂Me | F | F |
| F | H | 0 | F | F | F | F |
| N₃ | H | 1 | H | CO₂Me | F | F |
| N₃ | H | 0 | H | CO₂Me | F | F |
| F | H | 0 | F | Cl | F | F |
| F | H | 1 | H | CO₂Me | F | F |
| N₃ | H | 0 | F | Cl | F | F |
| CN | H | 2 | H | Me | F | F |
| CN | H | 0 | F | Cl | F | F |
| Cl | F | 2 | F | F | F | F |
| F | H | 1 | H | Cl | F | F |
| Cl | H | 2 | H | CO₂Me | F | F |
| F | F | 2 | H | Me | F | F |

### Table 2:

Table 2 consists of 126 compounds of the general formula I.1, where R¹² is trifluoromethoxy, R¹⁶ is hydrogen, and R³, R⁴, m, R⁵ and R⁶ have the values listed in Table 1. Thus compound 1 of Table 2 is the same as compound 1 of Table 1 except that in compound 1 of Table 2 R¹² is trifluoromethoxy instead of fluoro and R¹⁶ is hydrogen instead of fluoro. Similarly, compounds 2 to 126 of Table 2 are the same as compounds 2 to 126 of Table 1, respectively, except that in the compounds of Table 2 R¹² is trifluoromethoxy instead of fluoro and R¹⁶ is hydrogen instead of fluoro.

### Table 3:

Table 3 consists of 126 compounds of the general formula I.1, where R¹² is difluoromethoxy, R¹⁶ is hydrogen, and R³, R⁴, m, R⁵ and R⁶ have the values listed in Table 1. Thus compound 1 of Table 3 is the same as compound 1 of Table 1 except that in compound 1 of Table 3 R¹² is difluoromethoxy instead of fluoro and R¹⁶ is hydrogen instead of fluoro. Similarly, compounds 2 to 126 of Table 3 are the same as compounds 2 to 126 of Table 1, respectively, except that in the compounds of Table 3 R¹² is difluoromethoxy instead of fluoro and R¹⁶ is hydrogen instead of fluoro.

### Table 4:

Table 4 consists of 126 compounds of the general formula I.1, where R¹² is chloro and R³, R⁴, m, R⁵, R⁶ and R¹⁶ have the values listed in Table 1. Thus compound 1 of Table 4 is the same as compound 1 of Table 1 except that in compound 1 of Table 4 R¹² is chloro instead of fluoro. Similarly, compounds 2 to 126 of Table 4 are the same as compounds 2 to 126 of Table 1, respectively, except that in the compounds of Table 4 R¹² is chloro instead of fluoro.

### Table 5:

Table 5 consists of 126 compounds of the general formula I.2,

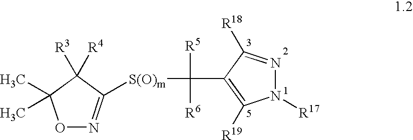

where R³, R⁴, m, R⁵ and R⁶ have the values listed in Table 1, R¹⁷ is Me, R¹⁸ is —CF₃ and R¹⁹ is —OCHF₂. Thus compound 1 of Table 5 is the same as compound 1 of Table 1 except that in compound 1 of Table 5 Y is 1-methyl-5-difluoromethoxy-3-trifluoromethyl-pyrazol-4-yl instead of 2,6-difluorophenyl. Similarly, compounds 2 to 126 of Table 5 are the same as compounds 2 to 126 of Table 1, respectively, except that in the compounds of Table 5 Y is 1-methyl-5-difluoromethoxy-3-trifluoromethyl-pyrazol-4-yl instead of 2,6-difluorophenyl.

### Table 6:

Table 6 consists of 126 compounds of the general formula I.2, where R¹⁹ is —OCH₂CF₃ and R³, R⁴, m, R⁵, R⁶, R¹⁷ and R¹⁸ have the values listed in Table 5. Thus compound 1 of Table 6 is the same as compound 1 of Table 5 except that in compound 1 of Table 6 R¹⁹ is —OCH₂CF₃ instead of —OCHF₂. Similarly, compounds 2 to 126 of Table 6 are the same as compounds 2 to 126 of Table 5, respectively, except that in the compounds of Table 6 R¹⁹ is —OCH₂CF₃ instead of —OCHF₂.

### Table 7:

Table 7 consists of 126 compounds of the general formula I.2, where R¹⁹ is —OCH₂CHF₂ and R³, R⁴, m, R⁵, R⁶, R¹⁷ and R¹⁸ have the values listed in Table 5. Thus compound 1 of Table 7 is the same as compound 1 of Table 5 except that in compound 1 of Table 7 R¹⁹ is —OCH₂CHF₂ instead of —OCHF₂. Similarly, compounds 2 to 126 of Table 7 are the same as compounds 2 to 126 of Table 5, respectively, except that in the compounds of Table 7 R¹⁹ is —OCH₂CHF₂ instead of —OCHF₂.

### Table 8:

Table 8 consists of 126 compounds of the general formula I.2, where R¹⁸ is —CHF₂ and R³, R⁴, m, R⁵, R⁶, R¹⁷ and R¹⁹ have the values listed in Table 5. Thus compound 1 of Table 8 is the same as compound 1 of Table 5 except that in compound 1 of Table 8 R¹⁸ is —CHF₂ instead of —CF₃. Similarly, compounds 2 to 126 of Table 8 are the same as compounds 2 to 126 of Table 5, respectively, except that in the compounds of Table 8 R¹⁸ is —CHF₂ instead of —CF₃.

### Table 9:

Table 9 consists of 126 compounds of the general formula I.2, where R¹⁸ is —CHF₂, R¹⁹ is —OCH₂CF₃ and R³, R⁴, m, R⁵, R⁶ and R¹⁷ have the values listed in Table 5. Thus compound 1 of Table 9 is the same as compound 1 of Table 5 except that in compound 1 of Table 9 R¹⁸ is —CHF₂ instead of —CF₃ and R¹⁹ is —OCH₂CF₃ instead of —OCHF₂. Similarly, compounds 2 to 126 of Table 9 are the same as compounds 2 to 126 of Table 5, respectively, except that in the compounds of Table 9 R¹⁸ is —CHF₂ instead of —CF₃ and R¹⁹ is —OCH₂CF₃ instead of —OCHF₂.

### Table 10:

Table 10 consists of 126 compounds of the general formula I.2, where R¹⁸ is —CHF₂, R¹⁹ is —OCH₂CHF₂ and R³, R⁴, m, R⁵, R⁶ and R¹⁷ have the values listed in Table 5. Thus compound 1 of Table 10 is the same as compound 1 of Table 5 except that in compound 1 of Table 10 R¹⁸ is —CHF₂ instead of —CF₃ and R¹⁹ is —OCH₂CHF₂ instead of —OCHF₂. Similarly, compounds 2 to 126 of Table 10 are the same as compounds 2 to 126 of Table 5, respectively, except that in the compounds of Table 10 R¹⁸ is —CHF₂ instead of —CF₃ and R¹⁹ is —OCH₂CHF₂ instead of —OCHF₂.

### Table 11:

Table 1 consists of 126 compounds of the general formula I.2, where R¹⁸ is —OCHF₂, R¹⁹ is —CF₃ and R³, R⁴, m, R⁵, R⁶ and R¹⁷ have the values listed in Table 5. Thus compound 1 of Table 11 is the same as compound 1 of Table 5 except that in compound 1 of Table 11 R¹⁸ is —OCHF₂ instead of —CF₃ and R¹⁹ is —CF₃ instead of —OCHF₂. Similarly, compounds 2 to 126 of Table 11 are the same as compounds 2 to 126 of Table 5, respectively, except that in the compounds of Table 11 R¹⁸ is —OCHF₂ instead of —CF₃ and R¹⁹ is —CF₃ instead of —OCHF₂.

### Table 12:

Table 12 consists of 126 compounds of the general formula I.2, where R¹⁸ is —OCH₂CF₃. R¹⁹ is —CF₃ and R³, R⁴, m, R⁵, R⁶ and R¹⁷ have the values listed in Table 5. Thus compound 1 of Table 12 is the same as compound 1 of Table 5 except that in compound 1 of Table 12 R¹⁸ is —OCH₂CF₃ instead of —CF₃ and R¹⁹ is —CF₃ instead of —OCHF₂. Similarly, compounds 2 to 126 of Table 12 are the same as compounds 2 to 126 of Table 5, respectively, except that in the compounds of Table 12 R¹⁸ is —OCH₂CF₃ instead of —CF₃ and R¹⁹ is —CF₃ instead of —OCHF₂.

### Table 13:

Table 13 consists of 126 compounds of the general formula I.2, where R¹⁸ is —OCH₂CHF₂, R¹⁹ is —CF₃ and R³, R⁴, m, R⁵, R⁶ and R¹⁷ have the values listed in Table 5. Thus compound 1 of Table 13 is the same as compound 1 of Table 5 except that in compound 1 of Table 13 R¹⁸ is —OCH₂CHF₂ instead of —CF₃ and R¹⁹ is —CF₃ instead of —OCHF₂. Similarly, compounds 2 to 126 of Table 13 are the same as compounds 2 to 126 of Table 5, respectively, except that in the compounds of Table 13 R¹⁸ is —OCH₂CHF₂ instead of —CF₃ and R¹⁹ is —CF₃ instead of —OCHF₂.

### Table 14:

Table 14 consists of 126 compounds of the general formula I.2, where R¹⁸ is —OCHF₂, R¹⁹ is —CHF₂ and R³, R⁴, m, R⁵, R⁶ and R¹⁷ have the values listed in Table 5. Thus compound 1 of Table 14 is the same as compound 1 of Table 5 except that in compound 1 of Table 14 R¹⁸ is —OCHF₂ instead of —CF₃ and R¹⁹ is —CHF₂ instead of —OCHF₂. Similarly, compounds 2 to 126 of Table 14 are the same as compounds 2 to 126 of Table 5, respectively, except that in the compounds of Table 14 R¹⁸ is —OCHF₂ instead of —CF₃ and R¹⁹ is —CHF₂ instead of —OCHF₂.

### Table 15:

Table 15 consists of 126 compounds of the general formula I.2, where R¹⁸ is —OCH₂CF₃, —R¹⁹ is —CHF₂ and R³, R⁴, m, R⁵, R⁶ and R¹⁷ have the values listed in Table 5. Thus compound 1 of Table 15 is the same as compound 1 of Table 5 except that in compound 1 of Table 15 R¹⁸ is —OCH₂CF₃ instead of —CF₃ and R¹⁹ is —CHF₂ instead of —OCHF₂. Similarly, compounds 2 to 126 of Table 15 are the same as compounds 2 to 126 of Table 5, respectively, except that in the compounds of Table 15 R¹⁸ is —OCH₂CF₃ instead of —CF₃ and R¹⁹ is —CHF₂ instead of —OCHF₂.

### Table 16:

Table 16 consists of 126 compounds of the general formula I.2, where R's is —OCH₂CHF₂, R¹⁹ is —CHF₂ and R³, R⁴, m, R⁵, R⁶ and R¹⁷ have the values listed in Table 5. Thus compound 1 of Table 16 is the same as compound 1 of Table 5 except that in compound 1 of Table 16 R¹⁸ is —OCH₂CHF₂ instead of —CF₃ and R¹⁹ is —CHF₂ instead of —OCHF₂. Similarly, compounds 2 to 126 of Table 16 are the same as compounds 2 to 126 of Table 5, respectively, except that in the compounds of Table 16 R¹⁸ is —OCH₂CHF₂ instead of —CF₃ and R¹⁹ is —CHF₂ instead of —OCHF₂.

### Table 17:

Table 17 consists of 126 compounds of the general formula I.2, where R¹⁷ is ethyl and R³, R⁴, m, R⁵, R⁶, R¹⁸ and R¹⁹ have the values listed in Table 5. Thus compound 1 of Table 17 is the same as compound 1 of Table 5 except that in compound 1 of Table 17 R¹⁷ is ethyl instead of methyl. Similarly, compounds 2 to 126 of Table 17 are the same as compounds 2 to 126 of Table 5, respectively, except that in the compounds of Table 17 R¹⁷ is ethyl instead of methyl.

### Table 18:

Table 1S consists of 126 compounds of the general formula I.2, where R¹⁷ is ethyl and R³, R⁴, m, R⁵, R⁶, R¹⁸ and R¹⁹ have the values listed in Table 6. Thus compound 1 of Table 18 is the same as compound 1 of Table 6 except that in compound 1 of Table 18 R¹⁷ is ethyl instead of methyl. Similarly, compounds 2 to 126 of Table 18 are the same as compounds 2 to 126 of Table 6, respectively, except that in the compounds of Table 18 R¹⁷ is ethyl instead of methyl.

### Table 19:

Table 19 consists of 126 compounds of the general formula I.2, where R¹⁷ is ethyl and R³, R⁴, m, R⁵, R⁶, R¹⁸ and R¹⁹ have the values listed in Table 7. Thus compound 1 of Table 19 is the same as compound 1 of Table 7 except that in compound 1 of Table 19 R¹⁷ is ethyl instead of methyl. Similarly, compounds 2 to 126 of Table 19 are the same as compounds 2 to 126 of Table 7, respectively, except that in the compounds of Table 19 R¹⁷ is ethyl instead of methyl.

### Table 20:

Table 20 consists of 126 compounds of the general formula I.2, where R¹⁷ is ethyl and R³, R⁴, m, R⁵, R⁶, R¹⁸ and R¹⁹ have the values listed in Table 11. Thus compound 1 of Table 20 is the same as compound 1 of Table 11 except that in compound 1 of Table 20 R¹⁷ is ethyl instead of methyl. Similarly, compounds 2 to 126 of Table 20 are the same as compounds 2 to 126 of Table 11, respectively, except that in the compounds of Table 20 R¹⁷ is ethyl instead of methyl.

### Table 21:

Table 21 consists of 126 compounds of the general formula I.2, where R¹⁷ is ethyl and R³, R⁴, m, R⁵, R⁶, R¹⁸ and R¹⁹ have the values listed in Table 12. Thus compound 1 of Table 21 is the same as compound 1 of Table 12 except that in compound 1 of Table 21 R⁷ is ethyl instead of methyl. Similarly, compounds 2 to 126 of Table 21 are the same as compounds 2 to 126 of Table 12, respectively, except that in the compounds of Table 21 R¹⁷ is ethyl instead of methyl.

### Table 22:

Table 22 consists of 126 compounds of the general formula I.2, where R¹⁷ is ethyl and R³, R⁴, m, R⁵, R⁶, R¹⁵ and R¹⁹ have the values listed in Table 13. Thus compound 1 of Table 22 is the same as compound 1 of Table 13 except that in compound 1 of Table 22 R¹⁷ is ethyl instead of methyl. Similarly, compounds 2 to 126 of Table 22 are the same as compounds 2 to 126 of Table 13, respectively, except that in the compounds of Table 22 R¹⁷ is ethyl instead of methyl.

### Table 23:

Table 23 consists of 126 compounds of the general formula I.2, where R¹⁹ is hydrogen and R³, R⁴, m, R⁵, R⁶, R¹⁷ and R¹⁸ have the values listed in Table 5. Thus compound 1 of Table 23 is the same as compound 1 of Table 5 except that in compound 1 of Table 23 R¹⁹ is hydrogen instead of —OCHF₂. Similarly, compounds 2 to 126 of Table 23 are the same as compounds 2 to 126 of Table 5, respectively, except that in the compounds of Table 23 R¹⁹ is hydrogen instead of —OCHF₂.

### Table 24:

Table 24 consists of 126 compounds of the general formula I.2, where R¹⁷ is ethyl and R³, R⁴, m, R⁵, R⁶, R¹⁸ and R¹⁹ have the values listed in Table 23. Thus compound 1 of Table 24 is the same as compound 1 of Table 23 except that in compound 1 of Table 24 R¹⁷ is ethyl instead of methyl. Similarly, compounds 2 to 126 of Table 24 are the same as compounds 2 to 126 of Table 23, respectively, except that in the compounds of Table 24 R¹⁷ is ethyl instead of methyl.

### Table 25:

Table 25 consists of 126 compounds of the general formula I.2, where R¹⁸ is —CHF₂ and R³, R⁴, m, R⁵, R⁶, R¹⁷ and R¹⁹ have the values listed in Table 23. Thus compound 1 of Table 25 is the same as compound 1 of Table 23 except that in compound 1 of Table 25 μg is —CHF₂ instead of —CF₃. Similarly, compounds 2 to 126 of Table 25 are the same as compounds 2 to 126 of Table 23, respectively, except that in the compounds of Table 25 R¹⁸ is —CHF₂ instead of —CF₃.

### Table 26:

Table 26 consists of 126 compounds of the general formula I.2, where R¹⁷ is ethyl and R³, R⁴, m, R⁵, R⁶, R¹⁸ and R¹⁹ have the values listed in Table 25. Thus compound 1 of Table 26 is the same as compound 1 of Table 25 except that in compound 1 of Table 26 R¹⁷ is ethyl instead of methyl. Similarly, compounds 2 to 126 of Table 26 are the same as compounds 2 to 126 of Table 25, respectively, except that in the compounds of Table 26 R¹⁷ is ethyl instead of methyl.

### Table 27:

Table 27 consists of 126 compounds of the general formula I.2, where R¹⁹ is hydrogen and R³, R⁴, m, R⁵, R⁶, R¹⁷ and R¹⁸ have the values listed in Table 12. Thus compound 1 of Table 27 is the same as compound 1 of Table 12 except that in compound 1 of Table 27 R¹⁹ is hydrogen instead of —CF₃. Similarly, compounds 2 to 126 of Table 27 are the same as compounds 2 to 126 of Table 12, respectively, except that in the compounds of Table 27 R¹⁹ is hydrogen instead of —CF₃.

### Table 28:

Table 28 consists of 126 compounds of the general formula I.2, where R¹⁷ is ethyl and R³, R⁴, m, R⁵, R⁶, R¹⁸ and R¹⁹ have the values listed in Table 27. Thus compound 1 of Table 28 is the same as compound 1 of Table 27 except that in compound 1 of Table 28 R¹⁷ is ethyl instead of methyl. Similarly, compounds 2 to 126 of Table 28 are the same as compounds 2 to 126 of Table 27, respectively, except that in the compounds of Table 28 R¹⁷ is ethyl instead of methyl.

### Table 29:

Table 29 consists of 126 compounds of the general formula I.2, where R¹⁹ is hydrogen and R³, R⁴, m, R⁵, R⁶, R¹⁷ and R¹⁸ have the values listed in Table 11. Thus compound 1 of Table 29 is the same as compound 1 of Table 11 except that in compound 1 of Table 29 R¹⁹ is hydrogen instead of —CF₃. Similarly, compounds 2 to 126 of Table 29 are the same as compounds 2 to 126 of Table 11, respectively, except that in the compounds of Table 27 R¹⁹ is hydrogen instead of —CF₃.

### Table 30:

Table 30 consists of 126 compounds of the general formula I.2, where R¹⁷ is ethyl and R³, R⁴, m, R⁵, R⁶, R¹⁸ and R¹⁹ have the values listed in Table 29. Thus compound 1 of Table 30 is the same as compound 1 of Table 29 except that in compound 1 of Table 30 R¹⁷ is ethyl instead of methyl. Similarly, compounds 2 to 126 of Table 30 are the same as compounds 2 to 126 of Table 29, respectively, except that in the compounds of Table 26 R¹⁷ is ethyl instead of methyl.

### Table 31:

Table 31 consists of 126 compounds of the general formula I.7,

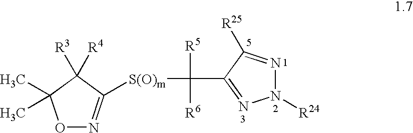

where R³, R⁴, m, R⁵ and R⁶ have the values listed in Table 5, R²⁴ is methyl and R²⁵ is —CF₃. Thus compound 1 of Table 31 is the same as compound 1 of Table 5 except that in compound 1 of Table 31 Y is 2-methyl-5-trifluoromethyl-1,2,3-triazol-4-yl instead of 1-methyl-5-difluoromethoxy-3-trifluoromethyl-pyrazol-4-yl. Similarly, compounds 2 to 126 of Table 31 are the same as compounds 2 to 126 of Table 5, respectively, except that in the compounds of Table 31 Y is 2-methyl-5-trifluoromethyl-1,2,3-triazol-4-yl instead of 1-methyl-5-difluoromethoxy-3-trifluoromethyl-pyrazol-4-yl.

### Table 32:

Table 32 consists of 126 compounds of the general formula I.7, where R³, R⁴, m, R⁵, R⁶, and R²⁴ have the values listed in Table 31 and R²⁵ is methyl. Thus compound 1 of Table 32 is the same as compound 1 of Table 31 except that in compound 1 of Table 32 R²⁵ is methyl instead of —CF₃. Similarly, compounds 2 to 126 of Table 32 are the same as compounds 2 to 126 of Table 31, respectively, except that in the compounds of Table 32 R²⁵ is methyl instead of —CF₃.

### Table 33:

Table 33 consists of 126 compounds of the general formula I.7, where R³, R⁴, m, R⁵, R⁶, and R²⁴ have the values listed in Table 31 and R²⁵ is ethyl. Thus compound 1 of Table 33 is the same as compound 1 of Table 31 except that in compound 1 of Table 33 R²⁵ is ethyl instead of —CF₃. Similarly, compounds 2 to 126 of Table 33 are the same as compounds 2 to 126 of Table 31, respectively, except that in the compounds of Table 33 R²⁵ is ethyl instead of —CF₃.

### Table 34:

Table 34 consists of 126 compounds of the general formula I.7, where R²⁴ is ethyl and R³, R⁴, m, R⁵, R⁶ and R²⁵ have the values listed in Table 31. Thus compound 1 of Table 34 is the same as compound 1 of Table 31 except that in compound 1 of Table 34 R²⁴ is ethyl instead of methyl. Similarly, compounds 2 to 126 of Table 34 are the same as compounds 2 to 126 of Table 21, respectively, except that in the compounds of Table 34 R²⁴ is ethyl instead of methyl.

### Table 35:

Table 35 consists of 126 compounds of the general formula I.7, where R²⁴ is ethyl and R³, R⁴, m, R⁵, R⁶ and R²⁵ have the values listed in Table 32. Thus compound 1 of Table 35 is the same as compound 1 of Table 32 except that in compound 1 of Table 35 R²⁴ is ethyl instead of methyl. Similarly, compounds 2 to 126 of Table 35 are the same as compounds 2 to 126 of Table 32, respectively, except that in the compounds of Table 35 R²⁴ is ethyl instead of methyl.

### Table 36:

Table 36 consists of 126 compounds of the general formula I.7, where R¹⁷ is ethyl and R³, R⁴, m, R⁵, R⁶ and R¹⁸ have the values listed in Table 33. Thus compound 1 of Table 36 is the same as compound 1 of Table 33 except that in compound 1 of Table 36 R¹⁷ is ethyl instead of methyl. Similarly, compounds 2 to 126 of Table 36 are the same as compounds 2 to 126 of Table 33, respectively, except that in the compounds of Table 36 R¹⁷ is ethyl instead of methyl.

### Methods of Halogenation, Alkylation and Oxidation

1) The compounds of formula I wherein R¹, R², R³, R⁴, R⁵, R⁶ and Y are as defined above, m is 1 or 2, and n is 1, can be prepared by processes known per se, by reacting e.g. the compounds of formula Ia

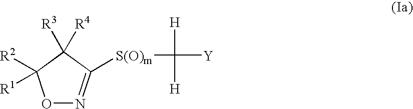

wherein R¹, R², R³, R⁴ and Y are as defined above, in a single step or stepwise in succession with compounds of the formula R⁵—X and/or R⁶—X, wherein R⁵ and R⁶ are as defined above and X is a suitable leaving group e.g. halide, such as bromide or iodide, a carboxylate, such as acetate, an alkylsulfonate, such as methylsulfonate, an arylsulfonate, such as p-toluenesulfonate, a haloalkylsulfonate, such as trifluoromethylsulfonate, an imide, such as succinimide, a sulfonimide, such as bis(phenylsulfonyl)imide, or an arylsulfinate, such as p-toluenesulfinate, in the presence of a base, e.g. an alkyl-lithium compound, such as methyl-lithium, n-butyl-lithium or tert-butyl-lithium, a lithium dialkylamide, such as lithium diisopropylamide, a metal hydride, preferably an alkali metal hydride, such as sodium hydride, or an alkali metal amide, such as sodium amide, a metal bis(tri(C₁-C₆alkyl)silyl)amide, such as lithium bis(trimethylsilyl)amide, a metal alkoxide, such as potassium tert-butoxide, or a phosphazene base, such as N′-tert-butyl-N,N,N′,N′,N″,N″-hexamethylphosphorimidic triamide (P₁-t-Bu), 1-tert-butyl-2,2,4,4,4-pentakis(dimethylamino)-2-lambda⁵,4lambda⁵-catenadi(phosphazene) (P₂-t-Bu), 1-ethyl-2,2,4,4,4-pentakis(dimethylamino)-2-lambda⁵4lambda⁵-catenadi(phosphazene) (P₂-Et) and 2-tert-butylimino-2-diethylamino-1,3-dimethyl-perhydro-1,3,2-diazaphosphorine (BEMP), optionally in the presence of a diluent, preferably an inert solvent, e.g. a hydrocarbon, an ether, such as tetrahydrofuran, an amide, such as N,N-dimethylformamide, or a halogenated hydrocarbon, such as dichloromethane, or mixtures thereof and optionally in the presence of a complexing agent, such as hexamethylphosphoramide or tetramethylethylenediamine in a temperature range of from −120° C. to 100° C., preferably from −80° C. to 50° C. Such processes are known in the literature and are described, for example, in J. Med. Chem., 2003 (46) 3021-3032; J. Org. Chem., 2003 (68) 1443-1446; J. Org. Chem., 2002 (67) 5216-5225, J. Org. Chem., 2002 (67) 3065-3071 and Heterocycles 2003 (59) 161-167.

2) The compounds of formula I wherein R¹, R², R³, R⁴, R⁵, R⁶ and Y are as defined above, m is 1 or 2, and n is 1, can be prepared by processes known per se, by reacting e.g. the compounds of formula Ib

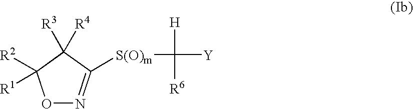

wherein R¹, R², R³, R⁴, R⁶ and Y are as defined above, with compounds of the formula R⁵—X, wherein R⁵ is as defined above and X is a suitable leaving group as defined in 1), in the presence of a base as defined in 1), optionally in the presence of a diluent as defined in 1), preferably an inert solvent, and optionally in the presence of a complexing agent as defined in 1) in a temperature range of from −120° C. to 100° C., preferably from −80° C. to 50° C.

3) The compounds of formula I wherein R¹, R², R³, R⁴, R⁵, R⁶ and Y are as defined above, m is 1 or 2, and n is 1, can be prepared by processes known per se, by reacting e.g. the compounds of formula Ic

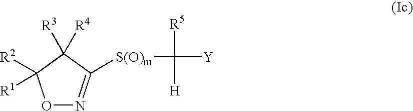

wherein R¹, R², R³, R⁴, R⁵ and Y are as defined above, with compounds of the formula R⁶—X, wherein R⁶ is as defined above and X is a suitable leaving group as defined in 1), in the presence of a base as defined in 1), optionally in the presence of a diluent as defined in 1) and optionally in the presence of a complexing agent as defined in 1) in a temperature range of from −120° C. to 100° C., preferably from −80° C. to 50° C.

4) The compounds of formula I wherein R¹, R², R³, R⁴, R⁵, R⁶ and Y are as defined above, m is 1 or 2, and n is 1, can, furthermore, be prepared by processes known per se

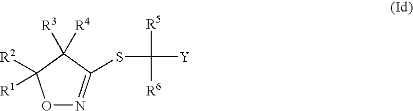

by starting from compounds of formula Id wherein R¹, R², R³, R⁴, R⁵, R⁶ and Y are as defined above, and reacting those compounds with a suitable organic or inorganic oxidising agent, e.g. a peroxy acid, such as 3-chloroperoxybenzoic acid, peracetic acid or hydrogen peroxide, an alkoxyperoxide or a periodate, such as sodium periodate, optionally in the presence of a diluent, such as a halogenated hydrocarbon, e.g. dichloro-methane or 1,2-dichloroethane, an alcohol, e.g. methanol, a polar solvent, e.g. N,N-dimethylformamide, water or acetic acid, or a mixture thereof. The reactions are usually carried out in a temperature range of from −80° C. to 150° C., preferably from −20° C. to 120° C. Such processes are known in the literature and are described e.g. in J. Org. Chem., 2003 (68) 3849-3859; J. Med. Chem., 2003 (46) 3021-3032; J. Org. Chem., 2003 (68) 500-511; Bioorg. Med. Chem., 1999 (9) 1837-1844. One equivalent of oxidizing agent is required to convert a sulfide to the corresponding sulfoxide. Two equivalents of oxidizing agent are required to convert a sulfide to the corresponding sulfone.

5) The compounds of formula I wherein R¹, R², R³, R⁴ and Y are as defined above, R⁵, R⁶ are as defined above but not hydrogen, m is 1 or 2, and n is 1, can also be prepared by reacting a compound of formula Ie

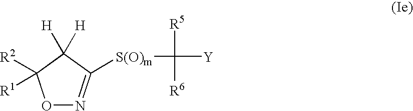

wherein R¹, R² and Y are as defined above, R⁵, R⁶ are as defined above but not hydrogen, and m is 1 or 2, in a single step or stepwise in succession with compounds of the formula R³—X and/or R⁴—X, wherein R³ and R⁴ are halogen or cyano and X is a suitable leaving group e.g. halide, such as bromide or iodide, or arylsulfonate, such as p-toluenesulfonate, if R³ and/or R⁴ are cyano or e.g. halide, such as bromide or iodide, or imide, such as succinimide, or sulfonimide, such as bis(phenylsulfonyl)imide, if R³ and/or R⁴ are halogen, in the presence of a base as defined in 1), optionally in the presence of a diluent as defined in 1), preferably an inert solvent, and optionally in the presence of a complexing agent as defined in 1) in a temperature range of from −120° C. to 100° C., preferably from −80° C. to 50° C.

6) The compounds of formula I wherein R¹, R², R³, R⁴ and Y are as defined above, R⁵, R⁶ are as defined above but not hydrogen, m is 1 or 2, and n is 1, can be prepared by processes known per se, by reacting e.g. the compounds of formula If

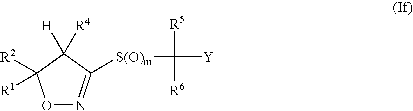

wherein R¹, R² and Y are as defined above, R⁵, R⁶ are as defined above but not hydrogen, and m is 1 or 2, with compounds of the formula R³—X, wherein R³ is as defined above and X is a suitable leaving group as defined in 1), in the presence of a base as defined in 1), optionally in the presence of a diluent as defined in 1), preferably an inert solvent, and optionally in the presence of a complexing agent as defined in 1) in a temperature range of from −120° C. to 100° C., preferably from −80° C. to 50° C.

7) The compounds of formula I wherein R¹, R², R³, R⁴ and Y are as defined above, R⁵, R⁶ are as defined above but not hydrogen, m is 1 or 2, and n is 1, can be prepared by processes known per se, by reacting e.g. the compounds of formula Ig

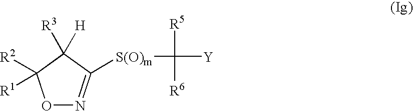

wherein R¹, R², R³ and Y are as defined above, R⁵, R⁶ are as defined above but not hydrogen, and m is 0 or 1, with compounds of the formula R⁴—X, wherein R⁴ is as defined above and X is a suitable leaving group as defined in 1), in the presence of a base as defined in 1), optionally in the presence of a diluent as defined in 1), preferably an inert solvent, and optionally in the presence of a complexing agent as defined in 1) in a temperature range of from −120° C. to 100° C., preferably from −80° C. to 50° C.

8) The compounds of formula Ie as defined in 5), can be prepared by processes known per se, by reacting e.g. a compound of formula Ih

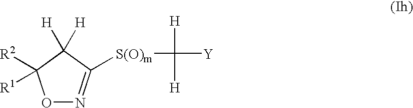

wherein R¹, R² and Y are as defined above, and m is 1 or 2, in a single step or stepwise in succession with compounds of the formula R⁵—X and/or R⁶—X, wherein R⁵ and R⁶ are as defined above and X is a suitable leaving group as defined in 1), in the presence of a base as defined in 1), optionally in the presence of a diluent as defined in 1), preferably an inert solvent, and optionally in the presence of a complexing agent as defined in 1) in a temperature range of from −120° C. to 100° C., preferably from −80° C. to 50° C. The compounds of formula Ih wherein R¹, R² and Y are as defined above are known compounds and can be prepared as described in e.g. US 2004/259734, US 2004/110749 and WO 06/024820.

9) The compounds of formula Id as defined in 4), can, furthermore, be prepared by reacting a compound of formula Ij,

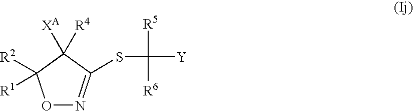

wherein R¹, R², R⁴, R⁵, R⁶ and Y are defined as above and X^{A} is a leaving group such as halide e.g. bromide or chloride, an alkylsulfonate, e.g. methylsulfonate, an arylsulfonate, e.g. p-toluenesulfonate, or a haloalkylsulfonate, e.g. trifluoromethylsulfonate, by reaction with a suitable salt of the formula II,

M-A (II)

wherein M is an organic cation, e.g. tetrabutyl ammonium, or an inorganic cation, such as a cation of an alkali metal, e.g. sodium, or caesium, or alkaline earth, e.g. magnesium, or a transition metal, e.g. silver, and A is an anion corresponding to R³ as defined above such as halide, e.g. fluoride, cyanide, azide or thiocyanate. This reaction may be carried out optionally in the presence of a diluent such as an ether, e.g. tetrahydrofuran, 1,4-dioxane, diethyl ether, or an amide, e.g. N,N-dimethylformamide, or dimethylsulfoxide, or acetonitrile or haloalkane, e.g. dichloromethane, or an aromatic compound, e.g. toluene, 1,2-dichlorobenzene, or water or mixture thereof. This reaction may be carried out optionally in the presence of a nucleophilic catalyst such as an iodide salt, e.g. sodium iodide, or a phase transfer catalyst such a crown ether, e.g. 18-crown-6, or a tetra alkyl ammonium salt, e.g. tetra butyl ammonium iodide. This reaction is usually carried out at temperature between −20° C. and 150° C., typically at a temperature between 15° C. and 100° C.

10) Alternatively, the compounds of formula Id as defined in 4), can be prepared by reacting a compound of formula Ij as defined in 9), by reaction with a suitable organo-metal reagent of formula II, wherein M is a metal such as an alkali metal, e.g. lithium, or a metal halide such as an alkaline earth halide, e.g. magnesium (II) monobromide as in a Grignard reagent, and A is an organic functionality such as an alkyl residue, or an alkenyl residue, or an alkynyl, e.g. ethynyl as in lithium acetylide. Such a reaction may be carried out in a solvent such as an ether, e.g. tetrahydrofuran, or 1,4-dioxane, or 1,2-dimethoxyethane, or an alkane, e.g. hexane, or dimethyl sulfoxide at temperatures between −120° C. and 120° C., typically between −100° C. and 100° C., optionally in the presence of a complexing agent, e.g. 1,3-dimethyl-3,4,5,6-tetrahydro-2(1H)-pyrimidinone (DMPU). Such reactions may be preformed under an inert atmosphere, e.g. nitrogen or argon, with the exclusion of moisture. Such a reaction may be carried out optionally in the presence of another metal or its salt, such as a transition metal or transition metal salt, e.g. palladium, or copper, or a mixture thereof with additionally a complexing agent for such a metal atom or cation such as a trisubstituted phosphine, e.g. triphenyl phosphine. Such a reaction may be carried out optionally in the presence of a nucleophilic catalyst, such as an iodide salt, e.g. sodium iodide or lithium iodide. The choice of reaction conditions may well affect the degree of oligomerisation of organometal reagents of formula XIX. Similar processes are known in the literature and described in Organic Letters, 2005 (7) 1785-1788; Tetrahedron Letters, 1991 (32) 3605; Tetrahedron Letters, 1991 (32) 1459; J. Am. Chem. Soc., 1990 (112) 5369.

### Coupling Methods

11) The compounds of formula Id as defined in 4) can be prepared by reacting a compound of formula III wherein R⁵, R⁶ and Y are as defined above and X^{A} is a leaving group as defined in 9), with thiourea, optionally in the presence of a diluent e.g. an alcohol, e.g. ethanol, optionally in the presence of an alkali iodide, e.g. sodium iodide or sodium bromide, in a temperature range of from −30° C. to 100° C., preferably from 0° C. to 80° C., to give an isothiourea intermediate of formula IV, which is reacted with a compound of formula V

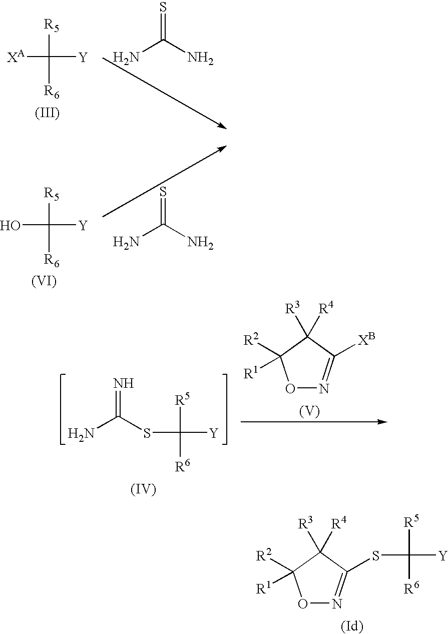

wherein R¹, R², R³ and R⁴ are as defined above, and X^{B} is a suitable leaving group such as halogen, e.g. chloro, an alkylsulfinyl group, an arylsulfinyl group, a haloalkylsulfinyl group, an alkylsulfonyl group, e.g. methylsulfonyl, an arylsulfonyl group, e.g. p-toluene-sulfonyl, a haloalkylsulfonyl group, e.g. trifluoromethylsulfonyl, or nitro, in the presence of a base, such as a carbonate, e.g. potassium carbonate, sodium carbonate or potassium bicarbonate, or a hydroxide, e.g. potassium hydroxide, or an alkoxide, e.g. sodium alkoxide, optionally in the presence of an alkali iodide, e.g. sodium iodide or sodium bromide, optionally in the presence of a diluent, such as an alcohol, e.g. ethanol, an ether, e.g. 1,4-dioxane or tetrahydrofuran, a polar solvent, e.g. water, acetonitrile or N,N-dimethylformamide, or a mixture of solvents, e.g. a mixture of 1,4-dioxane and water, in a temperature range of from 20° C. to 200° C., preferably from 50° C. to 150° C., optionally in the presence of an inert gas e.g. nitrogen, and optionally under microwave irradiation. Such processes are known in the literature and are described, for example, in WO 04/0131106.

12) A further method of preparing intermediates of formula IV, wherein R⁵, R⁶ and Y are as defined above, is to react a compound of formula VI, wherein R⁵, R⁶ and Y are as defined above, with thiourea in the presence of an acid, for example a mineral acid, such as hydrochloric acid or hydrobromic acid, or sulfuric acid, or an organic acid, such as trifluoroacetic acid, and optionally in the presence of a diluent, such as an ether, e.g. 1,4-dioxane or tetrahydrofuran, a polar solvent, e.g. water or N,N-dimethylformamide, or a mixture of solvents, e.g. a mixture of 1,4-dioxane and water, in a temperature range of from 20° C. to 270° C., preferably from 20° C. to 150° C., optionally under microwave irradiation. Such processes are known in the literature and are described, for example, in Buchwald and Neilsen, JACS, 110(10), 3171-3175 (1988); Frank and Smith, JACS, 68, 2103-2104 (1946); Vetter, Syn. Comm., 28, 3219-3233 (1998). The intermediate IV is then reacted with a compound of formula V as described in 11) to yield a compound of formula Id as described in 11).

13) A further method of preparing the compounds of formula Id as defined in 4) is to react compound of formula VII wherein R⁵, R⁶ and Y are as defined above,

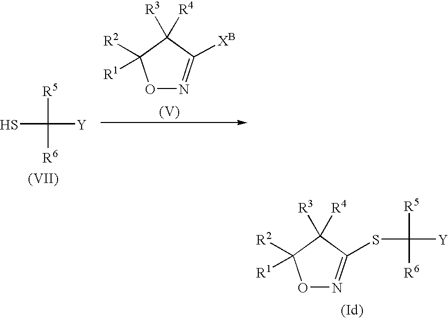

with a compound of formula V as defined in 11), in the presence of a base, e.g. potassium carbonate, optionally in the presence of a diluent, e.g. an amide, such as N,N-dimethyl-formamide, or an alcohol, such as ethanol, in a temperature range of from 0° C. to 100° C., preferably from 20° C. to 50° C., and optionally under an inert atmosphere, e.g. nitrogen. Such processes are known in the literature and are described, for example in WO 01/012613, WO 02/062770 and WO 04/010165.

14) Alternatively, the compounds of formula Id as defined in 4) can be prepared by reacting a compound of formula V as defined in 11) with thiourea, optionally in the presence of a diluent e.g. an alcohol, e.g. ethanol, in a temperature range of from −30° C. to 150° C., preferably from 0° C. to 80° C., to give an isothiourea intermediate of formula VIII,

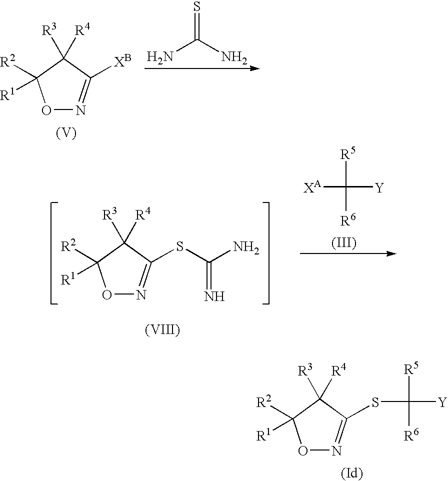

which is then reacted with a compound of formula III as defined in 11) in the presence of a base, such as a carbonate, e.g. potassium carbonate, sodium carbonate or potassium bicarbonate, or a hydroxide, e.g. potassium hydroxide, or an alkoxide, e.g. sodium alkoxide, optionally in the presence of a diluent, such as an alcohol, e.g. ethanol, a polar solvent, e.g. water or N,N-dimethylformamide, or a mixture of solvents, in a temperature range of from 0° C. to 200° C., preferably from 0° C. to 100° C. Such processes are known in the literature and are described, for example, in WO 05/095352.

15) A further method of preparing the compounds of formula Id as defined in 4) is to react an organometal reagent of the formula IX wherein R⁵, R⁶ and Y are as defined above and M^{B} is a group such as MgCl, MgBr, ZnBr or Li,

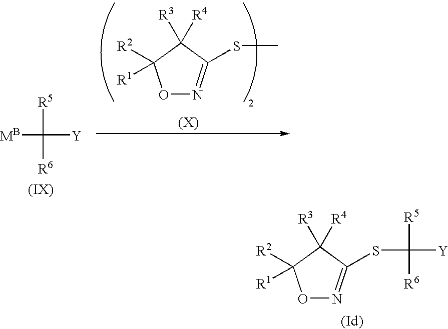

with a compound of formula X wherein R¹, R², R³ and R⁴ are as defined above optionally in the presence of a diluent, e.g. an ether, such as diethyl ether or tetrahydrofuran, in a temperature range of from −50° C. to 100° C., preferably from −20° C. to 50° C., and optionally under an inert atmosphere, e.g. nitrogen. The disulfide of formula X can be formed in situ or prepared separately by oxidation of the corresponding sulfide, which in turn is described in JP 2004/224714. Similar processes are known in the literature and are described, for example in J. Chem. Soc. Chem. Commun., 1991, 993-994, J. Chem. Soc. Perkin Trans. 1992 (24) 3371-3375, J. Org. Chem., 1989 (54) 2452-2453.

16) A further method of preparing the compounds of formula Id as defined in 4) is to react a compound of the formula IIIa wherein R⁵, R⁶ and Y are as defined above, and X^{D} is functional group that may be cleaved as a radical, e.g. a halogen, such as bromo or chloro,

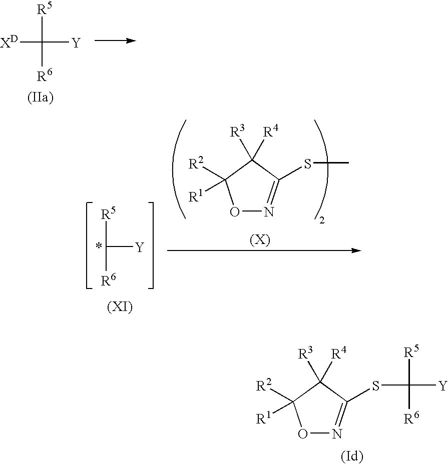

with a radical initiator or a precursor thereof and with a compound of formula X as defined in 15), optionally in the presence of a diluent, e.g. a polar solvent, such as water or N,N-dimethylformamide, or mixtures thereof, optionally in the presence of a base, e.g. a phosphate or hydrogen phosphate such as disodium hydrogenphosphate, a carbonate, e.g. potassium carbonate, sodium carbonate or potassium bicarbonate, in a temperature range of from −50° C. to 180° C., preferably from −20° C. to 50° C., and optionally under an inert atmosphere, e.g. nitrogen. As radical initiator or precursors can be used e.g. sodium dithionite or sodium hydroxymethanesulfinate.
Methods for making 4-halo-4,5-dihydro-isoxazole derivatives

17) The compounds of formula V as defined in 11), may be prepared by treating a compound of formula Va

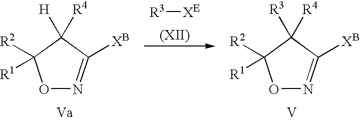

wherein R¹, R² and R⁴ are as defined above and X^{B} is as defined in 11), with a base and a compound of formula XII, R³—X^{E}, wherein R³ is defined as above and X^{E} is a suitable leaving group such as a halide, a perhaloalkyl, e.g. pentachloroethyl such as in hexa-chloroethane, an arylsulfonimide, e.g. benzenesulfonimide such as in N-fluorobenzene-sulfonimide (NFSI), an imide, e.g. succinimido such as in N-halosuccinimide, e.g. N-chlorosuccinimide (NCS), an arylsulfonyl, e.g. tosyl such as in tosylcyanide, or tertiary amine, e.g. diazo[2.2.2]byclooctane as in 1-(chloromethyl)-4-fluoro-1,4-diazoniabicyclo-[2.2.2]octane bis(tetrafluoroborate) (SELECTFLUOR). For halogenation, preferred reagents of formula XII are N-fluorobenzenesulfonimide (NFSI) or 1-(chloromethyl)-4-fluoro-1,4-diazoniabicyclo[2.2.2]octane bis(tetrafluoroborate) (SELECTFLUOR) for the fluorination, N-chlorosuccinimide (NCS) or hexachloroethane for the chlorination, N-bromosuccinimide (NBS) or phenyl trimethylamino tribromide (PTT) for the bromination and N-iodosuccinimide (NIS) for the iodination. These reactions are carried out in the presence of a suitable diluent such as an ether, e.g. tetrahydrofuran, or 1,4-dioxane, or diethyl ether, or 1,2-dimethoxy-ethane, or an amide, e.g. N,N-dimethylformamide, or a sulfoxide, e.g. dimethylsulfoxide. Such reactions may be carried out in the presence of alkoxide bases, e.g. potassium tert-butoxide, in a temperature range from −100° C. to 50° C., preferably from −80° C. to 0° C. Alternatively, the reactions are carried out in the presence of an alkyl-lithium compound, e.g. n-butyl lithium, optionally in the presence of a complexing agent, e.g. 1,3-dimethyl-3,4,5,6-tetrahydro-2(1H)-pyrimidinone (DMPU), in a temperature range from −100° C. to 50° C., preferably from −80° C. to 0° C. Alternatively, the reactions are carried out in the presence of a metal bis(tri(C₁-C₆alkyl)silyl)amide, e.g. sodium bis(trimethylsilyl)amide or potassium bis(trimethyl-silyl)amide, in a temperature range from −100° C. to 50° C., preferably from −80° C. to 0° C. Alternatively such reactions may be carried out using a phosphazene base, e.g. 1-tert-butyl-2,2,4,4,4-pentakis(dimethylamino)-2-lambda5-5,4-lambda5-5-catenadi-(phosphazene) (P₂-^{-t}Bu) or 1-ethyl-2,2,4,4,4-pentakis(dimethylamino)-2-lambda5-5,4 lambda5-5-catena-di-(phosphazene) (P₂-Et), or 2,8,9-triisobutyl-2,5,8,9-tetraaza-1-phosphabicyclo[3.3.3]undecane (Verkade's base), in a temperature range from 0° C. to 50° C., preferably from 0° C. to 30° C. Such reactions may be preformed under an inert atmosphere, e.g. nitrogen or argon, with the exclusion of moisture. The base may be added optionally to mixture of a compound of formula Va and a compound of formula XII in the presence of a suitable solvent at an appropriate temperature.

18) The compounds of formula Vb wherein R¹, R², R³ and R⁴ are as defined above and X^{F} is a suitable leaving group such as an alkylsulfinyl group, an arylsulfinyl group, a haloalkylsulfinyl group, an alkylsulfonyl group, e.g. methylsulfonyl, an arylsulfonyl group, e.g. phenylsulfonyl, or a haloalkylsulfonyl group, e.g. trifluoro-methylsulfonyl, may be prepared by treating a compound of formula Vc

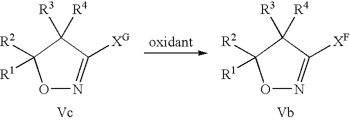

wherein R¹, R², R³ and R⁴ are as defined above and X^{G} is a group such as an alkyl-sulfanyl group, e.g. methylsulfanyl, an arylsulfanyl group, e.g. phenylsulfanyl, or a halo-alkylsulfanyl group, e.g. trifluoromethylsulfanyl, with a suitable organic or inorganic oxidising agent as defined in 4), optionally in the presence of a diluent as defined in 4), at a temperature of from −80° C. to 150° C., preferably from −20° C. to 120° C.

19) The compounds of formula Vd wherein R¹, R², R³ and R⁴ are as defined above and X^{H} is a group such as an alkylsulfanyl group, e.g. methylsulfanyl, an aryl-sulfanyl group, e.g. phenylsulfanyl, or a haloalkylsulfanyl group, e.g. trifluoromethyl-sulfanyl, halogen, such as chloro, or nitro, may prepared from compounds of formula Ve,

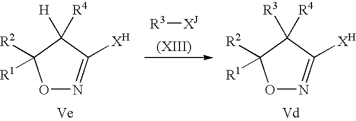

wherein R¹, R² and R⁴ are defined as above, and X^{H} is a group such as an alkylsulfanyl group, e.g. methylsulfanyl, an arylsulfanyl group, e.g. phenylsulfanyl, or a haloalkyl-sulfanyl group, e.g. trifluoromethylsulfanyl, halogen, such as chloro, or nitro, by reacting with a compound of formula R³—X^{J}, wherein R³ is defined as above and X^{J} is a functional group that may be cleaved to generate R³ as a radical, optionally in the presence of a diluent such as a halogenated hydrocarbon, e.g. dichloromethane, 1,2-dichloroethane, carbon tetrachloride, an ether, e.g. tetrahydrofuran, an aromatic compound, e.g. toluene, acetonitrile, an amide, e.g. N,N-dimethylformamide, water or a mixture thereof. The reactions are usually carried out in a temperature range from −50° C. to 120° C., preferably from −5° C. to 100° C. The reactions may be carried out optionally in the presence of light and or a radical initiator such as a peroxide, e.g. dibenzoylperoxide, or an azo compound, e.g. N,N′-azobis(isobutyronitrile). Suitable compounds of formula R³—X include compounds in which X is a succinimido group, in particular when R³ is a halogen, e.g. N-chlorosuccinimide and N-bromosuccinimide, or X is an alkoxy group, in particular when R³ is a halogen, e.g. t-butylhypochlorite. Similar processes are known in the literature and are described, e.g. Tetrahedron, 1999 (55) 4133-4152; European Journal of Medicinal Chemistry, 2002 (37) 933-944; Journal of Organic Chemistry, 2003 (68) 10187-10190; Journal of Organic Chemistry, 1988 (53), 5369-71.

20) The compounds of formula Vc wherein R¹, R², R³ and R⁴ are as defined above and X^{G} is a group as defined in 18), may prepared from a compound of formula Vf,

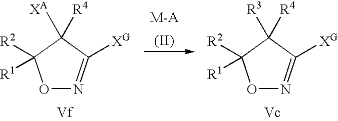

wherein R¹, R² and R⁴ are as defined above, X^{G} is as defined in 18) and X^{A} is a leaving group as defined in 9), by reaction with a suitable salt of the formula II as defined in 9). This reaction may be carried out optionally in the presence of a diluent as defined in 9), optionally in the presence of a nucleophilic catalyst as defined in 9) and in the temperature range as defined in 9).

21) Alternatively, the compounds of formula Vc wherein R¹, R², R³ and R⁴ are as defined above and X^{G} is a group as defined in 18), may be prepared from a compound of formula Vf as defined in 20), by reaction with a suitable organometal reagent of formula II as defined in 10). This reaction may be carried out in a solvent as defined in 10), in the temperature range as defined in 10), optionally in the presence of a complexing agent as defined in 10), optionally under an inert atmosphere as defined in 10), optionally in the presence of another metal or its salt as defined in 10) with additionally a complexing agent for such a metal atom or cation as defined in 10), optionally in the presence of a nucleophilic catalyst as defined in 10).

22) The compounds of formula Vc, wherein R¹, R², R³ and R⁴ are as defined above and X^{G} is group as defined in 18), may be prepared by reacting a compound of formula Vg,

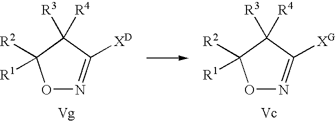

wherein R¹, R², R³ and R⁴ are as defined above and X^{D} is a halogen, e.g. chloro, with a thiol such as an alkyl-, e.g. methylthiol, or aryl-thiol, e.g. phenylthiol, or a haloalkylthiol, optionally in the presence of a base such as an organic base, e.g. triethylamine, or an inorganic base, e.g. potassium carbonate, sodium hydride. This reaction may be carried out optionally in the presence of a diluent such as an ether, e.g. tetrahydrofuran, 1,4-dioxane, diethyl ether, a ketone, e.g. butan-2-one, or an alcohol, e.g. ethanol. Suitable temperatures are between −20° C. and 150° C., preferably between 15° C. and 100° C.

23) The compounds of formula Vg, wherein R¹, R², R³ and R⁴ are as defined above and XD is a halogen, e.g. chloro, may be prepared from a compound of formula XIV,

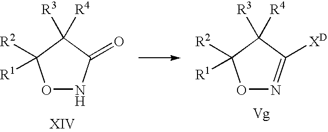

wherein R¹, R², R³ and R⁴ are as defined above, by reacting with a halogenating agent such as halophosphorous compound, e.g. phosphorous trichloride, phosphorous pentachloride, phosphorous oxychloride, phosphorous tribromide, or an alkylsulfonyl chloride, e.g. methylsulfonylchloride, or thionyl chloride or oxalylchloride. The reactions may be carried out optionally in the presence of a diluent such as a nitroalkane, e.g. nitro-methane, or a haloalkane, e.g. dichloromethane. Suitable temperatures are between −20° C. and 120° C., preferably between 15° C. and 100° C. Similar processes are known in the literature and are described in WO 2001/012613, WO 2002/062770, WO 2004/014138, WO 2005/095352.

24) The compounds of formula XIVa wherein R¹, R² and R³ are as described above and R⁴ is hydrogen, may be prepared from a compound of formula XV,

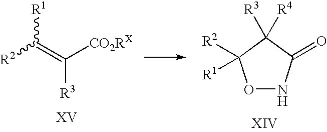

wherein R¹, R² and R³ are as defined above and R^{X} is alkyl, such as a C₁-C₄ lower alkyl, preferably ethyl, or methyl, optionally substituted alkyl, e.g. benzyl, or aryl, e.g. phenyl, or optionally substituted aryl, e.g. 4-nitrophenyl or pentafluorophenyl, by reaction with N-hydroxyurea or hydroxylamine in the presence of either an inorganic base such as a metal alkoxide, e.g. sodium ethoxide or sodium methoxide, or potassium tert-butoxide, or a metal hydride, e.g. sodium hydride, or a metal hydroxide, e.g. sodium hydroxide, or potassium hydroxide, or an organic base such as an amine, e.g. triethylamine. This reaction may be carried out optionally in the presence of a diluent such as an alcohol, e.g. methanol or ethanol, or an ether, e.g. tetrahydrofuran, 1,4-dioxane, diethyl ether, 1,2-dimethoxyethane, or an amide, e.g. N,N-dimethylformamide, or dimethylsulfoxide, or water or a mixture thereof. Suitable temperatures are between 0° C. and 120° C., preferably between 0° C. and 100° C. Similar processes are known in the literature and are described in Bull. Soc. Chim. France, 1976 (9-10, Pt. 2) 1589-1594.

25) The compounds of formula XV, wherein R¹, R² and R³ are as defined above and R^{X} is as defined in 24), are known in the literature and may be prepared processes such as those described in Synthesis 2004 (16) 2641-2644; Synthesis 2003 (4) 555-559; Angew. Chemie, Int. Ed. 2003 (42) 1397-1399; J. Fluorine Chem. 2002 (117) 161-166; Tetrahedron Letters 2001 (42) 243-245; European J. Org. Chem. 1998 (11) 2603-2607; Tetrahedron Asymmetry 1997 (82) 3745-3753; Phosphorus, Sulfur and Silicon and the Related Elements 1995 (105, Pt. 1-4) 205-12; Tetrahedron Letters 1995 (36) 3023-6; Helvetica Chimica Acta 1994 (77) 1480-4; Tetrahedron Letters 1993 (34) 7923-4; Tetrahedron 1988 (44) 7127-44; Tetrahedron Letters 1985 (26) 5991-4; Bulletin de la Societe Chimique de France (1985) (3), 455-62; Justus Liebigs Annalen der Chemie 1964 (679) 9-19.

26) The compounds of formula XIV or a salt thereof, wherein R¹, R², R³ and R⁴ are as defined above, may, furthermore, be prepared from a compound of formula XVI,

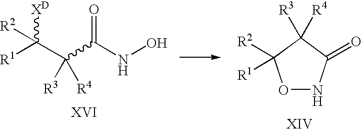

wherein R¹, R², R³ and R⁴ are as defined above and X^{D} is a suitable leaving group such as halide, e.g. chloride or bromide, in the presence of a inorganic base, such as an hydroxide salt, e.g. sodium hydroxide or potassium hydroxide, or a carbonate salt, e.g. potassium carbonate, an alkoxide salt, e.g. sodium methoxide, a metal hydride, sodium hydride. This reaction may be carried out optionally in the presence of a diluent such as an alcohol, e.g. methanol or ethanol, or water or a mixture thereof. Suitable temperatures are between 0° C. and 120° C., preferably between 15° C. and 100° C. Similar processes are known in the literature and are described in Zhumal Obshchei Khimii 1959 (29) 3417-24.

27) The compounds of formula XVI, wherein R¹, R², R³ and R⁴ are as defined above and X^{D} is as defined in 26), may be prepared from a compound of formula XVII,

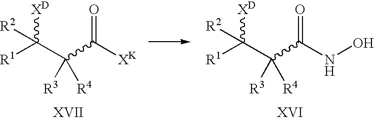

wherein R¹, R², R³ and R⁴ are as defined above and X^{D} is as defined in 26) and X^{K} is a suitable leaving group such as halide, e.g. chloride, by reaction with either hydroxyl-amine or an acid salt thereof, such as a mineral acid salt, e.g. hydrochloric acid as in hydroxylamine hydrochloride, in the presence of a inorganic base, such as an hydroxide salt, e.g. sodium hydroxide or potassium hydroxide, or a carbonate salt, e.g. potassium carbonate, an alkoxide salt, e.g. sodium methoxide, a metal hydride, such as sodium hydride. This reaction may be carried out optionally in the presence of a diluent such as an alcohol, e.g. methanol or ethanol, or water or a mixture thereof. Suitable temperatures are between −20° C. and 120° C., preferably between 0° C. and 100° C. Similar processes are known in the literature and are described in Zhurnal Obshchei Khimii 1959 (29) 3417-24.

28) The compounds of formula XIV or a salt thereof, wherein R¹, R², R³ and R⁴ are as defined above, may, furthermore, be prepared from a compound of formula XVIII,

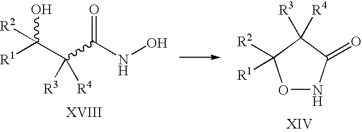

wherein R¹, R², R³ and R⁴ are as defined above, by a process as described in JP 2004/224714. Alternatively the compounds of formula XIV may be prepared from a compound of formula XVIII by reaction with a dehydrating agent such as a phosphorous oxide, e.g. phosphorous pentoxide, or arylsulfonyl halide, e.g. tosylchloride, or a carbonyl compound, e.g. diimidazolylketone or thionyl chloride or a combination of triphenylphosphine and diethyl azodicarboxylate. The reaction may be carried out optionally in the presence of a diluent such as a haloalkane, e.g. dichloromethane, or chloroform, or an aromatic compound, e.g. pyridine, or an amide, e.g. N,N-dimethyl-formamide at temperatures between −20° C. and 120° C., typically between −5° C. and 40° C. This reaction may be carried out optionally in the presence of either an inorganic base such as a carbonate, e.g. potassium carbonate, or an organic base such as an amine, triethylamine. Similar processes are known in the literature and are described in J. Heterocyclic Chemistry, 1990 (27) 275; J. Heterocyclic Chemistry, 1990 (27) 871; WO 2005/023820; JP 61161270; Journal of Medicinal Chemistry, 2005 (48) 4457-4468.

29) The compounds of formula V wherein R¹, R², R³ and R⁴ are as defined above and X^{B} is a suitable leaving group as defined in 11) may, alternatively, be prepared from a compound of formula XIV,

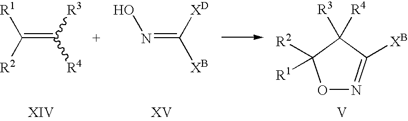

wherein R¹, R², R³ and R⁴ are as defined above, by reacting with a compound of formula XV wherein X^{B} is as defined above and X^{D} is a leaving group such as halide, e.g. chloride or bromide. Similar processes are known in the literature and are described in the Journal of Organic Chemistry, 1990 (55) 3045-51; Journal of Organic Chemistry, 19S7 (52) 2973-7; Journal of Organic Chemistry, 1983 (48) 1796-800; Journal of Organic Chemistry, 1981 (46) 5425-7; Journal of the American Chemical Society, 1979 (101) 1319-20.

30) The compounds of formula V wherein R¹, R², R³ and R⁴ are as defined above and X^{B} is a suitable leaving group as defined in 11), may be prepared by reacting a compound of formula XVI,

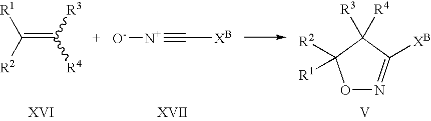

wherein R¹, R², R³ and R⁴ are as defined above, with a compound of formula XVII wherein X^{B} is as defined above. Similar processes are known in the literature and are described in Journal of Organic Chemistry, 1988 (53) 5369-71; Tetrahedron Letters, 1979 (2) 139-42; Journal of the American Chemical Society, 1979 (101) 1319-20.
General Methods for making Heterocyclic Intermediates

31) The compounds of formula III as defined in 11) can be prepared by reacting compounds of formula VI as defined in 12)

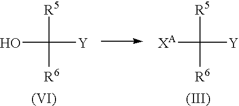

with a halogenating agent, such as hydrogen chloride, hydrogen bromide, phosphorous tribromide, phosphorous trichloride or thionyl chloride, or with an alkyl-, aryl- or halo-alkylsulfonyl chloride, such as methanesulfonyl chloride, p-toluenesulfonyl chloride or trifluoromethylsulfonyl chloride, or with a combination of carbon tetrabromide and triphenyl phosphine, optionally in the presence of an inert solvent, e.g. a halogenated hydrocarbon, such as dichloromethane, 1,2-dichloroethane or carbon tetrachloride, an ether, such as diethyl ether or tetrahydrofuran, or an acid, such as acetic acid, optionally in the presence of a base, e.g. an amine, such as triethyl amine, in a temperature range from −50° C. to 100° C., preferably from 0° C. to 50° C. Such processes are known in the literature and are described, for example, in J. Med. Chem. 2005 (48) 3438-3442, J. Org. Chem., 2005 (70) 2274-2284, Org. and Biomolecular Chem., 2005 (3) 1013-1024, Bioorg. Med. Chem. 2004 (13) 363-384, Tetrahedron Asymmetry 2004 (15) 3719-3722.

32) Alternatively, the compounds of formula IIa, wherein R⁵, R⁶ and Y are as defined above, and X^{D} is a leaving group such as halogen, e.g. bromo or chloro, can be prepared

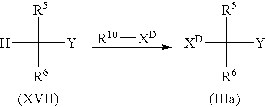

by reacting compounds of formula XVII wherein R⁵, R⁶ and Y are as defined above, with compounds of formula R¹⁰—X^{D}, wherein X^{D} is a leaving group such as halogen, e.g. bromo or chloro, and R¹⁰ is a functional group that may be cleaved to generate X^{D} as a radical, optionally in the presence of a diluent such as a halogenated hydrocarbon, e.g. dichloromethane, 1,2-dichloroethane or carbon tetrachloride, an ether, e.g. tetrahydro-furan, an aromatic compound, e.g. toluene, a polar solvent, e.g. acetonitrile, N,N-dimethylformamide or water, or a mixture thereof. The reactions are usually carried out in a temperature range from −50° C. to 120° C., preferably from −5° C. to 100° C. The reactions may be carried out optionally in the presence of light and or a radical initiator such as a peroxide, e.g. dibenzoylperoxide, or an azo compound, e.g. N,N′-azobis-(isobutyronitrile). Suitable compounds of formula R¹⁰—X^{A} include compounds in which R¹⁰ is a succinimido group, e.g. N-chlorosuccinimide and N-bromosuccinimide. Similar processes are known in the literature and are described, e.g. Tetrahedron, 1988 (44) 461-469; Journal of Organic Chemistry, 1981 (46) 679-686; J. Chem. Soc., Perkin Trans 1, 1985 (6), 1167-1170.

33) The compounds of formula VIa, wherein R⁶ is hydrogen or C₁-C₆alkyl and Y is as defined above, can be prepared

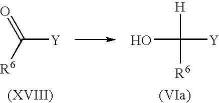

by reacting a compound of formula XVIII wherein R⁶ is hydrogen or C₁-C₆alkyl and Y is as defined above with a reducing agent, e.g. a metal hydride, such as diisobutyl aluminium hydride, lithium aluminium hydride, sodium borohydride, lithium boro-hydride, or diborane, optionally in the presence of an inert solvent, e.g. an ether, such as diethyl ether, 1,4-dioxane or tetrahydrofuran, an alcohol, such as methanol or ethanol, or an aromatic hydrocarbon, such as toluene. Such reactions are usually carried out in a temperature range from −50° C. to 100° C., preferably from 0° C. to 80° C. Such processes are known in the literature and are described, for example, in Tetrahedron Asymmetry, 2004 (15) 363-386; J. Med. Chem., 2002 (45) 19-31; Justus Liebigs Annalen der Chemie, 1978 (8) 1241-49.

34) Alternatively, the compounds of formula VIb, wherein Y is as defined above, can be prepared by reacting a compound of formula XIX,

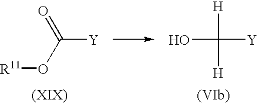

wherein Y is as defined above and R¹¹ is hydrogen or C₁-C₁₀alkyl, with a reducing agent, e.g. a metal hydride, such as diisobutyl aluminium hydride, lithium aluminium hydride, sodium borohydride, lithium borohydride, or diborane, optionally in the presence of an inert solvent, e.g. an ether, such as diethyl ether, 1,4-dioxane or tetrahydrofuran, an alcohol, such as methanol or ethanol, or an aromatic hydrocarbon, such as toluene. Such reactions are usually carried out in a temperature range from −50° C. to 100° C., preferably from 0° C. to 80° C. Such processes are known in the literature and are described, for example, in Tetrahedron Asymmetry 2004 (15) 3719-3722, J. Med. Chem., 2004 (47) 2176-2179, Heterocyclic Communications 2002 (8) 385-390, J. Antibiotics, 1995 (48) 1320-1329.

35) Additionally, the compounds of formula VIc wherein Y is as defined above and X^{D} is halogen, such as bromo or chloro, can be prepared from a compound of formula XX wherein Y is as defined above

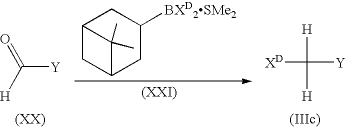

by reacting with a reagent of formula XXI wherein X^{D} is halogen, such as bromo or chloro, in the presence of a diluent such as a halogenated hydrocarbon such as dichloromethane, a hydrocarbon such as hexane, an alcohol such as ethanol, N—N-dimethylformamide, tetrahydrofuran or a mixture thereof. The preparation of aromatic benzyl halides is described in Tetrahedron Letts. 2000 (41) 5161-5164. The preparation of the reagent XXI is described in J. Org. Chem. 1980 (45) 384-389.

36) The preparation of pyrazoles, including methods of ring formation, is further described in EP 1364946, EP 1541561, WO 05/095352 and WO 05/105755; the preparation of triazoles, including methods of ring formation, is further discussed in GB 0603891.3.

The compounds of formula I according to the invention can be used as herbicides in unmodified form, as obtained in the synthesis, but they are generally formulated into herbicidal compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, micro-emulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO Specifications for Plant Protection Products, 5th Edition, 1999. Such formulations can either be used directly or they are diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof. The active ingredients can also be contained in very fine microcapsules consisting of a polymer. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art in this connection. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hex adecane, hexylene glycol, isoamyl acetate, isobornyl acetate, isooctane, isophorone, isopropylbenzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octa-decanoic acid, octylamine acetate, oleic acid, oleylamine, o-xylene, phenol, polyethylene glycol (PEG400), propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like. Water is generally the carrier of choice for diluting the concentrates. Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances, as described, for example, in CFR 180.1001. (c) & (d).

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quarternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in “McCutcheon's Detergents and Emulsifiers Annual” MC Publishing Corp., Ridgewood New Jersey, 1981.

Further adjuvants that can usually be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, tak-e-up enhancers, micronutrients, plasticisers, glidants, lubricants, dispersants, thickeners, antifreezes, microbicides, and also liquid and solid fertilisers.

The compositions according to the invention can additionally include an additive comprising an oil of vegetable or animal origin, a mineral oil, alkyl esters of such oils or mixtures of such oils and oil derivatives. The amount of oil additive in the composition according to the invention is generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil, olive oil or sunflower oil, emulsified vegetable oil, such as AMIGO® (Rhone-Poulenc Canada Inc.), alkyl esters of oils of vegetable origin, for example the methyl derivatives, or an oil of animal origin, such as fish oil or beef tallow. A preferred additive contains, for example, as active components essentially 80% by weight alkyl esters of fish oils and 15% by weight methylated rapeseed oil, and also 5% by weight of customary emulsifiers and pH modifiers. Especially preferred oil additives comprise alkyl esters of C₈-C₂₂ fatty acids, especially the methyl derivatives of C₁₂-C₁₈ fatty acids, for example the methyl esters of lauric acid, palmitic acid and oleic acid, being of importance. Those esters are known as methyl laurate (CAS-111-82-0), methyl palmitate (CAS-112-39-0) and methyl oleate (CAS-112-62-9). A preferred fatty acid methyl ester derivative is Emery® 2230 and 2231 (Cognis GmbH). Those and other oil derivatives are also known from the Compendium of Herbicide Adjuvants, 5th Edition, Southern Illinois University, 2000.

The application and action of the oil additives can be further improved by combination with surface-active substances, such as non-ionic, anionic or cationic surfactants. Examples of suitable anionic, non-ionic and cationic surfactants are listed on pages 7 and 8 of WO 97/34485. Preferred surface-active substances are anionic surfactants of the dodecylbenzylsulfonate type, especially the calcium salts thereof, and also non-ionic surfactants of the fatty alcohol ethoxylate type. Special preference is given to ethoxylated C₁₂-C₂₂ fatty alcohols having a degree of ethoxylation of from 5 to 40. Examples of commercially available surfactants are the Genapol types (Clariant AG). Also preferred are silicone surfactants, especially polyalkyl-oxide-modified heptamethyltriloxanes which are commercially available e.g. as Silwet L-77®, and also perfluorinated surfactants. The concentration of the surface-active substances in relation to the total additive is generally from 1 to 30% by weight. Examples of oil additives consisting of mixtures of oil or mineral oils or derivatives thereof with surfactants are Edenor ME SU®, Turbocharge® (Syngenta AG, CH) or ActipronC (BP Oil UK Limited, GB).

If desired, it is also possible for the mentioned surface-active substances to be used in the formulations on their own, that is to say without oil additives.

Furthermore, the addition of an organic solvent to the oil additive/surfactant mixture may contribute to an additional enhancement of action. Suitable solvents are, for example, Solvesso® (ESSO) or Aromatic Solvent® (Exxon Corporation). The concentration of such solvents can be from 16 to 80% by weight of the total weight. Oil additives that are present in admixture with solvents are described, for example, in U.S. Pat. No. 4,834,908. A commercially available oil additive disclosed therein is known by the name MERGE® (BASF Corporation). A further oil additive that is preferred according to the invention is SCORE® (Syngenta Crop Protection Canada).

In addition to the oil additives listed above, for the purpose of enhancing the action of the compositions according to the invention it is also possible for formulations of alkylpyrrolidones (e.g. Agrimax®) to be added to the spray mixture. Formulations of synthetic lattices, e.g. polyacrylamide, polyvinyl compounds or poly-1-p-menthene (e.g. Bond®, Courier® or Emerald®) may also be used. It is also possible for solutions that contain propionic acid, for example Eurogkem Pen-e-trate®, to be added to the spray mixture as action-enhancing agent.

The herbicidal compositions generally comprise from 0.1 to 99% by weight, especially from 0.1 to 95% by weight, compounds of formula I and from 1 to 99.9% by weight of a formulation adjuvant which preferably includes from 0 to 25% by weight of a surface-active substance. Whereas commercial products will preferably be formulated as concentrates, the end user will normally employ dilute formulations.

The rates of application of compounds of formula I may vary within wide limits and depend on the nature of the soil, the method of application (pre- or post-emergence; seed dressing; application to the seed furrow; no tillage application etc.), the crop plant, the grass or weed to be controlled, the prevailing climatic conditions, and other factors governed by the method of application, the time of application and the target crop. The compounds of formula I according to the invention are generally applied at a rate of from 10 to 2000 g/ha, especially from 50 to 1000 g/ha.

Preferred formulations have especially the following compositions (% percent by weight):

### Emulsifiable Concentrates:

| | |
|---|---|
| active ingredient: | 1 to 95%, preferably 60 to 90% |
| surface-active agent: | 1 to 30%, preferably 5 to 20% |
| liquid carrier: | 1 to 80%, preferably 1 to 35% |

### Dusts:

| | |
|---|---|
| active ingredient: | 0.1 to 10%, preferably 0.1 to 5% |
| solid carrier: | 99.9 to 90%, preferably 99.9 to 99% |

### Suspension Concentrates:

| | |
|---|---|
| active ingredient: | 5 to 75%, preferably 10 to 50% |
| water: | 94 to 24%, preferably 88 to 30% |
| surface-active agent: | 1 to 40%, preferably 2 to 30% |

### Wettable Powders:

| | |
|---|---|
| active ingredient: | 0.5 to 90%, preferably 1 to 80% |
| surface-active agent: | 0.5 to 20%, preferably 1 to 15% |
| solid carrier: | 5 to 95%, preferably 15 to 90% |

### Granules:

| | |
|---|---|
| active ingredient: | 0.1 to 30%, preferably 0.1 to 15% |
| solid carrier: | 99.5 to 70%, preferably 97 to 85% |

The following Examples further illustrate, but do not limit, the invention.
Formulation Examples for herbicides of formula I (%=% by weight)

| F1. Emulsifiable concentrates | a) | b) | c) | d) |
|---|---|---|---|---|
| active ingredient | 5% | 10% | 25% | 50% |
| calcium dodecylbenzenesulfonate | 6% | 8% | 6% | 8% |
| castor oil polyglycol ether | 4% | — | 4% | 4% |
| (36 mol of ethylene oxide) | | | | |
| octylphenol polyglycol ether | — | 4% | — | 2% |
| (7-8 mol of ethylene oxide) | | | | |
| NMP | — | — | 10% | 20% |
| arom. hydrocarbon mixture | 85% | 78% | 55% | 16% |
| C₉-C₁₂ | | | | |

Emulsions of any desired concentration can be obtained from such concentrates by dilution with water.

| F2. Solutions | a) | b) | c) | d) |
|---|---|---|---|---|
| active ingredient | 5% | 10% | 50% | 90% |
| 1-methoxy-3-(3-methoxy- | — | 20% | 20% | — |
| propoxy)-propane | | | | |
| polyethylene glycol MW 400 | 20% | 10% | — | — |
| NMP | — | — | 30% | 10% |
| arom. hydrocarbon mixture | 75% | 60% | — | — |
| C₉-C₁₂ | | | | |

The solutions are suitable for use in the form of microdrops.

| F3. Wettable powders | a) | b) | c) | d) |
|---|---|---|---|---|
| active ingredient | 5% | 25% | 50% | 80% |
| sodium lignosulfonate | 4% | — | 3% | — |
| sodium lauryl sulfate | 2% | 3% | — | 4% |
| sodium diisobutylnaphthalene- | — | 6% | 5% | 6% |
| sulfonate | | | | |
| octylphenol polyglycol ether | — | 1% | 2% | — |
| (7-8 mol of ethylene oxide) | | | | |
| highly dispersed silicic acid | 1% | 3% | 5% | 10% |
| kaolin | 88% | 62% | 35% | — |

The active ingredient is mixed thoroughly with the adjuvants and the mixture is thoroughly ground in a suitable mill, affording wettable powders which can be diluted with water to give suspensions of any desired concentration.

| F4. Coated granules | a) | b) | c) |
|---|---|---|---|
| active ingredient | 0.1% | 5% | 15% |
| highly dispersed silicic acid | 0.9% | 2% | 2% |
| inorganic carrier | 99.0% | 93% | 83% |
| (diameter 0.1-1 mm) | | | |
| e.g. CaCO₃ or SiO₂ | | | |

The active ingredient is dissolved in methylene chloride and applied to the carrier by spraying, and the solvent is then evaporated off in vacuo.

| F5. Coated granules | a) | b) | c) |
|---|---|---|---|
| active ingredient | 0.1% | 5% | 15% |
| polyethylene glycol MW 200 | 1.0% | 2% | 3% |
| highly dispersed silicic acid | 0.9% | 1% | 2% |
| inorganic carrier | 98.0% | 92% | 80% |
| (diameter 0.1-1 mm) | | | |
| e.g. CaCO₃ or SiO₂ | | | |

The finely ground active ingredient is uniformly applied, in a mixer, to the carrier moistened with polyethylene glycol. Non-dusty coated granules are obtained in this manner.

| F6. Extruder granules | a) | b) | c) | d) |
|---|---|---|---|---|
| active ingredient | 0.1% | 3% | 5% | 15% |
| sodium lignosulfonate | 1.5% | 2% | 3% | 4% |
| carboxymethylcellulose | 1.4% | 2% | 2% | 2% |
| kaolin | 97.0% | 93% | 90% | 79% |

The active ingredient is mixed and ground with the adjuvants, and the mixture is moistened with water. The mixture is extruded and then dried in a stream of air.

| F7. Dusts | a) | b) | c) |
|---|---|---|---|
| active ingredient | 0.1% | 1% | 5% |
| talcum | 39.9% | 49% | 35% |
| kaolin | 60.0% | 50% | 60% |

Ready-to-use dusts are obtained by mixing the active ingredient with the carriers and grinding the mixture in a suitable mill.

| F8. Suspension concentrates | a) | b) | c) | d) |
|---|---|---|---|---|
| active ingredient | 3% | 10% | 25% | 50% |
| ethylene glycol | 5% | 5% | 5% | 5% |
| nonylphenol polyglycol ether | — | 1% | 2% | — |
| (15 mol of ethylene oxide) | | | | |
| sodium lignosulfonate | 3% | 3% | 4% | 5% |
| carboxymethylcellulose | 1% | 1% | 1% | 1% |
| 37% aqueous formaldehyde | 0.2% | 0.2% | 0.2% | 0.2% |
| solution | | | | |
| silicone oil emulsion | 0.8% | 0.8% | 0.8% | 0.8% |
| water | 87% | 79% | 62% | 38% |

The finely ground active ingredient is intimately mixed with the adjuvants, giving a suspension concentrate from which suspensions of any desired concentration can be obtained by dilution with water.

The invention also relates to a method of controlling plants which comprises applying to the plants or to the locus thereof a herbicidally effective amount of a compound of formula I.

The invention also relates to a method of inhibiting plant growth which comprises applying to the plants or to the locus thereof a herbicidally effective amount of a compound of formula I.

The invention also relates to a method of selectively controlling grasses and weeds in crops of useful plants which comprises applying to the useful plants or locus thereof or to the area of cultivation a herbicidally effective amount of a compound of formula I.

Crops of useful plants in which the composition according to the invention can be used include cereals, for example barley and wheat, cotton, oilseed rape, maize, rice, soy beans, sugar beet and sugar cane, especially cereals and maize.

Crops can also include trees, such as palm trees, coconut trees or other nuts, and vines such as grapes.

The grasses and weeds to be controlled may be both monocotyledonous species, for example Agrostis, Alopecurus, Avena, Bromus, Cyperus, Digitaria, Echinochloa, Lolium, Monochoria, Rottboellia, Sagittaria, Scirpus, Setaria, Sida and Sorghum, and dicotyledonous species, for example Abutilon, Amaranthus, Chenopodium, Chrysanthemum, Galium, Ipomoea, Nasturtium, Sinapis, Solanum, Stellaria, Veronica, Viola and Xanthium.

Crops are to be understood as also including those crops which have been rendered tolerant to herbicides or classes of herbicides. (e.g. ALS-, GS-, EPSPS-, PPO- and BPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B13 (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood as being those which are obtained by conventional methods of breeding or genetic engineering and contain so-called output traits (e.g. improved storage stability, higher nutritional value and improved flavour).

Areas under cultivation include land on which the crop plants are already growing and land intended for cultivation with those crop plants.

The compounds of formula I according to the invention can also be used in combination with other herbicides. In particular, the following mixtures of the compound of formula I are important:

Mixtures of a compound of formula I with S-metolachlor (549) or a compound of formula I with metolachlor (548).

Mixtures of a compound of formula I with a triazine (e.g. compound of formula I+ametryn (20), compound of formula I+atrazine (37), compound of formula I+cyanazine (183), compound of formula I+dimethametryn (259), compound of formula I+metribuzin (554), compound of formula I+prometon (665), compound of formula I+prometryn (666), compound of formula I+propazine (672), compound of formula I+simazine (730), compound of formula I+simetryn (732), compound of formula I+terbumeton (774), compound of formula I+terbuthylazine (775), compound of formula I+terbutryn (776), compound of formula I+trietazine (831)). Particularly preferred are mixtures of a compound of formula I with atrazine, metribuzin, prometryn or with terbuthylazine.

Mixtures of a compound of formula I with an HPPD inhibitor (e.g. compound of formula I+isoxaflutole (479), compound of formula I+mesotrione (515), compound of formula I+sulcotrione (747), compound of formula I+tembotrione (CAS RN 335104-84-2), compound of formula I+topramezone (CAS RN 210631-68-8), compound of formula I+4-hydroxy-3-[[2-[(2-methoxyethoxy)methyl]-6-(trifluoromethyl)-3-pyridinyl]carbonyl]-bicyclo[3.2.1]oct-3-en-2-one (CAS RN 352010-68-5), compound of formula I+4-hydroxy-3-[[2-(3-methoxypropyl)-6-(difluoromethyl)-3-pyridinyl]carbonyl]-bicyclo[3.2.1]oct-3-en-2-one).

Mixtures of a compound of formula I with an HPPD inhibitor and a triazine.

Mixtures of a compound of formula I with glyphosate (419).

Mixtures of a compound of formula I with glyphosate and an HPPD inhibitor (e.g. compound of formula I+glyphosate+isoxaflutole, compound of formula I+glyphosate+mesotrione, compound of formula I+glyphosate+sulcotrione, compound of formula I+glyphosate+tembotrione, compound of formula I+glyphosate+topramezone, compound of formula I+glyphosate+4-hydroxy-3-[[2-[(2-methoxyethoxy)methyl]-6-(trifluoromethyl)-3-pyridinyl]carbonyl]-bicyclo[3.2.1]oct-3-en-2-one, compound of formula I+glyphosate+4-hydroxy-3-[[2-(3-methoxypropyl)-6-(difluoromethyl)-3-pyridinyl]carbonyl]-bicyclo[3.2.1]oct-3-en-2-one).

Mixtures of a compound of formula I with glufosinate-ammonium (418).

Mixtures of a compound of formula I with glufosinate-ammonium and an HPPD inhibitor (e.g. compound of formula I+glufosinate-ammonium+isoxaflutole, compound of formula I+glufosinate-ammonium+mesotrione, compound of formula I+glufosinate-ammonium+sulcotrione, compound of formula I+glufosinate-ammonium+tembotrione, compound of formula I+glufosinate-ammonium+topramezone, compound of formula I+glufosinate-ammonium+4-hydroxy-3-[[2-[(2-methoxyethoxy)methyl]-6-(trifluoromethyl)-3-pyridinyl]carbonyl]-bicyclo[3.2.1]oct-3-en-2-one, compound of formula I+glufosinate-ammonium+4-hydroxy-3-[[2-(3-methoxypropyl)-6-(difluoromethyl)-3-pyridinyl]carbonyl]-bicyclo[3.2.1]oct-3-en-2-one).

Mixtures of a compound of formula I with a triazolinone (e.g. compound of formula I+amicarbazone (21)).

Mixtures of a compound of formula I with an ALS inhibitor (e.g. compound of formula I+chlorsulfuron (147), compound of formula I+cinosulfuron (154), compound of formula I+cloransulam-methyl (164), compound of formula I+ethametsulfuron-methyl (306), compound of formula I+flazasulfuron (356), compound of formula I+foramsulfuron (402), compound of formula I+flumetsulam (374), compound of formula I+imazamethabenz-methyl (450), compound of formula I+imazamox (451), compound of formula I+imazapic (452), compound of formula I+imazapyr (453), compound of formula I+imazethapyr (455), compound of formula I+iodosulfuron-methyl-sodium (466), compound of formula I+metsulfuron-methyl (555), compound of formula I+nicosulfuron (577), compound of formula I+oxasulfuron (603), compound of formula I+primisulfuron-methyl (657), compound of formula I+prosulfuron (684), compound of formula I+pyrithiobac-sodium (709), compound of formula I+rimsulfuron (721), compound of formula I+sulfosulfuron (752), compound of formula I+thifensulfuron-methyl (thiameturon-methyl) (795), compound of formula I+triasulfuron (817), compound of formula I+tribenuron-methyl (822), compound of formula I+trifloxysulfuron-sodium (833), compound of formula I+thiencarbazone (BAY636)). Particularly preferred are mixtures of a compound of formula I with flazasulfuron, foramsulfuron, flumetsulam, imazapyr, imazethapyr, iodosulfuron-methyl-sodium, nicosulfuron, rimsulfuron, trifloxysulfuron-sodium or with 4-[(4,5-dihydro-3-methoxy-4-methyl-5-oxo)-1H-1,2,4-triazol-1-ylcarbonylsulfamoyl]-5-methylthiophene-3-carboxylic acid (BAY636).

Mixtures of a compound of formula I with a PPO inhibitor (e.g. compound of formula I+fomesafen (401), compound of formula I+flumioxazin (376), compound of formula I+sulfentrazone (749), compound of formula I+[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester) (CAS RN 353292-31-6). Particularly preferred are mixtures of a compound of formula I with flumioxazin, sulfentrazone or [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester.

Mixtures of a compound of formula I with paraquat dichloride (614).

Mixtures of a compound of formula I with pendimethalin (621) or a compound of formula I with trifluralin (836). Particularly preferred are mixtures of a compound of formula I with pendimethalin.

Mixtures of a compound of formula I with metamitron (521).

Mixtures of a compound of formula I with clomazone (159).

Mixtures of a compound of formula I with metazachlor (524).

Mixtures of a compound of formula I with clodinafop-propargyl (156) or a compound of formula I with pinoxaden (CAS RN 243973-20-8).

The mixing partners of the compound of formula I may also be in the form of esters or salts, as mentioned e.g. in The Pesticide Manual, 13^{th} Edition (BCPC), 2003. The reference to glufosinate-ammonium also applies to glufosinate, the reference to cloransulam-methyl also applies to cloransulam, and the reference to pyrithiobac-sodium also applies to pyrithiobac, etc.

The mixing ratio of the compound of formula I to the mixing partner is preferably from 1:100 to 1000:1.

The mixtures can advantageously be used in the above-mentioned formulations (in which case “active ingredient” relates to the respective mixture of compound of formula I with the mixing partner).

Furthermore, the compounds of formula I according to the invention can also be used in combination with other herbicides: compound of formula I+acetochlor (5), compound of formula I+acifluorfen-sodium (7), compound of formula I+aclonifen (8), compound of formula I+acrolein (10), compound of formula I+alachlor (14), compound of formula I+alloxydim (18), compound of formula I+allyl alcohol (CAS RN 107-18-6), compound of formula I+amidosulfuron (22), compound of formula I+aminopyralid (CAS RN 150114-71-9), compound of formula I+amitrole (aminotriazole) (25), compound of formula I+ammonium sulfamate (26), compound of formula I+anilofos (31), compound of formula I+asulam (36), compound of formula I+atraton (CAS RN 1610-17-9), compound of formula I+aviglycine (39), compound of formula I+azafenidin (CAS RN 68049-83-2), compound of formula I+azimsulfuron (43), compound of formula I+BAS 800H(CAS RN 372137-35-4), compound of formula I+BCPC(CAS RN 2164-13-8), compound of formula I+beflubutamid (55), compound of formula I+benazolin (57), compound of formula I+bencarbazone (CAS RN 173980-17-1), compound of formula I+benfluralin (59), compound of formula I+benfuresate (61), compound of formula I+bensulfuron-methyl (64), compound of formula I+bensulide (65), compound of formula I+bentazone (67), compound of formula I+benzfendizone (CAS RN 158755-95-4), compound of formula I+benzobicyclon (69), compound of formula I+benzofenap (70), compound of formula I+bifenox (75), compound of formula I+bilanafos (bialaphos) (77), compound of formula I+bispyribac-sodium (82), compound of formula I+borax (86), compound of formula I+bromacil (90), compound of formula I+bromobutide (93), compound of formula I+bromofenoxim (CAS RN 13181-17-4), compound of formula I+bromoxynil (95), compound of formula I+butachlor (100), compound of formula I+butafenacil (101), compound of formula I+butamifos (102), compound of formula I+butralin (105), compound of formula I+butroxydim (106), compound of formula I+butylate (108), compound of formula I+cacodylic acid (CAS RN 75-60-5), compound of formula I+calcium chlorate (CAS RN 10137-74-3), compound of formula I+cafenstrole (110), compound of formula I+carbetamide (117), compound of formula I+carfentrazone-ethyl (121), compound of formula I+CDEA (CAS RN 2315-36-8), compound of formula I+CEPC(CAS RN 587-56-4), compound of formula I+chlorbromuron (CAS RN 13360-45-7), compound of formula I+chlorflurenol-methyl (133), compound of formula I+chloridazon (134), compound of formula I+chlorimuron-ethyl (135), compound of formula I+chloroacetic acid (138), compound of formula I+chlorotoluron (143), compound of formula I+chlorpropham (144), compound of formula I+chlorthal-dimethyl (148), compound of formula I+cinidon-ethyl (152), compound of formula I+cinmethylin (153), compound of formula I+cisanilide (CAS RN 34484-77-0), compound of formula I+clefoxydim (CAS RN 211496-02-5), compound of formula I+clethodim (155), compound of formula I+clomeprop (160), compound of formula I+clopyralid (162), compound of formula I+CMA (CAS RN 5902-95-4), compound of formula I+4-CPB (CAS RN 3547-07-7), compound of formula I+CPMF, compound of formula I+4-CPP(CAS RN 3307-39-9), compound of formula I+CPPC(CAS RN 2150-32-5), compound of formula I+cresol (CAS RN 1319-77-3), compound of formula I+cumyluron (180), compound of formula I+cyanamide (182), compound of formula I+cyclanilide (186), compound of formula I+cycloate (187), compound of formula I+cyclosulfamuron (189), compound of formula I+cycloxydim (190), compound of formula I+cyhalofop-butyl (195), compound of formula I+2,4-D (211), compound of formula I+3,4-DA (CAS RN 588-22-7), compound of formula I+daimuron (213), compound of formula I+dalapon (214), compound of formula I+dazomet (216), compound of formula I+2,4-DB (217), compound of formula I+3,4-DB, compound of formula I+2,4-DEB (CAS RN 94-83-7), compound of formula I+desmedipham (225), compound of formula I+desmetryn (CAS RN 1014-69-3), compound of formula I+dicaniba (228), compound of formula I+dichlobenil (229), compound of formula I+ortho-dichlorobenzene (CAS RN 95-50-1), compound of formula I+para-dichlorobenzene (CAS RN 106-46-7), compound of formula I+dichlorprop (234), compound of formula I+dichlorprop-P (235), compound of formula I+diclofop-methyl (238), compound of formula I+diclosulam (241), compound of formula I+difenzoquat metilsulfate (248), compound of formula I+diflufenican (251), compound of formula I+diflufenzopyr (252), compound of formula I+dimefuron (256), compound of formula I+dimepiperate (257), compound of formula I+dimethachlor (258), compound of formula I+dimethenamid (260), compound of formula I+dimethenamid-P, compound of formula I+dimethipin (261), compound of formula I+dimethylarsinic acid (264), compound of formula I+dinitramine (268), compound of formula I+dinoterb (272), compound of formula I+diphenamid (274), compound of formula I+dipropetryn (CAS RN 4147-51-7), compound of formula I+diquat dibromide (276), compound of formula I+dithiopyr (280), compound of formula I+diuron (281), compound of formula I+DNOC (282), compound of formula I+3,4-DP(CAS RN 3307-41-3), compound of formula I+DSMA (CAS RN 144-21-8), compound of formula I+EBEP, compound of formula I+endothal (295), compound of formula I+EPTC (299), compound of formula I+esprocarb (303), compound of formula I+ethalfluralin (305), compound of formula I+ethephon (307), compound of formula I+ethofumesate (311), compound of formula I+ethoxyfen (CAS RN 188634-90-4), compound of formula I+ethoxyfen-ethyl (CAS RN 131086-42-5), compound of formula I+ethoxysulfuron (314), compound of formula I+etobenzanid (318), compound of formula I+fenoxaprop-P-ethyl (339), compound of formula I+fentrazamide (348), compound of formula I+ferrous sulfate (353), compound of formula I+flamprop, compound of formula I+flamprop-M (355), compound of formula I+florasulam (359), compound of formula I+fluazifop-butyl (361), compound of formula I+fluazifop-P-butyl (362), compound of formula I+fluazolate (isopropazol) (CAS RN 174514-07-9), compound of formula I+flucarbazone-sodium (364), compound of formula I+flucetosulfuron (CAS RN 412928-75-7), compound of formula I+fluchloralin (365), compound of formula I+flufenacet (BAY FOE 5043) (369), compound of formula I+flufenpyr-ethyl (371), compound of formula I+flumetralin (373), compound of formula I+flumiclorac-pentyl (375), compound of formula I+flumipropyn (flumipropin) (CAS RN 84478-52-4), compound of formula I+fluometuron (378), compound of formula I+fluoroglycofen-ethyl (380), compound of formula I+flupoxam (CAS RN 119126-15-7), compound of formula I+flupropacil (CAS RN 120890-70-2), compound of formula I+flupropanate (383), compound of formula I+flupyrsulfuron-methyl-sodium (384), compound of formula I+flurenol (387), compound of formula I+fluridone (388), compound of formula I+fluorochloridone (389), compound of formula I+fluoroxypyr (390), compound of formula I+flurtamone (392), compound of formula I+fluthiacet-methyl (395), compound of formula I+fosamine (406), compound of formula I+halosulfuron-methyl (426), compound of formula I+haloxyfop (427), compound of formula I+haloxyfop-P (428), compound of formula I+HC-252 (429), compound of formula I+hexazinone (440), compound of formula I+imazaquin (454), compound of formula I+imazosulfuron (456), compound of formula I+indanofan (462), compound of formula I+iodomethane (CAS RN 74-88-4), compound of formula I+ioxynil (467), compound of formula I+isoproturon (475), compound of formula I+isouron (476), compound of formula I+isoxaben (477), compound of formula I+isoxachlortole (CAS RN 141112-06-3), compound of formula I+isoxapyrifop (CAS RN 87757-18-4), compound of formula I+karbutilate (482), compound of formula I+lactofen (486), compound of formula I+lenacil (487), compound of formula I+linuron (489), compound of formula I+MAA (CAS RN 124-58-3), compound of formula I+MAMA (CAS RN 2321-53-1), compound of formula I+MCPA (499), compound of formula I+MCPA-thioethyl (500), compound of formula I+MCPB (501), compound of formula I+mecoprop (503), compound of formula I+mecoprop-P (504), compound of formula I+mefenacet (505), compound of formula I+mefluidide (507), compound of formula I+mesosulfuron-methyl (514), compound of formula I+metam (519), compound of formula I+metamifop (mefluoxafop) (520), compound of formula I+methabenzthiazuron (526), compound of formula I+methazole (CAS RN 20354-26-1), compound of formula I+methylarsonic acid (536), compound of formula I+methyldymron (539), compound of formula I+methyl isothiocyanate (543), compound of formula I+metobenzuron (547), compound of formula I+metobromuron (CAS RN 3060-89-7), compound of formula I+metosulam (552), compound of formula I+metoxuron (553), compound of formula I+MK-616 (559), compound of formula I+molinate (560), compound of formula I+monolinuron (562), compound of formula I+MSMA (CAS RN 2163-80-6), compound of formula I+naproanilide (571), compound of formula I+napropamide (572), compound of formula I+naptalam (573), compound of formula I+neburon (574), compound of formula I+nipyraclofen (CAS RN 99662-11-0), compound of formula I+n-methyl-glyphosate, compound of formula I+nonanoic acid (583), compound of formula I+norflurazon (584), compound of formula I+oleic acid (fatty acids) (593), compound of formula I+orbencarb (595), compound of formula I+orthosulfamuron (CAS RN 213464-77-8), compound of formula I+oryzalin (597), compound of formula I+oxadiargyl (599), compound of formula I+oxadiazon (600), compound of formula I+oxaziclomefone (604), compound of formula I+oxyfluorfen (610), compound of formula I+pebulate (617), compound of formula I+penoxsulam (622), compound of formula I+pentachlorophenol (623), compound of formula I+pentanochlor (624), compound of formula I+pentoxazone (625), compound of formula I+pethoxamid (627), compound of formula I+petrolium oils (628), compound of formula I+phenmedipham (629), compound of formula I+picloram (645), compound of formula I+picolinafen (646), compound of formula I+piperophos (650), compound of formula I+potassium arsenite (CAS RN 10124-50-2), compound of formula I+potassium azide (CAS RN 20762-80-1), compound of formula I+pretilachlor (656), compound of formula I+prodiamine (661), compound of formula I+profluazol (CAS RN 190314-43-3), compound of formula I+profoxydim (663), compound of formula I+prohexadione calcium (664), compound of formula I+propachlor (667), compound of formula I+propanil (669), compound of formula I+propaquizafop (670), compound of formula I+propham (674), compound of formula I+propisochlor (667), compound of formula I+propoxycarbazone-sodium (procarbazone-sodium) (679), compound of formula I+propyzamide (681), compound of formula I+prosulfocarb (683), compound of formula I+pyraclonil (pyrazogyl) (CAS RN 158353-15-2), compound of formula I+pyraflufen-ethyl (691), compound of formula I+pyrasulfotole (CAS RN 365400-11-9), compound of formula I+pyrazolynate (692), compound of formula I+pyrazosulfuron-ethyl (694), compound of formula I+pyrazoxyfen (695), compound of formula I+pyribenzoxim (697), compound of formula I+pyributicarb (698), compound of formula I+pyridafol (CAS RN 40020-01-7), compound of formula I+pyridate (702), compound of formula I+pyriftalid (704), compound of formula I+pyriminobac-methyl (707), compound of formula I+pyrimisulfan (CAS RN 221205-90-9), compound of formula I+pyroxasulfone (CAS RN 447399-55-5), compound of formula I+pyroxsulam (triflosulam) (CAS RN 422556-08-9), compound of formula I+quinclorac (712), compound of formula I+quinmerac (713), compound of formula I+quinoclamine (714), compound of formula I+quizalofop (717), compound of formula I+quizalofop-P (718), compound of formula I+sequestrene, compound of formula I+sethoxydim (726), compound of formula I+siduron (727), compound of formula I+SMA (CAS RN 3926-62-3), compound of formula I+sodium arsenite (CAS RN 7784-46-5), compound of formula I+sodium azide (CAS RN 26628-22-8), compound of formula I+sodium chlorate (734), compound of formula I+sulfometuron-methyl (751), compound of formula I+sulfosate (CAS RN 81591-81-3), compound of formula I+sulfuric acid (755), compound of formula I+tar oils (758), compound of formula I+2,3,6-TBA (759), compound of formula I+TCA-sodium (760), compound of formula I+tebutam (CAS RN 35256-85-0), compound of formula I+tebuthiuron (765), compound of formula I+tepraloxydim (771), compound of formula I+terbacil (772), compound of formula I+tefuryltrione (CAS RN 473278-76-1), compound of formula I+thenylchlor (789), compound of formula I+thidiazimin (CAS RN 123249-43-4), compound of formula I+thiazafluoron (CAS RN 25366-23-8), compound of formula I+thiazopyr (793), compound of formula I+thiobencarb (797), compound of formula I+tiocarbazil (807), compound of formula I+tralkoxydim (811), compound of formula I+tri-allate (816), compound of formula I+triaziflam (S119), compound of formula I+tricamba (CAS RN 2307-49-5), compound of formula I+triclopyr (827), compound of formula I+triflusulfuron-methyl (837), compound of formula I+trihydroxytriazine (CAS RN 108-80-5), compound of formula I+trinexapac-ethyl (CAS RN 95266-40-3) and compound of formula I+tritosulfuron (843).

The mixing partners of the compound of formula I may also be in the form of esters or salts, as mentioned e.g. in The Pesticide Manual, 13^{th} Edition (BCPC), 2003. The reference to acifluorfen-sodium also applies to acifluorfen, and the reference to bensulfuron-methyl also applies to bensulfuron, etc.

The mixing ratio of the compound of formula I to the mixing partner is preferably from 1:100 to 1000:1.

The mixtures can advantageously be used in the above-mentioned formulations (in which case “active ingredient” relates to the respective mixture of compound of formula I with the mixing partner).

The compounds of formula I according to the invention can also be used in combination with one or more safeners. Likewise, mixtures of a compound of formula I according to the invention with one or more further herbicides can also be used in combination with one or more safeners. The safeners can be AD 67 (MON 4660) (11), benoxacor (63), cloquintocet-mexyl (163), cyometrinil and the corresponding (Z) isomer, cyprosulfamide (CAS RN 221667-31-8), dichlormid (231), fenchlorazole-ethyl (331), fenclorim (332), flurazole (386), furilazole (413) and the corresponding R isomer, isoxadifen-ethyl (478), mefenpyr-diethyl (506), oxabetrinil (598), naphthalic anhydride (CAS RN 81-84-5) and N-isopropyl-4-(2-methoxy-benzoylsulfamoyl)-benzamide (CAS RN 221668-34-4). Particularly preferred are mixtures of a compound of formula I with benoxacor (i.e. compound of formula I+benoxacor).

The safeners of the compound of formula I may also be in the form of esters or salts, as mentioned e.g. in The Pesticide Manual, 13^{th} Edition (BCPC), 2003. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048, and the reference to fenchlorazole-ethyl also applies to fenchlorazole, etc.

Preferably the mixing ratio of compound of formula I to safener is from 100:1 to 1:10, especially from 20:1 to 1:1.

The mixtures can advantageously be used in the above-mentioned formulations (in which case “active ingredient” relates to the respective mixture of compound of formula I with the safener).

Preferred mixtures of a compound of formula I with further herbicides and safeners include:

Mixtures of a compound of formula I with a triazine and a safener.

Mixtures of a compound of formula I with glyphosate and a safener.

Mixtures of a compound of formula I with glufosinate and a safener.

Mixtures of a compound of formula I with isoxaflutole and a safener.

Mixtures of a compound of formula I with isoxaflutole and a triazine and a safener.

Mixtures of a compound of formula I with isoxaflutole and glyphosate and a safener.

Mixtures of a compound of formula I with isoxaflutole and glufosinate and a safener.

Mixtures of a compound of formula I with mesotrione and a safener.

Mixtures of a compound of formula I with mesotrione and a triazine and a safener.

Mixtures of a compound of formula I with mesotrione and glyphosate and a safener.

Mixtures of a compound of formula I with mesotrione and glufosinate and a safener.

Mixtures of a compound of formula I with sulcotrione and a safener.

Mixtures of a compound of formula I with sulcotrione and a triazine and a safener.

Mixtures of a compound of formula I with sulcotrione and glyphosate and a safener.

Mixtures of a compound of formula with sulcotrione and glufosinate and a safener.

The following Examples further illustrate, but do not limit, the invention.

### PREPARATION EXAMPLES

### 1) Methods for making 4-halo-5,5-dimethyl-4,5-dihydro-isoxazole derivatives

### Example I1

### Preparation of 4-bromo-3-methanesulfanyl-5,5-dimethyl-4,5-dihydro-isoxazole

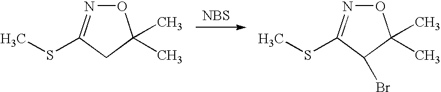

5,5-Dimethyl-3-methylsulfanyl-4,5-dihydro-isoxazole (3.8 g, 26.2 mmol) (prepared as described in e.g. US 2004/110749 and US 2004/259734) was dissolved in carbon tetrachloride (84 ml). N-bromo succinimide (5.82 g, 32.6 mmol) was added and the mixture was then irradiated under a 500W tungsten bulb. After 2 hours the reaction mixture was cooled to room temperature, filtered and concentrated. The residue was purified by column chromatography on silica gel (eluent: hexane/ethyl acetate) to give 4-bromo-3-methanesulfanyl-5,5-dimethyl-4,5-dihydro-isoxazole (4.074 g, 70% yield).

¹H-NMR (400 MHz, CDCl₃): 1.40 (s, 3H, Me), 1.63 (s, 3H, Me), 2.57 (s, 3H, Me), 4.7 (s, 1H, CH) ppm.

### Example I2

### Preparation of 4-bromo-3-methanesulfonyl-5,5-dimethyl-4,5-dihydro-isoxazole

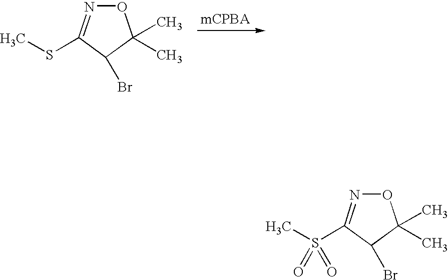

4-Bromo-5,5-dimethyl-3-methylsulfanyl-4,5-dihydro-isoxazole (2.415 g, 10.8 mmol) (Example I1) was dissolved in dichloromethane (120 ml) and 3-chloroperoxy-benzoic acid (mCPBA) (6.85 g, 23.8 mmol) was added. The reaction mixture was stirred at room temperature for 16 hours. The reaction mixture was quenched by addition of aqueous sodium metabisulfite (10%) and extracted with dichloromethane. The organic extract was washed with sodium hydroxide, dried over magnesium sulfate and concentrated. The residue was purified by column chromatography on silica gel (eluent: hexane/ethyl acetate) to give 4-bromo-3-methanesulfonyl-5,5-dimethyl-4,5-dihydro-isoxazole (2.122 g, 77% yield).

¹H-NMR (400 MHz, CDCl₃): 1.49 (s, 3H, Me), 1.75 (s, 3H, Me), 3.34 (s, 3H, Me), 5.12 (s, 1H, CH) ppm.

### Example I3

### Preparation of 4-fluoro-3-methanesulfonyl-5,5-dimethyl-4,5-dihydro-isoxazole

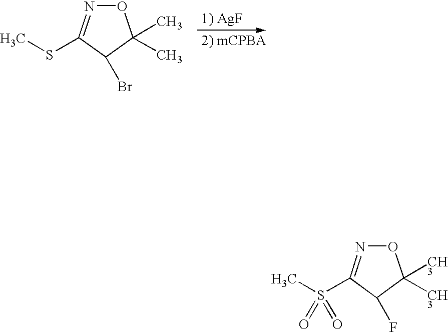

4-Bromo-5,5-dimethyl-3-methylsulfanyl-4,5-dihydro-isoxazole (0.55 g, 2.24 mmol) (Example I1) was dissolved in acetonitrile (20 ml). Water (0.5 ml) and silver fluoride (1.25 g, 11.2 mmol) were added and the reaction mixture was heated to 90° C. in the dark for 5 hours. After cooling the reaction mixture was filtered through a silica plug, the plug was washed with dichloromethane (120 ml). 3-Chloroperoxybenzoic acid (mCPBA) (1.562 g, 5.43 mmol) was added and the mixture was stored at room temperature for 16 hours. The reaction mixture was quenched by addition of aqueous sodium metabisulfite (10%) and extracted with dichloromethane. The organic extract was washed with aqueous sodium hydroxide (1N), dried over magnesium sulfate and concentrated. The residue was purified by column chromatography on silica gel (eluent: hexane/ethyl acetate) to give 4-fluoro-3-methanesulfonyl-5,5-dimethyl-4,5-dihydro-isoxazole (0.322 g, 67% yield over 2 steps).

¹H-NMR (400 MHz, CDCl₃): 1.40 (s, 3H, Me), 1.61 (d, 3H, Me), 3.28 (s, 3H, Me), 5.57 (d, 1H, CH) ppm.

### Example I4

### Preparation of 5,5-dimethyl-3-phenylsulfanyl-4,5-dihydro-isoxazole

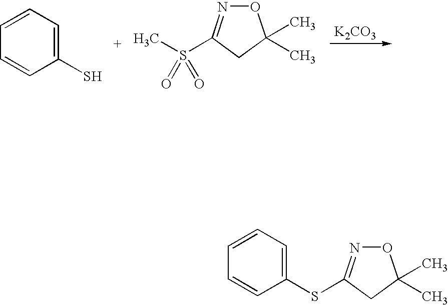

3-Methanesulfonyl-5,5-dimethyl-4,5-dihydro-isoxazole (8.05 g, 45.5 mmol) (prepared as described in e.g. US 2004/110749 and US 2004/259734), thiophenol (5.0 g, 45.5 mmol) and potassium carbonate (9.44 g, 68.3 mmol) were stirred in ethanol (150 ml) and heated to reflux for 2 hours. The reaction mixture was concentrated and the residue partitioned between dichloromethane and water. The organic extract was dried over magnesium sulfate and concentrated. The residue was purified by column chromatography on silica gel (eluent: hexane/ethyl acetate) to give 5,5-dimethyl-3-phenyl-sulfanyl-4,5-dihydro-isoxazole (8.355 g, 89% yield).

¹H-NMR (400 MHz, CDCl₃): 1.4 (s, 6H, Me), 2.71 (s, 2H, CH₂), 7.4 (m, 3H, CH), 7.55 (m, 2H, CH) ppm.

### Example I5

### Preparation of 3-benzenesulfonyl-4-chloro-5,5-dimethyl-4,5-dihydro-isoxazole

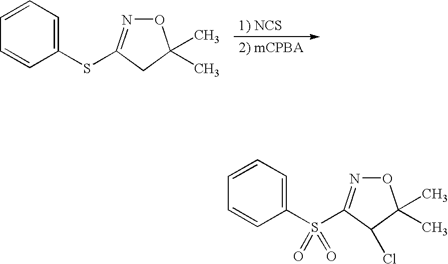

5,5-Dimethyl-3-phenylsulfanyl-4,5-dihydro-isoxazole (2.712 g, 13.1 mmol) (Example I4) was dissolved in carbon tetrachloride (42 ml). N-chloro succinimide (NCS) (2.176 g, 16.3 mmol) was added and the mixture was irradiated under a 500W tungsten bulb. After 4 hours the reaction mixture was cooled and filtered before the solvent was removed. The residue was dissolved in dichloromethane (120 ml) before 3-chloro-peroxybenzoic acid (mCPBA) (8.285 g, 28.8 mmol) was added. The reaction mixture was stirred at room temperature for 16 hours. The reaction mixture was quenched by addition of aqueous sodium metabisulfite (10%) and extracted with dichloromethane. The organic extract was washed with aqueous sodium hydroxide (1N), dried over magnesium sulfate and concentrated. The residue was purified by column chromatography on silica gel (eluent: hexane/ethyl acetate) to give 3-benzenesulfonyl-4-chloro-5,5-dimethyl-4,5-dihydro-isoxazole (0.335 g, 9% yield).

¹H-NMR (400 MHz, CDCl₃): 1.41 (s, 3H, Me), 1.6 (s, 3H, Me), 5.06 (s, 1H, CH), 7.62 (m, 2H, CH), 7.73 (m, 1H, CH), 8.1 (m, 2H, CH) ppm.

### Example I6

### Preparation of 3-benzenesulfonyl-5,5-dimethyl-4,5-dihydro-isoxazole

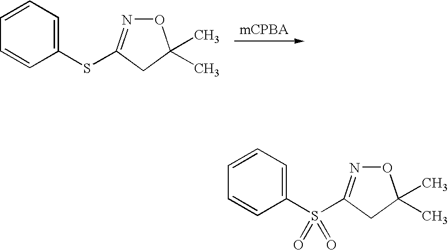

3-Chloroperoxybenzoic acid (10 g, 25 mmol) was added in portions over 1 hour to a solution of 5,5-dimethyl-3-phenylsulfany-4,5-dihydro-isoxazole (3.1 g, 15 mmol) (Example I4) in dry dichloromethane (100 ml) at 0° C. The mixture was stored at room temperature for 16 hours. The reaction mixture was quenched by addition of aqueous sodium metabisulfite (10%) (50 ml). The phases were separated and the organic phase was washed three times with aqueous sodium hydroxide (1M) and once with brine. The organic phase was dried over magnesium sulfate and concentrated to give 3-benzenesulfonyl-5,5-dimethyl-4,5-dihydro-isoxazole as a colourless oil (3.1 g, 87% yield), which was used without further purification.

¹H-NMR (400 MHz, CDCl₃): 1.45 (s, 6H, Me), 3.08 (s, 2H, CH₂), 7.6 (m, 2H, CH), 7.79 (m, 1H, CH), 8.02 (d, 2H, CH) ppm.

### Example I7

### Preparation of 3-benzenesulfonyl-4,4-dichloro-5,5-dimethyl-4,5-dihydro-isoxazole

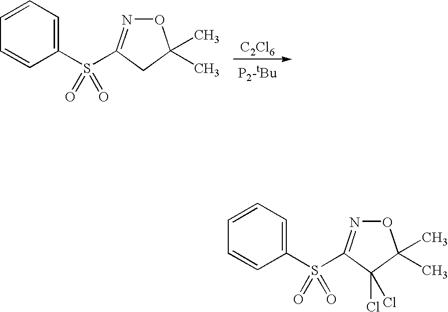

3-Benzenesulfonyl-5,5-dimethyl-4,5-dihydro-isoxazole (0.871 g, 3.64 nmol) (Example I6) was dissolved in tetrahydrofuran (50 ml) and hexachloroethane (0.949 g, 4.0 mmol) was added followed by 1-tert-butyl-2,2,4,4,4-pentakis(dimethylamino-2-lambda⁵-5,4-lambda⁵-5-catenadi(phosphazene) (P₂-tBu) (2M in THF) (2.0 ml, 4.0 mmol). After 2 hours the reaction mixture was concentrated. The residue was purified by column chromatography on silica gel (eluent: hexane/ethyl acetate) to give 3-benzenesulfonyl-4,4-dichloro-5,5-dimethyl-4,5-dihydro-isoxazole (0.541 g, 48% yield).

¹H-NMR (400 MHz, CDCl₃): 1.58 (s, 6H, Me), 7.63 (m, 2H, CH), 7.76 (m, 1H, CH), 8.12 (m, 2H, CH) ppm.

### Example I8

### Preparation of 3-benzenesulfonyl-4-fluoro-5,5-dimethyl-4,5-dihydro-isoxazole

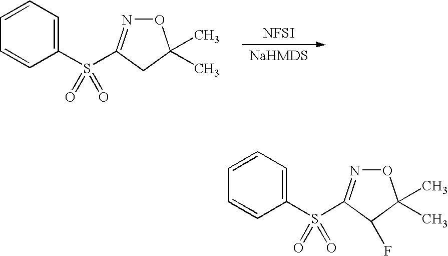

3-Benzenesulfonyl-5,5-dimethyl-4,5-dihydro-isoxazole (1.434 g, 6 mmol) (Example I6) was dissolved in dry tetrahydrofuran (50 ml) under nitrogen and the solution stirred and cooled to −70° C. N-Fluorodibenzenesulfonimide (2.24 g, 6 mmol) was then added in portions over 10 minutes to the cooled reaction mixture. Sodium hexamethyldisilazide (9 ml, 9 mmol) (1M in THF) was added dropwise to the reaction mixture at −70° C. to give a yellow solution. The reaction mixture was left in the freezer for 16 hours. The cold mixture was poured onto saturated aqueous ammonium chloride and extracted twice with ethyl acetate. The combined organic phases were washed with water and brine, dried over magnesium sulfate and concentrated to give a sticky yellow solid. The solid was recrystallised from isopropyl alcohol to give 3-benzenesulfonyl-4-fluoro-5,5-dimethyl-4,5-dihydro-isoxazole as a beige solid (0.65 g, yield 42%), which was used without further purification.

¹H-NMR (400 MHz, CDCl₃): 1.37 (s, 3H, Me), 1.5 (s, 3H, Me), 5.54 (d, 1H, CH), 7.62 (t, 2H, CH), 7.73 (t, 1H, CH), 8.08 (d, 2H, CH) ppm.

### 2) Methods for making Pyrazole Derivatives

The synthesis of 4-(bromomethyl)-5-(difluoromethoxy)-1-methyl-3-(trifluoro-methyl)-1H-pyrazole was described in WO 04/013106.

The synthesis of 4-(bromomethyl)-5-fluoro-1-methyl-3-trifluoromethyl-1H-pyrazole was described in US 2004/0110749.

The synthesis of 4-bromomethyl-3-difluoromethoxy-1-methyl-5-trifluoromethyl-1H-pyrazole was described in WO 06/024820.

The synthesis of [1,3-dimethyl-5-(2,2,2-trifluoro-ethoxy)-1H-pyrazol-4-yl]-methanol was described in WO 06/024820.

1-Methyl-5-(E)-propenyl-3-trifluoromethyl-1H-pyrazole-4-carbaldehyde is commercially available.

1-Methyl-5-(E)-propenyl-3-trifluoromethyl-1H-pyrazole-4-carbaldehyde was reduced as described in Example I10 to give [1-methyl-5-(E)-propenyl-3-trifluoromethyl-1H-pyrazol-4-yl]-methanol.

¹H-NMR (400 MHz, CDCl₃): 2.0 (dd, 3H, Me), 3.95 (s, 3H, Me), 4.6 (s, 2H, CH₂), 6.3 (m, 2H, CH) ppm.

[1-Methyl-5-(E)-propenyl-3-trifluoromethyl-1H-pyrazol-4-yl]-methanol was brominated as described in Example I17 to give 4-bromomethyl-1-methyl-5-(E)-propenyl-3-trifluoromethyl-1H-pyrazole.

¹H-NMR (400 MHz, CDCl₃): 2.02 (d, 3H, Me), 3.85 (s, 3H, Me), 4.5 (s, 2H, CH₂), 6.36 (m, 2H, CH) ppm.

### Example I9

### Preparation of 1-methyl-5-(2,2,2-trifluoro-ethoxy)-3-trifluoromethyl-1H-pyrazole-4-carbaldehyde

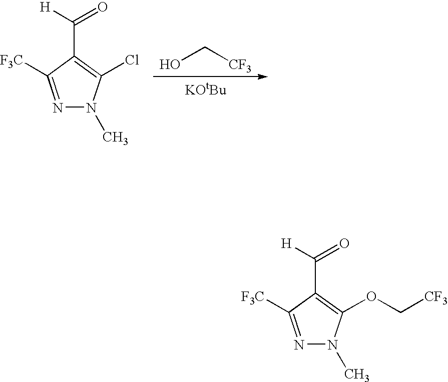

2,2,2-Trifluoroethanol (12.1 ml, 0.17 mol) was added dropwise to a solution of potassium tert-butoxide (1M in THF) (170 ml, 0.17 mol) in dry tetrahydrofuran (80 ml) at 10° C. Then 5-chloro-1-methyl-3-trifluoromethyl-1H-pyrazole-4-carbaldehyde (30 g, 0.14 mol) (prepared according to WO 04/014138) in tetrahydrofuran (40 ml) was added dropwise at 10-15° C. over 1 hour. At the end of the addition, the mixture was stirred at room temperature for one hour, then water (200 ml) and ethyl acetate (200 ml) were added. The phases were separated and the aqueous phase extracted three times with ethyl acetate. The combined organic extracts were washed with brine, dried over magnesium sulfate and concentrated to give 1-methyl-5-(2,2,2-trifluoro-ethoxy)-3-trifluoro-methyl-1H-pyrazole-4-carbaldehyde (35.9 g, 92% yield).

¹H-NMR (400 MHz, CDCl₃): 3.8 (s, 3H, Me), 4.9-5.0 (q, 2H, CH₂), 9.85 (s, 1H, CH) ppm.

### Example I10

### Preparation of [1-methyl-5-(2,2,2-trifluoro-ethoxy)-3-trifluoromethyl-1H-pyrazol-4-yl]-methanol

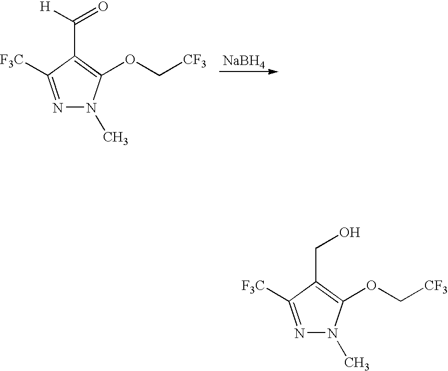

Sodium borohydride (2.95 g, 78 mmol) was added in portions to a solution of 1-methyl-5-(2,2,2-trifluoro-ethoxy)-3-trifluoromethyl-1H-pyrazole-4-carbaldehyde (21.5 g, 78 mmol) (Example I9) in methanol (200 ml) at 0° C. The solution was stirred at 8-15° C. for 2 hours, then concentrated and the residue partitioned between dichloromethane and water. The organic phase was washed with saturated aqueous sodium hydrogencarbonate and brine, dried over magnesium sulfate and concentrated to give [1-methyl-5-(2,2,2-trifluoro-ethoxy)-3-trifluoromethyl-1H-pyrazol-4-yl]-methanol as a white solid (20.5 g, 94% yield).

¹H-NMR (400 MHz, CDCl₃): 3.8 (s, 3H, Me), 4.5 (s, 2H, CH₂), 4.75 (q, 2H, CH₂) ppm.

### Example I11

### Preparation of 4-bromomethyl-1-methyl-5-(2,2,2-trifluoro-ethoxy)-3-trifluoromethyl-1H-pyrazole

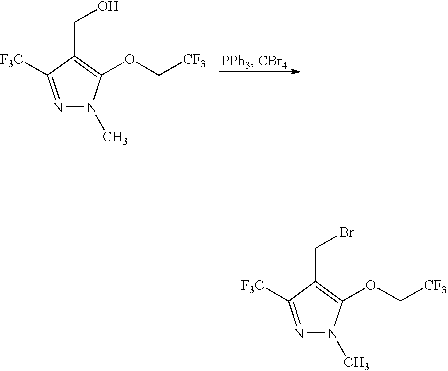

[1-Methyl-5-(2,2,2-trifluoro-ethoxy)-3-trifluoromethyl-1H-pyrazol-4-yl]-methanol (20.2 g, 73 mmol) (Example I10) was dissolved in dichloromethane (200 ml) and cooled to 0° C. before triphenyl phosphine (20.9 g, 80 mmol) and carbon tetrabromide (23.2 g, 70 mmol) were added. The mixture was stirred for 2 hours and then concentrated. The residue was purified by column chromatography on silica gel (eluent: 10% ethyl acetate in hexane) to give 4-bromomethyl-1-methyl-5-(2,2,2-trifluoro-ethoxy)-3-trifluoromethyl-1H-pyrazole as a yellow oil (21.53 g, 87% yield) which solidified partially on refrigeration.

¹H-NMR (400 MHz, CDCl₃): 3.75 (s, 3H, Me), 4.40 (s, 2H, CH₂), 4.68 (q, 2H, CH₂) ppm.

The following compounds were also prepared according to the methods in Example I9, Example I10 and Example I1

4-Bromomethyl-1-methyl-3-trifluoromethyl-5-(2,2,2-trifluoro-1-methyl-ethoxy)-1H-pyrazole was prepared using 1,1,1-trifluoro-propan-2-ol as reagent in Example I9.

4-Bromomethyl-5-(2-fluoro-1-methyl-ethoxy)-1-methyl-3-tri fluoromethyl-1H-pyrazole was prepared using 1-fluoro-propan-2-ol as reagent in Example I9.

4-Bromomethyl-5-(2-methoxy-ethoxy)-1-methyl-3-trifluoromethyl-1H-pyrazole was prepared using 2-methoxy-ethanol as reagent in Example I9.

### Example I12

### Alternative preparation of 4-bromomethyl-1-methyl-5-(2,2,2-trifluoro-ethoxy)-3-trifluoromethyl-1H-pyrazole

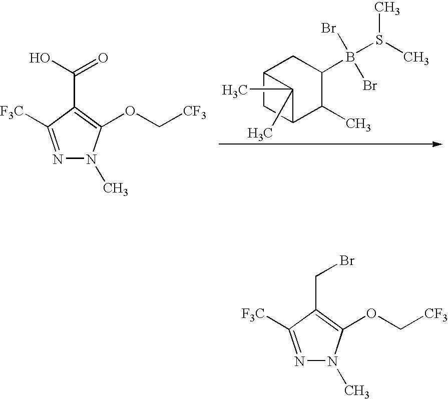

A solution of isopinocampheyl-boron dibromide dimethylsulfide complex (4.4 g, 12 mmol) (prepared according to J. Org. Chem. 1980 (45) 384-389) in dichloromethane (10 ml) was added over a period of 10 minutes to a solution of 1-methyl-5-(2,2,2-tri-fluoro-ethoxy)-3-trifluoromethyl-1H-pyrazole-4-carbaldehyde (3.0 g, 10.8 mmol) (Example I9) in dry hexane (15 ml). The reaction mixture was stirred at room temperature for 3 hours. The solid was removed by filtration and washed with hexane/dichloromethane (ratio 8:2, 2×10 ml). The combined organic phases were diluted with diethyl ether (50 ml), washed twice with water, then with brine, dried over magnesium sulfate and concentrated. The residue was purified by column chromatography on silica gel (eluent: 0-50% ethyl acetate in hexane) to give 4-bromomethyl-1-methyl-5-(2,2,2-trifluoroethoxy)-3-trifluoromethyl-1H-pyrazole as a colourless oil (3.05 g, 83% yield) which solidified on standing.

### Example I13

### Preparation of 4-chloromethyl-1-methyl-5-(2,2,2-trifluoro-ethoxy)-3-trifluoromethyl-1H-pyrazole

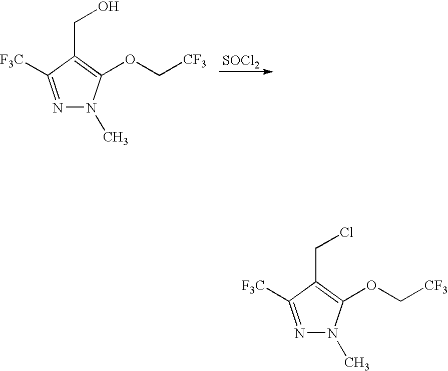

To a solution of [1-methyl-5-(2,2,2-trifluoro-ethoxy)-3-trifluoromethyl-1H-pyrazol-4-yl]-methanol (50 g, 0.14 mol) (Example I10) in dichloromethane (300 ml), was added thionyl chloride (17 ml, 0.17 mol) The mixture was stirred for 2 hours at room temperature before being concentrated. Twice the residue was taken up in toluene and was concentrated again to remove excess of thionyl chloride to give 4-chloromethyl-1-methyl-5-(2,2,2-trifluoro-ethoxy)-3-trifluoromethyl-1H-pyrazole (53.5 g), which was used without further purification.

### Example I14

### Preparation of 1,4-dimethyl-3-(2,2,2-trifluoro-ethoxy)-5-trifluoromethyl-1H-pyrazole

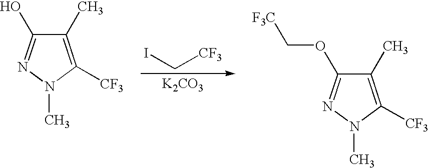

To a solution of 1,4-dimethyl-5-trifluoromethyl-1H-pyrazol-3-ol (3.0 g, 16.67 mmol) (prepared according to EP 370990) in N,N-dimethylformamide (90 ml), was added trifluoroethyl iodide (3.3 ml, 33.3 mmol) and potassium carbonate (4.6 g, 33.3 mmol). The mixture was stirred at room temperature for 3 days. More trifluoroethyl iodide (3.3 ml, 33.3 mmol) was added and the mixture stirred at room temperature for 2 days. More trifluoroethyl iodide (10 ml, 100 mmol) was added and the mixture stirred at room temperature for 2 days. The reaction mixture was quenched by addition of water and extracted with ethyl acetate. The organic extract was washed with brine, dried over magnesium sulfate and concentrated to give 1,4-dimethyl-3-(2,2,2-trifluoro-ethoxy)-5-trifluoromethyl-1H-pyrazole (809 mg, 16% yield).

¹H-NMR (400 MHz, CDCl₃): 2.03 (s, 3H, Me), 3.79 (s, 3H, Me), 4.56 (q, 2H, CH₂) ppm.

### Example I15

### Preparation of 4-bromomethyl-1-methyl-3-(2,2,2-trifluoro-ethoxy-5-trifluoromethyl-1H-pyrazole

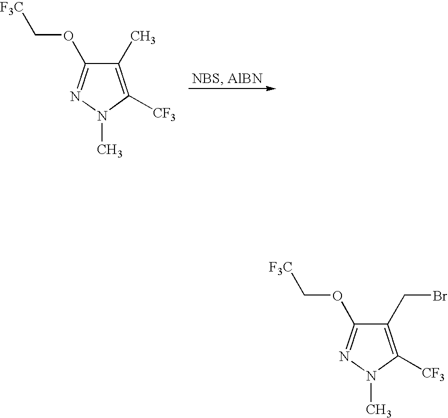

To a solution of 1,4-dimethyl-3-(2,2,2-trifluoro-ethoxy)-5-trifluoromethyl-1H-pyrazole (809 mg, 3.08 mmol) (Example I14) in carbon tetrachloride (10 ml), were added N-bromosuccinimide (NBS) (712 mg, 4.0 mmol) and azobisisobutyronitrile (AIBN) (50 mg, 3.08 mmol) under nitrogen. The reaction mixture was stirred at room temperature and irradiated with a UV lamp causing the reaction mixture to reflux in the heat of the lamp. After 30 minutes the reaction mixture was filtered and the solid was washed with dichloromethane. The combined filtrates were concentrated. The residue was triturated with 4: I hexane/ethyl acetate (50 ml) and the solid gained in this fashion was purified by column chromatography on silica gel (eluent: 20%-50% ethyl acetate in hexane) to give 4-bromomethyl-1-methyl-3-(2,2,2-trifluoro-ethoxy)-5-trifluoromethyl-1H-pyrazole (715 mg, 68% yield).

¹H-NMR (400 MHz, CDCl₃): 3.83 (s, 3H, Me), 4.39 (s, 2H, CH₂), 4.63 (q, 2H, CH₂) ppm.

### Example I16

### Preparation of 1-ethyl-3-trifluoromethyl-1H-pyrazole-4-carboxylic acid ethyl ester and 2-ethyl-3-trifluoromethyl-2H-pyrazole-4-carboxylic acid ethyl ester

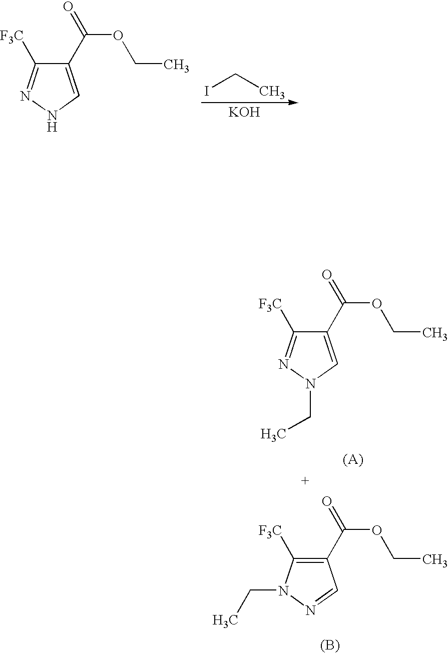

3-Trifluoromethyl-1H-pyrazole-4-carboxylic acid ethyl ester (3.0 g, 0.014 mol) was dissolved in ethanol (30 ml), and powdered potassium hydroxide (1.012 g, 0.018 mol) was added, followed by ethyl iodide (1.44 ml, 0.018 mol), dropwise via syringe. The pale yellow solution was heated to 75° C. and stirred for 1.5 hours. The reaction mixture was diluted with dichloromethane (12 ml) and filtered. The filtrate was concentrated to give a white solid which was purified by column chromatography on silica gel (eluent: 0-20% ethyl acetate in hexane) to give 2-ethyl-3-trifluoromethyl-2H-pyrazole-4-carboxylic acid ethyl ester (isomer B) (257 mg, 8% yield) as a clear oil and 1-ethyl-3-trifluoromethyl-1H-pyrazole-4-carboxylic acid ethyl ester (isomer A) (2.075 g, 61% yield) as a white solid.

### Isomer A ¹H-NMR (400 MHz, CDCl₃): 1.37 (t, 3H, Me), 1.55 (t, 3H, Me), 4.24 (q, 2H, CH₂), 4.32 (q, 2H, CH₂), 8.0 (s, 1H, CH) ppm.

### Isomer B ¹H-NMR (400 MHz, CDCl₃): 1.4 (t, 3H, Me), 1.5 (t, 3H, Me), 4.34 (q, 2H, CH₂), 4.4 (q, 2H, CH₂), 7.94 (s, 1H, CH) ppm.

1-Ethyl-3-trifluoromethyl-1H-pyrazole-4-carboxylic acid ethyl ester and 2-ethyl-3-trifluoromethyl-2H-pyrazole-4-carboxylic acid ethyl ester were reduced to the corresponding alcohols as described in Example I21.

### Example I17

### Preparation of 4-bromomethyl-1-ethyl-3-trifluoromethyl-1H-pyrazole

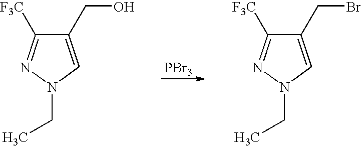

[1-Ethyl-3-trifluoromethyl-1H-pyrazol-4-yl]-methanol (691 mg, 3.56 mmol) was dissolved in diethyl ether (8 ml) and stirred under nitrogen. To this was added phosphorus tribromide (0.33 ml, 3.56 mmol). The reaction mixture was stirred for 5 hours at room temperature and stored at room temperature for 16 hours. The reaction mixture was quenched by addition of cold water and the mixture extracted with ethyl acetate twice, The combined organic extracts were dried over magnesium sulfate and concentrated. The residue was purified by column chromatography on silica gel (eluent: 0-20% ethyl acetate in hexane) to give 4-bromomethyl-1-ethyl-3-trifluoromethyl-1H-pyrazole as a clear oil (787 mg, 86% yield).

¹H-NMR (400 MHz, CDCl₃): 1.52 (t, 3H, Me), 4.19 (q, 2H, CH₂), 4.48 (s, 2H, CH₂), 7.55 (s, 1H, CH).

### Example I18

### Preparation of 1-allyl-3-trifluoromethyl-1H-pyrazole-4-carboxylic acid ethyl ester and 2-allyl-3-trifluoromethyl-H-2pyrazole-4-carboxylic acid ethyl ester

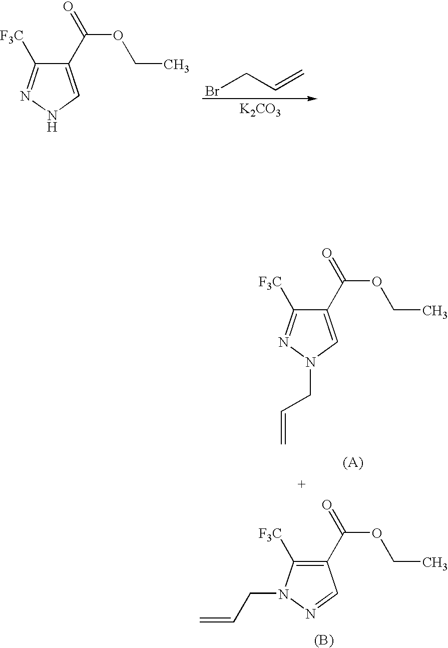

3-Trifluoromethyl-1H-pyrazole-4-carboxylic acid ethyl ester (500 mg, 2.4 mmol) was dissolved in acetone and stirred. To the solution was added potassium carbonate (498 mg, 3.6 mmol), in one portion, followed by dropwise addition of allyl bromide (0.31 ml, 3.6 mmol). The reaction mixture was stirred at room temperature for 5 hours. The reaction mixture was poured into water and extracted twice with ethyl acetate. The combined organic extracts were dried over magnesium sulfate and concentrated to give a 9:1 mixture of 1-allyl-3-trifluoromethyl-1H-pyrazole-4-carboxylic acid ethyl ester (isomer A) and 2-allyl-3-trifluoromethyl-2H-pyrazole-4-carboxylic acid ethyl ester (isomer B) (557 mg, 93% yield) as a yellow solid. Isomer A (major isomer) ¹H-NMR (400 MHz, CDCl₃): 1.39 (t, 3H, Me), 4.35 (q, 2H, CH₂), 4.8 (d, 2H, CH₂), 5.3-5.45 (dd, 2H, CH₂), 6.05 (m, 1H, CH), 8.01 (s, 1H, CH) ppm. Isomer B (minor isomer) ¹H-NMR (400 MHz, CDCl₃): 1.39 (t, 3H, Me), 4.35 (q, 2H, CH₁), 4.95 (d, 2H, CH₂), 5.1-5.3 (dd, 2H, CH₂), 6.05 (m, 1H, CH), 7.98 (s, 1H, CH) ppm.

The same method was used with propargyl bromide as reagent to give 1-propargyl-3-trifluoromethyl-1H-pyrazole-4-carboxylic acid ethyl ester.

The same method was used with cyclobutylmethyl bromide as reagent to give 1-cyclobutylmethyl-3-trifluoromethyl-1H-pyrazole-4-carboxylic acid ethyl ester.

The carboxylic acid ethyl esters were reduced to the corresponding alcohols as described in Example 121 and brominated as described in Example I17.

### Example I19

### Preparation of 1-(prop-2-yl)-3-trifluoromethyl-1H-pyrazole-4-carboxylic acid ethyl ester and 2-(prop-2-yl)-3-trifluoromethyl-2H-pyrazole-4-carboxylic acid ethyl ester

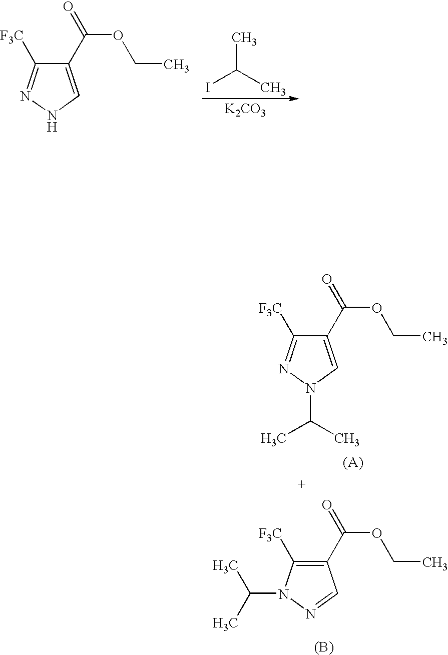

3-Trifluoromethyl-1H-pyrazole-4-carboxylic acid ethyl ester (2.5 g, 1.2 mmol) was dissolved in acetonitrile (25 ml) and stirred at room temperature. To the solution was added 2-iodopropane (1.8 ml, 1.8 mmol), followed by potassium carbonate (2.488 g, 1.8 mmol). The reaction mixture was heated to reflux and stirred for 6.5 hours. The reaction mixture was stored at room temperature for 16 hours. The reaction mixture was poured into water and extracted three times with ethyl acetate. The combined organic extracts were dried over magnesium sulfate and concentrated to give 1-(prop-2-yl)-3-trifluoromethyl-1H-pyrazole-4-carboxylic acid ethyl ester (isomer A) and 2-(prop-2-yl)-3-trifluoromethyl-2H-pyrazole-4-carboxylic acid ethyl ester (isomer B) (9:1 mixture) as a yellow oil which was purified by column chromatography on silica gel (eluent: 0-10% ethyl acetate in hexane). Isomer A was obtained as a white solid (1.724 g, 57% yield).

¹H-NMR (400 MHz, CDCl₃): 1.35 (t, 3H, Me), 1.55 (d, 6H, Me), 4.3 (q, 2H, CH₂), 4.55 (m, 1H, CH), 8.0 (s, 1H, CH) ppm.

1-(Prop-2-yl)-3-trifluoromethyl-1H-pyrazole-4-carboxylic acid ethyl ester was reduced to the corresponding alcohols as described in Example I21 and brominated as described in Example I17.

### Example I20

### Preparation of 1-methyl-4-trifluoromethyl-1H-pyrazole-3-carboxylic acid ethyl ester and 1-methyl-3-trifluoromethyl-1H-pyrazole-4-carboxylic acid ethyl ester

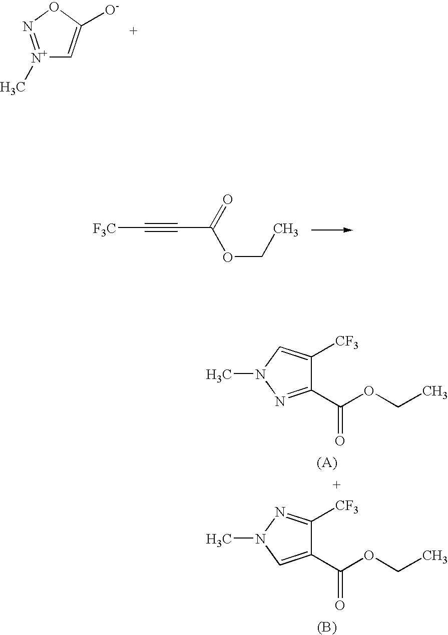

5-Hydroxy-3-methyl-[1,2,3]-oxadiazolium (3-methylsydnone) (prepared according to J. Heterocycl. Chem. (1996) 33, 719) (25.7 g, 256 mmol) was suspended in xylene (120 g) and heated to 100° C. 4,4,4-Trifluoro-but-2-ynoic acid ethyl ester (prepared according to Organic Syntheses (1992) 70, 246-255) (44.8 g, 270 mmol) was slowly added dropwise. The mixture was stirred at 100° C. for 4 hours and then concentrated. Ethyl acetate (10 ml) was added to the oil which caused the product to crystallise. The crystalline product was washed with 1:1 ethyl acetate/hexane (50 ml), then hexane (50 ml) and dried to give 1-methyl-4-trifluoromethyl-1H-pyrazole-3-carboxylic acid ethyl ester (isomer A) as white crystals (20.9 g, 36.6% yield). The mother liquor was concentrated and the residue purified by column chromatography on silica gel (eluent: ethyl acetate/cyclohexane) which gave more 1-methyl-4-trifluoromethyl-1H-pyrazole-3-carboxylic acid ethyl ester (isomer A) (14.3 g, 25.1% yield) and 1-methyl-3-trifluoromethyl-1H-pyrazole-4-carboxylic acid ethyl ester (isomer B) (10.0 g, 17.5% yield).

A H-NMR (400 MHz, CDCl₃): 1.41 (t, 3H, Me), 4.01 (s, 3H, Me), 4.44 (q, 2H, CH₂), 7.72 (s, 1H, CH) ppm.

### Example I21

### Preparation of (1-methyl-4-trifluoromethyl-1H-pyrazol-3-yl)-methanol

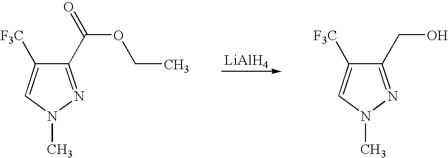

To a suspension of lithium aluminium hydride pellets (0.83 g, 21.9 mmol) in tetrahydrofuran (30 ml) under nitrogen at 0° C., was added slowly 1-methyl-4-trifluoro-methyl-1H-pyrazole-3-carboxylic acid ethyl ester (2.05 g, 8.75 mmol) dissolved in tetrahydrofuran (20 ml). The reaction mixture was stirred at room temperature for 3 hours. The reaction mixture was quenched by sequential addition of water (400 μl), aqueous sodium hydroxide (3M) (400 μl) and water (1.2 ml). The resulting sludge was filtered through celite and the solids washed with diethyl ether. The combined filtrates were concentrated to give (1-methyl-4-trifluoromethyl-1H-pyrazol-3-yl)-methanol (1.595 g, 100% yield).

¹H-NMR (400 MHz, CDCl₃): 3.90 (3H, s, Me), 4.75 (2H, s, CH₂), 7.62 (1H, s, CH) ppm.

(1-Methyl-4-trifluoromethyl-1H-pyrazol-3-yl)-methanol was chlorinated as described in Example I13 to give 3-chloromethyl-1-methyl-4-trifluoromethyl-1H-pyrazol-3-yl.

¹H-NMR (400 MHz, CDCl₃): 3.92 (3H, s, Me), 4.63 (2H, s, CH₂), 7.63 (1H, s, CH) ppm.

The same method was used with 1-methyl-3-trifluoromethyl-1H-pyrazole-4-carboxylic acid ethyl ester as starting material (Example I20) to give (1-methyl-3-trifluoromethyl-1H-pyrazol-4-yl)-methanol.

(1-Methyl-3-trifluoromethyl-1H-pyrazol-4-yl)-methanol was chlorinated as described in Example I13 to give 4-chloromethyl-1-methyl-3-trifluoromethyl-1H-pyrazol-3-yl.

### Example 122

### Preparation of 1-difluoromethyl-3-trifluoromethyl-1H-pyrazole-4-carboxylic acid ethyl ester and 2-difluoromethyl-3-trifluoromethyl-2H-pyrazole-4-carboxylic acid ethyl ester

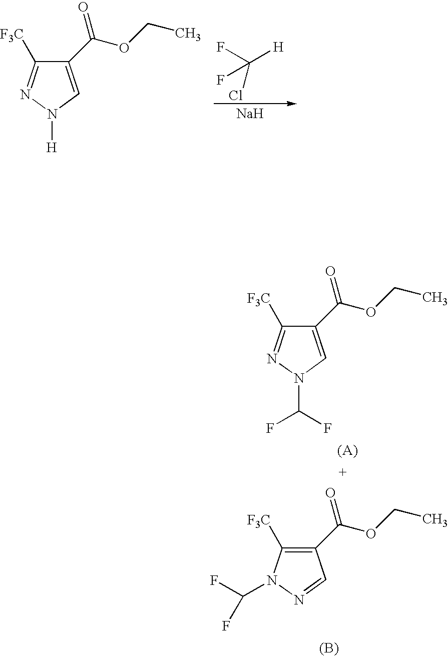

Sodium hydride (60% in mineral oil) (320 mg, 7.93 mmol) was washed with hexane (2×10 ml) and suspended in tetrahydrofuran (20 ml) in a three necked flask. 3-Trifluoromethyl-1H-pyrazole-4-carboxylic acid ethyl ester (1.5 g, 7.21 mmol) (prepared according to JP 2000/044541) in tetrahydrofuran (10 ml) was added dropwise and the solution stirred at room temperature for 30 minutes. Chlorodifluoromethane was bubbled through the solution for 5 minutes and then the mixture was stirred at room temperature for 3 hours, with additional chlorodifluoromethane bubbled through the solution after every hour for 5 minutes. The reaction mixture was stored at room temperature for 16 hours. The reaction mixture was quenched by addition of water and extracted with ethyl acetate. The organic extract was washed with water and brine, dried over magnesium sulfate and concentrated. The residue was purified by column chromatography on silica gel (eluent: 0-25% ethyl acetate in hexane) to give 1-difluoromethyl-3-trifluoromethyl-1H-pyrazole-4-carboxylic acid (isomer A) (870 mg, 47% yield) and 2-difluoromethyl-3-trifluoromethyl-2H-pyrazole-4-carboxylic acid (isomer B) (500 mg, 27% yield). Isomer A ¹H-NMR (400 MHz, CDCl₃): 1.37 (t, 3H, Me), 4.37 (q, 2H, CH₂), 7.37 (t, 1H, CH), 8.07 (s, 1H, CH) ppm.

Isomer B ¹H-NMR (400 MHz, CDCl₃): 1.37 (t, 3H, Me), 4.37 (q, 2H, CH₂), 7.22 (t, 1H, CH), 8.43 (s, 1H, CH) ppm.

1-Difluoromethyl-3-trifluoromethyl-1H-pyrazole-4-carboxylic acid ethyl ester and 2-difluoromethyl-3-trifluoromethyl-2H-pyrazole-4-carboxylic acid ethyl ester were reduced to the corresponding alcohols as described in Example I21 and brominated as described in Example I17.

### Example I23

### Preparation of 2,5-dimethyl-4-trifluoromethyl-2H-pyrazole-3-carboxylic acid ethyl ester and 1,5-dimethyl-4-trifluoromethyl-1H-pyrazole-3-carboxylic acid ethyl ester

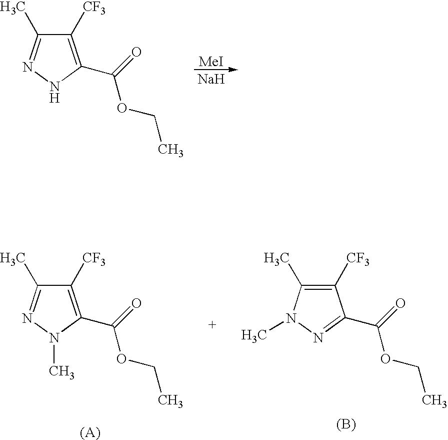

5-Methyl-4-trifluoromethyl-2H-pyrazole-3-carboxylic acid ethyl ester (synthesis according to Journal of Fluorine Chemistry 1994, 69, 253-256) (10.64 g, 47.9 mmol) and methyl iodide (13.59 g, 95.7 mmol) were dissolved in N,N-dimethylformamide (55 ml) and cooled to −5° C. Sodium hydride (1.44 g, 48 mmol) (80% purity) were added in portions at 5° C. The reaction mixture was stirred at room temperature for 18 hours then quenched by addition of water (220 ml). The mixture was extracted three times with ethyl acetate. The combined organic extracts were washed three times with brine, dried over magnesium sulfate and concentrated. The residue was purified by column chromatography on silica gel (gradient: 0-90% ethyl acetate in hexane) to give 2,5-dimethyl-4-trifluoromethyl-2H-pyrazole-3-carboxylic acid ethyl ester as a colourless oil (isomer A) (2.45 g, 21.7% yield) and a mixture of isomer B and starting material. This was purified by reverse phase column chromatography (eluent: 50% water in acetonitrile) to give 1,5-dimethyl-4-trifluoromethyl-1H-pyrazole-3-carboxylic acid ethyl ester as a colourless oil (isomer B) (2.86 g, 25.3% yield).

### Isomer A ¹H-NMR (400 MHz, CDCl₃): 1.40 (q, 3H, Me), 2.37 (s, 3H, Me), 4.08 (s, 3H, Me), 4.40 (q, 2H, CH₂) ppm.

### Isomer B ¹H-NMR (400 MHz, CDCl₃): 1.40 (t, 3H, Me), 2.43 (s, 3H, Me), 3.89 (s, 3H, Me), 4.42 (q, 2H, CH₂) ppm.

### Example I24

### Preparation of 2,5-dimethyl-4-trifluoromethyl-2H-pyrazole-3-carboxylic acid

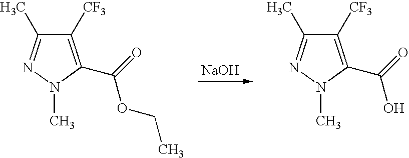

To a solution of 2,5-dimethyl-4-trifluoromethyl-2H-pyrazole-3-carboxylic acid ethyl ester (2.42 g, 10.2 mmol) in ethanol (59 ml) was added a solution of sodium hydroxide (0.478 g, 11.7 mmol) in water (11 ml) at 0° C. The reaction mixture was stirred at room temperature for 2.5 hours and then concentrated. The residue was dissolved in water (50 ml) and acidified by addition of aqueous hydrochloric acid (5.9 ml, 11.8 mmol) (2M). The mixture was stirred at room temperature for 10 minutes. The solid product was isolated by filtration, washed with water and dried to yield 2,5-dimethyl-4-trifluoro-methyl-2H-pyrazole-3-carboxylic acid as a white solid (1.92 g, 90% yield).

¹H-NMR (400 MHz, d₆-DMSO): 2.27 (s, 3H, Me), 4.00 (s, 3H, Me) ppm.

### Example I25

### Preparation of 2,5-dimethyl-4-trifluoromethyl-2H-pyrazole-3-carboxylic acid amide

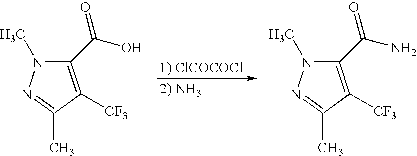

2,5-Dimethyl-4-trifluoromethyl-2H-pyrazole-3-carboxylic acid (5.0 g, 24 mmol) (Example I24) was suspended in dichloromethane (80 ml) and the solution cooled to 0° C., N,N-dimethylformamide (100 μl) was added, followed by dropwise addition of oxalyl chloride (2.3 ml, 26.4 mmol) and the solution was stirred at room temperature for 2 hours. More oxalyl chloride (0.5 ml, 5.7 mmol) added and stirred for further 1 hour. The solution was cooled to −78° C. and aqueous ammonia (12 ml) was added very slowly. The solution was stirred at room temperature for 1 hour and stored at room temperature for 16 hours. The reaction mixture was quenched by addition of aqueous ammonium hydroxide (28%) and concentrated. The residue was partitioned between water and ethyl acetate. The organic extract was washed with aqueous hydrochloric acid (2M), aqueous sodium hydroxide (2.5M), water and brine, dried over magnesium sulfate and concentrated to give 2,5-dimethyl-4-trifluoromethyl-2H-pyrazole-3-carboxylic acid amide (5.0 g, 100% yield).

¹H-NMR (400 MHz, CDCl₃): 2.36 (s, 3H, Me), 3.99 (s, 3H, Me), 6.11 (bs, 2H, NH₂) ppm.

### Example I26

### Preparation of 2,5-dimethyl-4-trifluoromethyl-2H-pyrazole-3-carbonitrile

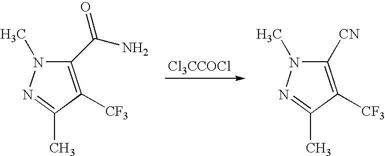

2,5-Dimethyl-4-trifluoromethyl-2H-pyrazole-3-carboxylic acid amide (5.0 g, 24.2 mmol) (Example I25) was suspended in dichloromethane (150 ml), triethylamine (8.1 ml, 58.0 mmol) was added and the solution cooled to 0° C. Trichloroacetyl chloride (3.2 ml, 29.0 mmol) was added dropwise and solution stirred for 30 minutes at 0° C. The reaction mixture was allowed to warm to room temperature and was stored at room temperature for 16 hours. The reaction mixture was quenched by addition of aqueous hydrochloric acid (2M) and extracted with dichloromethane. The organic extract was washed with water and aqueous sodium hydrogencarbonate (saturated), dried over magnesium sulfate and concentrated to give 2,5-dimethyl-4-trifluoromethyl-2H-pyrazole-3-carbonitrile (4.4 g, 96% yield).

¹H-NMR (400 MHz, CDCl₃): 2.38 (s, 3H, Me), 4.03 (s, 3H, Me) ppm.

2,5-Dimethyl-4-trifluoromethyl-2H-pyrazole-3-carbonitrile was brominated as described in Example I15 to give 5-bromomethyl-2-methyl-4-trifluoromethyl-2H-pyrazole-3-carbonitrile.

### Example I27

### Preparation of 5-methyl-3-trifluoromethyl-H-pyrazole

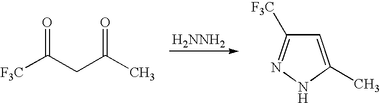

To a solution of trifluoroacetyl acetone (4.62 g, 30 mmol) in methanol (20 ml), was added slowly at 0° C. a solution of hydrazine (945 μl, 30 mmol). The reaction mixture was allowed to warm to room temperature and stirred at room temperature for 16 hours. The reaction mixture was concentrated to give 5-methyl-3-trifluoromethyl-1H-pyrazole as a yellow solid (4.5 g, 95% yield).

¹H-NMR (400 MHz, CDCl₃): 2.35 (s, 3H, Me), 6.32 (s, 1H, CH), 9.88 (bs, 1H, NH) ppm.

### Example I28

### Preparation of 1,5-dimethyl-3-trifluoromethyl-1H-pyrazole

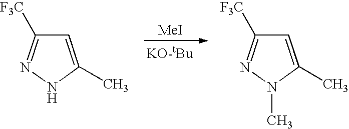

To a solution of 5-methyl-3-trifluoromethyl-1H-pyrazole (4.13 g, 27.5 mmol) (Example I27) in tetrahydrofuran (60 ml), was added slowly at room temperature potassium tert-butoxide (6.18 g, 55 mmol) and 15 minutes later methyl iodide (3.43 ml, 55 mmol). The reaction mixture was stirred for 1 hour before adding more potassium tert-butoxide (3.09 g, 27.5 mmol). After 30 minutes the reaction mixture was quenched by addition of water and extracted with dichloromethane. The organic extract was dried over magnesium sulfate and concentrated to give 1,5-dimethyl-3-trifluoromethyl-1H-pyrazole (3.55 g) which was used in the next step without further purification.

¹H-NMR (400 MHz, CDCl₃): 2.30 (s, 3H, Me), 3.82 (s, 3H, Me), 6.28 (s, 1H, CH) ppm. 1,5-Dimethyl-3-trifluoromethyl-1H-pyrazole was brominated as described in Example I15 to give 5-bromomethyl-1-methyl-3-trifluoromethyl-1H-pyrazole.

¹H-NMR (400 MHz, CDCl₃): 3.95 (s, 3H, Me), 4.45 (s, 2H, CH₂), 6.54 (s, 1H, CH) ppm.

### Example I29

### Preparation of 4-chloro-1,5-dimethyl-3-trifluoromethyl-1H-pyrazole

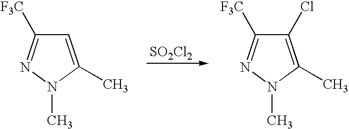

To a solution of 1,5-dimethyl-3-trifluoromethyl-1H-pyrazole (2.7 g, 16.45 mmol) (Example I28) in chloroform (100 ml) at 0° C., was added slowly sulfuryl chloride (2.64 ml, 32.90 mmol). The reaction mixture was stirred for 1 hour at room temperature, then was quenched by addition of water and extracted with dichloromethane. The organic extract was dried over magnesium sulfate and concentrated to yield 4-chloro-1,5-dimethyl-3-trifluoromethyl-1H-pyrazole (3.27 g, 100% yield).

¹H-NMR (400 MHz, CDCl₃): 2.28 (s, 3H, Me), 3.83 (s, 3H, Me) ppm.

4-Chloro-1,5-dimethyl-3-trifluoromethyl-1H-pyrazole was brominated as described in Example I15 to give 5-bromomethyl-4-chloro-1-methyl-3-trifluoromethyl-1H-pyrazole.

¹H-NMR (400 MHz, CDCl₃): 3.96 (s, 3H, Me), 4.44 (s, 2H, CH₂) ppm.

### Example I30

### Preparation of 5-(ethoxycarbonyl)-1,1-dimethyl-3-oxo-2,3-dihydro-1H-pyrazolium inner salt

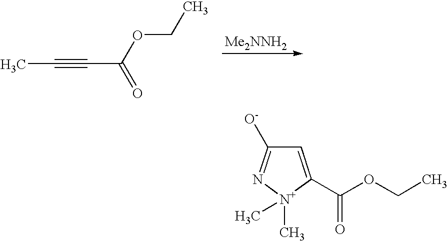

To a solution of diethyl acetylenedicarboxylate (10.67 ml, 67 mmol) in 1:1 ethanol/water (120 ml), was slowly added at 0° C. a solution of dimethyl hydrazine (6 ml, 80 mmol) in 1:1 ethanol/water (40 ml). The reaction mixture was stirred at 0° C. for 30 minutes, then allowed to warm to room temperature and stirred for 1 hour at room temperature. The reaction mixture was concentrated and the residue was partitioned between water and ethyl acetate. The aqueous layer was concentrated to give 5-(ethoxy-carbonyl)-1,1-dimethyl-3-oxo-2,3-dihydro-1H-pyrazolium inner salt (12.3 g, 62% yield).

¹H-NMR (400 MHz, CDCl₃): 1.34 (t, 3H, Me), 3.51 (s, 6H, Me), 4.40 (q, 2H, CH₂), 7.24 (s, 1H, CH) ppm.

### Example I31

### Preparation of 5-hydroxy-2-methyl-2H-pyrazole-3-carboxylic acid ethyl ester

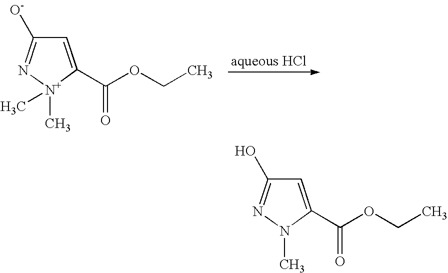

To 5-(ethoxycarbonyl)-1,1-dimethyl-3-oxo-2,3-dihydro-1H-pyrazolium inner salt (7.6 g, 41 mmol) (Example I30) was added aqueous hydrochloric acid (IN) (75 ml). The reaction mixture was stirred for 1.5 hours and was then extracted with dichloromethane. The aqueous layer was concentrated and the residue was taken in methanol and adsorbed onto silica gel. It was purified by column chromatography on silica gel (eluent: 50% ethyl acetate in hexane) to give 5-hydroxy-2-methyl-2H-pyrazole-3-carboxylic acid ethyl ester (6.97 g, 39% yield).

¹H-NMR (400 MHz, CDCl₃): 1.37 (t, 3H, Me), 4.01 (s, 3H, Me), 4.34 (q, 2H, CH₂), 6.15 (s, 1H, CH) ppm.

### Example I32

### Preparation of 5-difluoromethoxy-2-methyl-2H-pyrazole-3-carboxylic acid ethyl ester

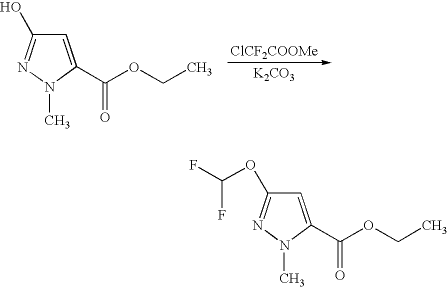

To a suspension of potassium carbonate (4 g, 29.2 mmol) in N,N-dimethyl-formamide (30 ml) was added 5-hydroxy-2-methyl-2H-pyrazole-3-carboxylic acid ethyl ester (1.576 g, 9.27 mmol) (Example I31). The reaction mixture was heated to 80° C. and methyl 2-chloro-2,2-difluoroacetate (2.9 ml, 27.8 mmol) was added over a period of 10 minutes. After the reaction mixture was stirred for 1 hour at 80° C., the reaction mixture was cooled to room temperature and quenched by addition of aqueous hydrochloric acid (1M). The mixture was extracted three times with diethyl ether the combined organic extracts were washed with water, dried over magnesium sulfate and concentrated. The residue was purified by column chromatography on silica gel (eluent: 5-30% ethyl acetate in hexane) to yield 5-difluoromethoxy-2-methyl-2H-pyrazole-3-carboxylic acid ethyl ester (2.04 g, 50% yield).

¹H-NMR (400 MHz, CDCl₃): 1.38 (t, 3H, Me), 4.08 (s, 3H, Me), 4.35 (q, 2H, CH₂), 6.42 (s, 1H, CH), 6.76 (t, 1H, CH) ppm.

5-Difluoromethoxy-2-methyl-2H-pyrazole-3-carboxylic acid ethyl ester was reduced as described in Example I21 to give [5-difluoromethoxy-2-methyl-2H-pyrazol-3-yl]-methanol.

¹H-NMR (400 MHz, CDCl₃): 3.79 (s, 3H, Me), 4.62 (s, 2H, CH₂), 5.83 (s, 1H, CH), 6.75 (t, 1H, CH) ppm.

[5-Difluoromethoxy-2-methyl-2H-pyrazol-3-yl]-methanol was brominated as described in Example I17 to give 3-bromomethyl-5-difluoromethoxy-2-methyl-2H-pyrazole.

¹H-NMR (400 MHz, CDCl₃): 3.80 (s, 3H, Me), 4.51 (s, 2H, CH₂), 5.91 (s, 1H, CH), 6.76 (t, 1H, CH) ppm.

### Example I33

### Preparation of 5-methoxy-2-methyl-2H-pyrazole-3-carboxylic acid ethyl ester

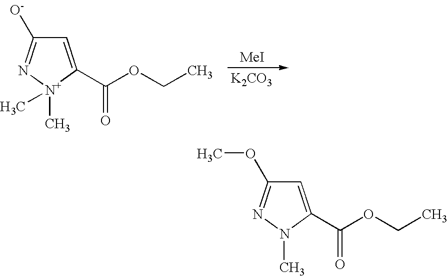

To 5-(ethoxycarbonyl)-1,1-dimethyl-3-oxo-2,3-dihydro-1H-pyrazolium inner salt (92 mg, 0.5 mmol) (Example I30) was added methyl iodide (1.5 ml, 1 mmol) and the potassium carbonate (138 mg, 1 mmol). The reaction mixture was heated for 30 minutes in a microwave at 100° C. The reaction mixture was concentrated and the residue partitioned between water and dichloromethane. The organic extract was dried over magnesium sulfate and concentrated. The residue was purified by column chromatography on silica gel (eluent: 5-30% ethyl acetate in hexane) to give 5-methoxy-2-methyl-2H-pyrazole-3-carboxylic acid ethyl ester (92 mg, 48% yield).

¹H-NMR (400 MHz, CDCl₃): 1.37 (t, 3H, Me), 3.88 (s, 3H, Me), 4.04 (s, 3H, Me), 4.32 (q, 2H, CH₂), 6.19 (s, 1H, CH) ppm.

5-Methoxy-2-methyl-2H-pyrazole-3-carboxylic acid ethyl ester was reduced as described in Example I21 to give [5-methoxy-2-methyl-2H-pyrazol-3-yl]-methanol.

¹H-NMR (400 MHz, CDCl₃): 3.73 (s, 3H, Me), 3.85 (s, 3H, Me), 4.58 (bs, 2H, CH₂), 5.60 (s, 1H, CH) ppm.

[5-Methoxy-2-methyl-2H-pyrazol-3-yl]-methanol was brominated as described in Example I17 to give 3-bromomethyl-5-methoxy-2-methyl-2H-pyrazole.

¹H-NMR (400 MHz, CDCl₃): 3.91 (s, 3H, Me), 3.98 (s, 3H, Me), 4.51 (s, 2H, CH₂), 5.77 (s, 1H, CH) ppm.

### 3) Methods for making Pyridine Derivatives

### Example I34

### Preparation of [2-methyl-6-trifluoromethylpyridin-3-yl]-methanol

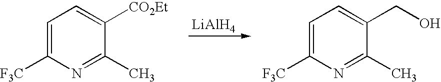

Ethyl 2-methyl-6-trifluoromethylnicotinate (4.66 g, 0.02 mol) (preparation according to Heterocycles 1997 (129) 46 and WO 01/0194339) in tetrahydrofuran was added slowly to a suspension of (1.9 g, 0.05 mol) of lithium aluminium hydride in tetrahydrofuran (150 ml) under nitrogen at room temperature. The reaction mixture was stirred at room temperature for 3 hours and was then quenched by sequential addition of water (0.9 ml), aqueous sodium hydroxide (0.9 ml) (3M), and water (2.7 ml). The sludge was filtered through celite. The residue was washed with diethyl ether and the washings combined with the filtrate. The combined liquors were concentrated under reduced pressure to give [2-methyl-6-trifluoromethylpyridin-3-yl]-methanol (3.92 g, 85% purity), which was used directly without further purification.

¹H-NMR (400 MHz, CDCl₃): 2.58 (s, 3H, Me), 4.81 (s, 2H, CH₁), 7.57 (d, 1H, CH), 7.94 (d, 1H, CH) ppm.

The same method was used with 2-chloro-nicotinic acid ethyl ester as starting material to give [2-chloro-pyridin-3-yl]-methanol.

### Example I35

### Preparation of 3-chloromethyl-2-methyl-6-trifluoromethylpyridine

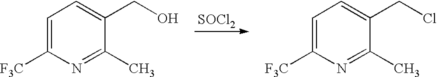

Thionyl chloride (1.6 ml, 2.2 mmol) was added slowly to a stirred solution of [2-methyl-6-trifluoromethylpyridin-3-yl]-methanol (3.5 g, 1.83 mmol) (Example I34) in dichloromethane (30 ml). After stirring at room temperature for 16 hours, the reaction mixture was concentrated under reduced pressure. The residue was taken up into toluene and concentrated under reduced pressure to remove excess thionyl chloride. The residue was purified by column chromatography on silica gel (eluent: ethyl acetate/hexane) to give the product, which was further purified by column chromatography on silica gel (eluent: ethyl acetate/hexane) to give 3-chloromethyl-2-methyl-6-trifluoromethyl-pyridine (2.18 g, 56% yield).

¹H-NMR (400 MHz, CDCl₃): 2.72 (s, 3H, Me), 4.64 (s, 2H, CH₂), 7.53 (d, 1H, CH), 7.83 (d, 1H, CH) ppm.

The same method was used with [2-chloro-pyridin-3-yl]-methanol as starting material to give 2-chloro-3-chloromethyl-pyridine.

### Example I36

### Preparation of 2-chloro-pyridine-3-carboxylic acid methyl ester

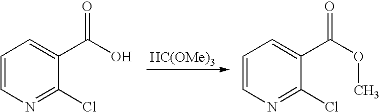

A mixture a 2-chloro nicotinic acid (2.0 g, 12.7 mmol), trimethyl orthoformate (8 ml) and methanol (10 drops) was heated to 150° C. for 20 minutes in a microwave. The solution was allowed to cool to room temperature, diluted with ethyl acetate and washed with aqueous sodium hydroxide (2M). The organic extract was dried over magnesium sulfate and concentrated. The residue was purified by column chromatography on silica gel (eluent: 0-40% ethyl acetate in hexane) to give 2-chloro-pyridine-3-carboxylic acid methyl ester as a colourless oil (1.453 g, 67% yield).

¹H-NMR (400 MHz, CDCl₃): 3.97 (3H, s, Me), 7.33 (1H, m, CH), 8.19 (1H, m, CH), 8.52 (1H, m, CH) ppm.

### Example I37

### Preparation of 2-methoxy-pyridine-3-carboxylic acid methyl ester

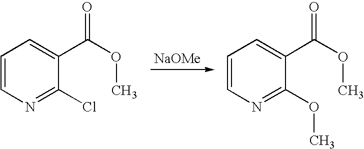

A mixture of 2-chloro-pyridine-3-carboxylic acid methyl ester (0.9 g, 5.24 mmol) (Example I36), methanol (4 ml) and sodium methoxide (25% w/w in methanol) (2.4 ml, 5.24 mmol) was heated at 150° C. for 20 minutes in a microwave. The solution was allowed to cool to room temperature, diluted with water and extracted with three times with ethyl acetate (3×20 ml). The combined organic extracts were dried over magnesium sulfate and concentrated to give 2-methoxy-pyridine-3-carboxylic acid methyl ester as a colourless oil (0.455 g, 52% yield).

¹H-NMR (400 MHz, CDCl₃): 3.9 (3H, s, Me), 4.06 (3H, s, Me), 6.95 (1H, m, CH), 8.15 (1H, m, CH), 8.31 (1H, m, CH) ppm.

### Example I38

### Preparation of [2-methoxy-pyridin-3-yl]-methanol

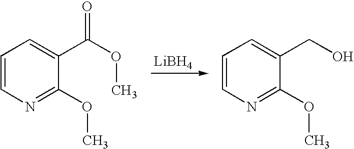

To a solution of 2-methoxy-pyridine-3-carboxylic acid methyl ester (1.021 g, 6.11 mmol) (Example I37) in anhydrous tetrahydrofuran (5 ml) was added in portions lithium borohydride (0.266 g, 1.22 mmol). The reaction mixture was heated to 70° C. for 2 hours. The reaction mixture was quenched by addition of aqueous ammonium chloride (saturated) and then extracted three times with ethyl acetate (3×20 ml). The combined organic extracts were dried over magnesium sulfate and concentrated to give [2-methoxy-pyridin-3-yl]-methanol as a colourless oil (0.849 g, 100% yield).

¹H-NMR (400 MHz, CDCl₃): 2.29 (1H, m, OH), 4.0 (3H, s, Me), 4.67 (2H, m, CH₂), 6.89 (1H, m, CH), 7.59 (1H, m, CH), 8.1 (1H, m, CH) ppm.

### Example I39

### Preparation of 3-bromomethyl-2-methoxy-pyridine

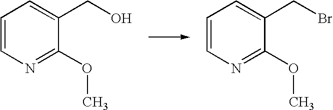

To a solution of [2-methoxy-pyridin-3-yl]-methanol (0.849 g, 6.11 mmol) (Example I38) in dichloromethane (30 ml) was added sequentially triphenyl phosphine (1.683 g, 6.42 mmol) and carbon tetrabromide (1.904 g, 5.81 mmol). The reaction mixture was stored at room temperature for 16 hours. The reaction mixture was concentrated and the residue purified by column chromatography on silica gel (eluent: 0-25% ethyl acetate in hexane) to give 3-bromomethyl-2-methoxy-pyridine as a colourless oil (0.511 g, 42% yield).

¹H-NMR (400 MHz, CDCl₃): 4.02 (3H, s, Me), 4.5 (2H, m, CH₂), 6.88 (1H, m, CH), 7.61 (1H, m, CH), 8.12 (1H, m, CH) ppm.

### 4) Methods for Making Pyrimidine Derivatives

### Example I40

### Preparation of 5-bromo-6-trifluoromethyl-3H-pyrimidin-4-one

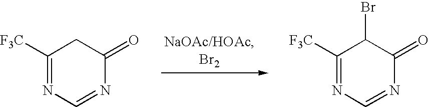

6-Trifluoromethyl-5H-pyrimidin-4-one (10 g, 61 mmol) was dissolved in acetic acid (100 ml). Sodium acetate (24.1 g, 177 mmol) was added and the reaction mixture stirred until all solids had dissolved. Bromine (10.7 g, 67 mmol) was added dropwise over 10 minutes and the reaction mixture was stirred at room temperature for 24 hours. The reaction mixture was concentrated and the residue extracted with ethyl acetate. The organic extract was washed with water, brine., dried over magnesium sulfate and concentrated to give 5-bromo-6-trifluoromethyl-3H-pyrimidin-4-one as a white solid (14.48 g, 97% yield), which was used without further purification.

¹H-NMR (400 MHz, CDCl₃): 8.35 (s, 1H, CH), 13.60 (s, 1H, CH) ppm.

### Example I41

### Preparation of 5-bromo-4-chloro-6-trifluoromethyl-pyrimidine

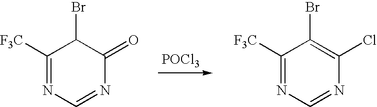

5-Bromo-6-trifluoromethyl-3H-pyrimidin-4-one (Example I40) (8.00 g, 32.9 mmol) was suspended in phosphorous oxychloride (6.00 g, 62.7 mmol). The mixture was heated at 110° C. for 3 hours. The reaction mixture was quenched by adding dropwise to hot water and the mixture was stirred until it had cooled to room temperature. The aqueous mixture was extracted with ethyl acetate. The organic extract was washed with water and brine, dried over magnesium sulfate and concentrated to give 5-bromo-4-chloro-6-trifluoromethyl-pyrimidine as viscous oil (8.50 g, 100% yield), which was used without further purification.

¹H-NMR (400 MHz, CDCl₃): 8.55 (s, 1H, CH) ppm.

### Example I42

### Preparation of 5-bromo-4-methoxy-6-trifluoromethyl-pyrimidine

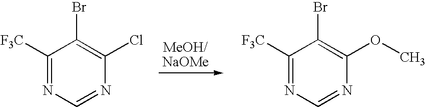

5-Bromo-4-chloro-6-trifluoromethyl-pyrimidine (Example I41) (4.00 g, 15.5 mmol) was dissolved in dry methanol (20 ml). Sodium methoxide (0.974 g, 17.5 mmol) was added and the mixture stirred at room temperature for 1 hour. The reaction mixture was quenched by addition of water and extracted with diethyl ether. The organic extract was washed with water and brine, dried over magnesium sulfate and concentrated. The residue was purified by column chromatography on silica gel (eluent: hexane/ethyl acetate) to give 5-bromo-4-methoxy-6-trifluoromethyl-pyrimidine (3.90 g, 93% yield).

¹H-NMR (400 MHz, CDCl₃): 4.12 (s, 3H, Me), 8.73 (s, 1H, CH) ppm.

### Example I43

### Preparation of 4-methoxy-6-trifluoromethyl-pyrimidine-5-carbaldehyde

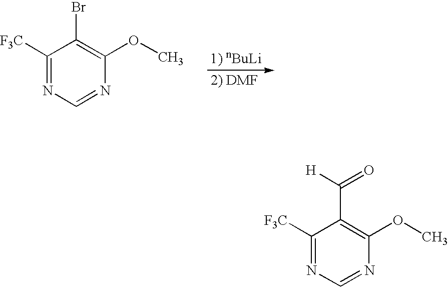

5-Bromo-4-methoxy-6-trifluoromethyl-pyrimidine (Example I42) (3.00 g, 11.6 mmol) was dissolved in dry tetrahydrofuran (45 ml) then cooled to −70° C. n-Butyl lithium (1.6M in hexanes) (10.5 ml, 16.8 mmol) was added at −70° C. over 10 minutes. The reaction mixture was stirred at −70° C. for 30 minutes before N,N-dimethylformamide (1.31 g, 18 mmol) was added in one portion. The reaction mixture was allowed to warm to 0° C. The reaction mixture was quenched by addition of aqueous hydrochloric acid (2M) (50 ml) and extracted with diethyl ether. The organic extract was washed with water and brine, dried over magnesium sulfate and concentrated. The residue was purified by column chromatography on silica gel (eluent: hexane/ethyl acetate) to give 4-methoxy-6-trifluoromethyl-pyrimidine-5-carbaldehyde (1.60 g, 65% yield).

¹H-NMR (400 MHz, CDCl₃): 4.15 (s, 3H, Me), 9.00 (s, 1H, CH), 10.20 (s, 1H, CH) ppm.

### Example I44

### Preparation of (4-methoxy-6-trifluoromethyl-pyrimidin-5-yl)-methanol

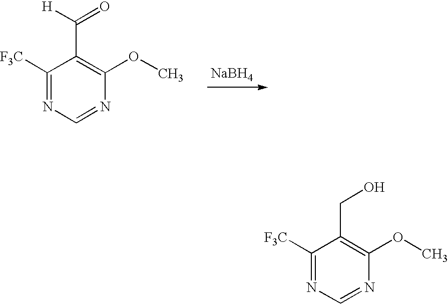

A solution of 4-methoxy-6-trifluoromethyl-pyrimidine-5-carbaldehyde (Example 143) (1.40 g, 6.8 mmol) in ethanol (25 ml) was cooled to 0° C. Sodium borohydride (0130 g, 3.4 mmol) was added in portions at 0° C. and the reaction mixture stirred for 60 minutes. The reaction mixture was quenched by addition of water, the pH of the solution adjusted to 7.0 by addition of aqueous citric acid (10%) and the mixture extracted with ethyl acetate. The organic extract was washed with water, dried over magnesium sulfate and concentrated to give (4-methoxy-6-trifluoromethyl-pyrimidin-5-yl)-methanol as colourless oil (0.463 g, 97% yield), which was used without further purification.

¹H-NMR (400 MHz, CDCl₃): 4.12 (s, 3H, Me), 4.80 (s, 2H, CH₂), 8.81 (s, 1H, CH) ppm.

### Example I45

### Preparation of 5-bromomethyl-4-methoxy-6-trifluoromethyl-pyrimidine

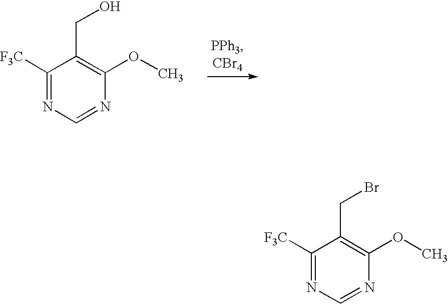

A solution of (4-methoxy-6-trifluoromethyl-pyrimindin-5yl)-methanol (Example 144) (4.5 g, 21.6 mmol) and triphenyl phosphine (6.10 g, 23.2 mmol) in dichloromethane (100 ml) was cooled to 0° C. A solution of carbon tetrabromide (7.7 g, 23.2 mmol) in dichloromethane (20 ml) was added dropwise over 10 minutes at 0° C. The mixture was stirred at room temperature for 16 hours. The reaction mixture was concentrated and the residue purified by column chromatography on silica gel (eluent: hexane/ethyl acetate) to give 5-bromomethyl-4-methoxy-6-trifluoromethyl-pyrimidine (4.64 g, 79% yield).

¹H-NMR (400 MHz, CDCl₃): 4.15 (s, 3H, Me), 4.53 (s, 2H, CH₁), 8.80 (s, 1H, CH) ppm.

### 5) Methods for Making Triazole Derivatives

The synthesis of 1-tert-butyl-1H-[1,2,3]triazole-4-carboxylic acid ethyl ester was described in Synthesis, 1985, 178-180.

1-tert-Butyl-1H-[1,2,3]triazole-4-carboxylic acid ethyl ester was reduced as described in Example I21 to give [1-tert-butyl-1H-[1,2,3]triazol-4-yl]-methanol.

¹H-NMR (400 MHz, CDCl₃): 1.65 (s, 9H, Me), 4.76 (s, 2H, CH₂), 7.60 (s, 1H, CH) ppm.

[1-tert-Butyl-1H-[1,2,3]triazol-4-yl]-methanol was brominated as described in Example I17 to give 4-bromomethyl-1-tert-butyl-1H-[1,2,3]triazole.

¹H-NMR (400 MHz, CDCl₃): 1.65 (s, 9H, Me), 4.58 (s, 2H, CH₂), 7.63 (s, 1H, CH) ppm.

The synthesis of 1,5-dimethyl-1H-[1,2,3]triazole-4-carboxylic acid ethyl ester was described in Journal of Organic Chemistry, 1993, 58(25), 7079-7083 and Synthetic Communications, 2004, 34(2), 369-376.

1,5-Dimethyl-1H-[1,2,3]triazole-4-carboxylic acid ethyl ester was reduced as described in Example I21 to give [1,5-dimethyl-1H-[1,2,3]triazol-4-yl]-methanol.

¹H-NMR (400 MHz, D₃COD): 2.38 (s, 3H, Me), 3.99 (s, 3H, Me), 4.64 (s, 2H, CH₂) ppm.

[1,5-Dimethyl-1H-[1,2,3]triazol-4-yl]-methanol was chlorinated as described in Example I13 to give 4-bromomethyl-1,5-dimethyl-1H-[1,2,3]triazole.

The synthesis of 5-chloromethyl-1,3-dimethyl-1H-[1,2,4]triazole was described in DE 3118258.

The synthesis of 5-chloromethyl-1-methyl-1H-[1,2,4]triazole was described in EP 421210.

The synthesis of 3-chloromethyl-1-methyl-1H-[1,2,4]triazole was described in EP 421210.

The synthesis of 3-chloromethyl-4,5-dimethyl-4H-[1,2,4]triazole was described in Synthesis, 2006, 156-160.

### Example I46

### Preparation of 3-(methyl-hydrazono)-butan-2-one oxime

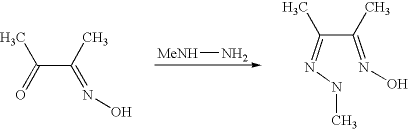

Butane-2,3-dione monooxime (2.6 g, 25.8 mmol) was dissolved in ethanol (100 ml) then methylhydrazine (1.13 g, 24.5 mmol) was added. The reaction mixture was stirred at 80° C. for 2 hours. Methylhydrazine (0.6 g, 12 mmol) was added and the reaction mixture stirred at 80° C. for 1.5 hours. Methylhydrazine (0.6 g, 12 mmol) was added and the reaction mixture stirred at 80° C. for another 1.5 hours, then at room temperature for 48 hours. The mixture was concentrated to leave 3.3 g of a pale yellow, crystalline solid which was used without further purification.

¹H-NMR (400 MHz, CDCl₃): 1.89 (s, 3H, Me), 2.11 (s, 3H, Me), 3.09 (s, 3H, Me), 4.8-5.3 (bs, 1H, OH) ppm.

### Example I47

### Preparation of 2,4,5-trimethyl-2H-[1,2,3]triazole 1-oxide

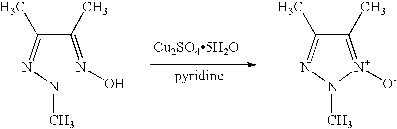

3-(Methyl-hydrazono)-butan-2-one oxime (3.3 g, 25.8 mmol) (Example I46) was dissolved in tetrahydrofuran (135 ml), then aqueous pyridine (15%) (157 ml, 0.29 mol) was added, followed by copper(II)-sulfate (15.7 g, slurry in 35 ml water). The reaction mixture was stirred at 80° C. for 2 hours, then cooled and extracted three times with ethyl acetate. The organic extracts were combined, washed once with aqueous copper(II) sulfate (10%) then dried over magnesium sulfate and concentrated. The residue was purified by column chromatography on silica gel (eluent: methanol/dichloromethane) to give the product as colourless liquid. (0.7 g, 21% yield).

¹H-NMR (400 MHz, CDCl₃): 2.19 (s, 3H, Me), 2.23 (s, 3H, Me), 3.93 (s, 3H, Me) ppm.

### Example I48

### Preparation of 5-bromomethyl-2,4-dimethyl-2H-[1,2,3]triazole 1-oxide

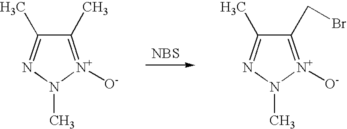

N-Bromosuccinimide (186 mg, 1.0 mmol) and N,N′-azobis(isobutyronitrile) (AIBN) (14 mg, 0.087 mmol) were added to 2,4,5-trimethyl-2H-[1,2,3]triazole 1-oxide (110 mg, 0.87 mmol) (Example I47) in carbon tetrachloride (7 ml). The mixture was heated to 70° C. for 1 hour then cooled to room temperature and filtered. The solvent was removed to give the product as brown gum which was used without further purification. ¹H-NMR (400 MHz, CDCl₃): 2.31 (3H, s, Me), 3.95 (3H, s, Me), 4.42 (2H, s, CH₂) ppm.

### Example I49

### Preparation of 4-bromomethyl-2,5-dimethyl-2H-[1,2,3]-triazole

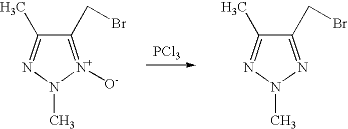

5-Bromomethyl-2,4-dimethyl-2H-[1,2,3]triazole 1-oxide (4.83 g, 23.3 mmol) (Example I48) was dissolved in carbon tetrachloride (50 ml), then phosphorous tri-chloride (6.8 ml, 77.8 mol) was added dropwise. The reaction mixture was stirred at 75° C. for 2.5 hours. The reaction mixture was quenched by slow addition into hot water, then cooled and diluted with cold water. The organic layer was separated and the aqueous layer was extracted twice times with dichloromethane. The organic layers were combined, washed with water and brine, dried over magnesium sulfate and concentrated. The residue was purified by column chromatography on silica gel (eluent: methanol/dichloromethane) to give the product as yellow oil (1.47 g, 33% yield).

¹H-NMR (400 MHz, CDCl₃): 2.3 (s, 3H, Me), 4.1 (s, 3H, Me), 5.5 (s, 2H, CH₂) ppm.

### Example I50

### Preparation of 1-(4-methoxy-benzyl)-5-methyl-1H-[1,2,3]-triazole-4-carboxylic acid ethyl ester and 3-(4-methoxy-benzyl)-5-methyl-3H-[1,2,3]triazole-4-carboxylic acid ethyl ester

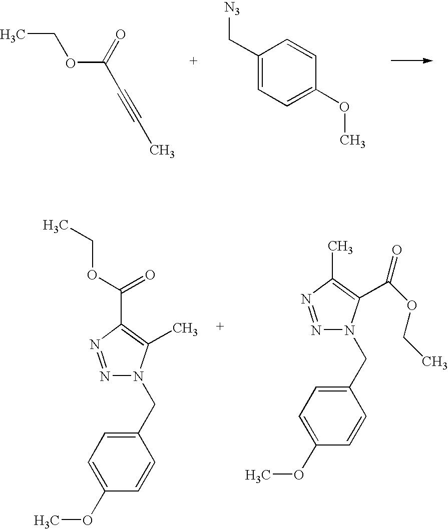

4-Methoxybenzyl azide (5.47 g, 33.6 mmol) was dissolved in toluene (20 ml) and methyl-2-butynoate (6.72 ml, 67.1 mmol) was added dropwise over 5 minutes at room temperature. The reaction mixture was heated to 10° C. for 14 hours. The solution was cooled to room temperature and the solvent was evaporated to yield a bright yellow liquid which was purified by column chromatography on silica gel (eluent: 0-50% ethyl acetate in hexane). The mixture of 1-(4-methoxy-benzyl)-5-methyl-1H-[1,2,3]triazole-4-carboxylic acid ethyl ester and 3-(4-methoxy-benzyl)-5-methyl-3H-[1,2,3]triazole-4-carboxylic acid ethyl ester was obtained as a bright yellow oil (7.35 g, 85% yield). Isomer 1 (major) ¹H NMR (400 MHz, CDCl₃): 2.46 (s, 3H, Me), 3.79 (s, 3H, Me), 3.94 (s, 3H, Me), 5.47 (s, 2H, CH₂), 6.82-6.88 (m, 2H, CH), 7.13 (d, 2H, CH) ppm. Isomer 2 (minor) ¹H NMR (400 MHz, CDCl₃): 2.51 (s, 3H, Me), 3.77 (s, 3H, Me), 3.90 (s, 3H, Me), 5.80 (s, 2H, CH₂), 6.82-6.88 (m, 2H, CH), 7.28 (d, 2H, CH) ppm.

The same method was used with pent-2-ynoic acid ethyl ester as the starting material to give a mixture of 5-ethyl-1-(4-methoxy-benzyl)-1H-[1,2,3]triazole-4-carboxylic acid ethyl ester and 5-ethyl-3-(4-methoxy-benzyl)-3H-[1,2,3]triazole-4-carboxylic acid ethyl ester.

Isomer I (major) ¹H NMR (400 MHz, CDCl₃): 1.00 (t, 3H, Me), 1.42 (t, 3H, Me), 2.88-2.96 (m, 4H, CH₂), 3.79 (s, 3H, Me), 4.42 (q, 2H, CH₂), 5.48 (s, 2H, CH₂), 6.82-6.87 (m, 2H, CH), 7.14 (d, 2H, CH) ppm.
Isomer 2 (minor) ¹H NMR (400 MHz, CDCl₃): 1.29 (t, 3H, Me), 1.36 (t, 3H, Me), 2.88-2.96 (m, 4H, CH₂), 3.78 (s, 3H, Me), 4.35 (q, 2H, CH₂), 5.81 (s, 2H, CH₂), 6.82-6.87 (m, 2H, CH), 7.28 (d, 2H, CH) ppm.

### Example I51

### Preparation of 5-methyl-2H-[1,2,3]triazole-4-carboxylic acid ethyl ester

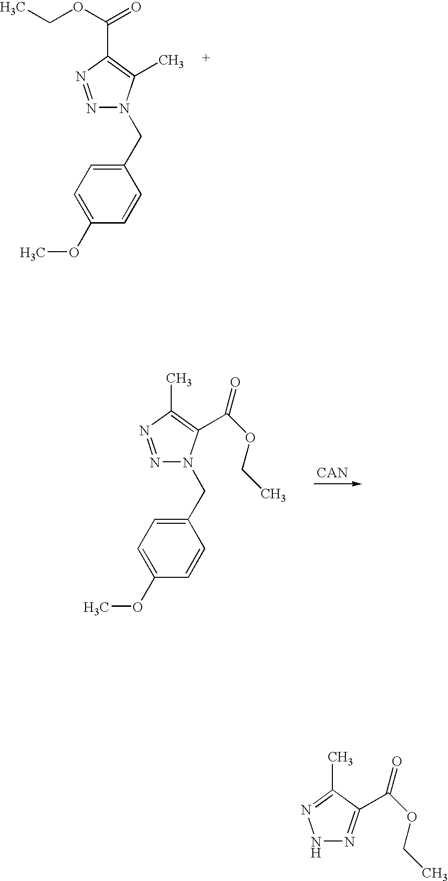

The mixture of 1-(4-methoxy-benzyl)-5-methyl-1H-[1,2,3]triazole-4-carboxylic acid ethyl ester and 3-(4-methoxy-benzyl)-5-methyl-3H-[1,2,3]triazole-4-carboxylic acid ethyl ester (Example I50) (7.35 g, 28.6 mmol) was dissolved in acetonitrile/water (9:1) (100 ml) and cerium(IV) ammonium nitrate (CAN) (31.4 g, 57.2 mmol) was added. The solution was stirred at room temperature for 16 hours. The solvent was evaporated and the residue diluted between water and ethyl acetate. The aqueous layer was separated from the organic layer and was extracted twice more with ethyl acetate. The organic extracts were combined, washed with brine, dried over magnesium sulfate and concentrated. Dichloromethane (50 ml) was added to the solid which caused a white solid to precipitate (1.77 g, 40% yield). The solid was isolated via filtration. The mother liquor was evaporated and again treated with dichloromethane (50 ml) which caused more white solid to precipitate. The solid was isolated via filtration and to both solids were dried to give 5-methyl-2H-[1,2,3]triazole-4-carboxylic acid ethyl ester (2.6 g).

¹H NMR (400 MHz, CDCl₃): 1.46 (t, 3H, Me), 2.61 (s, 3H, Me), 4.45 (q, 2H, CH₂) ppm.

The same method was used with the mixture of 5-ethyl-1-(4-methoxy-benzyl)-1H-[1,2,3]triazole-4-carboxylic acid ethyl ester and 5-ethyl-3-(4-methoxy-benzyl)-3H-[1,2,3]triazole-4-carboxylic acid ethyl ester as the starting material to give 5-ethyl-2H-[1,2,3]triazole-4-carboxylic acid ethyl ester.

¹H NMR (400 MHz, CDCl₃): 1.36 (t, 3H, Me), 1.42 (t, 3H, Me), 3.08 (q, 2H, CH₂), 4.44 (q, 2H, CH₂) ppm.

### Example I52

### Preparation of 2-ethyl-5-methyl-2H-[1,2,3]triazole-4-carboxylic acid ethyl ester, 3-ethyl-5-methyl-3H-[1,2,3]triazole-4-carboxylic acid ethyl ester and 1-ethyl-5-methyl-3H-[1,2,3]triazole-4-carboxylic acid ethyl ester

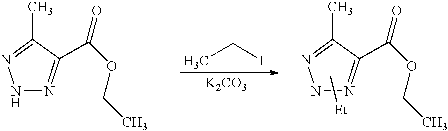

5-Methyl-2H-[1,2,3]triazole-4-carboxylic acid ethyl ester (1.77 g, 12.6 mmol) (Example I51) was dissolved in N,N-dimethylformamide (12 ml) and ethyl iodide (16.3 mmol, 1.31 ml) and potassium carbonate (2.26 g, 16.3 mmol) were added at 0° C. The reaction mixture was warmed to room temperature and stirred for 5 hours under an atmosphere of nitrogen. The solution was diluted between water and diethyl ether. The aqueous layer was separated from the organic layer and was extracted twice with diethyl ether. The organic extracts were combined, washed with brine, dried over magnesium sulfate and concentrated to give a mixture of 2-ethyl-5-methyl-2H-[1,2,3]triazole-4-carboxylic acid ethyl ester, 3-ethyl-5-methyl-3H-[1,2,3]triazole-4-carboxylic acid ethyl ester and 1-ethyl-5-methyl-3H-[1,2,3]triazole-4-carboxylic acid ethyl ester as a yellow oil (1.10 g, 52% yield).

Isomer I (major) ¹H NMR (400 MHz, CDCl₃): 1.36-1.58 (m, 6H, Me), 2.52 (s, 3H, Me), 4.32-4.49 (m, 4H, CH₂) ppm.
Isomer 2 (middle) ¹H NMR (400 MHz, CDCl₃): 1.36-1.58 (m, 6H, Me), 2.54 (s, 3H, Me), 4.32-4.49 (m, 2H, CH₂), 4.72 (q, 2H, CH₂) ppm.
Isomer 3 (minor) ¹H NMR (400 MHz, CDCl₃): 1.36-1.58 (m, 6H, Me), 2.59 (s, 3H, Me), 4.32-4.49 (m, 4H, CH₂) ppm.

The same method was used with 5-methyl-2H-[1,2,3]triazole-4-carboxylic acid ethyl ester as the starting material and iso-propyl iodide as reagent to give 5-methyl-2-prop-2-yl-2H-[1,2,3]triazole-4-carboxylic acid ethyl ester, 5-methyl-3-prop-2-yl-3H-[1,2,3]triazole-4-carboxylic acid ethyl ester and 5-methyl-1-prop-2-yl-3H-[1,2,3]triazole-4-carboxylic acid ethyl ester.

Isomer 1 (major) ¹H NMR (400 MHz, CDCl₃): 1.38-1.44 (m, 3H, Me), 1.56-1.63 (m, 6H, Me), 2.52 (s, 3H, Me), 4.31-4.45 (m, 2H, CH₂), 4.83 (sept, 1H, CH) ppm.
Isomer 2 (middle) ¹H NMR (400 MHz, CDCl₃): 1.38-1.44 (m, 3H, Me), 1.56-1.63 (m, 6H, Me), 2.53 (s, 3H, Me), 4.31-4.45 (m, 2H, CH₂), 5.44 (sept, 1H, CH) ppm.
Isomer 3 (minor) ¹H NMR (400 MHz, CDCl₃): 1.38-1.44 (m, 3H, Me), 1.56-1.63 (m, 6H, Me), 2.59 (s, 3H, Me), 4.31-4.45 (m, 2H, CH₂), 4.58 (sept, 1H, CH) ppm.

The same method was used with 5-ethyl-2H-[1,2,3]triazole-4-carboxylic acid ethyl ester as the starting material and methyl iodide as reagent to give 5-ethyl-2-methyl-2H-[1,2,3]triazole-4-carboxylic acid ethyl ester.

Isomer 1 ¹H NMR (400 MHz, CDCl₃): 1.28-1.32 (m, 3H, Me), 1.41 (t, 3H, Me), 2.95 (q, 2H, CH₂), 4.20 (s, 3H, Me), 4.38-4.44 (m, 2H, CH₂) ppm.

### Example I53

### Preparation of 1-(4-methoxy-benzyl)-5-trifluoromethyl-1H-[1,2,3]-triazole-4-carboxylic acid ethyl ester and 3-(4-methoxy-benzyl)-5-trifluoromethyl-3H-[1,2,3]-triazole-4-carboxylic acid ethyl ester

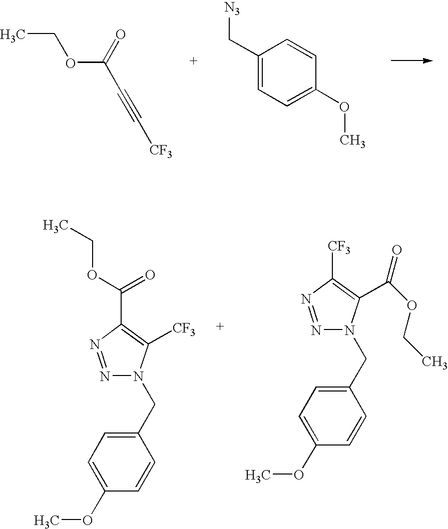

Ethyl 4,4,4-trifluoro-2-butynecarboxylate (1.65 g, 10.1 mmol) was added to 4-methoxybenzyl azide (preparation described in e.g. J. Chem. Soc., Perkin Trans. 1, 1982 (2), 627-630) (1.65 g, 9.9 mmol) in toluene (10 ml) and the mixture was stirred at room temperature for 16 hours. The mixture was concentrated and purified by column chromatography on silica gel (eluent: ethyl acetate/iso-hexane) to give a 1.4:1 mixture of 1-(4-methoxy-benzyl)-5-trifluoromethyl-1H-[1,2,3]triazole-4-carboxylic acid ethyl ester and 3-(4-methoxy-benzyl)-5-trifluoromethyl-3H-[1,2,3]triazole-4-carboxylic acid ethyl ester (2.85 g, 87% yield).

Isomer 1 (minor) ¹H-NMR (400 MHz, CDCl₃): 1.4 (t, 3H, Me), 3.8 (s, 3H, Me), 4.4 (q, 2H, CH₂), 5.85 (s, 2H, CH₂), 6.9 (d, 2H, CH), 7.35 (d, 2H, CH) ppm.
Isomer 2 (major) ¹H-NMR (400 MHz, CDCl₃): 1.4 (t, 3H, Me), 3.8 (s, 3H, Me), 4.45 (q, 2H, CH₂), 5.7 (s, 2H, CH₂), 6.85 (d, 2H, CH), 7.2 (d, 2H, CH) ppm.

### Example I54

### Preparation of 5-trifluoromethyl-2H-[1,2,3]-triazole-4-carboxylic acid ethyl ester

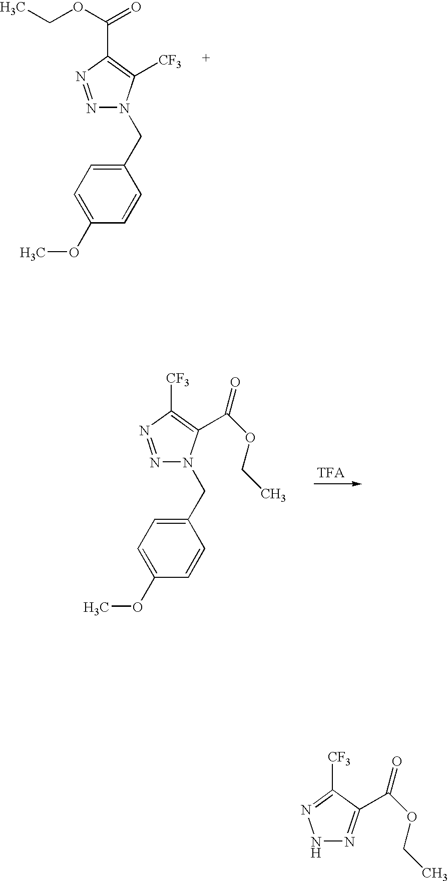

A 1.4:1 mixture of 1-(4-methoxy-benzyl)-5-trifluoromethyl-1H-[1,2,3]triazole-4-carboxylic acid ethyl ester and 3-(4-methoxy-benzyl)-5-trifluoromethyl-3H-[1,2,3]-triazole-4-carboxylic acid ethyl ester (2.85 g, 8.66 mmol) (Example I53) was dissolved in trifluoroacetic acid (TFA) (15 ml) and the mixture was heated to 60° C. for 2 hours. The mixture was concentrated and purified by column chromatography on silica gel (eluent: methanol/dichloromethane) to give 5-trifluoromethyl-2H-[1,2,3]triazole-4-carboxylic acid ethyl ester (2.1 g).

¹H-NMR (400 MHz, CDCl₃): 1.4 (t, 3H, Me), 4.5 (q, 2H, CH₂), 12.2-12.6 (bs, 1H, NH) ppm.

### Example I55

### Preparation of 2-methyl-5-trifluoromethyl-2H-[1,2,3]triazole-4-carboxylic acid ethyl ester, 3-methyl-5-trifluoromethyl-3H-[1,2,3]-triazole-4-carboxylic acid ethyl ester and 1-methyl-5-trifluoromethyl-3H-[1,2,3]-triazole-4-carboxylic acid ethyl ester

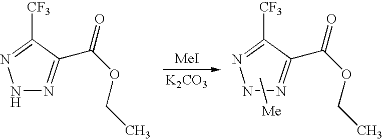

5-Trifluoromethyl-2H-[1,2,3]triazole-4-carboxylic acid ethyl ester (2.1 g, 10.0 mmol) (Example I54) was dissolved in acetonitrile (15 ml) and methyl iodide (1.12 ml, 12.0 mmol) and potassium carbonate (2.76 g, 20.0 mmol) was added at room temperature. The mixture was stirred at room temperature for 16 hours, then dichloromethane (50 ml) and water (50 ml) was added. The phases were separated and the aqueous was washed several times with dichloromethane. The organic layers were combined, dried over magnesium sulfate and concentrated. The residue was purified by column chromatography on silica gel (eluent: ethyl acetate/iso-hexane) to give 1.71 g of a 64:30:6 mixture of 2-methyl-5-trifluoromethyl-2H-[1,2,3]triazole-4-carboxylic acid ethyl ester, 3-methyl-5-trifluoromethyl-3H-[1,2,3]triazole-4-carboxylic acid ethyl ester and 1-methyl-5-trifluoromethyl-3H-[1,2,3]triazole-4-carboxylic acid ethyl ester.

Isomer I (major) ¹H-NMR (400 MHz, CDCl₃): 1.4 (t, 3H, Me), 4.32 (s, 3H, Me), 4.45 (q, 2H, CH₂) ppm.
Isomer 2 (middle) ¹H-NMR (400 MHz, CDCl₃): 1.4 (t, 3H, Me), 4.38 (s, 3H, Me), 4.45 (q, 2H, CH₂) ppm.
Isomer 2 (minor) ¹H-NMR (400 MHz, CDCl₃): 1.4 (t, 3H, Me), 4.30 (m, 3H, Me), 4.45 (q, 2H, CH₂) ppm.

The same method was used with ethyl iodide as reagent to give 2-ethyl-5-tri-fluoromethyl-2H-[1,2,3]triazole-4-carboxylic acid ethyl ester and 3-ethyl-5-trifluoro-methyl-3H-[1,2,3]triazole-4-carboxylic acid ethyl ester (3.4:1 mixture).

Isomer 1 (major) ¹H-NMR (400 MHz, CDCl₃): 1.4 (t, 3H, Me), 1.65 (t, 3H, Me), 4.45 (q, 2H, CH₂), 4.6 (q, 2H, CH₂) ppm.
Isomer 2 (minor) ¹H-NMR (400 MHz, CDCl₃): 1.4 (t, 3H, Me), 1.55 (t, 3H, Me), 4.45 (q, 2H, CH₂), 4.8 (q, 2H, CH₂) ppm.

The same method was used with isopropyl iodide as reagent to give 2-(prop-2-yl)-5-trifluoromethyl-2H-[1,2,3]triazole-4-carboxylic acid ethyl ester and 3-(prop-2-yl)-5-trifluoromethyl-3H-[1,2,3]triazole-4-carboxylic acid ethyl ester (4.5:1 mixture).

Isomer 1 (major) ¹H-NMR (400 MHz, CDCl₃): 1.4 (t, 3H, Me), 1.65 (d, 6H, Me), 4.45 (q, 2H, CH₂) ppm.
Isomer 2 (minor) ¹H-NMR (400 MHz, CDCl₃): 1.4 (t, 3H, Me), 1.7 (d, 6H, Me), 4.95 (q, 2H, CH₂) ppm.

The same method was used with cyclopentyl iodide as reagent to give 2-cyclo-pentyl-5-trifluoromethyl-2H-[1,2,3]triazole-4-carboxylic acid ethyl ester.

¹H-NMR (400 MHz, CDCl₃): 1.4 (t, 3H, Me), 1.7-1.8 (m, 2H, CH₂), 1.9-2.0 (m, 2H, CH₂), 2.2-2.3 (m, 4H, CH₂), 4.45 (q, 2H, CH₂), 5.1 (m, 1H, CH) ppm.

The same method was used with allyl iodide as reagent to give 2-allyl-5-tri-fluoromethyl-2H-[1,2,3]triazole-4-carboxylic acid ethyl ester and 3-allyl-5-trifluoro-methyl-3H-[1,2,3]triazole-4-carboxylic acid ethyl ester (4:1 mixture).

Isomer I (major) ¹H-NMR (400 MHz, CDCl₃): 1.4 (t, 3H, Me), 4.45 (q, 2H, CH₂), 5.1 (d, 2H, CH₂), 5.35-5.45 (m, 2H, CH), 6.0-6.1 (m, 1H, CH) ppm.

The same method was used with 2-methoxy-ethyl iodide as reagent to give 2-(2-methoxy-ethyl)-5-trifluoromethyl-2H-[1,2,3]triazole-4-carboxylic acid ethyl ester and 3-(2-methoxy-ethyl)-5-trifluoromethyl-3H-[1,2,3]triazole-4-carboxylic acid ethyl ester (4:1 mixture).

Isomer I (major) ¹H-NMR (400 MHz, CDCl₃): 1.4 (t, 3H, Me), 3.35 (s, 3H, Me), 3.9 (t, 2H, CH₂), 4.45 (q, 2H, CH₂), 4.7 (t, 2H, CH₂) ppm.
Isomer 2 (minor) ¹H-NMR (400 MHz, CDCl₃): 1.4 (t, 3H, Me), 3.3 (s, 3H, Me), 3.8 (t, 2H, CH₂), 4.45 (q, 2H, CH₂), 4.95 (t, 2H, CH₂) ppm.

The same method was used with cyclobutylmethyl iodide as reagent to give 2-(cyclobutyl-methyl)-5-trifluoromethyl-2H-[1,2,3]triazole-4-carboxylic acid ethyl ester and 3-(cyclobutyl-methyl)-5-trifluoromethyl-3H-[1,2,3]triazole-4-carboxylic acid ethyl ester (4:1 mixture).

Isomer I (major) ¹H-NMR (400 MHz, CDCl₃): 1.4 (t, 3H, Me), 1.8-2.0 (m, 4H, CH₂), 2.0-2.15 (m, 2H, CH₂), 2.95-3.1 (m, 1H, CH), 4.55 (q, 2H, CH₂), 4.65 (d, 2H, CH₂) ppm.
Isomer 2 (minor) ¹H-NMR (400 MHz, CDCl₃): 1.4 (t, 3H, Me), 1.8-2.0 (m, 4H, CH₂), 2.0-2.15 (m, 2H, CH₂), 2.85-2.95 (m, 1H, CH), 4.55 (q, 2H, CH₂), 4.78 (d, 2H, CH₂) ppm.

### Example I56

### Preparation of 5-hydroxy-1-(4-methoxy-benzyl)-1H-[1,2,3]triazole-4-carboxylic acid ethyl ester

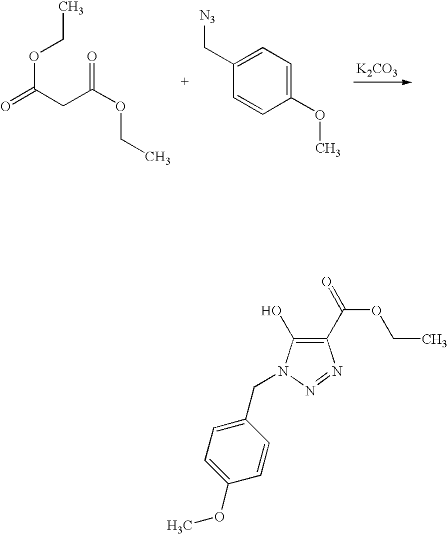

4-Methoxybenzyl azide (preparation described in e.g. J. Chem. Soc., Perkin Trans. 1, 1982 (2), 627-630) (42 g, 0.253 mol) was dissolved in dry dimethylsulfoxide (272 ml) and powdered potassium carbonate (139 g, 1 mol) was added. The reaction mixture was stirred at room temperature while diethyl malonate (56 g, 0.35 mol) was added. The reaction mixture was stirred at 40° C. for 72 hours. The reaction mixture was cooled to 0° C. and quenched by addition of aqueous hydrochloric acid (SM) (675 ml). The mixture was stirred at room temperature for 2 hours. The solid was isolated via filtration, washed with water and hexane and dried to give 5-hydroxy-1-(4-methoxy-benzyl)-1H-[1,2,3]triazole-4-carboxylic acid ethyl ester as an off white solid (47.5 g, 68% yield).

¹H NMR (400 MHz, CDCl₃): 1.29 (t, 3H, Me), 3.72 (s, 3H, Me), 4.25 (q, 2H, CH₂), 5.27 (s, 2H, CH₂), 7.07 (q, 4H, CH) ppm.

### Example I57

### Preparation of 5-methoxy-1-(4-methoxy-benzyl)-1H-[1,2,3]triazole-4-carboxylic acid ethyl ester

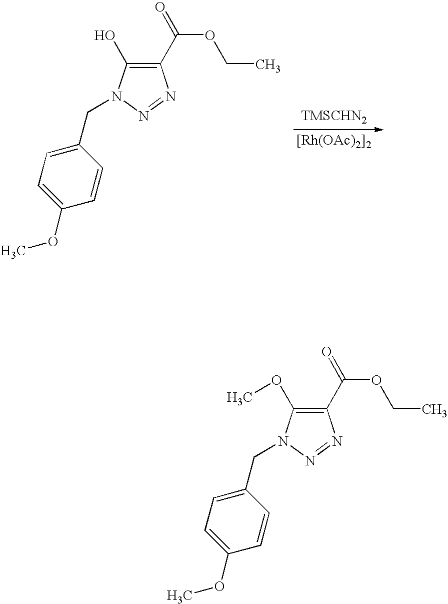

5-Hydroxy-1-(4-methoxy-benzyl)-1H-[1,2,3]triazole-4-carboxylic acid ethyl ester (26.18 g, 0.944 mol) (Example I56) was dissolved in dry N,N-dimethylformamide (300 ml) and rhodium(II) acetate dimer (300 mg) was added. The reaction mixture was cooled to 20° C. and (trimethylsilyl)diazomethane (TMSCHN₂) (2M in diethyl ether) (142 ml, 0.284 mol) was added over 2 hours and the reaction mixture stirred at room temperature for 16 hours. The reaction mixture was cooled to 0° C. and quenched by sequential addition of methanol (75 ml), glacial acetic acid (5 ml) and water (1 ml). The mixture was extracted three times with ethyl acetate. The combined organic extracts were washed three times with brine, dried over magnesium sulfate and concentrated. The residue was purified by column chromatography on silica gel (eluent: 10-55% ethyl acetate in hexane) to give 5-methoxy-1-(4-methoxy-benzyl)-1H-[1,2,3]triazole-4-carboxylic acid ethyl ester as a straw coloured oil (15.2 g, 55% yield).

¹H NMR (400 MHz, CDCl₃): 1.41 (q, 3H, Me), 3.80 (s, 3H, Me), 4.13 (s, 3H, Me), 4.41 (t, 2H, CH₂), 5.31 (s, 2H, CH₂), 7.1 (q, 4H, CH) ppm.

5-Methoxy-1-(4-methoxy-benzyl)-1H-[1,2,3]triazole-4-carboxylic acid ethyl ester was deprotected as described in Example I51 to give 5-methoxy-1H-[1,2,3]triazole-4-carboxylic acid ethyl ester.

¹H NMR (400 MHz, CDCl₃): 1.41 (t, 3H, Me), 4.11 (s, 3H, Me), 4.47 (q, 2H, CH₂) ppm.

5-Methoxy-1H-[1,2,3]triazole-4-carboxylic acid ethyl ester was methylated with methyl iodide according to the method described in Example I52 to give 5-methoxy-1-methyl-1H-[1,2,3]triazole-4-carboxylic acid ethyl ester (isomer A) and 5-methoxy-2-methyl-2H-[1,2,3]triazole-4-carboxylic acid ethyl ester (isomer B).

### Isomer A ¹H NMR (400 MHz, CDCl₃): 1.39 (q, 3H, Me), 4.15 (s, 3H, Me), 4.24 (s, 3H, Me), 4.40 (q, 2H, CH₂) ppm.

### Isomer B ¹H NMR (400 MHz, CDCl₃): 1.35 (t, 3H, Me), 3.99 (s, 3H, Me), 4.07 (s, 3H, Me), 4.36 (q, 2H, CH₂) ppm.

5-Hydroxy-1-(4-methoxy-benzyl)-1H-[1,2,3]triazole-4-carboxylic acid ethyl ester (26.18 g; 0.944 mol) (Example I56) was also reacted with ethyl 2-chloro-2,2-difluoro-acetate as described in Example I32 to give 5-difluoromethoxy-1-(4-methoxy-benzyl)-1H-[1,2,3]triazole-4-carboxylic acid ethyl ester.

5-Difluoromethoxy-1-(4-methoxy-benzyl)-1H-[1,2,3]triazole-4-carboxylic acid ethyl ester was deprotected as described in Example I51 to give 5-difluoromethoxy-1H-[1,2,3]triazole-4-carboxylic acid ethyl ester.

5-Difluoromethoxy-1H-[1,2,3]triazole-4-carboxylic acid ethyl ester was methylated with methyl iodide according to the method described in Example I52 to give 5-difluoromethoxy-1-methyl-1H-[1,2,3]triazole-4-carboxylic acid ethyl ester (isomer A) and 5-difluoromethoxy-2-methyl-2H-[1,2,3]triazole-4-carboxylic acid ethyl ester (isomer B).

### Example I58

### Preparation of 2-methyl-2H-[1,2,3]triazole-4-carboxylic acid methyl ester and 3-methyl-3H-[1,2,3]triazole-4-carboxylic acid methyl ester

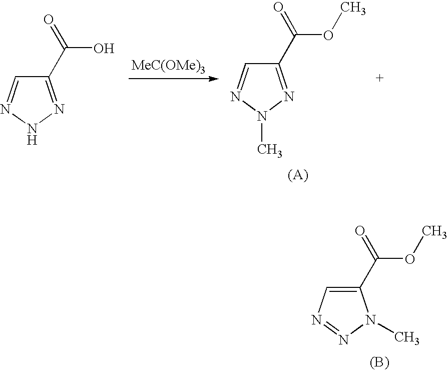

To a solution of 2H-[1,2,3]triazole-4-carboxylic acid (734 mg, 6.5 mmol) in toluene (10 ml), was added trimethylorthoacetate (2.48 ml, 19.5 mmol). The reaction mixture was heated at reflux for 16 hours. After cooling, the reaction mixture was concentrated. The residue was purified by column chromatography on silica gel (eluent: 10-30% ethyl acetate in hexane) to give 2-methyl-2H-[1,2,3]triazole-4-carboxylic acid methyl ester (isomer A) (40 mg, 4% yield) and 3-methyl-3H-[1,2,3]triazole-4-carboxylic acid methyl ester (isomer B) (223 mg, 24% yield).

Isomer A ¹H NMR (400 MHz, CDCl₃): 3.96 (s, 3H, Me), 4.28 (s, 3H, Me), 8.05 (s, 1H, CH) ppm.

2-Methyl-2H-[1,2,3]triazole-4-carboxylic acid methyl ester was reduced as described in Example I21 to give [2-methyl-2H-[1,2,3]triazol-4-yl]-methanol.

¹H-NMR (400 MHz, CDCl₃): 4.15 (s, 3H, Me), 4.74 (s, 2H, CH₂), 7.53 (s, 1H, CH) ppm.

[2-Methyl-2H-[1,2,3]triazol-4-yl]-methanol was brominated as described in Example I17 to give 4-bromomethyl-2-methyl-2H-[1,2,3]triazole.

¹H-NMR (400 MHz, CDCl₃): 4.18 (s, 3H, Me), 4.51 (s, 2H, CH₂), 7.58 (s, 1H, CH) ppm.

The same method was used with triethylorthoacetate as reagent to give 2-ethyl-2H-[1,2,3]triazole-4-carboxylic acid methyl ester (isomer A) and 3-ethyl-3H-[1,2,3]-triazole-4-carboxylic acid methyl ester (isomer B).

### Isomer A ¹H NMR (400 MHz, CDCl₃): 1.41 (t, 3H, Me), 1.60 (t, 3H, Me), 4.43 (q, 2H, CH₂), 4.55 (q, 2H, CH₂), 8.04 (s, 1H, CH) ppm.

2-Ethyl-2H-[1,2,3]triazole-4-carboxylic acid methyl ester was reduced as described in Example I21 to give [2-ethyl-2H-[1,2,3]triazol-4-yl]-methanol.

¹H-NMR (400 MHz, CDCl₃): 1.55 (t, 3H, Me), 4.44 (q, 2H, CH₂), 4.76 (s, 2H, CH₂), 7.55 (s, 1H, CH) ppm.

[2-Ethyl-2H-[1,2,3]triazol-4-yl]-methanol was brominated as described in Example I17 to give 4-bromomethyl-2-ethyl-2H-[1,2,3]triazole.

¹H-NMR (400 MHz, CDCl₃): 1.56 (t, 3H, Me), 4.45 (q, 2H, CH₂), 4.52 (s, 2H, CH₂), 7.59 (s, 1H, CH) ppm.

### Example I59

### Preparation of 4,5-dibromo-1H-[1,2,3]triazole

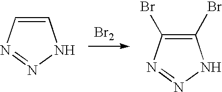

To a solution of 1H-[1,2,3]triazole (1.26 ml, 21.7 mmol) in water (10 ml) at 50° C., was added bromine (1.5 ml, 29 mmol). The reaction mixture was stirred at 50° C. for 1.5 hours. The white solid (2.375 g) was isolated via filtration and washed with water (5 ml). To the combined filtrates was added more bromine (1.5 ml, 29 mmol). The reaction mixture was stirred at room temperature for 20 hour. More white solid (1.83 g) was isolated via filtration and washed with water (5 ml). To the combined filtrates was added more bromine (1.5 ml, 29 mmol). The reaction mixture was stirred at room temperature for 20 hours. More white solid (375 mg) was isolated via filtration and washed with water (5 ml). The white solids were combined and dried to give 4,5-dibromo-1H-[1,2,3]-triazole (4.92 g, 93% yield). M.p. 194.7° C.

### Example I60

### Preparation of 4,5-dibromo-1-methyl-1H-[1,2,3]triazole and 4,5-dibromo-2-methyl-2H-[1,2,3]triazole

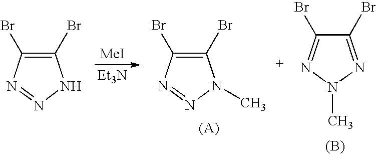

To a solution of 4,5-dibromo-1H-[1,2,3]triazole (2.26 g, 10 mmol) (Example I59) and triethyl amine (1.5 ml, 10 mmol) in dichloromethane (50 ml), was added methyl iodide (625 μl, 10 mmol). The reaction mixture was stirred at room temperature for 24 hours. More triethyl amine (0.75 ml, 5 mmol) and more methyl iodide (312 μl, 5 mmol) were added and the mixture was stirred for 3 hours. The reaction mixture was quenched with aqueous ammonium chloride (saturated, 15 ml). The organic extract was dried over magnesium sulfate and concentrated and the residue was purified by column chromatography on silica gel (eluent 10-30% ethyl acetate in hexane) to give 4,5-dibromo-2-methyl-2H-[1,2,3]triazole (isomer B) (625 mg, 26% yield) and 4,5-dibromo-1-methyl-1H-[1,2,3]triazole (isomer A) (825 mg, 34% yield).

### Isomer A ¹H-NMR (400 MHz, CDCl₃): 4.09 (s, 3H, Me) ppm.

### Isomer B ¹H-NMR (400 MHz, CDCl₃): 4.18 (s, 3H, Me) ppm.

### Example I61

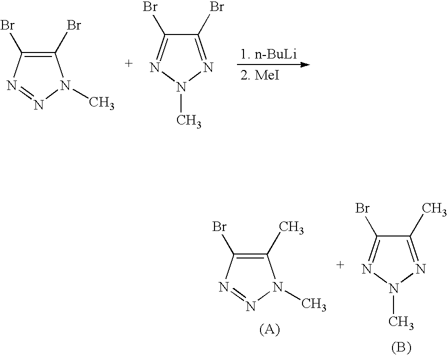

### Preparation of 4-bromo-1,5-dimethyl-1H-[1,2,3]triazole

To a solution of 4,5-dibromo-1-methyl-1H-[1,2,3]triazole and 4,5-dibromo-2-methyl-2H-[1,2,3]triazole (1.5 g, 6.23 mmol) (Example I60) in tetrahydrofuran (25 ml) at −80° C., was slowly added n-butyl lithium (2.5M in THF) (3 ml, 7.48 mmol) followed 20 minutes later by methyl iodide (775 μl, 12.46 mmol). The reaction mixture was stirred at −80° C. for 2 hours. The reaction mixture was allowed to warm to room temperature and quenched with aqueous hydrochloric acid (1M). The mixture was extracted with dichloromethane, the organic extract dried over magnesium sulfate and concentrated to give 4-bromo-1,5-dimethyl-1H-[1,2,3]triazole (isomer A) and 4-bromo-2,5-dimethyl-2H-[1,2,3]triazole (isomer B) (1.096 g, 89% yield).

### Isomer A ¹H-NMR (400 MHz, CDCl₃): 2.30 (s, 3H, Me), 4.00 (s, 3H, Me) ppm.

### Isomer B ¹H-NMR (400 MHz, CDCl₃): 2.24 (s, 3H, Me), 4.10 (s, 3H, Me) ppm.

4-Bromo-1,5-dimethyl-1H-[1,2,3]triazole and 4-bromo-2,5-dimethyl-2H-[1,2,3]triazole were brominated as described in Example I15 to give 4-bromo-5-bromo-methyl-1-methyl-1H-[1,2,3]triazole (isomer A) and 4-bromo-5-bromomethyl-2-methyl-2H-[1,2,3]triazole (isomer B).

### Isomer A ¹H-NMR (400 MHz, CDCl₃): 4.12 (s, 3H, Me), 4.45 (s, 2H, CH₂) ppm.

### Isomer B ¹H-NMR (400 MHz, CDCl₃): 4.17 (s, 3H, Me), 4.45 (s, 2H, CH₂) ppm.

### 6) Methods for Making Thiadiazole Derivatives

4-Methyl-[1,2,3]thiadiazole-5-carboxylic acid ethyl ester is commercially available.

4-Methyl-[1,2,3]thiadiazole-5-carboxylic acid ethyl ester was reduced as described in Example I0 to give [4-methyl-[1,2,3]thiadiazol-5-yl]-methanol.

¹H-NMR (400 MHz, CDCl₃): 2.60 (s, 3H, Me), 3.84 (bs, 1H, OH), 4.98 (s, 2H, CH₂) ppm.

[4-Methyl-[1,2,3]thiadiazol-5-yl]-methanol was brominated as described in Example I17 to give 5-bromomethyl-4-methyl-[1,2,3]thiadiazole.

¹H-NMR (400 MHz, CDCl₃): 2.70 (s, 3H, Me), 4.70 (s, 2H, CH₂) ppm.

### 7) Coupling Methods and Oxidations

### Example P1

### Preparation of 4-bromo-3-(5-difluoromethoxy-1-methyl-3-trifluoromethyl-1H-pyrazol-4-ylmethylsulfanyl)-5,5-dimethyl-4,5-dihydro-isoxazole

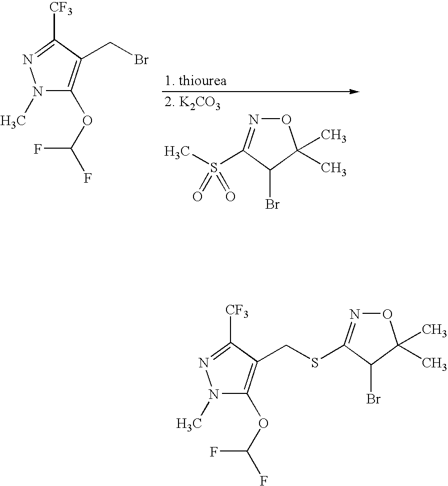

4-Bromomethyl-5-difluoromethoxy-1-methyl-3-trifluoromethyl-1H-pyrazole (0.7 g, 2.36 mmol) (prepared as described in WO 2004/013106), was stirred in ethanol (30 ml) before thiourea (0.189 g, 2.48 mmol) was added and it was stirred at room temperature for 1 hour. 4-Bromo-3-methanesulfonyl-5,5-dimethyl-4,5-dihydro-isoxazole (0.632 g, 2.48 mmol) and potassium carbonate (0.343 g, 2.48 mmol) were added and the mixture was heated to 70° C. for 4 hours. The solvent was removed and the residue absorbed onto silica gel before being purified by column chromatography on silica gel (eluent: hexane/ethyl acetate) to give Compound No. 2.04 of Table 52 (0.473 g, 46% yield).

The following compounds were synthesised as described in Example P1 with 4-bromo-3-methanesulfonyl-5,5-dimethyl-4,5-dihydro-isoxazole as reagent:

### Compound No. 1.01 and Compound No. 1.04 of Table 51.

The following compounds were synthesised as described in Example P1 with 3-benzenesulfonyl-4-chloro-5,5-dimethyl-4,5-dihydro-isoxazole as reagent:

### Compound No. 1.06 of Table 51.

The following compound was synthesised as described in Example P1 with 3-benzenesulfonyl-4,4-dichloro-5,5-dimethyl-4,5-dihydro-isoxazole as reagent:

### Compound No. 1.08 of Table 51.

The following compounds were synthesised as described in Example P1 with 4-fluoro-3-methanesulfonyl-5,5-dimethyl-4,5-dihydro-isoxazole as reagent:

### Compound No. 1.10 of Table 51,

Compound No. 2.16, Compound No. 2.22, Compound No. 2.28, Compound No. 2.35, Compound No. 2.38, Compound No. 2.41, Compound No. 2.45, Compound No. 2.48, Compound No. 2.51, Compound No. 2.54, Compound No. 2.57, Compound No. 2.64, Compound No. 2.67, Compound No. 2.70 of Table 52,

### Compound No. 4.06 of Table 54,

### Compound No. 5.01 of Table 55,

### Compound No. 6.01, Compound No. 6.05, Compound No. 6.08, Compound No. 6.11 of Table 56,

Compound No. 7.01, Compound No. 7.07, Compound No. 7.11, Compound No. 7.15, Compound No. 7.18, Compound No. 7.21, Compound No. 7.24, Compound No. 7.28, Compound No. 7.31, Compound No. 7.34, Compound No. 7.37, Compound No. 7.42, Compound No. 7.45, Compound No. 7.50 and Compound No. 7.53 of Table 57, Compound No. 9.01 of Table 59,

### Compound No. 10.01 of Table 60, and

### Compound No. 11.01 of Table 61.

### Example P2

### Preparation of 4-bromo-3-(5-difluoromethoxy-1-methyl-3-trifluoromethyl-1H-pyrazol-4-ylmethylsulfinyl)-5,5-dimethyl-4,5-dihydro-isoxazole

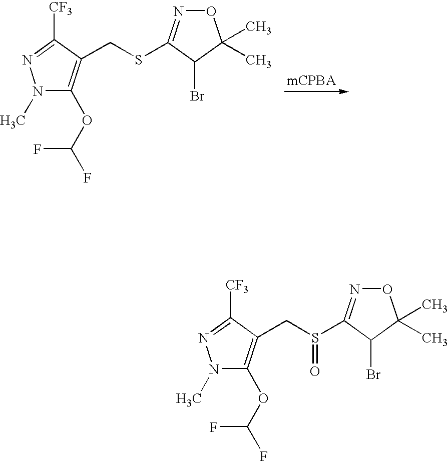

A solution of 4-bromo-3-(5-difluoromethoxy-1-methyl-3-trifluoromethyl-1H-pyrazol-4-ylmethylsulfanyl)-5,5-dimethyl-4,5-dihydro-isoxazole (0.15 g, 0.343 mmol) (Compound No. 2.04 of Table 52) in dichloromethane (25 ml) was cooled to 0° C. and 3-chloroperoxybenzoic acid (mCPBA) (0.119 g, 0.412 mmol) was added in portions. After stirring for 30 minutes at 0° C. the reaction mixture was quenched by addition of aqueous sodium metabisulfite (10%). The mixture was extracted with dichloromethane and the organic extract washed with aqueous sodium hydroxide (1M), dried over magnesium sulfate and concentrated. The residue was purified by column chromatography on silica gel (eluent: hexane/ethyl acetate) to give Compound No. 2.05 of Table 52 as a 7:2 mixture of diastereoisomers (0.137 g, 88% yield).

### Example P3

### Preparation of 4-Bromo-3-(5-difluoromethoxy-1-methyl-3-trifluoromethyl-1H-pyrazol-4-ylmethylsulfonyl)-5,5-dimethyl-4,5-dihydro-isoxazole

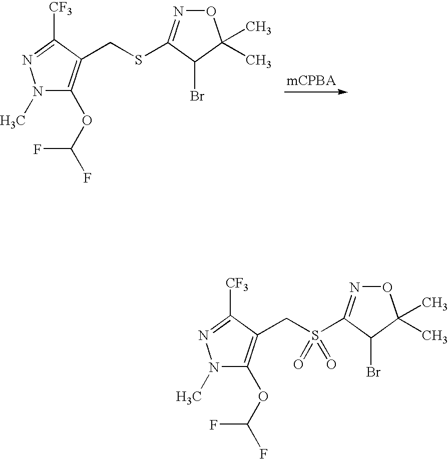

A solution of 4-bromo-3-(5-difluoromethoxy-1-methyl-3-trifluoromethyl-1H-pyrazol-4-ylmethylsulfanyl)-5,5-dimethyl-4,5-dihydro-isoxazole (0.234 g, 0.535 mmol) (Compound No. 2.04 of Table 52) in dichloromethane (30 ml) and 3-chloroperoxy-benzoic acid (0.339 g, 1.18 mmol) was added in portions. After stirring at room temperature for 16 hours the reaction mixture was quenched by addition of aqueous sodium metabisulfite (10%). The mixture was extracted with dichloromethane and the organic extract washed with aqueous sodium hydroxide (1M), dried over magnesium sulfate and concentrated. The residue was purified by column chromatography on silica gel (eluent: hexane/ethyl acetate) to give Compound No. 2.06 of Table 52 (0.164 g, 65% yield).

### Example P4

### Preparation of 4-fluoro-5,5-dimethyl-3-[1-methyl-5-(2,2,2-trifluoro-ethoxy)-3-trifluoromethyl-1H-pyrazol-4-ylmethylsulfanyl]-4,5-dihydro-isoxazole

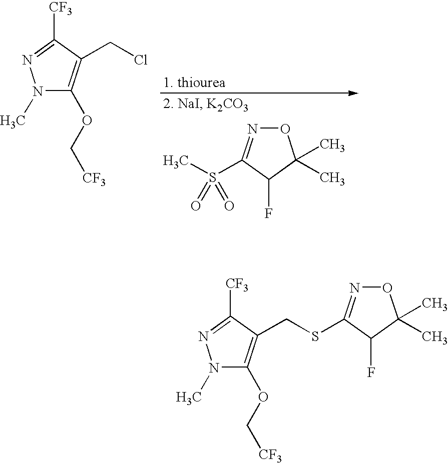

4-Chloromethyl-1-methyl-5-(2,2,2-trifluoroethoxy)-3-trifluoromethyl-1H-pyrazole (0.587 g, 1.98 mmol) (prepared as described in WO 2004/013106), was stirred in ethanol (100 ml) before the thiourea (0.132 g, 1.73 mmol) was added and it was stirred at room temperature for 1 hour. 4-Fluoro-3-methanesulfonyl-5,5-dimethyl-4,5-dihydro-isoxazole (0.322 g, 1.65 mmol), sodium iodide (0.259 g, 1.73 mmol) and potassium carbonate (0.239 g, 1.73 mmol) were added and it was heated to 70° C. for 4 hours. The solvent was removed and the residue absorbed onto silica before being purified by column chromatography on silica gel (eluent: hexane/ethyl acetate) to give Compound No. 2.07 of Table 52 (0.546 g, 76% yield).

The following compound was synthesised as described in Example P4 with 4-bromo-3-methanesulfonyl-5,5-dimethyl-4,5-dihydro-isoxazole as reagent:

Compound No. 2.01 of Table 52.

The following compound was synthesised as described in Example P4 with 3-benzenesulfonyl-4-chloro-5,5-dimethyl-4,5-dihydro-isoxazole as reagent:

Compound No. 2.13 of Table 52.

The following compound was synthesised as described in Example P4 with 3-benzenesulfonyl-4,4-dichloro-5,5-dimethyl-4,5-dihydro-isoxazole as reagent:

Compound No. 2.19 of Table 52.

### Example P5

### Preparation of 3-[chloro-(2-trifluoromethoxy-phenyl)-methanesulfonyl]-4-fluoro-5,5-dimethyl-4,5-dihydro-isoxazole

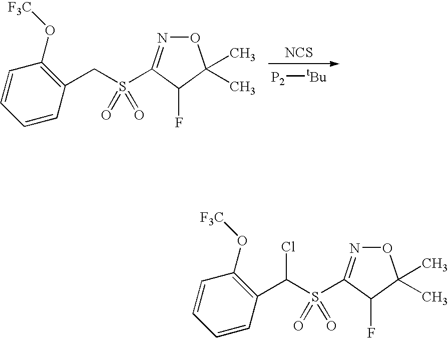

4-Fluoro-5,5-dimethyl-3-(2-trifluoromethoxy-phenylmethanesulfonyl)-4,5-dihydro-isoxazole (0.11 g, 0.31 mmol) (prepared from trifluoromethoxybenzyl bromide and 4-fluoro-3-methanesulfonyl-5,5-dimethyl-4,5-dihydro-isoxazole as described in Example P1 followed by oxidation as described in Example P3) was dissolved in tetrahydrofuran (5 ml). 1-tert-butyl-2,2,4,4,4-pentakis(dimethylamino-2-lambda⁵-5,4-lambda⁵-5-catenadi(phosphazene) (P₂-tBu) (2M in THF) (0.16 ml, 0.31 mmol) was added followed by N-chloro succinimide (0.043 g, 0.31 mmol) and the reaction mixture stirred at room temperature for 1.5 hours. The solvent was removed and the residue purified by column chromatography on silica gel (eluent: hexane/ethyl acetate) to give Compound No. 1.13 of Table 51 (0.035 g, 29% yield).

### Example P6

### Preparation of 4-chloro-5,5-dimethyl-3-[1-methyl-5-(2,2,2-trifluoro-ethoxy)-3-trifluoromethyl-1H-pyrazol-4-ylmethylsulfonyl]-4,5-dihydro-isoxazole

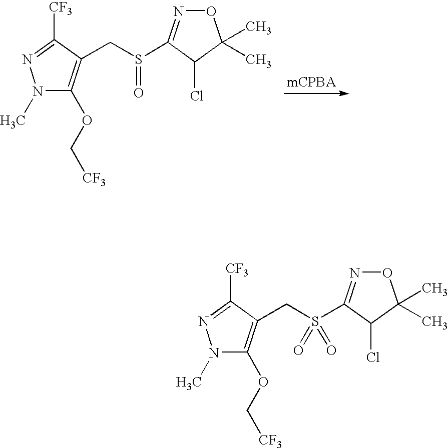

4-Chloro-5,5-dimethyl-3-[1-methyl-5-(2,2,2-trifluoro-ethoxy)-3-trifluoromethyl-1H-pyrazol-4-ylmethylsulfinyl]-4,5-dihydro-isoxazole (0.084 g, 0.176 mmol) (Compound No. 2.14 in Table 52, 3:2 mixture of diastereoisomers) was dissolved in dichloromethane (15 ml) and 3-chloroperoxybenzoic acid (0.101 g, 0.352 mmol) was added. After stirring for 2 days at room temperature the reaction mixture was quenched by addition of aqueous sodium metabisulfite (10%) and extracted with dichloromethane. The organic extract was washed with aqueous sodium hydroxide (1M), dried over magnesium sulfate and concentrated. The residue was purified by column chromatography on silica gel (eluent: hexane/ethyl acetate) to give Compound No. 2.15 of Table 52 (0.057 g, 65% yield, 85% purity).

### Example P7

### Preparation of 3-[(2,6-difluoro-phenyl)-difluoro-methanesulfonyl]-5,5-dimethyl-4,5-dihydro-isoxazole

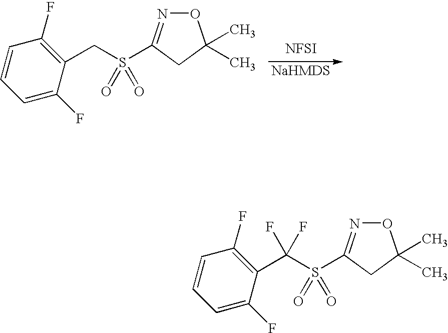

Sodium hexamethyldisilazide (1M in THF) (53.1 ml, 0.053 mol) was added dropwise to a solution of 3-(2,6-difluoro-phenylmethanesulfonyl)-5,5-dimethyl-4,5-dihydro-isoxazole (15.42 g, 0.0534 mol) (prepared as described in WO 2001/012613) in dry tetrahydrofuran (340 ml) at −70° C. under nitrogen. After 15 minutes N-fluorobenzene-sulfonimide (16.83 g, 0.071 mol) was added in portions. After a further 1 hour, sodium hexamethyldisilazide (45 ml, 0.045 mol) (1M in THF) was added dropwise to the stirred reaction mixture at −70° C. After 15 minutes, N-fluorobenzenesulfonimide (16.83 g, 0.071 mol) was added in portions. Further portions of N-fluorobenzenesulfonimide were added until analysis by GLC indicated that fluorination of the benzylic position was complete. Saturated aqueous ammonium chloride (30 ml) was added and the reaction mixture was then allowed to warm to room temperature. The mixture was concentrated under reduced pressure at 40° C. and the residue partitioned between ethyl acetate and water. The aqueous phase was further extracted with ethyl acetate and the combined organic phases were washed twice with saturated aqueous sodium hydrogencarbonate and once with water and brine, dried over magnesium sulfate and concentrated to give a beige solid. The product was further purified by recrystallisation twice from isopropanol to give a beige solid (4.5 g, 25% yield), which was used without further purification.

### Example P8

### Preparation of 3-[(2,6-difluoro-phenyl)-difluoro-methanesulfonyl]-4-fluoro-5,5-dimethyl-4,5-dihydro-isoxazole

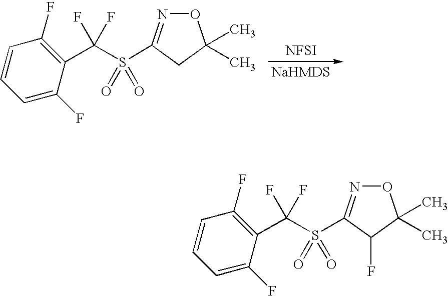

Sodium hexamethyldisilazide (3.5 ml, 3.5 mmol) (1M in THF) was added dropwise to a solution containing 3-[(2,6-difluoro-phenyl)-difluoro-methanesulfonyl]-5,5-dimethyl-4,5-dihydro-isoxazole (1.08 g, 3.3 mmol) and N-fluorobenzenesulfonimide (1.12 g, 3.5 mmol) in dry tetrahydrofuran (25 ml) under nitrogen at −70° C. More sodium hexamethyldisilazide (3.5 ml, 3.5 mmol) (1M in THF) was added dropwise to the stirred reaction mixture at −70° C. After storing for 16 hours in the freezer, the reaction mixture was quenched by pouring into saturated aqueous ammonium chloride at room temperature and then extracted with ethyl acetate. The combined ethyl acetate extracts were washed with water and brine, dried over magnesium sulfate, and concentrated to give a yellow semi-solid. The product was triturated with dichloromethane. The dichloromethane extract was further purified by column chromatography on silica gel (eluent: 20% ethyl acetate in hexane) to give Compound No. 1.18 of Table 51 as white solid (0.175 g, 15% yield).

Compound No. 1.12 of Table 51 and Compound No. 3.07 of Table 53 were prepared analogously.

### Example P9

### Preparation of 3-(2,6-difluoro-phenylmethanesulfonyl)-4-fluoro-5,5-dimethyl-4,5-dihydro-isoxazole

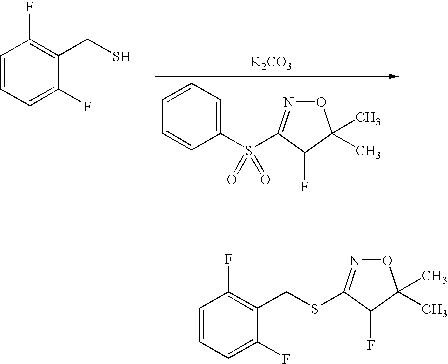

2,6-Difluorobenzyl thiol (0.2 g, 1.2 mmol) (can be prepared from 2,6-difluoro-benzylbromide as described in J. Fluorine Chem., 1986, 399-414) and potassium carbonate (0.5 g, 3.6 mmol) were added to a solution of 3-benzenesulfonyl-4-fluoro-5,5-dimethyl-4,5-dihydro-isoxazole (0.15 g, 0.58 mmol) in ethanol (5 ml) in a microwave tube. The mixture was heated in a sealed microwave tube (130° C. for 6 minutes). After cooling, the reaction mixture was concentrated under reduced pressure and then partitioned with water and ethyl acetate. The aqueous phase was then extracted with ethyl acetate and the combined organic phases were washed with water and brine, dried with magnesium sulfate and concentrated to give a pale yellow oil (0.22 g). The oil was dissolved in a little ethyl acetate and applied to a silica column and then further purified by column chromatography on silica gel (eluent: 10% ethyl acetate in hexane). Fractions containing the product, which were free from starting material, were combined and then further purified by column chromatography on silica gel (eluent: 20% ethyl acetate in hexane) to give Compound No. 1.14 of Table 51 as a colourless oil (0.1 g, 62% yield).

### Example P10

### Alternative Preparation of 4-fluoro-5,5-dimethyl-3-[1-methyl-5-(2,2,2-trifluoro-ethoxy)-3-trifluoromethyl-1H-pyrazol-4-ylmethylsulfanyl]-4,5-dihydro-isoxazole

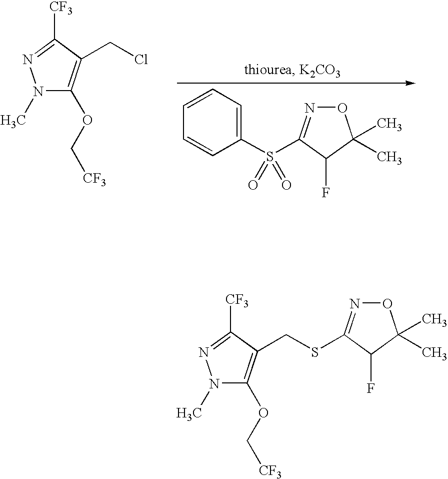

Thiourea (60 mg, 0.8 mmol), 4-chloromethyl-1-methyl-5-(2,2,2-trifluoroethoxy)-3-trifluoromethyl-1H-pyrazole (206 mg, 0.7 mmol) and potassium carbonate (0.2 g, 1.4 mmol) were added sequentially to a stirred mixture of 3-benzenesulfonyl-4-fluoro-5,5-dimethyl-4,5-dihydro-isoxazole (150 mg, 0.8 mmol) in ethanol (5 ml) at room temperature in a microwave tube. The mixture was heated in a sealed microwave tube at 130° C. for 6 minutes. After heating for a further 4 minutes at 130° C., no sulfone was detected. After cooling, the mixture was concentrated under reduced pressure and partitioned between water and ethyl acetate. The aqueous phase was further extracted with ethyl acetate. The combined ethyl acetate extracts were washed with water and brine, dried over magnesium sulfate and concentrated to give a pale yellow oil. The oil was dissolved in a small quantity of ethyl acetate and then further purified by column chromatography on silica gel (eluent: 10% ethyl acetate in hexane). Fractions free from starting materials, were combined, concentrated under reduced pressure and further purified by column chromatography on silica gel (eluent: 20% ethyl acetate in hexane) to give Compound 2.07 of Table 52 as a colourless oil which solidified on standing (0.1 g, 31% yield), M.p. 50-51° C.

### Example P11

### Preparation of 3-(4-fluoro-5,5-dimethyl-4,5-dihydro-isoxazol-3-yl-sulfanylmethyl)-2-methyl-6-trifluoromethyl-pyridine

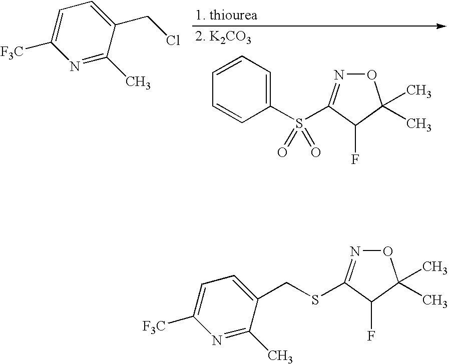

Thiourea (84 mg, 1.1 mmol) was added slowly to a solution of 3-chloromethyl-2-methyl-6-trifluoromethylpyridine (236 mg, 1.13 mol) in ethanol (7 ml). The mixture was stirred for a further 2 hours and then stored at room temperature for 16 hours. 3-Benzene-sulfonyl-4-fluoro-5,5-dimethyl-4,5-dihydro-isoxazole (283 mg, 1.1 mmol) and potassium carbonate (0.15 g, 1.08 mmol) were added successively to the reaction mixture. After stirring for a further 1 hour at room temperature the reaction mixture was heated at reflux for 4 hours. After cooling, the reaction mixture was concentrated, the residue partitioned between water and ethyl acetate and the aqueous phase extracted with ethyl acetate. The combined organic extracts were washed with water and brine, dried over magnesium sulfate and concentrated to give a yellow oil. The oil was dissolved in a small quantity of ethyl acetate and purified by column chromatography on silica gel (eluent: 20% ethyl acetate in hexane) to give Compound 3.01 of Table 53 as a colourless oil (0.2 g, 56% yield).

Compound No. 3.04 of Table 53 and Compound No. 8.01 were prepared analogously.

### Example P12

### Preparation 3-(4-fluoro-5,5-dimethyl-4,5-dihydro-isoxazol-3-ylsulfanyl-methyl)-1-methyl-4-trifluoromethyl-1H-pyrazole

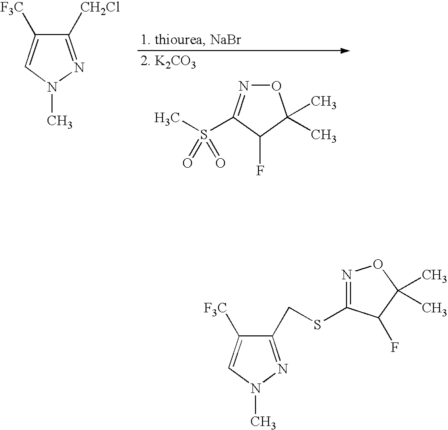

Thiourea (126 mg, 1.65 mmol) and sodium bromide (155 mg, 1.52 mmol) were added to a solution of 3-chloromethyl-1-methyl-4-trifluoromethyl-1H-pyrazole (300 mg, 1.51 mmol) (Example I21) in ethanol (1% water) (10 ml). The reaction mixture was heated at reflux with stirring. After 3 hours, 5,5-dimethyl-4-fluoro-3-phenylsulfonyl-isoxazoline (381 mg, 1.52 mmol) and potassium carbonate (417 mg, 3.02 mmol) were added and the reaction mixture was heated at reflux for 16 hours. After cooling, the mixture was diluted with water and then extracted with ethyl acetate. The combined organic layers were washed with brine, dried over magnesium sulfate and concentrated. The residue was purified by chromatography on silica gel (eluent: ethyl acetate/hexane) to give Compound 4.01 of Table 54 (345 mg, 73% yield).

Compound No. 2.32 of Table 52 was prepared analogously.

### Example P13

### 3-(1-(4-fluoro-5,5-dimethyl-4,5-dihydro-isoxazol-3-ylsulfonyl)-eth-1 yl)-1-methyl-4-trifluoromethyl-1H-pyrazole

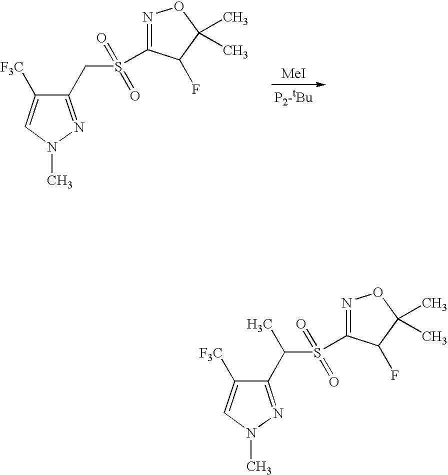

1-tert-butyl-2,2,4,4,4-pentakis(dimethylamino-2-lambda⁵-5,4-lambda⁵-5-catena-di(phosphazene) (P₂-tBu) (72 μl, 0.15 mmol) (2M in THF) was added slowly to a solution of 3-(4-fluoro-5,5-dimethyl-4,5-dihydro-isoxazol-3-ylsulfonylmethyl)-1-methyl-4-trifluoromethyl-1H-pyrazole (50 mg, 0.145 mmol) (Compound 4.03 of Table 53) in tetrahydrofuran (5 ml) under nitrogen at room temperature. After stirring for a further 10 minutes, methyl iodide (18 μl, 0.29 mmol) was added. After stirring for a further 30 minutes, the reaction mixture was quenched by the addition of water and dichloromethane. The organic extract was dried over magnesium sulfate and concentrated. The residue was purified by column chromatography on silica gel (eluent:ethyl acetate/hexane) to give Compound 4.04 of Table 54 as 3:2-mixture of diastereomers (161 mg, 90% yield).

Compound No. 2.12 and Compound No. 2.27 of Table 52. were prepared analogously.

### Example P14

### 3-(1-(4-fluoro-5,5-dimethyl-4,5-dihydro-isoxazol-3-ylsulfonyl)-2-methyl-eth-1-yl)-1-methyl-4-trifluoromethyl-1H-pyrazole

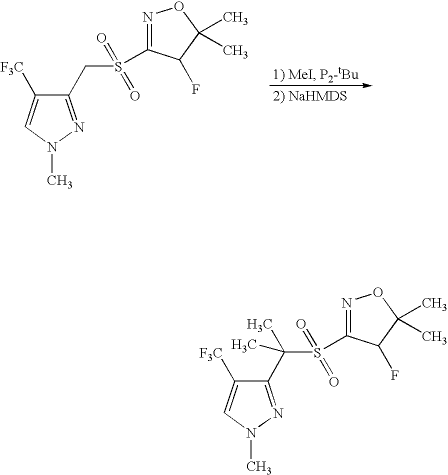

1-tert-butyl-2,2,4,4,4-pentakis(dimethylamino-2-lambda⁵-5,4-lambda⁵-5-catena-di(phosphazene) (P₂-tBu) (145 μl, 0.29 mmol) (2M in THF) was added slowly to a solution of 3-(4-fluoro-5,5-dimethyl-4,5-dihydro-isoxazol-3-ylsulfonylmethyl)-1-methyl-4-trifluoromethyl-1H-pyrazole (50 mg, 0.145 mmol) (Compound 4.03 of Table 54) and methyl iodide (36 μl, 0.58 mmol) in tetrahydrofuran under nitrogen at room temperature. After the addition of one equivalent of base and stirring for 4 hours, sodium hexamethyldisilazide (290 μl, 0.053 mol) (1M in THF) was added slowly to the reaction mixture. After stirring for a further 30 minutes, the reaction mixture was quenched by addition of water and dichloromethane. The chlorinated layer was separated, dried over magnesium sulfate and concentrated. The residue was purified by column chromatography on silica gel (eluent: ethyl acetate/hexane) to give Compound 4.05 of Table 54 (238 mg, 90% yield).

### Example P15

### Preparation of 3-[chloro-(1,3-dimethyl-1H-[1,2,4]triazol-5-yl)-methane-sulfonyl]-4-fluoro-5,5-dimethyl-4,5-dihydro-isoxazole and 3-[dichloro-(1,3-dimethyl-1H-[1,2,4]triazol-5-yl)-methanesulfonyl]-4-fluoro-5,5-dimethyl-4,5-dihydro-isoxazole

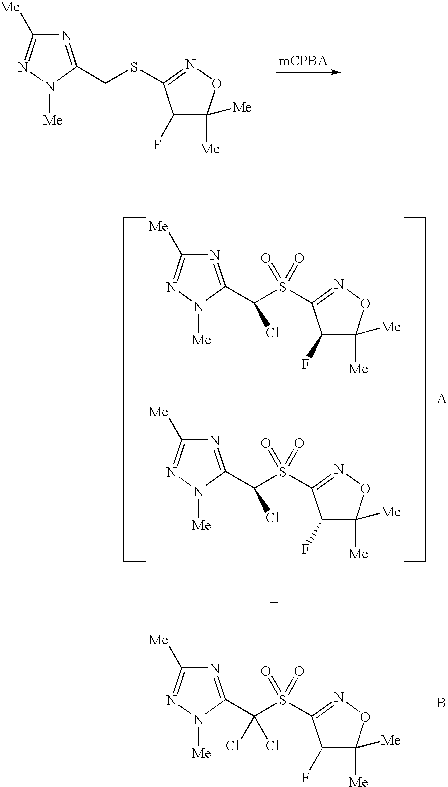

3-Chloroperoxybenzoic acid (mCPBA) (0.3 g, 1.7 mmol) was added in portions to a solution 3-[(1,3-dimethyl-1H-[1,2,4]triazol-5-yl)-methanesulfanyl]-4-fluoro-5,5-dimethyl-4,5-dihydro-isoxazole (0.13 g, 0.5 mmol) in dry dichloromethane (10 ml), which was cooled by an ice bath. The reaction mixture was allowed to warm to room temperature and was stored at room temperature for 72 hours. The suspension was further diluted with dichloromethane (20 ml) to give a clear solution, which was washed with aqueous sodium metabisulfite (10%), twice with aqueous sodium hydroxide (1M) and finally with brine. The solution was dried over magnesium sulfate and concentrated to give a colourless gum which was purified by column chromatography on silica gel (eluent: 1:4 ethyl acetate/hexane) to give Compound 8.06 of Table 58 as a colourless gum (10 mg, 7% yield) and Compound 8.05 of Table 58 as a white solid (18 mg, 14% yield). Compound 8.05 of Table 58 was obtained as a mixture of a racemic diastereomers in a 3:2 ratio.

### Example P16

### Preparation of 3-[chloro-(1,3-dimethyl-1H-[1,2,4]triazol-5-yl)-methane-sulfonyl]-4-fluoro-5,5-dimethyl-4,5-dihydro-isoxazole and 3-[(1,3-dimethyl-1H-[1,2,4]-triazol-5-yl)-methanesulfonyl]-4-fluoro-5,5-dimethyl-4,5-dihydro-isoxazole

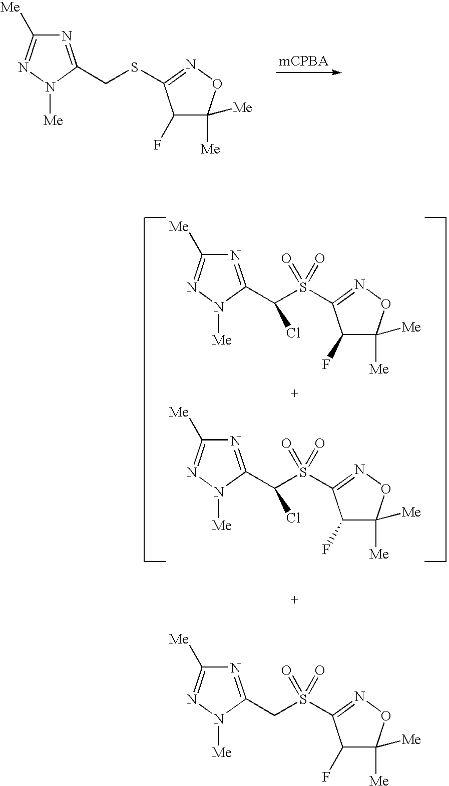

3-Chloroperoxybenzoic acid (mCPBA) (0.5 g, 0.29 mmol) was added in portions to a solution of 3-[(1,3-dimethyl-1H-[1,2,4]triazol-5-yl)-methanesulfanyl]-4-fluoro-5,5-dimethyl-4,5-dihydro-isoxazole (0.17 g, 0.66 mmol) in dry dichloromethane (10 ml), which was cooled by an ice bath. The reaction mixture was allowed to warm to room temperature and was stored at room temperature for 72 hours. The suspension was further diluted with dichloromethane (20 ml) to give a clear solution, which was washed with aqueous sodium metabisulfite (10%), twice with aqueous sodium hydroxide (1M) and finally with brine. The solution was dried over magnesium sulfate and concentrated to give a white solid which was purified by column chromatography on silica gel (eluent: 1:1 ethyl acetate/hexane) to give Compound 8.04 of Table 58 as a white solid (0.065 g, 33% yield) and Compound 8.05 of Table 58 (8 mg, 4% yield as a white solid. Compound 8.05 of Table 58 was obtained as a racemic mixture of diastereomers in a 3:2 ratio.

### Example P17

### Preparation of 1,3-dimethyl-4-(4-fluoro-5,5-dimethyl-4,5-dihydro-isoxazol-3-ylsulfanylmethyl)-5-(2,2,2-trifluoro-ethoxy)-1H-pyrazole

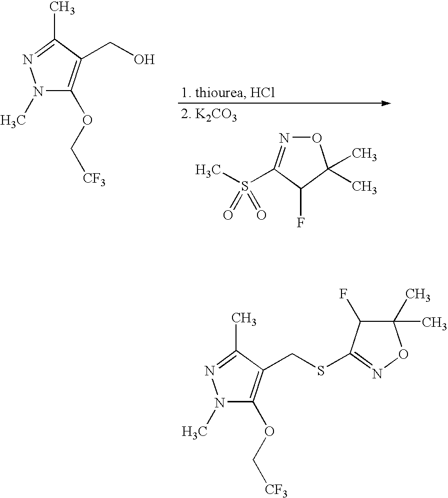

[1,3-Dimethyl-5-(2,2,2-trifluoro-ethoxy)-1H-pyrazol-4-yl]-methanol prepared according to WO 06/024820) (503 mg, 2.25 mmol), thiourea (205 mg, 2.69 mmol), concentrated hydrochloric acid (36%) (HCl (36%) (716 μl, 7.07 mmol), water (2.5 ml) and 1,4-dioxane (2.5 ml) were added to a 20 ml microwave tube and the reaction mixture was heated in the microwave for 10 minutes at 130° C. The reaction mixture was allowed to cool down before adding potassium carbonate (1.40 g, 10.0 mmol), 4-fluoro-3-methanesulfonyl-5,5-dimethyl-4,5-dihydro-isoxazole (657 mg, 3.37 mmol), 1,4-dioxane (1 ml) and water (1 ml). The reaction mixture was heated in the microwave for 16 minutes at 150° C. The reaction mixture was allowed to cool down before partitioning between ethyl acetate and water. The organic extract was washed with brine, dried over magnesium sulfate and concentrated. The residue was purified by column chromatography on silica gel (eluent: 0-50% ethyl acetate in hexane) to give Compound No. 2.60 of Table 52 (396 mg, 53% yield) as a colourless oil.

### Example P18

### Preparation of 4,5-dimethyl-3-(4-fluoro-5,5-dimethyl-4,5-dihydro-isoxazol-3-ylsulfanylmethyl)-4H-[1,2,4]triazole

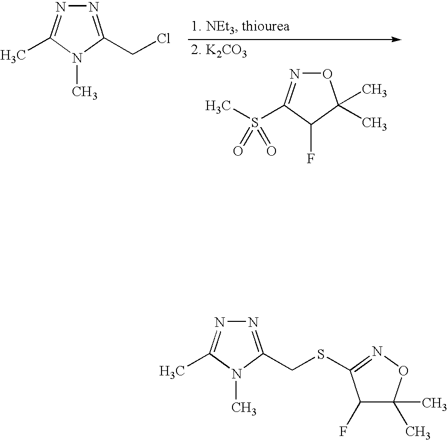

To a solution of 3-chloromethyl-4,5-dimethyl-4H-[1,2,4]triazole (preparation according to Synthesis, 2006, 156-160) (564 mg, 3.1 mmol) in ethanol (20 ml) was added sequentially triethyl amine (432 μl, 3.1 mmol) and thiourea (236 mg, 3.1 mmol). The reaction mixture was heated to reflux for 1 hour. The reaction mixture was allowed to cool to room temperature before the addition of 4-fluoro-3-methanesulfonyl-5,5-di-methyl-4,5-dihydro-isoxazole (665 mg, 3.3 mmol) and potassium carbonate (591 mg, 3.6 mmol). The reaction mixture was heated to reflux for 6 hours and then stored at room temperature for 72 hours. The mixture was concentrated and the residue purified by column chromatography on silica gel (eluent: 0-20% methanol in chloromethane) to give Compound No. 13.01 of Table 63 as an oil (636 mg, 80% yield).

Compound No. 8.07 of Table 58 and Compound No. 12.01 of Table 62 were prepared analogously.

### Example P19

### Preparation of 4-[difluoro-(4-fluoro-5,5-dimethyl-4,5-dihydro-isoxazole-3-sulfinyl)-methyl]-5-difluoromethoxy-2-methyl-2H-[1,2,3]triazole

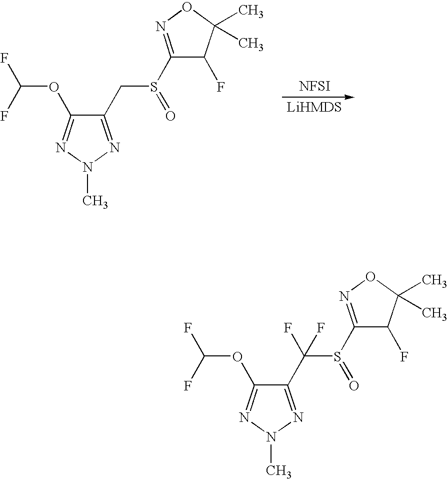

4-Difluoromethoxy-5-(4-fluoro-5,5-dimethyl-4,5-dihydro-isoxazole-3-sulfinylmethyl)-2-methyl-2H-[1,2,3]triazole (Compound No. 7.59, Table 57) (102 mg, 0.32 mmol) and N-fluorobenzenesulfonimide (209 mg, 0.63 mmol) were dissolved in dry tetrahydrofuran (7.6 ml), cooled to −78° C. and lithium hexamethyldisilazide (LiHMDS) (0.5 ml, 1M in THF) was added slowly with stirring. The reaction was stirred at −78° C. for 2 hours and then quenched by addition of saturated aqueous ammonium chloride and extracted with ethyl acetate. The organic extracts were washed with brine, dried over magnesium sulfate and concentrated. The residue was purified by column chromatography on silica (eluent: ethyl acetate/hexane) to give Compound No. 7.61 of Table 57 as 7:3 mixture of diastereoisomers (47.7 mg, 42% yield).

Compound No. 7.05 and Compound No. 7.48 of Table 57 were prepared analogously.

### Example P20

### Preparation of 4-[difluoro-(4-fluoro-5,5-dimethyl-4,5-dihydro-isoxazole-3-sulfonyl)-methyl]-5-difluoromethoxy-2-methyl-2H-[1,2,3]triazole

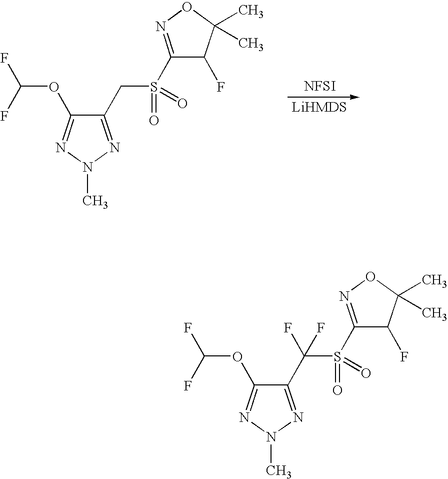

4-Difluoromethoxy-5-(4-fluoro-5,5-dimethyl-4,5-dihydro-isoxazole-3-sulfonyl-methyl)-2-methyl-2H-[1,2,3]triazole (124 mg, 0.36 mmol) and N-fluorobenzene-sulfonimide (228 mg, 0.72 mmol) were dissolved in dry dichloromethane (3 ml), cooled to −30° C. and lithium hexamethyldisilazide (LiHMDS) (0.73 ml, 0.73 mmol) (1M in THF) was added slowly. The reaction was stirred at −30° C. for 15 minutes then stirred without cooling until a precipitate began to form. The reaction was diluted with dichloromethane (6 ml) and quenched by addition of saturated aqueous ammonium chloride (0.4 nil) was added and the reaction stirred for 10 minutes. The reaction was filtered through celite, the filtrate shaken with water (3 ml) then passed through a phase separating cartridge. The solvent was removed from the filtrate under vacuum. The residue was purified by column chromatography on silica (eluent: ethyl acetate/hexane) to give Compound No. 7.62 of Table 57 as a straw coloured gum (123 mg, 90% yield).

Compound No. 7.06, Compound No. 7.49 and Compound No. 7.57 of Table 57 were prepared analogously.

The compounds mentioned in the following Tables can be prepared in analogous manner.

TABLE 51
Compounds of formula I.1

I.1

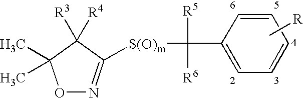

| | | | | | | | M.p. | NMR data |
|---|---|---|---|---|---|---|---|---|
| No | R | m | R³ | R ⁴ | R⁵ | R⁶ | [° C.] | (CDCl₃, 400 MHz) |
| 1.01 | 2,6-di-F | 0 | Br | H | H | H | oil | 1.4 (s, 3H, Me), 1.65 (s, |
| | | | | | | | | 3H, Me), 4.4 (s, 2H, |
| | | | | | | | | CH₂), 4.73 (s, 1H, CH), |
| | | | | | | | | 6.9 (m, 2H, CH), 7.27 |
| | | | | | | | | (m, 1H, CH). |
| 1.02 | 2,6-di-F | 1 | Br | H | H | H | 97 | 4:1-Mixture of diastereo- |
| | | | | | | | | mers; Diastereomer 1 |
| | | | | | | | | (major isomer): 1.5 (s, |
| | | | | | | | | 3H, Me), 1.75 (s, 3H, |
| | | | | | | | | Me), 4.6 (d, 1H, CH₂), |
| | | | | | | | | 4.63 (d, 1H, CH₂), 5.21 |
| | | | | | | | | (s, 1H, CH), 7.0 (m, 2H, |
| | | | | | | | | CH), 7.38 (m, 1H, CH). |
| | | | | | | | | Diastereomer 2 (minor |
| | | | | | | | | isomer): 1.48 (s, 3H, |
| | | | | | | | | Me), 1.75 (s, 3H, Me), |
| | | | | | | | | 4.67 (d, 1H, CH₂), 4.86 |
| | | | | | | | | (d, 1H, CH₂), 5.25 (s, |
| | | | | | | | | 1H, CH), 7.0 (m, 2H, |
| | | | | | | | | CH), 7.38 (m, 1H, CH). |
| 1.03 | 2,6-di-F | 2 | Br | H | H | H | 125 | 1.47 (s, 3H, Me), 1.75 (s, |
| | | | | | | | | 3H, Me), 4.84 (s, 2H, |
| | | | | | | | | CH₂), 5.11 (s, 1H, CH), |
| | | | | | | | | 7.00 (m, 2H, CH), 7.4 |
| | | | | | | | | (m, 1H, CH). |
| 1.04 | 2-OCF₃ | 0 | Br | H | H | H | oil | 1.38 (s, 3H, Me), 1.62 (s, |
| | | | | | | | | 3H, Me), 4.35 (d, 1H, |
| | | | | | | | | CH₂), 4.4 (d, 1H, CH₂), |
| | | | | | | | | 4.68 (s, 1H, CH), 7.25 |
| | | | | | | | | (m, 2H, CH), 7.32 (m, |
| | | | | | | | | 1H, CH), 7.55 (m, 1H, |
| | | | | | | | | CH). |
| 1.05 | 2-OCF₃ | 2 | Br | H | H | H | solid | 1.4 (s, 3H, Me), 1.73 (s, |
| | | | | | | | | 3H, Me), 4.79 (d, 1H, |
| | | | | | | | | CH₂), 4.82 (d, 1H, |
| | | | | | | | | CH₂), 5.08 (s, 1H, CH), |
| | | | | | | | | 7.35 (m, 2H, CH), 7.48 |
| | | | | | | | | (m, 1H, CH), 7.6 (d, 1H, |
| | | | | | | | | CH). |
| 1.06 | 2-OCF₃ | 0 | Cl | H | H | H | | 1.37 (s, 3H, Me), 1.55 (s, |
| | | | | | | | | 3H, Me), 4.34 (d, 1H, |
| | | | | | | | | CH₂), 4.38 (d, 1H, |
| | | | | | | | | CH₂), 4.59 (s, 1H, CH), |
| | | | | | | | | 7.25 (m, 1H, CH), 7.29 |
| | | | | | | | | (m, 1H, CH), 7.32 (m, |
| | | | | | | | | 1H, CH), 7.56 (m, 1H, |
| | | | | | | | | CH). |
| 1.07 | 2-OCF₃ | 1 | Cl | H | H | H | solid | 3:1-Mixture of diastereo- |
| | | | | | | | | mers; Diastereomer 1 |
| | | | | | | | | (major isomer): 1.4 (s, |
| | | | | | | | | 3H, Me), 1.66 (d, 3H, |
| | | | | | | | | Me), 4.4 (d, 1H, CH₂), |
| | | | | | | | | 4.78 (d, 1H, CH₂), 5.09 |
| | | | | | | | | (s, 1H, CH), 7.33 (m, |
| | | | | | | | | 2H, CH), 7.43 (m, 1H, |
| | | | | | | | | CH), 7.52 (m, 1H, CH). |
| | | | | | | | | Diastereomer 2 (minor |
| | | | | | | | | isomer): 1.42 (s, 3H, |
| | | | | | | | | Me), 1.68 (d, 3H, Me), |
| | | | | | | | | 4.63 (d, 1H, CH₂), 4.69 |
| | | | | | | | | (d, 1H, CH₂), 5.22 (s, |
| | | | | | | | | 1H, CH), 7.32 (m, 2H, |
| | | | | | | | | CH), 7.44 (m, 1H, |
| | | | | | | | | CH), 7.5 (m, 1H, CH). |
| 1.08 | 2-OCF₃ | 0 | Cl | Cl | H | H | | 1.6 (s, 6H, Me), 4.35 (s, |
| | | | | | | | | 2H, CH₂), 7.2-7.3 (m, |
| | | | | | | | | 2H, CH), 7.3-7.4 (m, |
| | | | | | | | | 1H, CH), 7.5-7.6 (m, |
| | | | | | | | | 1H, CH). |
| 1.09 | 2-OCF₃ | 2 | Cl | Cl | H | H | oil | 1.6 (s, 6H, Me), 4.85 (s, |
| | | | | | | | | 2H, CH₂), 7.2-7.3 (m, |
| | | | | | | | | 2H, CH), 7.3-7.4 (m, 1H, |
| | | | | | | | | CH), 7.5-7.6 (m, 1H, |
| | | | | | | | | CH). |
| 1.10 | 2-OCF₃ | 0 | F | H | H | H | | 1.3 (s, 3H, Me), 1.5 (d, |
| | | | | | | | | 3H, Me), 4.37 (s, 2H, |
| | | | | | | | | CH₂), 5.01 (d, 1H, CH), |
| | | | | | | | | 7.29 (m, 2H, CH), 7.34 |
| | | | | | | | | (m, 1H, CH), 7.58 (m, |
| | | | | | | | | 1H, CH). |
| 1.11 | 2-OCF₃ | 2 | F | H | H | H | 74 | 1.35(s, 3H, Me), 1.59 (d, |
| | | | | | | | | 3H, Me), 4.68 (d, 1H, |
| | | | | | | | | CH₂), 4.71 (d, 1H, |
| | | | | | | | | CH₂), 5.45 (d, 1H, CH), |
| | | | | | | | | 7.35 (m, 2H, CH), 7.49 |
| | | | | | | | | (m, 1H, CH), 7.6 (m, |
| | | | | | | | | 1H, CH). |
| 1.12 | 2-OCF₃ | 2 | F | H | F | F | 71- | 1.47 (s, 3H, Me), 1.65 |
| | | | | | | | 72 | (d, 3H, Me), 4.9 (d, 1H, |
| | | | | | | | | CH), 7.4-7.5 (m, 2H, |
| | | | | | | | | CH), 7.65-7.75 (m, 1H, |
| | | | | | | | | CH), 7.75-7.8 (m, 1H, |
| | | | | | | | | CH). |
| 1.13 | 2-OCF₃ | 2 | F | H | Cl | H | gum | 2:1-Mixture of diastereo- |
| | | | | | | | | mers; Diastereomer 1 |
| | | | | | | | | (major isomer): 1.49 (s, |
| | | | | | | | | 3H, Me), 1.65 (d, 3H, |
| | | | | | | | | Me), 5.57 (d, 1H, CH), |
| | | | | | | | | 6.38 (s, 1H, CH), 7.37 |
| | | | | | | | | (m, 1H, CH), 7.45 |
| | | | | | | | | (m, 1H, CH), 7.59 |
| | | | | | | | | (m, 1H, CH), 8.0 (m, |
| | | | | | | | | 1H, CH). |
| | | | | | | | | Diastereomer 2 (minor |
| | | | | | | | | isomer): 1.35 (s, 3H, |
| | | | | | | | | Me), 1.61 (d, 3H, Me), |
| | | | | | | | | 5.4 (d, 1H, CH), 6.42 (s, |
| | | | | | | | | 1H, CH), 7.37 (m, 1H, |
| | | | | | | | | CH), 7.45 (m, 1H, CH), |
| | | | | | | | | 7.59 (m, 1H, CH), 8.0 |
| | | | | | | | | (m, 1H, CH). |
| 1.14 | 2,6-di-F | 0 | F | H | H | H | oil | 1.34 (s, 3H, Me), 1.5 (m, |
| | | | | | | | | 3H, Me), 4.49 (s, 2H, |
| | | | | | | | | CH₂), 4.99-5.14 (d, |
| | | | | | | | | 1H, CH), 6.9 (m, 2H, |
| | | | | | | | | CH), 7.25 (m, 1H, CH). |
| 1.15 | 2,6-di-F | 1 | F | H | H | H | 109- | Diastereomer 1: |
| | | | | | | | 111 | 1.4 (s, 3H, Me), 1.6 (m, |
| | | | | | | | | 3H, Me), 4.5 (d, 1H, |
| | | | | | | | | CH₂), 4.6 (d, 1H, CH₂), |
| | | | | | | | | 5.8 (d, 1H, CH), 7.0 (m, |
| | | | | | | | | 2H, CH), 7.38 (m, 1H, |
| | | | | | | | | CH). |
| 1.16 | 2,6-di-F | 1 | F | H | H | H | 100- | Diastereomer 2: |
| | | | | | | | 102 | 1.39 (s, 3H, Me), 1.6 |
| | | | | | | | | (m, 3H, Me), 4.59 (s, |
| | | | | | | | | 2H, CH₂), 5.6 (d, 1H, |
| | | | | | | | | CH), 6.98 (m, 2H, CH), |
| | | | | | | | | 7.37 (m, 1H, CH). |
| 1.17 | 2,6-di-F | 2 | F | H | H | H | 98- | 1.4 (s, 3H, Me), 1.6 (m, |
| | | | | | | | 99 | 3H, Me), 4.71 (s, 2H, |
| | | | | | | | | CH₂), 5.44-5.59 (d, |
| | | | | | | | | 1H, CH), 7.0 (t, 2H, |
| | | | | | | | | CH), 7.42 (m, 1H, CH). |
| 1.18 | 2,6-di-F | 2 | F | H | F | F | 115- | 1.47 (s, 3H, Me), 1.66 |
| | | | | | | | 116 | (m, 3H, Me), 5.50 (d, |
| | | | | | | | | 1H, CH), 7.07 (m, |
| | | | | | | | | 2H, CH), 7.6 (m, 1H, |
| | | | | | | | | CH). |
| 1.19 | 2-OCF₃ | 1 | F | H | H | H | 104- | Diastereomer A: |
| | | | | | | | 105 | 1.38 (s, 3H, Me), 1.6 (d, |
| | | | | | | | | 3H, Me), 4.47 (d, 1H, |
| | | | | | | | | CH₂), 4.5 (d, 1H, CH₂), |
| | | | | | | | | 5.78 (d, 1H, CH), 7.26- |
| | | | | | | | | 7.36 (m, 2H, CH), 7.38- |
| | | | | | | | | 7.46 (m, 2H, CH). |
| 1.20 | 2-OCF₃ | 1 | F | H | H | H | 76- | Diastereomer B: |
| | | | | | | | 77 | 1.32 (s, 3H, Me), 1.58 |
| | | | | | | | | (d, 3H, Me), 4.35 (d, 1H, |
| | | | | | | | | CH₂), 4.7 (d, 1H, CH₂), |
| | | | | | | | | 5.45 (d, 1H, CH), 7.3- |
| | | | | | | | | 7.35 (m, 2H, CH), 7.4- |
| | | | | | | | | 7.45 (m, 1H, CH), 7.5- |
| | | | | | | | | 7.55 (m, 1H, CH). |

Key:
Me = methyl; s = singlet; m = multiplet; d = doublet.

TABLE 52
Compounds of formula I.2

| | |
|---|---|
| | I.2 |
| 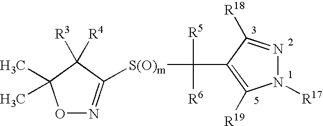 | |

| | | | | | | | | | M.p. | ¹H-NMR (CDCl₃, |
|---|---|---|---|---|---|---|---|---|---|---|
| No | m | R³ | R⁴ | R⁵ | R⁶ | R¹⁷ | R¹⁸ | R¹⁹ | [° C.] | 400 MHz) |
| 2.01 | 0 | Br | H | H | H | Me | CF₃ | OCH₂ | oil | 1.4 (s, 3H, Me), 1.55 |
| | | | | | | | | CF₃ | | (s, 3H, Me), 3.78 (s, |
| | | | | | | | | | | 3H, Me), 4.23 (d, |
| | | | | | | | | | | 1H, CH₂), 4.3 (d, |
| | | | | | | | | | | 1H, CH₂), 4.62 (q, |
| | | | | | | | | | | 2H, CH₂), 4.69 (s, |
| | | | | | | | | | | 1H, CH). |
| 2.02 | 1 | Br | H | H | H | Me | CF₃ | OCH₂ | Solid | 3:1-Mixture of |
| | | | | | | | | CF₃ | | diastereomers; |
| | | | | | | | | | | Diastereomer 1 |
| | | | | | | | | | | (major isomer): |
| | | | | | | | | | | 1.5 (s, 3H, Me), 1.74 |
| | | | | | | | | | | (s, 3H, Me), 3.81 (s, |
| | | | | | | | | | | 3H, Me), 4.35 (d, 1H, |
| | | | | | | | | | | CH₂), 4.4 (d, 1H, |
| | | | | | | | | | | CH₂), 4.68 (m, 1H, |
| | | | | | | | | | | CH₂), 4.81 (m, 1H, |
| | | | | | | | | | | CH₂), 5.2 (s, 1H, CH). |
| | | | | | | | | | | Diastereomer 2 |
| | | | | | | | | | | (minor isomer): |
| | | | | | | | | | | 1.5 (s, 3H, Me), 1.74 |
| | | | | | | | | | | (s, 3H, Me), 3.81 (s, |
| | | | | | | | | | | 3H, Me), 4.18 (m, |
| | | | | | | | | | | 2H, CH₂), 4.82 (m, |
| | | | | | | | | | | 2H, CH₂), 5.25 (s, |
| | | | | | | | | | | 1H, CH). |
| 2.03 | 2 | Br | H | H | H | Me | CF₃ | OCH₂ | 126 | 1.5 (s, 3H, Me), 1.78 |
| | | | | | | | | CF₃ | | (s, 3H, Me), 3.85 (s, |
| | | | | | | | | | | 3H, Me), 4.6 (d, 1H, |
| | | | | | | | | | | CH₂), 4.7 (q, 2H, |
| | | | | | | | | | | CH₂), 4.71 (d, 1H, |
| | | | | | | | | | | CH₂), 5.15 (s, 1H, CH). |
| 2.04 | 0 | Br | H | H | H | Me | CF₃ | OCHF₂ | oil | 1.41 (s, 3H, Me), |
| | | | | | | | | | | 1.64 (s, 3H, Me), |
| | | | | | | | | | | 3.82 (s, 3H, Me), |
| | | | | | | | | | | 4.21 (d, 1H, CH₂), |
| | | | | | | | | | | 4.26 (d, 1H, CH₂), |
| | | | | | | | | | | 4.7 (s, 1H, CH), 6.7 |
| | | | | | | | | | | (t, 1H, CH). |
| 2.05 | 1 | Br | H | H | H | Me | CF₃ | OCHF₂ | oil | 7:2-Mixture of |
| | | | | | | | | | | diastereomers; |
| | | | | | | | | | | Diastereomer 1 |
| | | | | | | | | | | (major isomer): |
| | | | | | | | | | | 1.5 (s, 3H, Me), 1.75 |
| | | | | | | | | | | (s, 3H, Me), 3.88 (s, |
| | | | | | | | | | | 3H, Me), 4.35 (d, |
| | | | | | | | | | | 1H, CH₂), 4.41 (d, |
| | | | | | | | | | | 1H, CH₂), 5.2 (s, 1H, |
| | | | | | | | | | | CH), 6.95 (m, 1H, CH). |
| | | | | | | | | | | Diastereomer 2 |
| | | | | | | | | | | (minor isomer). |
| | | | | | | | | | | 1.49 (s, 3H, Me), |
| | | | | | | | | | | 1.75 (s, 3H, Me), |
| | | | | | | | | | | 3.88 (s, 3H, Me), |
| | | | | | | | | | | 4.24 (d, 1H, CH₂), |
| | | | | | | | | | | 4.8 (d, 1H, CH₂), |
| | | | | | | | | | | 5.22 (s, 1H, CH), |
| | | | | | | | | | | 7.03 (m, 1H, CH). |
| 2.06 | 2 | Br | H | H | H | Me | CF₃ | OCHF₂ | oil | 1.49 (s, 3H, Me), |
| | | | | | | | | | | 1.75 (s, 3H, Me), 3.9 |
| | | | | | | | | | | (s, 3H, Me), 4.61 (d, |
| | | | | | | | | | | 1H, CH₂), 4.7 (d, 1H, |
| | | | | | | | | | | CH₂), 5.15 (s, 1H, CH), |
| | | | | | | | | | | 6.85 (t, 1H, CH). |
| 2.07 | 0 | F | H | H | H | Me | CF₃ | OCH₂ | 50-51 | 1.33 (s, 3H, Me), |
| | | | | | | | | CF₃ | | 1.51 (m, 3H, Me), |
| | | | | | | | | | | 3.8 (s, 3H, Me), 4.25 |
| | | | | | | | | | | (d, 1H, CH₂), 4.29 |
| | | | | | | | | | | (d, 1H, CH₂), 4.62 |
| | | | | | | | | | | (q, 2H, CH₂), 5.04 |
| | | | | | | | | | | (d, 1H, CH). |
| 2.08 | 1 | F | H | H | H | Me | CF₃ | OCH₂ | gum | 2:3-Mixture of |
| | | | | | | | | CF₃ | | diastereomers; |
| | | | | | | | | | | Diastereomer 1 |
| | | | | | | | | | | (minor isomer): |
| | | | | | | | | | | 1.42 (s, 3H, Me), |
| | | | | | | | | | | 1.61 (d, 3H, Me), |
| | | | | | | | | | | 3.83 (s, 3H, Me), |
| | | | | | | | | | | 4.31 (d, 1H, CH₂), |
| | | | | | | | | | | 4.35 (d, 1H, CH₂), |
| | | | | | | | | | | 4.7 (m, 1H, CH₂), |
| | | | | | | | | | | 4.82 (m, 1H, CH₂), |
| | | | | | | | | | | 5.55 (d, 1H, CH). |
| | | | | | | | | | | Diastereomer 2 |
| | | | | | | | | | | (major isomer): |
| | | | | | | | | | | 1.41 (s, 3H, Me), |
| | | | | | | | | | | 1.61 (d, 3H, Me), |
| | | | | | | | | | | 3.83 (s, 3H, Me), |
| | | | | | | | | | | 4.08 (d, 1H, CH₂), |
| | | | | | | | | | | 4.45 (d, 1H, CH₂), |
| | | | | | | | | | | 4.7 (m, 1H, CH₂), |
| | | | | | | | | | | 4.82 (m, 1H, CH₂), |
| | | | | | | | | | | 5.75 (d, 1H, CH). |
| 2.09 | 1 | F | H | H | H | Me | CF₃ | OCH₂ | solid | Diastereomer 1: |
| | | | | | | | | CF₃ | | 1.42 (s, 3H, Me), |
| | | | | | | | | | | 1.61 (d, 3H, Me), |
| | | | | | | | | | | 3.83 (s, 3H, Me), |
| | | | | | | | | | | 4.31 (d, 1H, CH₂), |
| | | | | | | | | | | 4.35 (d, 1H, CH₂), |
| | | | | | | | | | | 4.7 (m, 1H, CH₂), |
| | | | | | | | | | | 4.82 (m, 1H, CH₂), |
| | | | | | | | | | | 5.55 (d, 1H, CH). |
| 2.10 | 1 | F | H | H | H | Me | CF₃ | OCH₂ | oil | Diastereomer 2: |
| | | | | | | | | CF₃ | | 1.41 (s, 3H, Me), |
| | | | | | | | | | | 1.61 (d, 3H, Me), |
| | | | | | | | | | | 3.83 (s, 3H, Me), |
| | | | | | | | | | | 4.08 (d, 1H, CH₂), |
| | | | | | | | | | | 4.45 (d, 1H, CH₂), |
| | | | | | | | | | | 4.7 (m, 1H, CH₂), |
| | | | | | | | | | | 4.82 (m, 1H, CH₂), |
| | | | | | | | | | | 5.75 (d, 1H, CH). |
| 2.11 | 2 | F | H | H | H | Me | CF₃ | OCH₂ | 78-79 | 1.42 (s, 3H, Me), |
| | | | | | | | | CF₃ | | 1.62 (d, 3H, Me), |
| | | | | | | | | | | 3.85 (s, 3H, Me), |
| | | | | | | | | | | 4.52 (d, 1H, CH₂), |
| | | | | | | | | | | 4.57 (d, 1H, CH₂), |
| | | | | | | | | | | 4.7 (q, 2H, CH₂), |
| | | | | | | | | | | 5.56 (d, 1H, CH). |
| 2.12 | 2 | F | H | Me | H | Me | CF₃ | OCH₂ | oil | 1:1-Mixture of |
| | | | | | | | | CF₃ | | diastereomers |
| | | | | | | | | | | 1.3 (s, 6H, Me), 1.60 |
| | | | | | | | | | | (d, 3H, Me), 1.65 (d, |
| | | | | | | | | | | 3H, Me), 1.95 (d, |
| | | | | | | | | | | 6H, Me), 3.8 (s, 6H, |
| | | | | | | | | | | Me), 4.3-4.4 (m, 1H, |
| | | | | | | | | | | CH₂), 4.6-4.8 (m, |
| | | | | | | | | | | 4H, CH₂ and CH), |
| | | | | | | | | | | 4.9-5.0 (m, 1H, CH₂), |
| | | | | | | | | | | 5.4 (d, 1H, CH), 5.5 (dd, |
| | | | | | | | | | | 1H, CH). |
| 2.13 | 0 | Cl | H | H | H | Me | CF₃ | OCH₂ | oil | 1.4 (s, 3H, Me), 1.6 |
| | | | | | | | | CF₃ | | (s, 3H, Me), 3.79 (s, |
| | | | | | | | | | | 3H, Me), 4.24 (d, |
| | | | | | | | | | | 1H, CH₂), 4.29 (d, |
| | | | | | | | | | | 1H, CH₂), 4.62 (s, |
| | | | | | | | | | | 1H, CH), 4.63 (q, |
| | | | | | | | | | | 2H, CH₂). |
| 2.14 | 1 | Cl | H | H | H | Me | CF₃ | OCH₂ | oil | 7:3-Mixture of |
| | | | | | | | | CF₃ | | diastereomers; |
| | | | | | | | | | | Diastereomer 1 |
| | | | | | | | | | | (major isomer): |
| | | | | | | | | | | 1.49 (s, 3H, Me), 1.7 |
| | | | | | | | | | | (s, 3H, Me), 3.82 (s, |
| | | | | | | | | | | 3H, Me), 4.35 (d, 1H, |
| | | | | | | | | | | CH₂), 4.4 (d, 1H, |
| | | | | | | | | | | CH₂), 4.69 (m, 1H, |
| | | | | | | | | | | CH₂), 4.82 (m, 1H, |
| | | | | | | | | | | CH₂), 5.11 (s, 1H, CH). |
| | | | | | | | | | | Diastereomer 2 |
| | | | | | | | | | | (minor isomer): |
| | | | | | | | | | | 1.47 (s, 3H, Me), 1.7 |
| | | | | | | | | | | (s, 3H, Me), 3.82 (s, |
| | | | | | | | | | | 3H, Me), 4.15 (d, 1H, |
| | | | | | | | | | | CH₂), 4.7 (d, 1H, |
| | | | | | | | | | | CH₂), 4.75 (m, 1H, |
| | | | | | | | | | | CH₂), 4.82 (m, 1H, |
| | | | | | | | | | | CH₂), 5.21 (s, 1H, CH). |
| 2.15 | 2 | Cl | H | H | H | Me | CF₃ | OCH₂ | 111-112 | 1.49 (s, 3H, Me), 1.7 |
| | | | | | | | | CF₃ | | (d, 3H, Me), 3.85 (s, |
| | | | | | | | | | | 3H, Me), 4.6 (d, 1H, |
| | | | | | | | | | | CH₂), 4.66 (d, 1H, |
| | | | | | | | | | | CH₂), 4.71 (q, 2H, |
| | | | | | | | | | | CH₂), 5.11 (d, 1H, CH). |
| 2.16 | 0 | Cl | H | H | H | Me | CF₃ | OCHF₂ | | 1.39 (s, 3H, Me), |
| | | | | | | | | | | 1.56 (s, 3H, Me), |
| | | | | | | | | | | 3.84 (s, 3H, Me), 4.1 |
| | | | | | | | | | | (d, 1H, CH₂), 4.26 |
| | | | | | | | | | | (d, 1H, CH₂), 4.6 (s, |
| | | | | | | | | | | 1H, CH), 6.71 (t, 1H, CH). |
| 2.17 | 1 | Cl | H | H | H | Me | CF₃ | OCHF₂ | Solid | 3:1-Mixture of |
| | | | | | | | | | | diastereomers; |
| | | | | | | | | | | Diastereomer 1 |
| | | | | | | | | | | (major isomer): |
| | | | | | | | | | | 1.48 (s, 3H, Me), |
| | | | | | | | | | | 1.69 (s, 3H, Me), |
| | | | | | | | | | | 3.88 (s, 3H, Me), |
| | | | | | | | | | | 4.34 (d, 1H, CH₂), |
| | | | | | | | | | | 4.41 (d, 1H, CH₂), |
| | | | | | | | | | | 5.11 (s, 1H, CH), |
| | | | | | | | | | | 6.95 (dd, 1H, CH). |
| | | | | | | | | | | Diastereomer 2 |
| | | | | | | | | | | (minor isomer): |
| | | | | | | | | | | 1.46 (s, 3H, Me), 1.7 |
| | | | | | | | | | | (s, 3H, Me), 3.88 (s, |
| | | | | | | | | | | 3H, Me), 4.21 (d, |
| | | | | | | | | | | 1H, CH₂), 4.72 (d, |
| | | | | | | | | | | 1H, CH₂), 5.2 (s, 1H, |
| | | | | | | | | | | CH), 7.01 (dd, 1H, CH). |
| 2.18 | 2 | Cl | H | H | H | Me | CF₃ | OCHF₂ | oil | 1.46 (s, 3H, Me), 1.7 |
| | | | | | | | | | | (s, 3H, Me), 3.89 (s, |
| | | | | | | | | | | 3H, Me), 4.6 (d, 1H, |
| | | | | | | | | | | CH₂), 4.66 (d, 1H, |
| | | | | | | | | | | CH₂), 5.1 (s, 1H, |
| | | | | | | | | | | CH), 6.82 (t, 1H, CH). |
| 2.19 | 0 | Cl | Cl | H | H | Me | CF₃ | OCH₂ | | 1.52 (s, 3H, Me), 1.6 |
| | | | | | | | | CF₃ | | (s, 3H, Me), 3.8 (s, |
| | | | | | | | | | | 3H, Me), 4.28 (s, |
| | | | | | | | | | | 2H, CH₂), 4.62 (q, |
| | | | | | | | | | | 2H, CH₂). |
| 2.20 | 1 | Cl | Cl | H | H | Me | CF₃ | OCH₂ | 56 | 1.69 (s, 6H, Me), |
| | | | | | | | | CF₃ | | 3.82 (s, 3H, Me), |
| | | | | | | | | | | 4.35 (d, 1H, CH₂), |
| | | | | | | | | | | 4.65 (d, 1H, CH₂), |
| | | | | | | | | | | 4.74 (m, 1H, CH₂), |
| | | | | | | | | | | 4.87 (m, 1H, CH₂). |
| 2.21 | 2 | Cl | Cl | H | H | Me | CF₃ | OCH₂ | solid | 1.65 (s, 6H, Me), |
| | | | | | | | | CF₃ | | 3.85 (s, 3H, Me), |
| | | | | | | | | | | 4.68 (s, 2H, CH₂), |
| | | | | | | | | | | 4.71 (q, 2H, CH₂). |
| 2.22 | 0 | F | H | H | H | Me | CF₃ | OCHF₂ | 35-37 | 1.31 (s, 3H, Me), |
| | | | | | | | | | | 1.49 (m, 3H, Me) |
| | | | | | | | | | | 3.82 (s, 3H, Me), |
| | | | | | | | | | | 4.24 (s, 2H, CH₂), |
| | | | | | | | | | | 5.02 (d, 1H, CH), |
| | | | | | | | | | | 6.69 (t, 1H, CH). |
| 2.23 | 1 | F | H | H | H | Me | CF₃ | OCHF₂ | 111-113 | 3:5-Mixture of |
| | | | | | | | | | | Diastereomers; |
| | | | | | | | | | | Diastereomer A |
| | | | | | | | | | | (minor isomer): |
| | | | | | | | | | | 1.42 (s, 3H, Me), |
| | | | | | | | | | | 1.62 (m, 3H, Me), |
| | | | | | | | | | | 3.85 (s, 3H, Me), |
| | | | | | | | | | | 4.15 (d, 1H, CH₂), |
| | | | | | | | | | | 4.52 (d, 1H, CH₂), |
| | | | | | | | | | | 5.75 (d, 1H, CH), |
| | | | | | | | | | | 6.94 (t, 1H, CH). |
| | | | | | | | | | | Diastereomer B |
| | | | | | | | | | | (major isomer): |
| | | | | | | | | | | 1.42 (s, 3H, Me), |
| | | | | | | | | | | 1.62 (m, 3H, Me), |
| | | | | | | | | | | 3.85 (s, 3H, Me), |
| | | | | | | | | | | 4.30 (d, 1H, CH₂), |
| | | | | | | | | | | 4.37 (d, 1H, CH₂), |
| | | | | | | | | | | 5.55 (d, H, CH), |
| | | | | | | | | | | 6.98 (t, 1H, CH). |
| 2.24 | 1 | F | H | H | H | Me | CF₃ | OCHF₂ | 141-142 | Diastereomer A: |
| | | | | | | | | | | 1.42 (s, 3H, Me), |
| | | | | | | | | | | 1.62 (m, 3H, Me), |
| | | | | | | | | | | 3.85 (s, 3H, Me), |
| | | | | | | | | | | 4.15 (d, 1H, CH₂), |
| | | | | | | | | | | 4.52 (d, 1H, CH₂), |
| | | | | | | | | | | 5.75 (d, 1H, CH), |
| | | | | | | | | | | 6.94 (t, 1H, CH). |
| 2.25 | 1 | F | H | H | H | Me | CF₃ | OCHF₂ | 88-89 | Diastereomer B: |
| | | | | | | | | | | 1.42 (s, 3H, Me), |
| | | | | | | | | | | 1.62 (m, 3H, Me), |
| | | | | | | | | | | 3.85 (s, 3H, Me), |
| | | | | | | | | | | 4.30 (d, 1H, CH₂), |
| | | | | | | | | | | 4.37 (d, 1H, CH₂), |
| | | | | | | | | | | 5.55 (d, 1H, CH), |
| | | | | | | | | | | 6.98 (t, 1H, CH). |
| 2.26 | 2 | F | H | H | H | Me | CF₃ | OCHF₂ | 93-95 | 1.42 (s, 3H, Me), 1.6 |
| | | | | | | | | | | (m, 3H, Me) 3.89 (s, |
| | | | | | | | | | | 3H, Me), 4.57 (s, 2H, |
| | | | | | | | | | | CH₂), 5.60 (d, 1H, |
| | | | | | | | | | | CH), 6.83 (t, 1H, CH). |
| 2.27 | 2 | F | H | Me | H | Me | CF₃ | OCHF₂ | gum | 1:1-Mixture of |
| | | | | | | | | | | diastereomers |
| | | | | | | | | | | 1.4 (s, 3H, Me), 1.6 |
| | | | | | | | | | | (s, 3H, Me), 1.88 |
| | | | | | | | | | | (dd, 3H, Me), 3.89 |
| | | | | | | | | | | (s, 3H, Me), 4.72 |
| | | | | | | | | | | (dq, 1H, CH), 5.5 |
| | | | | | | | | | | (dd, 1H, CH), 6.92 |
| | | | | | | | | | | (dt, 1H, CH). |
| 2.28 | 0 | F | H | H | H | Me | OCHF₂ | CF₃ | oil | 1.33 (s, 3H, Me), 1.5 |
| | | | | | | | | | | (m, 3H, Me), 3.89 (s, |
| | | | | | | | | | | 3H, Me), 4.21 (s, 2H, |
| | | | | | | | | | | CH₂), 5.03 (d, 1H, |
| | | | | | | | | | | CH), 6.89 (t, 1H, CH). |
| 2.29 | 1 | F | H | H | H | Me | OCHF₂ | CF₃ | gum | Diastereomer 1: |
| | | | | | | | | | | 1.38 (s, 3H, Me), |
| | | | | | | | | | | 1.60 (m, 3H, Me), |
| | | | | | | | | | | 3.93 (s, 3H, Me), 4.3 |
| | | | | | | | | | | (d, 1H, CH₂), 4.37 |
| | | | | | | | | | | (d, 1H, CH₂), 5.75 |
| | | | | | | | | | | (d, 1H, CH) 6.85 (t, |
| | | | | | | | | | | 1H, CH). |
| 2.30 | 1 | F | H | H | H | Me | OCHF₂ | CF₃ | gum | Diastereomer 2: |
| | | | | | | | | | | 1.38 (s, 3H, Me), |
| | | | | | | | | | | 1.60 (m, 3H, Me), |
| | | | | | | | | | | 3.93 (s, 3H, Me), |
| | | | | | | | | | | 4.38 (s, 2H, CH₂) |
| | | | | | | | | | | 5.54 (d, 1H, CH), |
| | | | | | | | | | | 6.89 (t, 1H, CH). |
| 2.31 | 2 | F | H | H | H | Me | OCHF₂ | CF₃ | gum | 1.40 (s, 3H, Me), |
| | | | | | | | | | | 1.60 (m, 3H, Me), |
| | | | | | | | | | | 3.93 (s, 3H, Me), |
| | | | | | | | | | | 4.51 (s, 2H, CH₂), |
| | | | | | | | | | | 5.42-5.58 (d, 1H, |
| | | | | | | | | | | CH), 6.70-7.07 (t, |
| | | | | | | | | | | 1H, CH). |
| 2.32 | 0 | F | H | H | H | Me | CF₃ | H | oil | 1.31 (s, 3H, Me), |
| | | | | | | | | | | 1.48 (d, 3H, Me), |
| | | | | | | | | | | 3.91 (s, 3H, Me), |
| | | | | | | | | | | 4.22 (dd, 2H, CH₂), |
| | | | | | | | | | | 5.00 (d, 1H, CH), |
| | | | | | | | | | | 7.54 (s, 1H, CH). |
| 2.33 | 1 | F | H | H | H | Me | CF₃ | H | gum | 4:3-Mixture of |
| | | | | | | | | | | diastereomers; |
| | | | | | | | | | | Diastereomer 1 |
| | | | | | | | | | | (major isomer): |
| | | | | | | | | | | 1.35 (s, 3H, Me), 1.6 |
| | | | | | | | | | | (d, 3H, Me), 4.95 (s, |
| | | | | | | | | | | 3H, Me), 4.3 (d, 1H, |
| | | | | | | | | | | CH₂), 4.4 (d, 1H, |
| | | | | | | | | | | CH₂), 5.7 (d, 1H, |
| | | | | | | | | | | CH), 7.5 (s, 1H, CH). |
| | | | | | | | | | | Diastereomer 2 |
| | | | | | | | | | | (minor isomer): |
| | | | | | | | | | | 1.3 (s, 3H, Me), 1.55 |
| | | | | | | | | | | (d, 3H, Me), 4.95 (s, |
| | | | | | | | | | | 3H, Me), 4.3 (d, 1H, |
| | | | | | | | | | | CH₂), 4.5 (d, 1H, |
| | | | | | | | | | | CH₂), 5.45 (d, 1H, |
| | | | | | | | | | | CH), 7.7 (s, 1H, CH). |
| 2.34 | 2 | F | H | H | H | Me | CF₃ | H | 122-123 | 1.3 (s, 3H, Me), 1.5 |
| | | | | | | | | | | (d, 3H, Me), 3.95 (s, |
| | | | | | | | | | | 3H, Me), 4.55 (s, 2H, |
| | | | | | | | | | | CH₂), 5.5 (d, 1H, |
| | | | | | | | | | | CH), 7.7 (s, 1H, CH). |
| 2.35 | 0 | F | H | H | H | Me | CF₃ | OCH | solid | 1:1-Mixture of |
| | | | | | | | | MeCF₃ | | diastereomers |
| | | | | | | | | | | 1.32 (s, 6H, Me), 1.5 |
| | | | | | | | | | | (d, 6H, Me), 1.6 (m, |
| | | | | | | | | | | 6H, Me), 3.75 (s, |
| | | | | | | | | | | 6H, Me), 4.2 (d, 1H, |
| | | | | | | | | | | CH₂), 4.25 (s, 2H, |
| | | | | | | | | | | CH₂), 4.4 (d, 1H, |
| | | | | | | | | | | CH₂), 4.7-4.8 (m, |
| | | | | | | | | | | 2H, CH), 4.95 (d, 1H, |
| | | | | | | | | | | CH), 5.1 (d, 1H, CH). |
| 2.37 | 2 | F | H | H | H | Me | CF₃ | OCH | solid | 7:1-Mixture of |
| | | | | | | | | MeCF₃ | | diastereomers; |
| | | | | | | | | | | Diastereomer 1 |
| | | | | | | | | | | (major isomer): |
| | | | | | | | | | | 1.4 (s, 3H, Me), 1.6 |
| | | | | | | | | | | (s, 6H, Me), 3.8 (s, |
| | | | | | | | | | | 3H, Me), 4.5 (d, 1H, |
| | | | | | | | | | | CH₂), 4.55 (d, 1H, |
| | | | | | | | | | | CH₂), 4.95-5.05 (m, |
| | | | | | | | | | | 1H, CH), 5.55 (d, 1H, CH). |
| | | | | | | | | | | Diastereomer 2 |
| | | | | | | | | | | (minor isomer): |
| | | | | | | | | | | 1.4 (s, 3H, Me), 1.62 |
| | | | | | | | | | | (s, 6H, Me), 3.78 (s, |
| | | | | | | | | | | 3H, Me), 4.5 (d, 1H, |
| | | | | | | | | | | CH₂), 4.55 (d, 1H, |
| | | | | | | | | | | CH₂), 5.2-5.28 (m, |
| | | | | | | | | | | 1H, CH), 5.75 (d, 1H, CH). |
| 2.38 | 0 | F | H | H | H | Me | CF₃ | OCH | solid | 1:1-Mixture of |
| | | | | | | | | MeC | | diastereomers |
| | | | | | | | | H₂F | | 1.32 (s, 6H, Me), |
| | | | | | | | | | | 1.4-1.45 (m, 6H, |
| | | | | | | | | | | Me), 1.5 (d, 6H, |
| | | | | | | | | | | Me), 3.75 (s, 6H, |
| | | | | | | | | | | Me), 4.2 (d, 1H, |
| | | | | | | | | | | CH₂), 4.25 (s, 2H, |
| | | | | | | | | | | CH₂), 4.3 (d, 1H, |
| | | | | | | | | | | CH₂), 4.4-4.5 (m, |
| | | | | | | | | | | 2H, CH₂), 4.5-4.7 |
| | | | | | | | | | | (m, 4H, CH₂ and |
| | | | | | | | | | | CH), 4.95 (d, 1H, |
| | | | | | | | | | | CH), 5.1 (d, 1H, CH). |
| 2.41 | 0 | F | H | H | H | Me | OCH₂CF₃ | CF₃ | gum | 1.32 (s, 3H, Me), |
| | | | | | | | | | | 1.48 (m, 3H, Me), |
| | | | | | | | | | | 3.82 (s, 3H, Me), 4.2 |
| | | | | | | | | | | (s, 2H, CH₂), 4.61 |
| | | | | | | | | | | (q, 2H, CH₂), 4.95- |
| | | | | | | | | | | 5.1 (d, 1H, CH). |
| 2.42 | 1 | F | H | H | H | Me | OCH₂CF₃ | CF₃ | 100-101 | Diastereomer 1: |
| | | | | | | | | | | 1.37 (s, 3H, Me), |
| | | | | | | | | | | 1.58 (m, 3H, Me), |
| | | | | | | | | | | 3.87 (s, 3H, Me), |
| | | | | | | | | | | 4.25-4.40 (q, 2H, |
| | | | | | | | | | | CH₂), 4.62 (m, 2H, |
| | | | | | | | | | | CH₂), 5.66-5.80 (d, |
| | | | | | | | | | | 1H, CH). |
| 2.43 | 1 | F | H | H | H | Me | OCH₂CF₃ | CF₃ | 98-99 | Diastereomer 2: |
| | | | | | | | | | | 1.37 (s, 3H, Me), |
| | | | | | | | | | | 1.58 (m, 3H, Me), |
| | | | | | | | | | | 3.87 (s, 3H, Me), |
| | | | | | | | | | | 4.37 (s, 2H, CH₂), |
| | | | | | | | | | | 4.61 (m, 2H, CH₂), |
| | | | | | | | | | | 5.48-5.62 (d, 1H, CH). |
| 2.44 | 2 | F | H | H | H | Me | OCH₂CF₃ | CF₃ | 130-132 | 1.39 (s, 3H, Me), 1.58 |
| | | | | | | | | | | (d, 3H, Me), 3.88 (s, |
| | | | | | | | | | | 3H, Me), 4.51 (s, 2H, |
| | | | | | | | | | | CH₂), 4.7-4.8 |
| | | | | | | | | | | (m, 2H, CH₂), |
| | | | | | | | | | | 5.49 (d, 1H, CH). |
| 2.45 | 0 | F | H | H | H | Et | CF₃ | H | | 1.3 (s, 3H, Me), 1.5 |
| | | | | | | | | | | (d, 3H, Me), 1.5 (t, |
| | | | | | | | | | | 3H, Me), 4.18 (q, |
| | | | | | | | | | | 2H, CH₂), 4.24 (q, |
| | | | | | | | | | | 2H, CH₂), 5.00 (d, |
| | | | | | | | | | | 1H, CH), 7.58 (s, 1H, CH). |
| 2.46 | 1 | F | H | H | H | Et | CF₃ | H | gum | 1:1-Mixture of |
| | | | | | | | | | | diastereomers; |
| | | | | | | | | | | Diastereomer 1:1.35 |
| | | | | | | | | | | (d, 3H, Me), 1.55 |
| | | | | | | | | | | (m, 6H, Me), 4.24 |
| | | | | | | | | | | (q, 2H, CH₂), 4.35 |
| | | | | | | | | | | (q, 2H, CH₂), 5.4 (d, |
| | | | | | | | | | | 1H, CH), 7.5 (s, 1H, |
| | | | | | | | | | | CH). Diastereomer |
| | | | | | | | | | | 2: 1.45 (d, 3H, Me), |
| | | | | | | | | | | 1.55 (m, 6H, Me), |
| | | | | | | | | | | 4.24 (q, 2H, CH₂), |
| | | | | | | | | | | 4.45 (q, 2H, CH₂), |
| | | | | | | | | | | 5.7 (d, 1H, CH), 7.66 |
| | | | | | | | | | | (s, 1H, CH). |
| 2.47 | 2 | F | H | H | H | Et | CF₃ | H | 107-109 | 1.38 (s, 3H, Me), |
| | | | | | | | | | | 1.55 (t, 6H, Me), |
| | | | | | | | | | | 4.22 (q, 2H, CH₂), |
| | | | | | | | | | | 4.59 (s, 2H, CH₂), |
| | | | | | | | | | | 5.45 (d, 1H, CH), |
| | | | | | | | | | | 7.72 (s, 1H, CH). |
| 2.48 | 0 | F | H | H | H | —CH₂ | CF₃ | H | gum | 1.32 (s, 3H, Me), 1.5 |
| | | | | | | C≡CH | | | | (d, 3H, Me), 2.58 (t, |
| | | | | | | | | | | 1H, CH), 4.24 (q, |
| | | | | | | | | | | 2H, CH₂), 4.95 (d, |
| | | | | | | | | | | 2H, CH₂), 5.02 (d, |
| | | | | | | | | | | 1H, CH), 7.82 (s, 1H, CH). |
| 2.49 | 1 | F | H | H | H | —CH₂ | CF₃ | H | 55-60 | 1:1-Mixture of |
| | | | | | | C≡CH | | | | diastereomers; |
| | | | | | | | | | | Diastereomer 1: |
| | | | | | | | | | | 1.35 (s, 3H, Me), 1.6 |
| | | | | | | | | | | (dd, 3H, Me), 2.62 |
| | | | | | | | | | | (m, 1H, CH), 4.35 |
| | | | | | | | | | | (q, 2H, CH₂), 5.0 (m, |
| | | | | | | | | | | 2H, CH₂), 5.45 (d, |
| | | | | | | | | | | 1H, CH), 7.8 (s, 1H, |
| | | | | | | | | | | CH). Diastereomer 2: |
| | | | | | | | | | | 1.4 (s, 3H, Me), 1.62 |
| | | | | | | | | | | (dd, 3H, Me), 2.62 |
| | | | | | | | | | | (m, 1H, CH), 4.45 |
| | | | | | | | | | | (q, 2H, CH₂), 5.0 (m, |
| | | | | | | | | | | 2H, CH₂), 5.65 (d, |
| | | | | | | | | | | 1H, CH), 7.92 (s, 1H, CH). |
| 2.50 | 2 | F | H | H | H | —CH₂ | CF₃ | H | gum | 1.38 (s, 3H, Me), |
| | | | | | | C≡CH | | | | 1.58 (d, 3H, Me), |
| | | | | | | | | | | 2.62 (m, 1H, CH), |
| | | | | | | | | | | 4.6 (s, 2H, CH₂), 5.0 |
| | | | | | | | | | | (m, 2H, CH₂), 5.46 |
| | | | | | | | | | | (d, 1H, CH), 8.0 (s, |
| | | | | | | | | | | 1H, CH). |
| 2.51 | 0 | F | H | H | H | —CH₂ | CF₃ | H | | 1.3 (s, 3H, Me), 1.5 |
| | | | | | | CH═CH₂ | | | | (d, 3H, Me), 4.24 (q, |
| | | | | | | | | | | 2H, CH₂), 4.74 (d, |
| | | | | | | | | | | 2H, CH₂), 5.04 (d, |
| | | | | | | | | | | 1H, CH), 5.28 (q, 2H, |
| | | | | | | | | | | CH₂), 6.0 (m, 1H, |
| | | | | | | | | | | CH), 7.58 (s, 1H, CH). |
| 2.52 | 1 | F | H | H | H | —CH₂ | CF₃ | H | gum | 1:1-Mixture of |
| | | | | | | CH═CH₂ | | | | diastereomers; |
| | | | | | | | | | | Diastereomer 1: |
| | | | | | | | | | | 1.35 (d, 3H, Me), |
| | | | | | | | | | | 1.57 (dd, 3H, Me), |
| | | | | | | | | | | 4.35 (q, 2H, CH₂), |
| | | | | | | | | | | 4.78 (m, 2H, CH₂), |
| | | | | | | | | | | 5.3 (m, 2H, CH₂), |
| | | | | | | | | | | 5.45 (d, 1H, CH), 6.0 |
| | | | | | | | | | | (m, 1H, CH), 7.52 (s, |
| | | | | | | | | | | 1H, CH). |
| | | | | | | | | | | Diastereomer 2: |
| | | | | | | | | | | 1.38 (d, 3H, Me), |
| | | | | | | | | | | 1.59 (dd, 3H, Me), |
| | | | | | | | | | | 4.45 (q, 2H, CH₂), |
| | | | | | | | | | | 4.78 (m, 2H, CH₂), |
| | | | | | | | | | | 5.35 (m, 2H, CH₂), |
| | | | | | | | | | | 5.72 (d, 1H, CH), 6.0 |
| | | | | | | | | | | (m, 1H, CH), 7.7 (s, |
| | | | | | | | | | | 1H, CH). |
| 2.53 | 2 | F | H | H | H | —CH₂ | CF₃ | H | 57-59 | 1.38 (s, 3H, Me), 1.6 |
| | | | | | | CH═CH₂ | | | | (d, 3H, Me), 4.6 (s, |
| | | | | | | | | | | 2H, CH₂), 4.8 (d, |
| | | | | | | | | | | 2H, CH₂), 5.3 (d, |
| | | | | | | | | | | 1H, CH₂), 5.4 (d, |
| | | | | | | | | | | 1H, CH₂), 5.45 (d, |
| | | | | | | | | | | 1H, CH), 6.0 (m, 1H, |
| | | | | | | | | | | CH), 7.72 (s, 1H, CH). |
| 2.54 | 0 | F | H | H | H | Me | CF₃ | F | oil | 1.32 (s, 3H, Me), |
| | | | | | | | | | | 1.49 (d, 3H, Me), |
| | | | | | | | | | | 3.80 (s, 3H, Me), |
| | | | | | | | | | | 4.19 (s, 2H, CH₂), |
| | | | | | | | | | | 5.01 (d, 1H, CH). |
| 2.55 | 1 | F | H | H | H | Me | CF₃ | F | 74 | 1.40 (s, 3H, Me), |
| | | | | | | | | | | 1.58 (d, 3H, Me), |
| | | | | | | | | | | 3.84 (s, 3H, Me), |
| | | | | | | | | | | 4.35 (s, 1H, CH₂), |
| | | | | | | | | | | 4.37 (s, 1H, CH₂), |
| | | | | | | | | | | 5.55 (d, 1H, CH). |
| 2.56 | 2 | F | H | H | H | Me | CF₃ | F | gum | 1.40 (s, 3H, Me), |
| | | | | | | | | | | 1.59 (d, 3H, Me), |
| | | | | | | | | | | 3.85 (s, 3H, Me), |
| | | | | | | | | | | 4.50 (s, 2H, CH₂), |
| | | | | | | | | | | 5.51 (d, 1H, CH). |
| 2.57 | 0 | F | H | H | H | Me | CF₃ | —OCH₂ | oil | 1.33 (s, 3H, Me), |
| | | | | | | | | CH₂O | | 1.49 (d, 3H, Me), |
| | | | | | | | | CH₃ | | 3.42 (s, 3H, Me), |
| | | | | | | | | | | 3.66-3.70 (m, 2H, |
| | | | | | | | | | | CH₂), 3.75 (s, 3H, |
| | | | | | | | | | | Me), 4.25 (s, 2H, |
| | | | | | | | | | | CH₂), 4.32-4.37 (m, |
| | | | | | | | | | | 2H, CH₂), 5.02 (d, |
| | | | | | | | | | | 1H, CH). |
| 2.58 | 1 | F | H | H | H | Me | CF₃ | —OCH₂ | oil | 3:2-Mixture of |
| | | | | | | | | CH₂O | | diastereomers; |
| | | | | | | | | CH₃ | | Diastereomer 1: |
| | | | | | | | | | | 1.39 (s, 3H, Me), |
| | | | | | | | | | | 1.60 (d, 3H, Me), |
| | | | | | | | | | | 3.42 (s, 3H, Me), |
| | | | | | | | | | | 3.63-3.75 (m, 2H, |
| | | | | | | | | | | CH₂), 3.79 (s, 3H, |
| | | | | | | | | | | Me) 4.30-4.50 (m, |
| | | | | | | | | | | 4H, CH₂), 5.55 (d, |
| | | | | | | | | | | 1H, CH). |
| | | | | | | | | | | Diastereomer 2: |
| | | | | | | | | | | 1.39 (s, 3H, Me), |
| | | | | | | | | | | 1.59 (d, 3H, Me), |
| | | | | | | | | | | 3.42 (s, 3H, Me), |
| | | | | | | | | | | 3.63-3.75 (m, 2H, |
| | | | | | | | | | | CH₂), 3.79 (s, 3H, |
| | | | | | | | | | | Me) 4.30-4.50 (m, |
| | | | | | | | | | | 4H, CH₂), 5.74 (d, |
| | | | | | | | | | | 1H, CH). |
| 2.59 | 2 | F | H | H | H | Me | CF₃ | —OCH₂ | oil | 1.40 (s, 3H, Me), |
| | | | | | | | | CH₂O | | 1.60 (d, 3H, Me), |
| | | | | | | | | CH₃ | | 3.43 (s, 3H, Me), |
| | | | | | | | | | | 3.70-3.73 (m, 2H, |
| | | | | | | | | | | CH₂), 3.81 (s, 3H, |
| | | | | | | | | | | Me), 4.39-4.41 (m, |
| | | | | | | | | | | 2H, CH₂), 4.56 (d, |
| | | | | | | | | | | 1H, CH₂), 4.59 (d, |
| | | | | | | | | | | 1H, CH₂), 5.52 (d, |
| | | | | | | | | | | 1H, CH). |
| 2.60 | 0 | F | H | H | H | Me | Me | —OCH₂ | oil | 1.33 (s, 3H, Me), |
| | | | | | | | | CF₃ | | 1.49 (d, 3H, Me), |
| | | | | | | | | | | 2.22 (s, 3H, Me), |
| | | | | | | | | | | 3.65 (s, 3H, Me), |
| | | | | | | | | | | 4.13 (s, 2H, CH₂), |
| | | | | | | | | | | 4.51 (q, 2H, CH₂), |
| | | | | | | | | | | 5.02 (d, 1H, CH). |
| 2.61 | 1 | F | H | H | H | Me | Me | —OCH₂ | oil | Diastereomer A: |
| | | | | | | | | CF₃ | | 1.40 (s, 3H, Me), |
| | | | | | | | | | | 1.59 (d, 3H, Me), |
| | | | | | | | | | | 2.24 (s, 3H, Me), |
| | | | | | | | | | | 3.68 (s, 3H, Me), |
| | | | | | | | | | | 3.95 (d, 1H, CH₂), |
| | | | | | | | | | | 4.37 (d, 1H, CH₂), |
| | | | | | | | | | | 4.51-4.71 (m, 2H, CH₂), |
| | | | | | | | | | | 5.74 (d, 1H, CH). |
| 2.62 | 1 | F | H | H | H | Me | Me | —OCH₂ | oil | Diastereomer B: |
| | | | | | | | | CF₃ | | 1.38 (s, 3H, Me), |
| | | | | | | | | | | 1.58 (d, 3H, Me), |
| | | | | | | | | | | 2.23 (s, 3H, Me), |
| | | | | | | | | | | 3.68 (s, 3H, Me), |
| | | | | | | | | | | 4.19 (d, 1H, CH₂), |
| | | | | | | | | | | 4.24 (d, 1H, CH₂), |
| | | | | | | | | | | 4.51-4.71 (m, 2H, CH₂), |
| | | | | | | | | | | 5.51 (d, 1H, CH). |
| 2.63 | 2 | F | H | H | H | Me | Me | —OCH₂ | oil | 1.41 (s, 3H, Me), |
| | | | | | | | | CF₃ | | 1.60 (d, 3H, Me), |
| | | | | | | | | | | 2.25 (s, 3H, Me), |
| | | | | | | | | | | 3.70 (s, 3H, Me), |
| | | | | | | | | | | 4.34 (d, 1H, CH₂), |
| | | | | | | | | | | 4.39 (d, 1H, CH₂), |
| | | | | | | | | | | 4.61 (q, 2H, CH₂), |
| | | | | | | | | | | 5.53 (d, 1H, CH). |
| 2.64 | 0 | F | H | H | H | Me | CF₃ | —CH═CHCH₃ | | 1.5 (d, 3H, Me), 2.0 |
| | | | | | | | | | | (d, 6H, Me), 3.88 (s, |
| | | | | | | | | | | 3H, Me), 4.3 (q, 2H, |
| | | | | | | | | | | CH₂), 5.03 (d, 1H, |
| | | | | | | | | | | CH), 6.25 (m, 2H, CH). |
| 2.65 | 1 | F | H | H | H | Me | CF₃ | —CH═CHCH₃ | solid | 1:1-Mixture of |
| | | | | | | | | | | diastereomers; |
| | | | | | | | | | | Diastereomer 1: |
| | | | | | | | | | | 1.6 (m, 6H, Me), |
| | | | | | | | | | | 1.98 (dd, 3H, Me), |
| | | | | | | | | | | 3.9 (s, 3H, Me), 4.4 |
| | | | | | | | | | | (q, 2H, CH₂), 5.65 |
| | | | | | | | | | | (d, 1H, CH), 6.35 |
| | | | | | | | | | | (m, 2H, CH). |
| | | | | | | | | | | Diastereomer 2: |
| | | | | | | | | | | 1.6 (m, 6H, Me), 2.0 |
| | | | | | | | | | | (dd, 3H, Me), 3.9 (s, |
| | | | | | | | | | | 3H, Me), 4.35 (q, |
| | | | | | | | | | | 2H, CH₂), 5.75 (d, |
| | | | | | | | | | | 1H, CH), 6.4 (m, 2H, CH). |
| 2.66 | 2 | F | H | H | H | Me | CF₃ | —CH≡CHCH₃ | 107-109 | 1.6 (m, 6H, Me), 2.0 |
| | | | | | | | | | | (dd, 3H, Me), 3.9 (s, |
| | | | | | | | | | | 3H, Me), 4.6 (s, 2H, |
| | | | | | | | | | | CH₂), 5.52 (d, 1H, |
| | | | | | | | | | | CH), 6.32 (m, 2H, CH). |
| 2.67 | 0 | F | H | H | H | CHF₂ | H | CF₃ | oil | 1.31 (s, 3H, Me), |
| | | | | | | | | | | 1.48 (d, 3H, Me), |
| | | | | | | | | | | 4.28 (s, 2H, CH₂), |
| | | | | | | | | | | 5.01 (d, 1H, CH), |
| | | | | | | | | | | 7.26 (t, 1H, CH), |
| | | | | | | | | | | 7.81 (s, 1H, CH). |
| 2.68 | 1 | F | H | H | H | CHF₂ | H | CF₃ | solid | Diastereomer A: |
| | | | | | | | | | | 1.30 (s, 3H, Me), |
| | | | | | | | | | | 1.56 (d, 3H, Me), |
| | | | | | | | | | | 4.45 (d, 1H, CH₂), |
| | | | | | | | | | | 4.64 (d, 1H, CH₂), |
| | | | | | | | | | | 5.42 (d, 1H, CH), |
| | | | | | | | | | | 7.30 (t, 1H, CH), |
| | | | | | | | | | | 7.79 (s, 1H, CH). |
| | | | | | | | | | | Diastereomer B: |
| | | | | | | | | | | 1.38 (s, 3H, Me), |
| | | | | | | | | | | 1.58 (d, 3H, Me), |
| | | | | | | | | | | 4.40 (d, 1H, CH₂), |
| | | | | | | | | | | 4.51 (d, 1H, CH₂), |
| | | | | | | | | | | 5.73 (d, 1H, CH), |
| | | | | | | | | | | 7.30 (t, 1H, CH), |
| | | | | | | | | | | 7.70 (s, 1H, CH). |
| 2.69 | 2 | F | H | H | H | CHF₂ | H | CF₃ | solid | 1.37 (s, 3H, Me), |
| | | | | | | | | | | 1.57 (d, 3H, Me), |
| | | | | | | | | | | 4.65 (s, 2H, CH₂), |
| | | | | | | | | | | 5.48 (d, 1H, CH), |
| | | | | | | | | | | 7.30 (t, 1H, CH), |
| | | | | | | | | | | 7.85 (s, 1H, CH). |
| 2.70 | 0 | F | H | H | H | CHF₂ | CF₃ | H | oil | 1.30 (s, 3H, Me), |
| | | | | | | | | | | 1.48 (d, 3H, Me), |
| | | | | | | | | | | 4.24 (s, 2H, CH₂), |
| | | | | | | | | | | 5.00 (d, 1H, CH), |
| | | | | | | | | | | 7.15 (t, 1H, CH), |
| | | | | | | | | | | 8.01 (s, 1H, CH). |
| 2.71 | 1 | F | H | H | H | CHF₂ | CF₃ | H | solid | Diastereomer A: |
| | | | | | | | | | | 1.30 (s, 3H, Me), |
| | | | | | | | | | | 1.55 (d, 3H, Me), |
| | | | | | | | | | | 4.34 (d, 1H, CH₂), |
| | | | | | | | | | | 4.54 (d, 1H, CH₂), |
| | | | | | | | | | | 5.43 (d, 1H, CH), |
| | | | | | | | | | | 7.19 (t, 1H, CH), |
| | | | | | | | | | | 8.12 (s, 1H, CH). |
| | | | | | | | | | | Diastereomers B: |
| | | | | | | | | | | 1.38 (s, 3H, Me), |
| | | | | | | | | | | 1.57 (d, 3H, Me), |
| | | | | | | | | | | 4.31 (d, 1H, CH₂), |
| | | | | | | | | | | 4.42 (d, 1H, CH₂), |
| | | | | | | | | | | 5.71 (d, 1H, CH), |
| | | | | | | | | | | 7.18 (t, 1H, CH), |
| | | | | | | | | | | 7.99 (s, 1H, CH). |
| 2.72 | 2 | F | H | H | H | CHF₂ | CF₃ | H | solid | 1.37 (s, 3H, Me), |
| | | | | | | | | | | 1.56 (d, 3H, Me), |
| | | | | | | | | | | 4.61 (s, 2H, CH₂), |
| | | | | | | | | | | 5.47 (d, 1H, CH), |
| | | | | | | | | | | 7.20 (t, 1H, CH), |
| | | | | | | | | | | 8.16 (s, 1H, CH). |
| 2.73 | 0 | F | H | H | H | ⁱPr | CF₃ | H | | 1.3 (s, 3H, Me), 1.5 |
| | | | | | | | | | | (s, 6H, Me), 1.6 (s, |
| | | | | | | | | | | 3H, Me), 4.22 (s, |
| | | | | | | | | | | 2H, CH₂), 4.5 (m, |
| | | | | | | | | | | 1H, CH), 5.0 (d, 1H, |
| | | | | | | | | | | CH), 7.55 (s, 1H, CH). |
| 2.74 | 1 | F | H | H | H | ⁱPr | CF₃ | H | solid | 1.4:1-Mixture of |
| | | | | | | | | | | diastereomers |
| | | | | | | | | | | Major diastereomer: |
| | | | | | | | | | | 1.3 (s, 3H, Me), 1.52 |
| | | | | | | | | | | (s, 3H, Me), 1.54 (s, |
| | | | | | | | | | | 3H, Me), 1.55 (d, 3H, |
| | | | | | | | | | | Me), 4.3 (d, 1H, CH₂), |
| | | | | | | | | | | 4.51 (d, 1H, CH₂), |
| | | | | | | | | | | 4.5-4.58 (m, 1H, CH), |
| | | | | | | | | | | 5.4 (d, 1H, CH), 7.7 (s, |
| | | | | | | | | | | 1H, CH). |
| | | | | | | | | | | Minor diastereomer: |
| | | | | | | | | | | 1.37 (s, 3H, Me), 1.5 |
| | | | | | | | | | | (s, 3H, Me), 1.53 (s, |
| | | | | | | | | | | 3H, Me), 1.59 (d, 3H, |
| | | | | | | | | | | Me), 4.3 (d, 1H, CH₂), |
| | | | | | | | | | | 4.4 (d, 1H, CH₂), |
| | | | | | | | | | | 4.5-4.58 (m, 1H, CH), |
| | | | | | | | | | | 5.73 (d, 1H, CH), 7.52 |
| | | | | | | | | | | (s, 1H, CH). |
| 2.75 | 2 | F | H | H | H | ⁱPr | CF₃ | H | gum | 1.48 (s, 3H, Me), |
| | | | | | | | | | | 1.5-1.6 (m, 9H, Me), |
| | | | | | | | | | | 4.55 (m, 1H, CH), |
| | | | | | | | | | | 4.6 (s, 2H, CH₂), |
| | | | | | | | | | | 5.45 (d, 1H, CH), |
| | | | | | | | | | | 7.72 (s, 1H, CH). |

Key:
Me = methyl; s = singlet; m = multiplet; d = doublet; t = triplet; q = quartet; dd = double doublet; dt = double triplet; dq = double quartet.

TABLE 53
Compounds of formula I.3

| | |
|---|---|
| | I.3 |
| 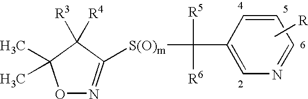 | |

| | | | | | | | M.p. | NMR data |
|---|---|---|---|---|---|---|---|---|
| No | R | m | R³ | R⁴ | R⁵ | R⁶ | [° C.] | (CDCl₃, 400 MHz) |
| 3.01 | 2-Me-6- | 0 | F | H | H | H | gum | 1.3 (s, 3H, Me), 1.48 |
| | CF₃ | | | | | | | (m, 3H, Me) 2.7 (s, |
| | | | | | | | | 3H, Me) 4.33 (s, 2H, |
| | | | | | | | | CH₂), 4.95-5.07 (d, |
| | | | | | | | | 1H, CH), 7.5 (d, 1H, |
| | | | | | | | | CH), 7.88 (d, 1H, CH). |
| 3.02 | 2-Me-6- | 1 | F | H | H | H | 137- | 1:1-Mixture of |
| | CF₃ | | | | | | 138 | diastereomers |
| | | | | | | | | 1.32 (s, 3H, Me), 1.40 (s, |
| | | | | | | | | 3H, Me), 1.58 (m, 3H, |
| | | | | | | | | Me), 1.61 (m, 3H, Me), |
| | | | | | | | | 2.73 (s, 3H, Me), 2.75 (s, |
| | | | | | | | | 3H, Me), 4.38-4.68 (m, |
| | | | | | | | | 4H, CH₂), 5.38-5.51 (d, |
| | | | | | | | | 1H, CH), 5.71-5.85 (d, |
| | | | | | | | | 1H, CH), 7.57 (d, 1H, |
| | | | | | | | | CH), 7.73 (d, 1H, CH), |
| | | | | | | | | 7.57 (d, 1H, CH), 7.83, |
| | | | | | | | | (d, 1H, CH). |
| 3.03 | 2-Me-6- | 2 | F | H | H | H | 99- | 1.4 (s, 3H, Me), 1.61 (m, |
| | CF₃ | | | | | | 101 | 3H, Me) 2.78 (s, 3H, |
| | | | | | | | | Me), 4.7 (s, 2H, CH₂), |
| | | | | | | | | 5.45-5.58 (d, 1H, CH), |
| | | | | | | | | 7.58 (d, 1H, CH), 7.88 |
| | | | | | | | | (d, 1H, CH). |
| 3.04 | 2-Cl | 0 | F | H | H | H | gum | 1.27 (s, 3H, Me), 1.46 |
| | | | | | | | | (d, 3H, Me), 4.36 (s, 2H, |
| | | | | | | | | CH₂), 4.98 (d, 1H, CH), |
| | | | | | | | | 7.21 (dd, 1H, CH), 7.88 |
| | | | | | | | | (dd, 1H, CH), 8.32 (dd, |
| | | | | | | | | 1H, CH). |
| 3.05 | 2-Cl | 1 | F | H | H | H | solid | Minor diastereomer |
| | | | | | | | | (45%): 1.37 (s, 3H, Me), |
| | | | | | | | | 1.59 (d, 3H, Me), 4.52 |
| | | | | | | | | (s, 2H, CH₂), 5.76 (d, |
| | | | | | | | | 1H, CH), 7.27 (dd, 1H, |
| | | | | | | | | CH), 7.67 (dd, 1H, CH), |
| | | | | | | | | 8.41 (dd, 1H, CH). |
| | | | | | | | | Major diastereomer |
| | | | | | | | | (55%): 1.35 (s, 3H, Me), |
| | | | | | | | | 1.56 (d, 3H, Me), 4.41 |
| | | | | | | | | (d, 1H, CH₂), 4.80 (d, |
| | | | | | | | | 1H, CH₂), 5.46 (d, 1H. |
| | | | | | | | | CH), 7.30 (dd, 1H, CH), |
| | | | | | | | | 7.81 (dd, 1H, CH), 8.43 |
| | | | | | | | | (dd, 1H, CH). |
| 3.06 | 2-Cl | 2 | F | H | H | H | solid | 1.37 (s, 3H, Me), 1.57 |
| | | | | | | | | (d, 3H, Me), 4.78 (d, 1H, |
| | | | | | | | | CH₂), 4.82 (d, 1H, |
| | | | | | | | | CH₂), 5.45 (d, 1H, CH), |
| | | | | | | | | 7.32 (dd, 1H, CH), 7.91 |
| | | | | | | | | (dd, 1H, CH), 8.44 (dd, |
| | | | | | | | | 1H, CH). |
| 3.07 | 2-OMe | 2 | F | H | F | F | solid | 1.45 (s, 3H, Me), 1.65 |
| | | | | | | | | (d, 3H, Me), 4.03 (s, 3H, |
| | | | | | | | | Me), 5.48 (d, 1H, CH), |
| | | | | | | | | 7.05 (m, 1H, CH), 7.9 |
| | | | | | | | | (m, 1H, CH), 8.4 (m, |
| | | | | | | | | 1H, CH). |

Key:
Me = methyl; s = singlet; m = multiplet; d = doublet; d = double doublet.

TABLE 54
Compounds of formula I.4

| | |
|---|---|
| | I.4 |
| 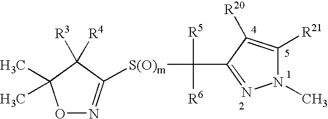 | |

| | | | | | | | | M.p. | ¹H-NMR |
|---|---|---|---|---|---|---|---|---|---|
| No | m | R³ | R⁴ | R⁵ | R⁶ | R²⁰ | R²¹ | [° C.] | (CDCl₃, 400 MHz) |
| 4.01 | 0 | F | H | H | H | CF₃ | H | oil | 1.33 (s, 3H, Me), 1.49 (d, |
| | | | | | | | | | 3H, Me), 3.89 (s, 3H, Me), |
| | | | | | | | | | 4.37 (s, 2H, CH₂), 5.12 (d, |
| | | | | | | | | | 1H, CH), 7.61 (s, 1H, CH). |
| 4.02 | 1 | F | H | H | H | CF₃ | H | solid | 2:3-Mixture of diastereomers; |
| | | | | | | | | | Diastereomer 1 (minor isomer): |
| | | | | | | | | | 1.39 (s, 3H, Me), 1.56-1.60 |
| | | | | | | | | | (m, 3H, Me), 3.94 (s, 3H, |
| | | | | | | | | | Me), 4.40-4.63 (m, 2H, |
| | | | | | | | | | CH₂), 5.77 (d, 1H, CH), |
| | | | | | | | | | 7.67 (bs, 1H, CH). |
| | | | | | | | | | Diastereomer 2 (major isomer): |
| | | | | | | | | | 1.42 (s, 3H, Me), 1.56-1.60 |
| | | | | | | | | | (m, 3H, Me), 3.90 (s, 3H, |
| | | | | | | | | | Me), 4.40-4.63 (m, 2H, |
| | | | | | | | | | CH₂), 5.67 (d, 1H, CH), |
| | | | | | | | | | 7.67 (bs, 1H, CH). |
| 4.03 | 2 | F | H | H | H | CF₃ | H | solid | 1.43 (s, 3H, Me), 1.59 (d, |
| | | | | | | | | | 3H, Me), 3.93 (s, 3H, Me), |
| | | | | | | | | | 4.72 (dd, 2H, CH₂), 5.49 (d, |
| | | | | | | | | | 1H, CH), 7.70 (s, 1H, CH). |
| 4.04 | 2 | F | H | Me | H | CF₃ | H | solid | 2:3-Mixture of diastereomers; |
| | | | | | | | | | Diastereomer 1 (minor isomer): |
| | | | | | | | | | 1.44 (s, 3H, Me), 1.58 (d, |
| | | | | | | | | | 3H, Me), 1.85 (d, 3H, Me), |
| | | | | | | | | | 3.93 (s, 3H, Me), 4.86 (q, |
| | | | | | | | | | 1H, CH), 5.42 (d, 1H, CH), |
| | | | | | | | | | 7.69 (s, 1H, CH). |
| | | | | | | | | | Diastereomer 2 (major isomer): |
| | | | | | | | | | 1.35 (s, 3H, Me), 1.56 (d, |
| | | | | | | | | | 3H, Me), 1.89 (d, 3H, Me), |
| | | | | | | | | | 3.93 (s, 3H, Me), 4.72 (q, |
| | | | | | | | | | 1H, CH), 5.43 (d, 1H, CH), |
| | | | | | | | | | 7.68 (s, 1H, CH). |
| 4.05 | 2 | F | H | Me | Me | CF₃ | H | solid | 1.41 (s, 3H, Me), 1.53 (d, |
| | | | | | | | | | 3H, Me), 1.94 (s, 3H, Me), |
| | | | | | | | | | 1.98 (s, 3H, Me), 3.87 (s, |
| | | | | | | | | | 3H, Me), 5.22 (d, 1H, CH), |
| | | | | | | | | | 7.70 (s, 1H, CH). |
| 4.06 | 0 | F | H | H | H | CF₃ | —CN | oil | 1.31 (s, 3H, Me), 1.47 (d, |
| | | | | | | | | | 3H, Me), 4.06 (s, 3H; Me), |
| | | | | | | | | | 4.36 (s, 2H, CH₂), 5.06 (d, |
| | | | | | | | | | 1H, CH). |
| 4.07 | 1 | F | H | H | H | CF₃ | —CN | oil | Diastereomer 1: |
| | | | | | | | | | 1.39 (s, 3H, Me), 1.58 (d, |
| | | | | | | | | | 3H, Me), 4.12 (s, 3H, Me), |
| | | | | | | | | | 4.42 (d, 1H, CH₂), 4.60 (d, |
| | | | | | | | | | 1H, CH₂), 5.77 (d, 1H, CH). |
| 4.08 | 1 | F | H | H | H | CF₃ | —CN | oil | Diastereomer 2: |
| | | | | | | | | | 1.41 (s, 3H, Me), 1.57 (d, |
| | | | | | | | | | 3H, Me), 4.09 (s, 3H, Me), |
| | | | | | | | | | 4.54 (d, 1H, CH₂), 4.64 (d, |
| | | | | | | | | | 1H, CH₂), 5.58 (d, 1H, CH). |
| 4.09 | 2 | F | H | H | H | CF₃ | —CN | solid | 1.41 (s, 3H, Me), 1.58 (d, |
| | | | | | | | | | 3H, Me), 4.11 (s, 3H, Me), |
| | | | | | | | | | 4.74 (s, 2H, CH₂), 5.49 (d, |
| | | | | | | | | | 1H, CH). |

Key:
Me = methyl; s = singlet; m = multiplet; d = doublet; bs = broad signal.

TABLE 55
Compounds of formula I.5

| | |
|---|---|
| | I.5 |
| 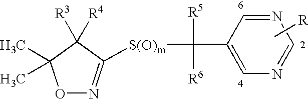 | |

| | | | | | | | M.p. | NMR data |
|---|---|---|---|---|---|---|---|---|
| No | R | m | R³ | R⁴ | R⁵ | R⁶ | [° C.] | (CDCl₃, 400 MHz) |
| 5.01 | 2-OCH₃, | 0 | F | H | H | H | | 1.36 (s, 3H, Me), 1.5 |
| | -6-CF₃ | | | | | | | (d, 3H, Me), 4.11 (s, |
| | | | | | | | | 3H, Me), 4.5 (s, 2H, |
| | | | | | | | | CH₂), 5.01 (d, 1H, |
| | | | | | | | | CH), 8.8 (s, 1H, CH). |
| 5.02 | 2-OCH₃, | 1 | F | H | H | H | 97- | 1.40-1.65 (m, 6H, |
| | -6-CF₃ | | | | | | 101 | Me), 4.15 (s, 3H, Me), |
| | | | | | | | | 4.35-4.8 (m, 2H, |
| | | | | | | | | CH₂), 5.5-5.85 (m, |
| | | | | | | | | 1H, CH), 8.88 (s, 1H, |
| | | | | | | | | CH). |
| 5.03 | 2-OCH₃, | 2 | F | H | H | H | 129- | 1.4 (s, 3H, Me), 1.61 |
| | -6-CF₃ | | | | | | 131 | (d, 3H, Me), 4.1 (s, |
| | | | | | | | | 3H, Me), 4.95 (s, 2H, |
| | | | | | | | | CH₂), 5.5 (d, 1H, |
| | | | | | | | | CH), 8.87 (s, 1H, CH). |

Key:
Me = methyl; s = singlet; m = multiplet; d = doublet.

TABLE 56
Compounds of formula I.6

| | |
|---|---|
| | I.6 |
| 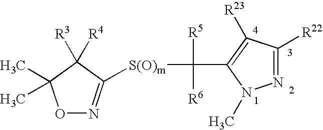 | |

| | | | | | | | | M.p. | ¹H-HMR |
|---|---|---|---|---|---|---|---|---|---|
| No | m | R³ | R⁴ | R⁵ | R⁶ | R²² | R²³ | [° C.] | (CDCl₃, 400 MHz) |
| 6.01 | 0 | F | H | H | H | CF₃ | H | gum | 1.31 (s, 3H, Me), 1.48 (d, |
| | | | | | | | | | 3H, Me), 3.95 (s, 3H, Me), |
| | | | | | | | | | 4.31 (d, 1H, CH₂), 4.34 (d, |
| | | | | | | | | | 1H, CH₂), 5.01 (d, 1H, CH), |
| | | | | | | | | | 6.55 (s, 1H, CH). |
| 6.02 | 1 | F | H | H | H | CF₃ | H | solid | Minor diastereomer (42%): |
| | | | | | | | | | 1.40 (s, 3H, Me), 1.55 (d, |
| | | | | | | | | | 3H, Me), 4.00 (s, 3H, Me), |
| | | | | | | | | | 4.38 (d, 1H, CH₂), 4.55 (d, |
| | | | | | | | | | 1H, CH₂), 5.76 (d, 1H, CH), |
| | | | | | | | | | 6.59 (s, 1H, CH). |
| | | | | | | | | | Major diastereomer (58%): |
| | | | | | | | | | 1.25 (s, 3H, Me), 1.59 (d, |
| | | | | | | | | | 3H, Me), 4.00 (s, 3H, Me), |
| | | | | | | | | | 4.44 (d, 1H, CH₂), 4.62 (d, |
| | | | | | | | | | 1H, CH₂), 5.34 (d, 1H, CH), |
| | | | | | | | | | 6.58 (s, 1H, CH). |
| 6.03 | 1 | F | H | H | H | CF₃ | H | solid | Minor diastereomer (42%): |
| | | | | | | | | | 1.41 (s, 3H, Me), 1.61 (d, |
| | | | | | | | | | 3H, Me), 4.00 (s, 3H, Me), |
| | | | | | | | | | 4.35 (d, 1H, CH₂), 4.62 (d, |
| | | | | | | | | | 1H, CH₂), 5.77 (d, 1H, CH). |
| | | | | | | | | | Major diastereomer (58%): |
| | | | | | | | | | 1.40 (s, 3H, Me), 1.59 (d, |
| | | | | | | | | | 3H, Me), 4.03 (s, 3H, Me), |
| | | | | | | | | | 4.56 (d, 1H, CH₂), 4.59 (d, |
| | | | | | | | | | 1H, CH₂). 5.48 (d, 1H, CH). |
| 6.04 | 2 | F | H | H | H | CF₃ | H | solid | 1.36 (s, 3H, Me), 1.59 (d, |
| | | | | | | | | | 3H, Me), 4.03 (s, 3H, Me), |
| | | | | | | | | | 4.66 (d, 1H, CH₂), 4.72 (d, |
| | | | | | | | | | 1H, CH₂), 5.45 (d, 1H, CH), |
| | | | | | | | | | 6.62 (s, 1H, CH). |
| 6.05 | 0 | F | H | H | H | CF₃ | Cl | gum | 1.33 (s, 3H, Me), 1.50 (d, |
| | | | | | | | | | 3H, Me), 3.98 (s, 3H, Me), |
| | | | | | | | | | 4.37 (s, 2H, CH₂), 5.04 (d, |
| | | | | | | | | | 1H, CH). |
| 6.07 | 2 | F | H | H | H | CF₃ | Cl | solid | 1.41 (s, 3H, Me), 1.58 (d, |
| | | | | | | | | | 3H, Me), 4.07 (s, 3H, Me), |
| | | | | | | | | | 4.70 (s, 2H, CH₂), 5.47 (d, |
| | | | | | | | | | 1H, CH). |
| 6.08 | 0 | F | H | H | H | —OCHF₂ | H | gum | 1.31 (s, 3H, Me), 1.49 (d, |
| | | | | | | | | | 3H, Me), 3.78 (s, 3H, Me), |
| | | | | | | | | | 4.24 (d, 1H, CH₂), 4.27 (d, |
| | | | | | | | | | 1H, CH₂), 5.01 (d, 1H, CH), |
| | | | | | | | | | 5.91 (s, 1H, CH), 6.76 (t, |
| | | | | | | | | | 1H, CH). |
| 6.09 | 1 | F | H | H | H | —OCHF₂ | H | solid | Minor diastereomer (43%): |
| | | | | | | | | | 1.39 (s, 3H, Me), 1.59 (d, |
| | | | | | | | | | 3H, Me), 3.83 (s, 3H, Me), |
| | | | | | | | | | 4.32 (d, 1H, CH₂), 4.47 (d, |
| | | | | | | | | | 1H, CH₂), 5.75 (d, 1H, CH), |
| | | | | | | | | | 5.95 (s, 1H, CH), 6.77 (t, |
| | | | | | | | | | 1H, CH). Major |
| | | | | | | | | | diastereomer (57%): 1.29 (s, |
| | | | | | | | | | 3H, Me), 1.55 (d, 3H, Me), |
| | | | | | | | | | 3.82 (s, 3H, Me), 4.36 (d, |
| | | | | | | | | | 1H, CH₂), 4.55 (d, 1H, |
| | | | | | | | | | CH₂), 5.37 (d, 1H, CH), |
| | | | | | | | | | 5.95 (s, 1H, CH), 6.77 (t, |
| | | | | | | | | | 1H, CH). |
| 6.10 | 2 | F | H | H | H | —OCHF₂ | H | solid | 1.36 (s, 3H, Me), 1.59 (d, |
| | | | | | | | | | 3H, Me), 3.85 (s, 3H, Me), |
| | | | | | | | | | 4.57 (d, 1H, CH₂), 4.65 (d, |
| | | | | | | | | | 1H, CH₂), 5.45 (d, 1H, CH), |
| | | | | | | | | | 5.98 (s, 1H, CH), 6.77 (t, |
| | | | | | | | | | 1H, CH). |
| 6.11 | 0 | F | H | H | H | —OCH₃ | H | | 1.32 (s, 3H, Me), 1.49 (d, |
| | | | | | | | | | 3H, Me), 3.73 (s, 3H, Me), |
| | | | | | | | | | 3.85 (s, 3H, Me), 4.22 (d, |
| | | | | | | | | | 1H, CH₂), 4.26 (d, 1H, |
| | | | | | | | | | CH₂), 5.00 (d, 1H, CH), |
| | | | | | | | | | 5.68 (s, 1H, CH). |
| 6.12 | 1 | F | H | H | H | —OCH₃ | H | gum | Minor diastereomer (43%): |
| | | | | | | | | | 1.39 (s, 3H, Me), 1.60 (d, |
| | | | | | | | | | 3H, Me), 3.77 (s, 3H, Me), |
| | | | | | | | | | 3.86 (s, 3H, Me), 4.31 (d, |
| | | | | | | | | | 1H, CH₂), 4.43 (d, 1H, |
| | | | | | | | | | CH₂), 5.71 (s, 1H, CH), 5.75 |
| | | | | | | | | | (d, 1H, CH). |
| | | | | | | | | | Major diastereomer (57%): |
| | | | | | | | | | 1.29 (s, 3H, Me), 1.56 (d, |
| | | | | | | | | | 3H, Me), 3.77 (s, 3H, Me), |
| | | | | | | | | | 3.85 (s, 3H, Me), 4.31 (d, |
| | | | | | | | | | 1H, CH₂), 4.52 (d, 1H, |
| | | | | | | | | | CH₂), 5.37 (d, 1H, CH), |
| | | | | | | | | | 5.72 (s, 1H, CH). |
| 6.13 | 2 | F | H | H | H | —OCH₃ | H | solid | 1.36 (s, 3H, Me), 1.59 (d, |
| | | | | | | | | | 3H, Me), 3.80 (s, 3H, Me), |
| | | | | | | | | | 3.86 (s, 3H, Me), 4.54 (d, |
| | | | | | | | | | 1H, CH₂), 4.63 (d, 1H, |
| | | | | | | | | | CH₂), 5.43 (d, 1H, CH), |
| | | | | | | | | | 5.75 (s, 1H, CH). |

Key:
Me = methyl; s = singlet; d = doublet; t = triplet.

TABLE 57
Compounds of formula I.7

| | |
|---|---|
| | I.7 |
| 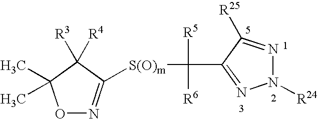 | |

| | | | | | | | | | |
|---|---|---|---|---|---|---|---|---|---|
| | | | | | | | | M.p. | |
| No | m | R³ | R⁴ | R⁵ | R⁶ | R²⁴ | R²⁵ | [° C.] | ¹H-NMR (CDCl₃, 400 MHz) |

| | | | | | | | | | |
|---|---|---|---|---|---|---|---|---|---|
| 7.01 | 0 | F | H | H | H | Me | Me | gum | 1.31 (s, 3H, Me), 1.49 (m, 3H, |
| | | | | | | | | | Me), 2.30 (s, 3H, Me), 4.10 (s, |
| | | | | | | | | | 3H, Me), 4.30 (m, 2H, CH₂), |
| | | | | | | | | | 5.0-5.15 (d, 1H, CH). |
| 7.02 | 1 | F | H | H | H | Me | Me | 115- | Diastereomer 1: |
| | | | | | | | | 17 | 1.38 (s, 3H, Me), 1.58 (m, 3H, |
| | | | | | | | | | Me), 2.28 (s, 3H, Me), 4.11 (s, |
| | | | | | | | | | 3H, Me), 4.33-4.50 (dd, 2H, |
| | | | | | | | | | CH₂), 5.68-5.82 (d, 1H, CH). |
| 7.03 | 1 | F | H | H | H | Me | Me | 105- | Diastereomer 2: |
| | | | | | | | | 107 | 1.39 (s, 3H, Me), 1.58 (m, 3H, |
| | | | | | | | | | Me), 2.33 (s, 3H, Me), 4.1 (s, |
| | | | | | | | | | 3H, Me), 4.38-4.58 (dd, 2H, |
| | | | | | | | | | CH₂), 5.44-5.59 (d, 1H, CH). |
| 7.04 | 2 | F | H | H | H | Me | Me | 140- | 1.4 (s, 3H, Me), 1.59 (m, 3H, |
| | | | | | | | | 142 | Me), 2.35 (s, 3H, Me), 4.11 (s, |
| | | | | | | | | | 3H, Me), 4.62 (dd, 2H, CH₂), |
| | | | | | | | | | 5.35-5.5 (d, 1H, CH). |
| 7.05 | 1 | F | H | F | F | Me | Me | oil | 1:1-Mixture of diastereomers: |
| | | | | | | | | | 1.38 (s, 3H, Me), 1.40 (s, 3H, |
| | | | | | | | | | Me), 1.55-1.60 (m, 6H, Me), |
| | | | | | | | | | 2.41 (t, 3H, Me), 2.43 (t, 3H, |
| | | | | | | | | | Me), 4.15 (s, 3H, Me), 4.16 (s, |
| | | | | | | | | | 3H, Me), 5.47 (d, 1H, CH), |
| | | | | | | | | | 5.55 (d, 1H, CH). |
| 7.06 | 2 | F | H | F | F | Me | Me | 80- | 1.45 (s, 3H, Me), 1.65 (d, 3H, |
| | | | | | | | | 81 | Me), 2.46 (s, 3H, Me), 4.21 (s, |
| | | | | | | | | | 3H, Me), 5.47 (d, 1H, CH). |
| 7.07 | 0 | F | H | H | H | —CH₂CH═CH₂ | CF₃ | oil | 1.32 (s, 3H, Me), 1.48 (d, 3H, |
| | | | | | | | | | Me), 4.42 (s, 2H, CH₂), 5.01- |
| | | | | | | | | | 5.04 (m, 2H, CH₂), 5.08 (d, |
| | | | | | | | | | 1H, CH), 5.31-5.38 (m, 2H, |
| | | | | | | | | | CH₂), 6.00-6.10 (m, 1H, CH). |
| 7.08 | 1 | F | H | H | H | —CH₂CH═CH₂ | CF₃ | 58- | Diastereomer 1: |
| | | | | | | | | 59 | 1.39 (s, 3H, Me), 1.58 (d, 3H, |
| | | | | | | | | | Me), 4.51 (d, 1H, CH₂), 4.63 |
| | | | | | | | | | (d, 1H, CH₂), 5.07 (d, 2H, |
| | | | | | | | | | CH₂), 5.33-5.40 (m, 2H, CH₂), |
| | | | | | | | | | 5.76 (d, 1H, CH), 6.01-6.10 |
| | | | | | | | | | (m, 1H, CH). |
| 7.09 | 1 | F | H | H | H | —CH₂CH═CH₂ | CF₃ | oil | Diastereomer 2: |
| | | | | | | | | | 1.41 (s, 3H, Me), 1.57 (d, 3H, |
| | | | | | | | | | Me), 4.57 (d, 1H, CH₂), 4.69 |
| | | | | | | | | | (d, 1H, CH₂), 5.05 (d, 2H, |
| | | | | | | | | | CH₂), 5.31-5.40 (m, 2H, CH₂), |
| | | | | | | | | | 5.55 (d, 1H, CH), 6.00-6.10 |
| | | | | | | | | | (m, 1H, CH). |
| 7.10 | 2 | F | H | H | H | —CH₂CH═CH₂ | CF₃ | oil | 1.41 (s, 3H, Me), 1.58 (d, 3H, |
| | | | | | | | | | Me), 4.79 (s, 2H, CH₂), 5.07 |
| | | | | | | | | | (d, 2H, CH₂), 5.32-5.40 (m, |
| | | | | | | | | | 2H, CH₂), 4.57 (d, 1H, CH), |
| | | | | | | | | | 6.00-6.01 (m, 1H, CH). |
| 7.11 | 0 | F | H | H | H | cyclo- | CF₃ | oil | 1.32 (s, 3H, Me), 1.5 (d, 3H, |
| | | | | | | pentyl | | | Me), 1.71 (m, 2H, CH₂), 1.9 |
| | | | | | | | | | (m, 2H, CH₂), 2.19 (m, 4H, |
| | | | | | | | | | CH₂), 4.41 (s, 2H, CH₂), 4.99 |
| | | | | | | | | | (m, 1H, CH), 5.07 (d, 1H, |
| | | | | | | | | | CH). |
| 7.12 | 1 | F | H | H | H | cyclo- | CF₃ | 80 | DiastereomerB: |
| | | | | | | pentyl | | | 1.4 (s, 3H, Me), 1.59 (d, 3H, |
| | | | | | | | | | Me), 1.73 (m, 2H, CH₂), 1.91 |
| | | | | | | | | | (m, 2H, CH₂), 2.2 (m, 4H, |
| | | | | | | | | | CH₂), 4.52 (d, 1H, CH₂), 4.6 |
| | | | | | | | | | (d, 1H, CH₂), 5.03 (m, 1H, |
| | | | | | | | | | CH), 5.76 (d, 1H, CH). |
| 7.13 | 1 | F | H | H | H | cyclo- | CF₃ | solid | 3:2 Mixture of diastereomers; |
| | | | | | | pentyl | | | Diastereomer A (major |
| | | | | | | | | | isomer): |
| | | | | | | | | | 1.4 (s, 3H, Me), 1.6 (d, 3H, |
| | | | | | | | | | Me), 1.74 (m, 2H, CH₂), 1.9 |
| | | | | | | | | | (m, 2H, CH₂), 2.2 (m, 4H, |
| | | | | | | | | | CH₂), 4.57 (d, 1H, CH₂), 4.6 |
| | | | | | | | | | (d, 1H, CH₂), 5.02 (m, 1H, |
| | | | | | | | | | CH), 5.57 (d, 1H, CH). |
| | | | | | | | | | Diastereomer B (minor |
| | | | | | | | | | isomer): |
| | | | | | | | | | 1.4 (s, 3H, Me), 1.59 (d, 3H, |
| | | | | | | | | | Me), 1.73 (m, 2H, CH₂), 1.91 |
| | | | | | | | | | (m, 2H, CH₂), 2.2 (m, 4H, |
| | | | | | | | | | CH₂), 4.52 (d, 1H, CH₂), 4.6 |
| | | | | | | | | | (d, 1H, CH₂), 5.03 (m, 1H, |
| | | | | | | | | | CH), 5.76 (d, 1H, CH). |
| 7.14 | 2 | F | H | H | H | cyclo- | CF₃ | oil | 1.4 (s, 3H, Me), 1.6 (d, 3H, |
| | | | | | | pentyl | | | Me), 1.73 (m, 2H, CH₂), 1.9 |
| | | | | | | | | | (m, 2H, CH₂), 2.2 (m, 4H, |
| | | | | | | | | | CH₂), 4.77 (s, 2H, CH₂), 5.05 |
| | | | | | | | | | (m, 1H, CH), 5.47 (d, 1H, |
| | | | | | | | | | CH). |
| 7.15 | 0 | F | H | H | H | —CH₂^{c}Bu | CF₃ | oil | 1.32 (s, 3H, Me), 1.5 (d, 3H, |
| | | | | | | | | | Me), 1.9 (m, 4H, CH₂), 2.1 |
| | | | | | | | | | (m, 2H, CH₂), 2.93 (m, 1H, |
| | | | | | | | | | CH), 4.42 (s, 2H, CH₂), 4.43 |
| | | | | | | | | | (d, 2H, CH₂), 5.07 (d, m, |
| | | | | | | | | | CH). |
| 7.16 | 1 | F | H | H | H | —CH₂^{c}Bu | CF₃ | oil | Diastereomer 1 (major |
| | | | | | | | | | isomer): |
| | | | | | | | | | 1.41 (s, 3H, Me), 1.59 (d, 3H, |
| | | | | | | | | | Me), 1.9 (m, 4H, CH₂), 2.1 |
| | | | | | | | | | (m, 2H, CH₂), 2.94 (m, 1H, |
| | | | | | | | | | CH), 4.45 (d, 2H, CH₂), 4.57 |
| | | | | | | | | | (d, 1H, CH₂), 4.68 (d, 1H, |
| | | | | | | | | | CH₂), 5.57 (d, 1H, CH). |
| | | | | | | | | | Diastereomer 2 (mmor |
| | | | | | | | | | isomer): |
| | | | | | | | | | 1.40 (s, 3H, Me), 1.6 (d, 3H, |
| | | | | | | | | | Me), 1.9 (m, 4H, CH₂), 2.1 |
| | | | | | | | | | (m, 2H, CH₂), 2.94 (m, 1H, |
| | | | | | | | | | CH), 4.46 (d, 2H, CH₂), 4.51 |
| | | | | | | | | | (d, 1H, CH₂), 4.61 (d, 1H, |
| | | | | | | | | | CH₂), 5.74 (d, 1H, CH). |
| 7.17 | 2 | F | H | H | H | —H₂^{c}Bu | CF₃ | oil | 1.4 (s, 3H, Me), 1.6 (d, 3H, |
| | | | | | | | | | Me), 1.9 (m, 4H, CH₂), 2.1 |
| | | | | | | | | | (m, 2H, CH₂), 2.94 (m, 1H, |
| | | | | | | | | | CH), 4.48 (s, 2H, CH₂), 4.79 |
| | | | | | | | | | (d, 2H, CH₂), 5.48 (d, 1H, |
| | | | | | | | | | CH). |
| 7.18 | 0 | F | H | H | H | —CH₂CH₂OCH₃ | CF₃ | oil | 1.32 (s, 3H, Me), 1.48 (d, 3H, |
| | | | | | | | | | Me), 3.34 (s, 3H, Me), 3.90 (t, |
| | | | | | | | | | 2H, CH₂), 4.43 (s, 2H, CH₂), |
| | | | | | | | | | 4.59 (t, 2H, CH₂), 5.08 (d, 1H, |
| | | | | | | | | | CH). |
| 7.19 | 1 | F | H | H | H | —CH₂CH₂OCH₃ | CF₃ | oil | 3:2-Mixture of diastereomers; |
| | | | | | | | | | Diastereomer A (major |
| | | | | | | | | | isomer): |
| | | | | | | | | | 1.41 (s, 3H, Me), 1.57 (d, 3H, |
| | | | | | | | | | Me), 3.34 (s, 3H, Me), 3.89- |
| | | | | | | | | | 3.92 (m, 2H, CH₂), 4.49-4.71 |
| | | | | | | | | | (m, 4H, CH₂), 5.57 (d, 1H, |
| | | | | | | | | | CH). |
| | | | | | | | | | Diastereomer B (minor |
| | | | | | | | | | isomer): |
| | | | | | | | | | 1.39 (s, 3H, Me), 1.58 (d, 3H, |
| | | | | | | | | | Me), 3.35 (s, 3H, Me), 3.89- |
| | | | | | | | | | 3.92 (m, 2H, CH₂), 4.49-4.71 |
| | | | | | | | | | (m, 4H, CH₂), 5.76 (d, 1H, |
| | | | | | | | | | CH). |
| 7.20 | 2 | F | H | H | H | —CH₂CH₂OCH₃ | CF₃ | oil | 1.41 (s, 3H, Me), 1.58 (d, 3H, |
| | | | | | | | | | Me), 3.34 (s, 3H, Me), 3.91 (t, |
| | | | | | | | | | 2H, CH₂), 4.64 (t, 2H, CH₂), |
| | | | | | | | | | 4.78 (s, 2H, CH₂), 5.48 (d, 1H, |
| | | | | | | | | | CH). |
| 7.21 | 0 | F | H | H | H | Me | H | | 1.30 (s, 3H, Me), 1.49 (d, 3H, |
| | | | | | | | | | Me), 4.16 (s, 3H, Me), 4.33 (s, |
| | | | | | | | | | 2H, CH₂), 5.04 (d, 1H, CH), |
| | | | | | | | | | 7.57 (s, 1H, CH). |
| 7.22 | 1 | F | H | H | H | Me | H | solid | Minor diastereomer (43%): |
| | | | | | | | | | 1.38 (s, 3H, Me), 1.58 (d, 3H, |
| | | | | | | | | | Me), 4.20 (s, 3H, Me), 4.45 (d, |
| | | | | | | | | | 1H, CH₂), 4.54 (d, 1H, CH₂), |
| | | | | | | | | | 5.78 (d, 1H, CH), 7.55 (s, 1H, |
| | | | | | | | | | CH). |
| | | | | | | | | | Major diastereomer (57%): |
| | | | | | | | | | 1.33 (s, 3H, Me), 1.56 (d, 3H, |
| | | | | | | | | | Me), 4.19 (s, 3H, Me), 4.44 (d, |
| | | | | | | | | | 1H, CH₂), 4.63 (d, 1H, CH₂), |
| | | | | | | | | | 5.41 (d, 1H, CH), 7.64 (s, 1H, |
| | | | | | | | | | CH). |
| 7.23 | 2 | F | H | H | H | Me | H | solid | 1.37 (s, 3H, Me), 1.58 (d, 3H, |
| | | | | | | | | | Me), 4.20 (s, 3H, Me), 4.67 (d, |
| | | | | | | | | | 1H, CH₂), 4.72 (d, 1H, CH₂), |
| | | | | | | | | | 5.43 (d, 1H, CH), 7.70 (s, 1H, |
| | | | | | | | | | CH). |
| 7.24 | 0 | F | H | H | H | ⁱPr | CF₃ | | 1.33 (s, 3H, Me), 1.49 (d, 3H, |
| | | | | | | | | | Me), 1.58 (d, 6H, Me), 4.42 (s, |
| | | | | | | | | | 2H, CH₂), 4.83 (sept, 1H, |
| | | | | | | | | | CH), 5.08 (d, 1H, CH). |
| 7.25 | 1 | F | H | H | H | ⁱPr | CF₃ | 90- | Diastereomer A: |
| | | | | | | | | 91 | 1.39 (s, 3H, Me), 1.58-1.61 |
| | | | | | | | | | (m, 9H, Me), 4.52 (d, 1H, |
| | | | | | | | | | CH₂), 4.62 (d, 1H, CH₂), 4.86 |
| | | | | | | | | | (sept, 1H, CH), 5.77 (d, 1H, |
| | | | | | | | | | CH). |
| 7.26 | 1 | F | H | H | H | ⁱPr | CF₃ | oil | Diastereomer B: |
| | | | | | | | | | 1.41 (s, 3H, Me), 1.58-1.60 |
| | | | | | | | | | (m, 9H, Me), 4.57 (d, 1H, |
| | | | | | | | | | CH₂), 4.68 (d, 1H, CH₂), 4.85 |
| | | | | | | | | | (sept, 1H, CH), 5.58 (d, 1H, |
| | | | | | | | | | CH). |
| 7.27 | 2 | F | H | H | H | ⁱPr | CF₃ | oil | 1.41 (s, 3H, Me), 1.55-1.60 |
| | | | | | | | | | (m, 9H, Me), 4.77 (s, 2H, |
| | | | | | | | | | CH₂), 4.87 (sept, 1H, CH), |
| | | | | | | | | | 5.47 (d, 1H, CH). |
| 7.28 | 0 | F | H | H | H | Et | CF₃ | oil | 1.33 (s, 3H, Me), 1.58 (t, 3H, |
| | | | | | | | | | Me), 1.49 (d, 3H, Me), 4.42 (s, |
| | | | | | | | | | 2H, CH₂), 4.48 (q, 2H, CH₂), |
| | | | | | | | | | 5.08 (d, 1H, CH). |
| 7.30 | 2 | F | H | H | H | Et | CF₃ | oil | 1.41 (s, 3H, Me), 1.52-1.66 |
| | | | | | | | | | (m, 6H, Me), 4.52 (q, 2H, |
| | | | | | | | | | CH₂), 4.78 (s, 2H, CH₂), 5.48 |
| | | | | | | | | | (d, 1H, CH). |
| 7.31 | 0 | F | H | H | H | Me | Et | oil | 1.26 (t, 3H, Me), 1.28 (s, 3H, |
| | | | | | | | | | Me), 1.49 (d, 3H, Me), 2.69 |
| | | | | | | | | | (q, 2H, CH₂), 4.10 (s, 3H, |
| | | | | | | | | | Me), 4.30 (s, 2H, CH₂), 5.07 |
| | | | | | | | | | (d, 1H, CH). |
| 7.32 | 1 | F | H | H | H | Me | Et | oil | 1:1-Mixture of diastereomers: |
| | | | | | | | | | 1.27 (t, 3H, Me), 1.27 (t, 3H, |
| | | | | | | | | | Me), 1.38 (s, 3H, Me), 1.39 (s, |
| | | | | | | | | | 3H, Me), 1.56 (d, 3H, Me), |
| | | | | | | | | | 1.57 (d, 3H, Me), 2.64-2.74 |
| | | | | | | | | | (m, 4H, CH₂), 4.10 (s, 3H, |
| | | | | | | | | | Me), 4.13 (s, 3H, Me), 4.34- |
| | | | | | | | | | 4.58 (m, 4H, CH₂), 5.55 (d, |
| | | | | | | | | | 1H, CH), 5.76 (d, 1H, CH). |
| 7.33 | 2 | F | H | H | H | Me | Et | 81- | 1.30 (t, 3H, Me), 1.40 (s, 3H, |
| | | | | | | | | 82 | Me), 1.59 (d, 3H, Me), 2.73 |
| | | | | | | | | | (q, 2H, Me), 4.13 (s, 2H, |
| | | | | | | | | | CH₂), 4.59 (d, 1H, CH₂), 4.66 |
| | | | | | | | | | (d, 1H, CH₂), 5.43 (d, 1H, |
| | | | | | | | | | CH) |
| 7.34 | 0 | F | H | H | H | Et | H | gum | 1.30 (s, 3H, Me), 1.49 (d, 3H, |
| | | | | | | | | | Me), 1.55 (t, 3H, Me), 4.34 (s, |
| | | | | | | | | | 2H, CH₂), 4.43 (q, 2H, CH₂), |
| | | | | | | | | | 5.04 (d, 1H, CH), 7.58 (s, 1H, |
| | | | | | | | | | CH). |
| 7.35 | 1 | F | H | H | H | Et | H | solid | Minor diastereomer (42%): |
| | | | | | | | | | 1.38 (s, 3H, Me), 1.52-1.60 |
| | | | | | | | | | (m, 6H, Me), 4.42-4.50 (m, |
| | | | | | | | | | 2H, CH₂), 4.55 (d, 1H, CH₂), |
| | | | | | | | | | 5.76 (d, 1H, CH₂), 7.56 (s, 1H, |
| | | | | | | | | | CH). |
| | | | | | | | | | Major diastereomer (58%): |
| | | | | | | | | | 1.33 (s, 3H, Me), 1.52-1.60 |
| | | | | | | | | | (m, 6H, Me), 4.42-4.50 (m, |
| | | | | | | | | | 2H, CH₂), 4.64 (d, 1H, CH₂), |
| | | | | | | | | | 5.43 (d, 1H, CH₂), 7.65 (s, 1H, |
| | | | | | | | | | CH). |
| 7.36 | 2 | F | H | H | H | Et | H | solid | 1.36 (s, 3H, Me), 1.55 (t, 3H, |
| | | | | | | | | | Me), 1.58 (d, 3H, Me), 4.47 |
| | | | | | | | | | (q, 2H, CH₂), 4.68 (d, 1H, |
| | | | | | | | | | CH₂), 4.73 (d, 1H, CH₂), 5.42 |
| | | | | | | | | | (d, 1H, CH), 7.70 (s, 1H, CH). |
| 7.37 | 0 | F | H | H | H | ⁱPr | Me | oil | 1.31 (s, 3H, Me), 1.48 (d, 3H, |
| | | | | | | | | | Me), 1.52 (d, 6H, Me), 2.30 (s, |
| | | | | | | | | | 3H, Me), 4.28 (d, 1H, CH₂), |
| | | | | | | | | | 4.33 (d, 1H, CH₂), 4.70 (sept, |
| | | | | | | | | | 1H, CH), 5.07 (d, 1H, CH). |
| 7.38 | 1 | F | H | H | H | ⁱPr | Me | 86- | 3:2-Mixture of diastereomers; |
| | | | | | | | | 87 | Diastereomer A (major |
| | | | | | | | | | isomer): |
| | | | | | | | | | 1.39 (s, 3H, Me), 1.50-1.64 |
| | | | | | | | | | (m, 9H, Me), 2.34 (s, 3H, Me), |
| | | | | | | | | | 4.38-4.59 (m, 2H, CH₂), 4.66- |
| | | | | | | | | | 4.79 (m, 1H, CH), 5.55 (s, 1H, |
| | | | | | | | | | CH). |
| | | | | | | | | | Diastereomer B (minor |
| | | | | | | | | | isomer): |
| | | | | | | | | | 1.38 (s, 3H, Me), 1.50-1.64 |
| | | | | | | | | | (m, 9H, Me), 2.30 (s, 3H, Me), |
| | | | | | | | | | 4.38-4.59 (m, 2H, CH₂), 4.66- |
| | | | | | | | | | 4.79 (m, 1H, CH), 5.76 (s, 1H, |
| | | | | | | | | | CH). |
| 7.39 | 2 | F | H | H | H | ⁱPr | Me | 94- | 1.39 (s, 3H, Me), 1.52 (d, 6H, |
| | | | | | | | | 95 | Me), 1.57 (d, 3H, Me), 2.36 (s, |
| | | | | | | | | | 3H, Me), 4.59 (d, 1H, CH₂), |
| | | | | | | | | | 4.64 (d, 1H, CH₂), 4.73 (sept, |
| | | | | | | | | | 1H, CH), 5.42 (d, 1H, CH). |
| 7.42 | 0 | F | H | H | H | Me | CF₃ | oil | 3:2-Mixture of diastereomers; |
| | | | | | | | | | Diastereomer A (major |
| | | | | | | | | | isomer): |
| | | | | | | | | | 1.4 (s, 3H, Me), 1.58 (d, 3H, |
| | | | | | | | | | Me), 4.22 (s, 3H, Me), 4.55 (d, |
| | | | | | | | | | 1H, CH₂), 4.78 (d, 1H, CH₂), |
| | | | | | | | | | 5.55 (d, 1H, CH). |
| | | | | | | | | | Diastereomer B (minor |
| | | | | | | | | | isomer): |
| | | | | | | | | | 1.38 (s, 3H, Me), 1.58 (d, 3H, |
| | | | | | | | | | Me), 4.24 (s, 3H, Me), 4.45 (d, |
| | | | | | | | | | 1H, CH₂), 4.6 (d, 1H, CH₂), |
| | | | | | | | | | 5.75 (d, 1H, CH). |
| 7.43 | 1 | F | H | H | H | Me | CF₃ | oil | 1.4 (s, 3H, Me), 1.58 (d, 3H, |
| | | | | | | | | | Me), 4.25 (s, 3H, Me), 4.73 (d, |
| | | | | | | | | | 1H, CH₂), 4.78 (d, 1H, CH₂), |
| | | | | | | | | | 5.48 (d, 1H, CH). |
| 7.44 | 2 | F | H | H | H | Me | CF₃ | 117- | 1.32 (s, 3H, Me), 1.5 (d, 3H, |
| | | | | | | | | 118 | Me), 4.2 (s, 3H, Me), 4.4 (s, |
| | | | | | | | | | 2H, CH₂), 5.1 (d, 1H, CH). |
| 7.45 | 0 | F | H | H | H | Me | OCH₃ | oil | 1.32 (s, 3H, Me), 1.49 (d, 3H, |
| | | | | | | | | | Me), 3.95 (s, 3H, Me), 3.99 (s, |
| | | | | | | | | | 3H, Me), 4.24 (q, 2H, CH₂), |
| | | | | | | | | | 5.11 (d, 1H, CH). |
| 7.46 | 1 | F | H | H | H | Me | OCH₃ | 138 | Major diastereomer (62%): |
| | | | | | | | | | 1.39 (s, 3H, Me), 1.55-1.6 (m, |
| | | | | | | | | | 3H, Me), 3.94 (s, 3H, Me), |
| | | | | | | | | | 4.00 (s, 3H, Me), 4.43 (q, 2H, |
| | | | | | | | | | CH₂), 5.60 (d, 2H, CH). |
| | | | | | | | | | Minor diastereomer (38%): |
| | | | | | | | | | 1.39 (s, 3H, Me), 1.55-1.6 (m, |
| | | | | | | | | | 3H, Me), 3.94 (s, 3H, Me), |
| | | | | | | | | | 4.02 (s, 3H, Me), 4.39 (q, 2H, |
| | | | | | | | | | CH₂), 5.75 (d, 1H, CH). |
| 7.47 | 2 | F | H | H | H | Me | OCH₃ | 126 | 1.40 (s, 3H, Me), 1.59 (d, 3H, |
| | | | | | | | | | Me), 3.95 (s, 3H, Me), 4.02 (s, |
| | | | | | | | | | 3H, Me), 4.57 (q, 2H, CH₂), |
| | | | | | | | | | 5.45 (d, 1H, CH). |
| 7.48 | 1 | F | H | F | F | Me | OCH₃ | gum | 1.49 (s, 3H, Me), 1.58 (d, 3H, |
| | | | | | | | | | Me), 3.99 (s, 3H, Me), 4.10 (s, |
| | | | | | | | | | 3H, Me), 5.47 (d, 1H, CH). |
| 7.49 | 2 | F | H | F | F | Me | OCH₃ | 117- | 1.44 (s, 3H, Me), 1.64 (d, 3H, |
| | | | | | | | | 120 | Me), 4.01 (s, 3H, Me), 4.12 (s, |
| | | | | | | | | | 3H, Me), 5.47 (d, 1H, CH). |
| 7.50 | 0 | F | H | H | H | Me | Br | | 1.33 (s, 3H, Me), 1.49 (d, 3H, |
| | | | | | | | | | Me), 4.15 (s, 3H, Me), 4.30 (q, |
| | | | | | | | | | 2H, CH₂), 5.08 (d, 1H, CH). |
| 7.51 | 1 | F | H | H | H | Me | Br | gum | Minor diastereomer (40%): |
| | | | | | | | | | 1.40 (s, 3H, Me), 1.59 (d, 3H, |
| | | | | | | | | | Me), 4.19 (s, 3H, Me), 4.39 (d, |
| | | | | | | | | | 1H, CH₂), 4.51 (d, 1H, CH₂), |
| | | | | | | | | | 5.77 (d, 1H, CH). |
| | | | | | | | | | Major diastereomer (60%): |
| | | | | | | | | | 1.41 (s, 3H, Me), 1.58 (d, 3H, |
| | | | | | | | | | Me), 4.17 (s, 3H, Me), 4.45 (d, |
| | | | | | | | | | 1H, CH₂), 4.57 (d, 1H, CH₂), |
| | | | | | | | | | 5.58 (d, 1H, CH). |
| 7.52 | 2 | F | H | H | H | Me | Br | solid | 1.41 (s, 3H, Me), 1.59 (d, 3H, |
| | | | | | | | | | Me), 4.20 (s, 3H, Me), 4.66 (s, |
| | | | | | | | | | 2H, CH₂), 5.47 (d, 1H, CH). |
| 7.53 | 0 | F | H | H | H | Et | Me | oil | 1.31 (s, 3H, Me), 1.48 (d, 3H, |
| | | | | | | | | | Me), 1.52 (t, 3H, Me), 2.30 (s, |
| | | | | | | | | | 3H, Me), 4.30 (s, 2H, CH₂), |
| | | | | | | | | | 4.35 (q, 2H, CH₂), 5.07 (d, |
| | | | | | | | | | 1H, CH). |
| 7.54 | 1 | F | H | H | H | Et | Me | | 4:1-Mixture of diastereomers; |
| | | | | | | | | | Diastereomer A (major |
| | | | | | | | | | isomer): |
| | | | | | | | | | 1.39 (s, 3H, Me), 1.53 (t, 3H, |
| | | | | | | | | | Me), 1.58 (d, 3H, Me), 2.30 (s, |
| | | | | | | | | | 3H, Me), 4.32-4.41 (m, 3H, |
| | | | | | | | | | CH₂), 4.48 (d, 1H, CH₂), 5.76 |
| | | | | | | | | | (d, 1H, CH). |
| | | | | | | | | | Diastereomer B: |
| | | | | | | | | | 1.39 (s, 3H, Me), 1.51 (t, 3H, |
| | | | | | | | | | Me), 1.56 (d, 3H, Me), 2.34 (s, |
| | | | | | | | | | 3H, Me), 4.36 (q, 2H, CH₂), |
| | | | | | | | | | 4.39 (d, 1H, CH₂), 4.57 (d, |
| | | | | | | | | | 1H, CH₂), 5.53 (d, 1H, CH). |
| 7.55 | 1 | F | H | H | H | Et | Me | 79 | Diastereomer B: |
| | | | | | | | | | 1.39 (s, 3H, Me), 1.51 (t, 3H, |
| | | | | | | | | | Me), 1.56 (d, 3H, Me), 2.34 (s, |
| | | | | | | | | | 3H, Me), 4.36 (q, 2H, CH₂), |
| | | | | | | | | | 4.39 (d, 1H, CH₂), 4.57 (d, |
| | | | | | | | | | 1H, CH₂), 5.53 (d, 1H, CH). |
| 7.56 | 2 | F | H | H | H | Et | Me | 118 | 1.39 (s, 3H, Me), 1.51 (t, 3H, |
| | | | | | | | | | Me), 1.58 (d, 3H, Me), 2.36 (s, |
| | | | | | | | | | 3H, Me), 4.38 (q, 2H, CH₂), |
| | | | | | | | | | 4.59 (d, 1H, CH₂), 4.65 (d, |
| | | | | | | | | | 1H, CH₂), 5.42 (d, 1H, CH). |
| 7.57 | 2 | F | H | F | F | Et | Me | 75- | 1.45 (s, 3H, Me), 1.58 (t, 3H, |
| | | | | | | | | 76 | Me), 1.64 (d, 3H, Me), 2.47 (s, |
| | | | | | | | | | 3H, Me), 4.47 (q, 2H, CH₂), |
| | | | | | | | | | 5.47 (d, 1H, CH). |
| 7.58 | 0 | F | H | H | H | Me | OCHF₂ | | 1.32 (s, 3H, Me), 1.49 (d, 3H, |
| | | | | | | | | | Me), 4.08 (s, 3H, Me), 4.27 (s, |
| | | | | | | | | | 2H, CH₂), 5.09 (d, 1H, CH), |
| | | | | | | | | | 6.78 (t, 1H, CH). |
| 7.59 | 1 | F | H | H | H | Me | OCHF₂ | gum | 6:4-Mixture of diastereomers. |
| | | | | | | | | | Major diastereoisomer: |
| | | | | | | | | | 1.41 (s, 3H, Me), 1.57-1.60 |
| | | | | | | | | | (m, 3H, Me), 4.09 (s, 3H, Me), |
| | | | | | | | | | 4.47 (q, 2H, CH₂), 5.54 (d, |
| | | | | | | | | | 1H, CH), 6.80 (t, 1H, CH). |
| | | | | | | | | | Minor diastereoisomer: |
| | | | | | | | | | 1.39 (s, 3H, Me), 1.57-1.60 |
| | | | | | | | | | (m, 3H, Me), 4.11 (s, 3H, Me), |
| | | | | | | | | | 4.42 (q, 2H, CH₂), 5.75 (d, |
| | | | | | | | | | 1H, CH), 6.90 (t, 1H, CH). |
| 7.60 | 2 | F | H | H | H | Me | OCHF₂ | gum | 1.41 (s, 3H, Me), 1.60 (d, 3H, |
| | | | | | | | | | Me), 4.12 (q, 2H, CH₂), 4.62 |
| | | | | | | | | | (s, 3H, Me), 5.48 (d, 1H, CH), |
| | | | | | | | | | 6.80 (t, 1H, CH). |
| 7.61 | 1 | F | H | F | F | Me | OCHF₂ | gum | 7:3-Mixture of diastereomers. |
| | | | | | | | | HF₂ | Major diastereomer: |
| | | | | | | | | | 1.37 (s, 3H, Me), 1.60 (d, 3H, |
| | | | | | | | | | Me), 4.18 (s, 3H, Me), 5.6 (d, |
| | | | | | | | | | 1H, CH), 6.87 (t, 1H, CH). |
| | | | | | | | | | Minor diastereomer: |
| | | | | | | | | | 1.4 (s, 3H, Me), 4.17 (s, 3H, |
| | | | | | | | | | Me), 4.17 (s, 3H, Me), 5.47 (d, |
| | | | | | | | | | 1H, CH), 6.84 (t, 1H, CH). |
| 7.62 | 2 | F | H | F | F | Me | OCHF₂ | gum | 1.45 (s, 3H, Me), 1.64 (d, 3H, |
| | | | | | | | | | Me), 4.21 (s, 3H, Me), 5.47 (d, |
| | | | | | | | | | 1H, CH), 6.88 (t, 1H, CH). |

Key:
Me = methyl; s = singlet; m = multiplet; d = doublet; dd = double doublet; t = triplet; q = quartet; sept = septet.

TABLE 58
Compounds of formula I.8

| | |
|---|---|
| | I.8 |
| 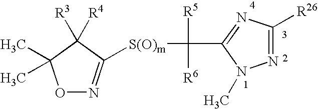 | |

| | | | | | | | M.p. | |
|---|---|---|---|---|---|---|---|---|
| No | m | R³ | R⁴ | R⁵ | R⁶ | R²⁶ | [° C.] | ¹H-NMR (CDCl₃, 400 MHz) |
| 8.01 | 0 | F | H | H | H | Me | gum | 1.3 (s, 3H, Me), 1.5 (m, 3H, |
| | | | | | | | | Me), 2.35 (s, 3H, Me), 3.87 (s, |
| | | | | | | | | 3H, Me), 4.34 (s, 2H, CH₂), |
| | | | | | | | | 5.05-5.2 (d, 1H, CH). |
| 8.02 | 1 | F | H | H | H | Me | 67- | Diastereomer 1: |
| | | | | | | | 68 | 1.40 (s, 3H, Me), 1.60 (m, 3H, |
| | | | | | | | | Me), 2.39 (s, 3H, Me), 3.89 (s, |
| | | | | | | | | 3H, Me), 4.4-4.63 (q, 2H, |
| | | | | | | | | CH₂), 5.70-5.85 (d, 1H, CH). |
| 8.03 | 1 | F | H | H | H | Me | 112- | Diastereomer 2: |
| | | | | | | | 114 | 1.46 (s, 3H, Me), 1.57 (m, 3H, |
| | | | | | | | | Me), 2.35 (s, 3H, Me), 3.91 (s, |
| | | | | | | | | 3H, Me), 4.43-4.70 (q, 2H, |
| | | | | | | | | CH₂), 5.37-5.50 (d, 1H, CH). |
| 8.04 | 2 | F | H | H | H | Me | 182- | 1.48 (s, 3H, Me), 1.60 (m, 3H, |
| | | | | | | | 184 | Me), 2.33 (s, 3H, Me), 3.95 (s, |
| | | | | | | | | 3H, Me), 4.68-4.82 (q, 2H, |
| | | | | | | | | CH₂), 5.33-5.49 (d, 1H, CH). |
| 8.05 | 2 | F | H | Cl | H | Me | 140- | 3:2 -Mixture of diastereomers: |
| | | | | | | | 141 | Major Isomer 1: |
| | | | | | | | | 1.51 (s, 3H, Me), 1.60 (m, 3H, |
| | | | | | | | | Me), 2.39 (s, 3H, Me), 4.06 (s, |
| | | | | | | | | 3H, Me), 5.44-5.59 (d, 1H, |
| | | | | | | | | CH), 6.14 (s, 1H, CH). |
| | | | | | | | | Minor Isomer 2: |
| | | | | | | | | 1.47 (s, 3H, Me), 1.60 (m, 3H, |
| | | | | | | | | Me), 2.37 (s, 3H, Me), 4.06 (s, |
| | | | | | | | | 3H, Me), 5.38-5.52 (d, 1H, |
| | | | | | | | | CH), 6.22 (s, 1H CH). |
| 8.06 | 2 | F | H | Cl | Cl | Me | gum | 1.45 (s, 3H, Me), 1.61 (m, 3H, |
| | | | | | | | | Me), 2.33 (s, 3H, Me), 4.17 (s, |
| | | | | | | | | 3H, Me), 5.50-5.66 (d, 1H, |
| | | | | | | | | CH). |
| 8.07 | 0 | F | H | H | H | H | oil | 1.3 (s, 3H, Me), 1.48, (d, 3H, |
| | | | | | | | | Me), 3.94 (s, 3H, Me), 4.39 (s, |
| | | | | | | | | 7.83 (s, 1H, CH). |
| 8.08 | 1 | F | H | H | H | H | solid | Diastereomer A: |
| | | | | | | | | 1.44 (s, 3H, Me), 1.56 (d, 3H, |
| | | | | | | | | Me), 4.00 (s, 3H, Me), 4.51 (d, |
| | | | | | | | | 1H, CH₂), 4.74 (d, 1H, CH₂), |
| | | | | | | | | 5.39 (d, 1H, CH), 7.90 (s, 1H, |
| | | | | | | | | CH). |
| | | | | | | | | Diastereomer B: |
| | | | | | | | | 1.41 (s, 3H, Me), 1.58 (d, 3H, |
| | | | | | | | | Me), 3.96 (s, 3H, Me), 4.48 (d, |
| | | | | | | | | 1H, CH₂), 4.66 (d, 1H, CH₂), |
| | | | | | | | | 5.78 (d, 1H, CH), 7.93 (s, 1H, |
| | | | | | | | | CH). |
| 8.09 | 2 | F | H | H | H | H | 109- | 1.45 (s, 3H, Me), 1.59 (d, 3H, |
| | | | | | | | 112 | Me), 4.03 (s, 3H, Me), 4.75 (d, |
| | | | | | | | | 1H, CH₂), 4.86 (d, 1H, CH₂), |
| | | | | | | | | 5.39 (d, 1H, CH), 7.89 (s, 1H, |
| | | | | | | | | CH). |

Key:
Me = methyl; s = singlet; m = multiplet; d = doublet; q = quartet.

TABLE 59
Compounds of formula I.9

| | |
|---|---|
| | I.9 |
| 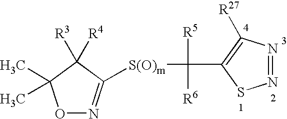 | |

| | | | | | | | M.p. | ¹H-NMR |
|---|---|---|---|---|---|---|---|---|
| No | m | R³ | R⁴ | R⁵ | R⁶ | R²⁷ | [° C.] | (CDCl₃, 400 MHz) |
| 9.01 | 0 | F | H | H | H | Me | oil | 1.29 (s, 3H, Me), 1.48 (d, |
| | | | | | | | | 3H, Me), 2.71 (s, 3H, Me), |
| | | | | | | | | 4.46 (d, 1H, CH₂), 4.52 (d, |
| | | | | | | | | 1H, CH₂), 4.99 |
| | | | | | | | | (d, 1H, CH). |
| 9.02 | 1 | F | H | H | H | Me | solid | 1.38 (s, 3H, Me), 1.57 (d, |
| | | | | | | | | 3H, Me), 2.76 (s, 3H, Me), |
| | | | | | | | | 4.64 (d, 1H, CH₂), 4.73 (d, |
| | | | | | | | | 1H, CH₂), 5.65 (d, |
| | | | | | | | | 1H, CH). |
| 9.03 | 1 | F | H | H | H | Me | solid | 1.05 (s, 3H, Me), 1.51 (d, |
| | | | | | | | | 3H, Me), 2.74 (s, 3H, Me), |
| | | | | | | | | 4.71 (d, 1H, CH₂), 4.76 (d, |
| | | | | | | | | 1H, CH₂), 5.20 (d, |
| | | | | | | | | 1H, CH). |
| 9.04 | 2 | F | H | H | H | Me | oil | 1.36 (s, 3H, Me), 1.58 (d, |
| | | | | | | | | 3H, Me), 2.76 (s, 3H, Me), |
| | | | | | | | | 4.86 (d, 1H, CH₂), 4.93 (d, |
| | | | | | | | | 1H, CH₂), 5.47 (d, |
| | | | | | | | | 1H, CH). |

Key:
Me = methyl; s = singlet; d = doublet.

TABLE 60
Compounds of formula I.10

| | |
|---|---|
| | I.10 |
| 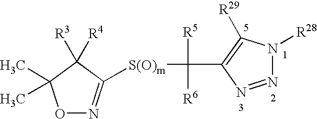 | |

| | | | | | | | | M.p. | |
|---|---|---|---|---|---|---|---|---|---|
| No | m | R³ | R⁴ | R⁵ | R⁶ | R²⁸ | R²⁹ | [° C.] | ¹H-NMR (CDCl₃, 400 MHz) |
| 10.01 | 0 | F | H | H | H | ^{t}Bu | H | oil | 1.28 (s, 3H, Me), 1.47 (d, |
| | | | | | | | | | 3H, Me), 1.65 (s, 9H, Me), |
| | | | | | | | | | 4.35 (d, 1H, CH₂), 4.39 (d, |
| | | | | | | | | | 1H, CH₂), 5.05 (d, 1H, CH), |
| | | | | | | | | | 7.62 (s, 1H, CH). |
| 10.02 | 1 | F | H | H | H | ^{t}Bu | H | solid | Diastereomer A: |
| | | | | | | | | | 1.36 (s, 3H, Me), 1.53 (d, |
| | | | | | | | | | 3H, Me), 1.68 (s, 9H, Me), |
| | | | | | | | | | 4.45 (d, 1H, CH₂), 4.66 (d, |
| | | | | | | | | | 1H, CH₂), 5.30 (d, 1H, CH), |
| | | | | | | | | | 7.77 (s, 1H, CH). |
| | | | | | | | | | Diastereomer B: |
| | | | | | | | | | 1.36 (s, 3H, Me), 1.57 (d, |
| | | | | | | | | | 3H, Me), 1.68 (s, 9H, Me), |
| | | | | | | | | | 4.49 (d, 1H, CH₂), 4.59 (d, |
| | | | | | | | | | 1H, CH₂), 5.72 (d, 1H, CH), |
| | | | | | | | | | 7.67 (s, 1H, CH). |
| 10.03 | 2 | F | H | H | H | ^{t}Bu | H | solid | 1.39 (s, 3H, Me), 1.54 (d, |
| | | | | | | | | | 3H, Me), 1.68 (s, 9H, Me), |
| | | | | | | | | | 4.73 (d, 1H, CH₂), 4.78 (d, |
| | | | | | | | | | 1H, CH₂), 5.38 (d, 1H, CH), |
| | | | | | | | | | 7.83 (s, 1H, CH). |
| 10.04 | 0 | F | H | H | H | Me | Me | gum | 1.27 (s, 3H, Me), 1.46 (d, |
| | | | | | | | | | 3H, Me), 2.30 (s, 3H, Me), |
| | | | | | | | | | 3.92 (s, 3H, Me), 4.27 (d, |
| | | | | | | | | | 1H, CH₂), 4.33 (d, 1H, |
| | | | | | | | | | CH₂), 5.09 (d, 1H, CH). |
| 10.05 | 1 | F | H | H | H | Me | Me | solid | 9:7-Mixture of |
| | | | | | | | | | diastereomers: |
| | | | | | | | | | Diastereoisomer A (major): |
| | | | | | | | | | 1.40 (s, 3H, Me), 1.58 (d, |
| | | | | | | | | | 3H, Me), 2.33 (s, 3H, Me), |
| | | | | | | | | | 3.97 (s, 3H, Me), 4.45 (d, |
| | | | | | | | | | 1H, CH₂), 4.51 (d, 1H, |
| | | | | | | | | | CH₂), 5.77 (d, 1H, CH). |
| | | | | | | | | | Diastereoisomer B (minor): |
| | | | | | | | | | 1.45 (s, 3H, Me), 1.54 (d, |
| | | | | | | | | | 3H, Me), 2.36 (s, 3H, Me), |
| | | | | | | | | | 3.97 (s, 3H, Me), 4.39 (d, |
| | | | | | | | | | 1H, CH₂), 4.62 (d, 1H, |
| | | | | | | | | | CH₂), 5.46 (d, 1H, CH). |
| 10.06 | 2 | F | H | H | H | Me | Me | solid | 1.44 (s, 3H, Me), 1.56 (d, |
| | | | | | | | | | 3H, Me), 2.39 (s, 3H, Me), |
| | | | | | | | | | 3.98 (s, 3H, Me), 4.61 (d, |
| | | | | | | | | | 1H, CH₂), 4.70 (d, 1H, |
| | | | | | | | | | CH₂), 5.40 (d, 1H, CH). |

Key:
Me = methyl; s = singlet; d = doublet.

TABLE 61
Compounds of formula I.11

| | |
|---|---|
| | I.11 |
| 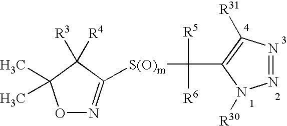 | |

| | | | | | | | | M.p. | |
|---|---|---|---|---|---|---|---|---|---|
| No | m | R³ | R⁴ | R⁵ | R⁶ | R³⁰ | R³¹ | [° C.] | ¹H-NMR (CDCl₃, 400 MHz) |
| 11.01 | 0 | F | H | H | H | Me | Br | gum | 1.31 (s, 3H, Me), 1.49 (d, |
| | | | | | | | | | 3H, Me), 4.14 (s, 3H, Me), |
| | | | | | | | | | 4.35 (s, 2H, CH₂), 5.03 (d, |
| | | | | | | | | | 1H, CH). |
| 11.02 | 1 | F | H | H | H | Me | Br | solid | Minor diastereomer (40%): |
| | | | | | | | | | 1.41 (s, 3H, Me), 1.62 (d, |
| | | | | | | | | | 3H, Me), 4.16 (s, 3H, Me), |
| | | | | | | | | | 4.34 (d, 1H, CH₂), 4.58 (d, |
| | | | | | | | | | 1H, CH₂), 5.76 (d, 1H, CH). |
| | | | | | | | | | Major diastereomer (60%): |
| | | | | | | | | | 1.40 (s, 3H, Me), 1.59 (d, |
| | | | | | | | | | 3H, Me), 4.19 (s, 3H, Me), |
| | | | | | | | | | 4.56 (d, 1H, CH₂), 4.59 (d, |
| | | | | | | | | | 1H, CH₂), 5.47 (d, 1H, CH). |
| 11.03 | 2 | F | H | H | H | Me | Br | solid | 1.41 (s, 3H, Me), 1.60 (d, |
| | | | | | | | | | 3H, Me), 4.23 (s, 3H, Me), |
| | | | | | | | | | 4.71 (s, 2H, CH₂), 5.47 (d, |
| | | | | | | | | | 1H, CH). |
| 11.04 | 0 | F | H | H | H | ⁱPr | Me | | 1.35 (s, 3H, Me), 1.5 (d, 3H, |
| | | | | | | | | | Me), 1.62 (m, 6H, Me), 2.35 |
| | | | | | | | | | (s, 3H, Me), 4.32 (s, 2H, |
| | | | | | | | | | CH₂), 5.14-5.21 (m, 1H, |
| | | | | | | | | | CH), 5.0 (d, 1H, CH). |
| 11.05 | 2 | F | H | H | H | ⁱPr | Me | 114- | 1.39 (s, 3H, Me), 1.59 (d, |
| | | | | | | | | 115 | 3H, Me), 1.62 (d, 3H, Me), |
| | | | | | | | | | 1.63 (d, 3H, Me), 2.35 (s, |
| | | | | | | | | | 3H, Me), 4.62 (d, 1H, CH₂), |
| | | | | | | | | | 4.67 (d, 1H, CH₂), 4.69 (m, |
| | | | | | | | | | 1H, CH), 5.49 (d, 1H, CH). |
| 11.06 | 2 | F | H | H | H | —CH₂CH═CH₂ | Me | oil | 1.39 (s, 3H, Me), 1.59 (d, |
| | | | | | | | | | 3H, Me), 2.35 (s, 3H, Me), |
| | | | | | | | | | 4.64 (d, 1H, CH₂), 4.69 (d, |
| | | | | | | | | | m, CH₂), 5.14-5.21 (m, 3H, |
| | | | | | | | | | CH₂), 5.34 (d, m, CH₂), |
| | | | | | | | | | 5.49 (d, 1H, CH), 5.92-6.01 |
| | | | | | | | | | (m, 1H, CH). |

Key:
Me = methyl; s = singlet; m = multiplet; d = doublet.

TABLE 62
Compounds of formula I.12

| | |
|---|---|
| | I.12 |
| 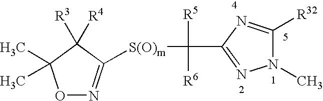 | |

| | | | | | | | M.p. | ¹H-NMR (CDCl₃, |
|---|---|---|---|---|---|---|---|---|
| No | m | R³ | R⁴ | R⁵ | R⁶ | R³² | [° C.] | 400 MHz) |
| 12.01 | 0 | F | H | H | H | H | oil | 1.32 (s, 3H, Me), 1.48 |
| | | | | | | | | (d, 3H, Me), 3.89 (s, 3H, |
| | | | | | | | | Me), 4.36 (s, 2H, CH₂), |
| | | | | | | | | 5.17 (d, 1H, CH), 7.97 |
| | | | | | | | | (s, 1H, CH). |
| 12.02 | 1 | F | H | H | H | H | 97-99 | Diastereomer A: |
| | | | | | | | | 1.43 (s, 3H, Me), 1.57 |
| | | | | | | | | (d, 3H, Me), 3.91 (s, 3H, |
| | | | | | | | | Me), 4.52 (d, 1H, CH₂), |
| | | | | | | | | 4.64 (d, 1H, CH₂), 5.65 |
| | | | | | | | | (d, 1H, CH), 8.00 (s, 1H, |
| | | | | | | | | CH). |
| | | | | | | | | Diastereomer B: |
| | | | | | | | | 1.39 (s, 3H, Me), 1.58 |
| | | | | | | | | (d, 3H, Me), 3.93 (s, 3H, |
| | | | | | | | | Me), 4.46 (d, 1H, CH₂), |
| | | | | | | | | 4.61 (d, 1H, CH₂), 5.80 |
| | | | | | | | | (d, 1H, CH), 8.03 (s, 1H, |
| | | | | | | | | CH). |
| 12.03 | 2 | F | H | H | H | H | 109- | 1.45 (s, 3H, Me), 1.59 |
| | | | | | | | 112 | (d, 3H, Me), 3.94 (s, 3H, |
| | | | | | | | | Me), 4.71 (d, 1H, CH₂), |
| | | | | | | | | 4.78 (d, 1H, CH₂), 5.50 |
| | | | | | | | | (d, 1H, CH), 8.03 (s, 1H, |
| | | | | | | | | CH). |

Key:
Me = methyl; s = singlet; d = doublet.

TABLE 63
Compounds of formula I.13

| | |
|---|---|
| | I.13 |
| 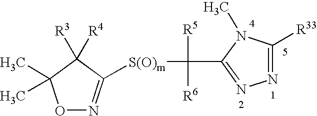 | |

| | | | | | | | M.p. | ¹H-NMR |
|---|---|---|---|---|---|---|---|---|
| No | m | R³ | R⁴ | R⁵ | R⁶ | R³³ | [° C.] | (CDCl₃, 400 MHz) |
| 13.01 | 0 | F | H | H | H | Me | oil | 1.30 (s, 3H, Me), 1.45 (d, |
| | | | | | | | | 3H, Me), 2.44 (s, 3H, Me), |
| | | | | | | | | 3.59 (s, 3H, Me), 4.34 (d, |
| | | | | | | | | 1H, CH₂), 4.47 (d, 1H, |
| | | | | | | | | CH₂), 5.20 (d, 1H, CH). |
| 13.02 | 1 | F | H | H | H | Me | oil | Diastereomer A: |
| | | | | | | | | 1.50 (s, 3H, Me), 1.55 (d, |
| | | | | | | | | 3H, Me), 2.48 (s, 3H, Me), |
| | | | | | | | | 3.64 (s, 3H, Me), 4.46 (d, |
| | | | | | | | | 1H, CH₂), 4.72 (d, 1H, |
| | | | | | | | | CH₂), 5.32 (d, 1H, CH). |
| | | | | | | | | Diastereomers B: |
| | | | | | | | | 1.42 (s, 3H, Me), 1.59 (d, |
| | | | | | | | | 3H, Me), 2.47 (s, 3H, Me), |
| | | | | | | | | 3.63 (s, 3H, Me), 4.54 (d, |
| | | | | | | | | 1H, CH₂), 4.62 (d, 1H, |
| | | | | | | | | CH₂), 5.79 (d, 1H, CH). |
| 13.03 | 2 | F | H | H | H | Me | 148- | 1.50 (s, 3H, Me), 1.56 (d, |
| | | | | | | | 151 | 3H, Me), 2.48 (s, 3H, Me), |
| | | | | | | | | 3.69 (s, 3H, Me), 4.71 (d, |
| | | | | | | | | 1H, CH₂), 4.85 (d, 1H, |
| | | | | | | | | CH₂), 5.34 (d, 1H, CH). |

Key:
Me = methyl; s = singlet; d = doublet.

TABLE 64
Compounds of formula I.14

| | |
|---|---|
| | I.14 |
| 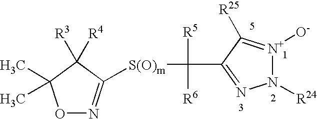 | |

| | | | | | | | | M.p. | |
|---|---|---|---|---|---|---|---|---|---|
| No | m | R³ | R⁴ | R⁵ | R⁶ | R²⁴ | R²⁵ | [° C.] | ¹H-NMR (CDCl₃, 400 MHz) |
| 14.01 | 0 | F | H | H | H | Me | Me | | 1.29 (s, 3H, Me), 1.48 (d, 3H, |
| | | | | | | | | | Me), 2.33 (s, 3H, Me), 3.94 (s, |
| | | | | | | | | | 3H, Me), 4.24 (d, 1H, CH₂), |
| | | | | | | | | | 4.27 (d, 1H, CH₂), 5.07 (d, |
| | | | | | | | | | 1H, CH). |
| 14.02 | 2 | F | H | H | H | Me | Me | | 1.45 (s, 3H, Me), 1.58 (d, 3H, |
| | | | | | | | | | Me), 2.37 (s, 3H, Me), 3.94 (s, |
| | | | | | | | | | 3H, Me), 4.59 (d, 1H, CH₂), |
| | | | | | | | | | 4.70 (d, 1H, CH₂), 5.58 (d, |
| | | | | | | | | | m, CH). |

Key:
Me = methyl; s = singlet; d = doublet.

### Biological Examples

### Example B1

### Herbicidal Action

Monocotyledonous and dicotyledonous test plants were sown in sterilised standard soil in seed trays each having 96 cells. After one day (pre-emergence) under controlled conditions in a climatic chamber (cultivation at 23/17° C., day/night; 13 hours light; 50-60% humidity; after application at 24/19° C., day/night), the plants were treated with an aqueous spray solution of 1000 mg/l of the active ingredient used (including 10% DMSO as solvent). The plants were grown on in the climatic chamber until the test was evaluated (10=total damage to plant, 0=no damage to plant) after 9 or 13 days.

TABLE B1a
Application pre-emergence

| Comp. No. | [g/ha] | DIGSA | AGSTE | POATR | AMARE | SETIT |
|---|---|---|---|---|---|---|
| 1.11 | 1000 | 10 | 10 | 10 | 7 | 8 |
| 1.12 | 1000 | 9 | 10 | 0 | 9 | 8 |
| 1.13 | 1000 | 9 | 10 | 10 | 9 | 9 |
| 1.14 | 1000 | 9 | 10 | 2 | 9 | 8 |
| 1.15 | 1000 | 9 | 10 | 10 | 9 | 9 |
| 1.16 | 1000 | 10 | 10 | 10 | 8 | 6 |
| 1.17 | 1000 | 8 | 10 | 10 | 10 | 8 |
| 1.18 | 1000 | 10 | 10 | 8 | 10 | 9 |
| 2.02 | 1000 | 10 | 10 | 5 | 7 | 7 |
| 2.05 | 1000 | 8 | 9 | 0 | 2 | 9 |
| 2.06 | 1000 | 8 | 10 | 9 | 7 | 7 |
| 2.08 | 1000 | 10 | 10 | 10 | 10 | 10 |
| 2.09 | 1000 | 9 | 10 | 10 | 9 | 10 |
| 2.10 | 1000 | 10 | 10 | 10 | 8 | 9 |
| 2.11 | 1000 | 9 | 10 | 10 | 10 | 4 |
| 2.12 | 1000 | 9 | 10 | 10 | 6 | 9 |
| 2.14 | 1000 | 9 | 10 | 10 | 9 | 9 |
| 2.15 | 1000 | 8 | 10 | 10 | 8 | 6 |
| 2.17 | 1000 | 10 | 10 | 10 | 6 | 9 |
| 2.18 | 1000 | 5 | 10 | 9 | 8 | 8 |
| 2.20 | 1000 | 9 | 5 | 8 | 4 | 7 |
| 2.22 | 1000 | 9 | 10 | 9 | 9 | 9 |
| 2.23 | 1000 | 10 | 10 | 10 | 10 | 10 |
| 2.24 | 1000 | 10 | 10 | 10 | 10 | 10 |
| 2.25 | 1000 | 10 | 10 | 10 | 10 | 10 |
| 2.27 | 1000 | 10 | 10 | 10 | 10 | 10 |
| 2.29 | 1000 | 0 | 9 | 10 | 8 | 9 |
| 2.30 | 1000 | 10 | 10 | 9 | 9 | 9 |
| 2.31 | 1000 | 10 | 10 | 10 | 10 | 10 |
| 2.33 | 1000 | 9 | 10 | 9 | 6 | 9 |
| 2.34 | 1000 | 10 | 10 | 10 | 10 | 9 |
| 2.37 | 1000 | 9 | 10 | 10 | 8 | 9 |
| 2.42 | 1000 | 8 | 0 | 8 | 8 | 8 |
| 2.43 | 1000 | 9 | 0 | 10 | 8 | 8 |
| 2.44 | 1000 | 7 | 0 | 10 | 10 | 10 |
| 2.46 | 1000 | 9 | 10 | 10 | 10 | — |
| 2.47 | 1000 | 9 | 10 | 10 | 10 | — |
| 2.49 | 1000 | 10 | 10 | 10 | 10 | — |
| 2.50 | 1000 | 10 | 10 | 10 | 10 | — |
| 2.52 | 1000 | 9 | 10 | 9 | 9 | — |
| 2.53 | 1000 | 9 | 10 | 10 | 10 | — |
| 2.54 | 1000 | 6 | 10 | 10 | 9 | — |
| 2.56 | 1000 | 10 | 10 | 10 | 10 | — |
| 2.58 | 1000 | 10 | 10 | 10 | 10 | — |
| 2.59 | 1000 | 10 | 10 | 10 | 10 | — |
| 2.61 | 1000 | 9 | 10 | 4 | 6 | — |
| 2.63 | 1000 | 8 | 10 | 10 | 10 | — |
| 3.02 | 1000 | 9 | 10 | 10 | 7 | 8 |
| 3.03 | 1000 | 0 | 10 | 10 | 10 | 9 |
| 3.04 | 1000 | 8 | 8 | 7 | 2 | — |
| 3.05 | 1000 | 8 | 9 | 10 | 10 | — |
| 3.06 | 1000 | 0 | 10 | 10 | 8 | — |
| 4.01 | 1000 | 9 | 0 | 0 | 8 | 9 |
| 4.02 | 1000 | 9 | 10 | 10 | 9 | 9 |
| 4.03 | 1000 | 10 | 10 | 10 | 8 | 7 |
| 4.04 | 1000 | 9 | 10 | 8 | 9 | 9 |
| 4.07 | 1000 | 9 | 10 | 10 | 9 | — |
| 4.08 | 1000 | 9 | 10 | 8 | 5 | — |
| 4.09 | 1000 | 7 | 10 | 4 | 9 | — |
| 5.02 | 1000 | 10 | 10 | 10 | 10 | — |
| 5.03 | 1000 | 8 | 10 | 10 | 10 | — |
| 6.01 | 1000 | 8 | 9 | 0 | 0 | — |
| 6.02 | 1000 | 9 | 10 | 10 | 10 | — |
| 6.03 | 1000 | 9 | 10 | 10 | 8 | — |
| 6.04 | 1000 | 9 | 10 | 10 | 9 | — |
| 6.05 | 1000 | 5 | 10 | 4 | 0 | — |
| 6.07 | 1000 | 9 | 10 | 10 | 7 | — |
| 6.08 | 1000 | 8 | 9 | 9 | 3 | — |
| 6.09 | 1000 | 9 | 10 | 9 | 10 | — |
| 6.10 | 1000 | 0 | 10 | 9 | 7 | — |
| 6.12 | 1000 | 10 | 10 | 10 | 10 | — |
| 6.13 | 1000 | 9 | 10 | 10 | 10 | — |
| 7.01 | 1000 | 8 | 10 | 0 | 0 | 8 |
| 7.02 | 1000 | 9 | 10 | 10 | 0 | 9 |
| 7.03 | 1000 | 10 | 8 | 10 | 6 | 9 |
| 7.04 | 1000 | 10 | 10 | 10 | 10 | 9 |
| 7.08 | 1000 | 10 | 10 | 10 | 10 | — |
| 7.09 | 1000 | 10 | 10 | 10 | 10 | — |
| 7.10 | 1000 | 10 | 10 | 10 | 10 | — |
| 7.12 | 1000 | 9 | 10 | 10 | 10 | — |
| 7.13 | 1000 | 9 | 10 | 8 | 4 | — |
| 7.14 | 1000 | 9 | 10 | 8 | 8 | — |
| 7.15 | 1000 | 9 | 8 | 0 | 9 | — |
| 7.18 | 1000 | 8 | 7 | 0 | 8 | — |
| 7.19 | 1000 | 9 | 6 | 10 | 10 | — |
| 7.20 | 1000 | 10 | 10 | 10 | 10 | — |
| 7.22 | 1000 | 8 | 9 | 9 | 0 | — |
| 7.23 | 1000 | 9 | 10 | 9 | 5 | — |
| 7.25 | 1000 | 9 | 10 | 10 | 9 | — |
| 7.26 | 1000 | 5 | 9 | 6 | 0 | — |
| 7.27 | 1000 | 10 | 9 | 0 | 1 | — |
| 7.31 | 1000 | 7 | 9 | 0 | 0 | — |
| 7.32 | 1000 | 9 | 10 | 9 | 7 | — |
| 7.33 | 1000 | 10 | 10 | 10 | 10 | — |
| 7.34 | 1000 | 8 | 9 | 8 | 6 | — |
| 7.35 | 1000 | 9 | 10 | 9 | 8 | — |
| 7.36 | 1000 | 9 | 9 | 10 | 8 | — |
| 7.38 | 1000 | 7 | 10 | 7 | 6 | — |
| 7.39 | 1000 | 9 | 10 | 8 | 8 | — |
| 7.43 | 1000 | 10 | 10 | 10 | 10 | — |
| 8.02 | 1000 | 10 | 10 | 10 | 4 | — |
| 8.03 | 1000 | 9 | 10 | 8 | 0 | — |
| 8.05 | 1000 | 9 | 10 | 8 | 10 | — |
| 11.02 | 1000 | 9 | 10 | 10 | 5 | — |
| 11.03 | 1000 | 9 | 10 | 10 | 3 | — |

DIGSA = Digitaria sanguinalis; AGSTE = Agrostis-tenius; POATR = Poa trivialis; AMARE = Amaranthus retroflexus; SETIT = Setaria italica.

1. A compound of formula 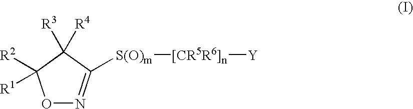
wherein
R¹ and R² are each independently of the other hydrogen, C₁-C₁₀alkyl, C₁-C₁₀haloalkyl, C₃-C₈cycloalkyl or C₃-C₈cycloalkyl-C₁-C₃alkyl, or
R¹ and R² together with the carbon atom to which they are bonded form a C₃-C₇ring,
R³ is halogen, azide, cyano, —SCN, C₂-C₁₀alkynyl, C₂-C₁₀alkenyl, formyl, C₁-C₁₀alkoxy, C₁-C₁₀alkylsulfanyl, C₁-C₁₀haloalkoxy, C₁-C₁₀haloalkylsulfanyl,
R⁴ is hydrogen, C₁-C₁₀alkyl, C₁-C₁₀haloalkyl, C₃-C₈cycloalkyl-C₁-C₁₀alkyl, C₁-C₆alkoxy-C₁-C₁₀alkyl or C₃-C₈cycloalkyl, halogen, azide, cyano, —SCN, C₂-C₁₀alkynyl, C₂-C₁₀alkenyl, formyl, C₁-C₁₀alkoxy, C₁-C₁₀alkylsulfanyl, C₁-C₁₀haloalkoxy, C₁-C₁₀halo-alkylsulfanyl,
or
R² with R⁴ and together with the carbon atoms to which they are bonded form a C₃-C₈ring;
R⁵ and R⁶ are each independently of the other hydrogen, cyano, C₁-C₆alkyl, C₁-C₆alkoxycarbonyl, halogen or C₁-C₆haloalkyl;
m is 0, 1 or 2;
n is 1, 2 or 3;
Y is phenyl, naphthyl or tetrahydronaphthyl, which is optionally substituted by one to five substituents independently selected from C₁-C₆alkyl, C₃-C₆cycloalkyl, C₁-C₆haloalkyl, C₁-C₆hydroxyalkyl, C₁-C₆alkoxy-C₁-C₆alkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, benzyloxycarbonyl, nitro, cyano, formyl, carboxyl, halogen, azido, thiocyanato, tri(C₁-C₆alkyl)silyl, mercapto, phenylthio, phenylsulfinyl, —SF₅, C₁-C₆alkylthio, C₁-C₆haloalkylthio, C₁-C₆haloalkylsulfinyl, C₁-C₆haloalkylsulfonyl, C₁-C₆alkylsulfinyl, C₁-C₆alkylsulfonyl, benzylsulfonyl or benzyl-sulfonyl substituted by one to five R⁹, phenylsulfonyl or phenylsulfonyl substituted by one to five R⁹, hydroxyl, C₁-C₆alkoxy, C₃-C₆cycloalkyloxy wherein one of the CH₂ groups is optionally replaced by an oxygen atom, C₁-C₆haloalkoxy, C₂-C₆alkenyloxy, C₂-C₆alkynyloxy, C₁-C₆alkylsulfonyloxy, C₁-C₆haloalkylsulfonyloxy, phenoxy or phenoxy substituted by one to five R⁹, benzyloxy or benzyloxy substituted by one to five R⁹, —CONH—SO₂—C₁-C₆alkyl, —CONH—SO₂—C₁-C₆haloalkyl, —NH—SO₂—C₁-C₆alkyl, —NH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —OCO—C₁-C₆alkyl, —OCO—C₁-C₆haloalkyl, —OCO-phenyl or —OCO-phenyl substituted by one to five R⁹, —OCONH—C₁-C₆alkyl, —OCONH—C₁-C₆haloalkyl, —OCONH-phenyl or —OCONH-phenyl substituted by one to five R⁹, or by one of the following groups Z, with Z= 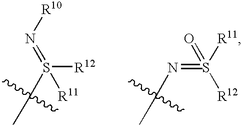
R¹⁰ is hydrogen, formyl, cyano, nitro, C₁-C₆alkylsulfonyl, C₁-C₁₀alkyl, C₁-C₁₀haloalkyl, C₁-C₁₀alkylcarbonyl, C₁-C₁₀haloalkylcarbonyl, C₁-C₁₀alkoxycarbonyl, and R¹¹ and R¹² are independently of each other C₁-C₁₀alkyl, C₁-C₁₀haloalkyl, C₁-C₁₀cyclo-alkyl, C₁-C₁₀cycloalkylalkyl, C₁-C₁₀alkoxyalkyl, or by —CONR⁷R⁸ wherein R⁷ and R³ are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R⁷ and R³ form a C₃-C₈alkylene group which optionally contains one oxygen or sulfur atom or one to two amino or C₁-C₆alkylamino groups, or
Y is a 5- to 10-membered aromatic or non-aromatic heterocycle containing one to three nitrogen, oxygen or sulfur atoms, which is optionally benzo-fused, and which is optionally substituted by one to four substituents independently selected from C₁-C₆alkyl, C₃-C₆cycloalkyl, C₃-C₆cycloalkyl-C₁-C₆alkyl, C₁-C₆haloalkyl, C₁-C₆hydroxyalkyl, C₁-C₆alkoxy-C₁-C₆alkyl, C₂-C₆alkenyl, C₂-C₆alkynyl, C₂-C₆haloalkenyl, C₁-C₆alkylcarbonyl, C₁-C₆haloalkylcarbonyl, C₁-C₆alkoxycarbonyl, nitro, cyano, formyl, carboxyl, halogen, azido, thiocyanato, tri(C₁-C₆alkyl)silyl, mercapto, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to five R⁹, phenylsulfonyl or phenylsulfonyl substituted by one to five R⁹, hydroxyl, C₁-C₆alkoxy, C₁-C₆alkoxy-C₁-C₆alkoxy, C₃-C₆cycloalkyloxy wherein one of the CH₂ groups is optionally replaced by an oxygen atom, C₁-C₆haloalkoxy, C₂-C₆alkenyloxy, C₂-C₆alkynyloxy, C₁-C₆alkylsulfonyloxy, C₁-C₆haloalkylsulfonyloxy, phenoxy or phenoxy substituted by one to five R⁹, benzyloxy or benzyloxy substituted by one to five R⁹, —CONH—SO₂—C₁-C₆alkyl, —CONH—SO₂—C₁-C₆haloalkyl, —NH—SO₂—C≡C₆alkyl, —NH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —OCO—C₁-C₆alkyl, —OCO—C₁-C₆haloalkyl, —OCO-phenyl or —OCO-phenyl substituted by one to five R⁹, —OCONH—C₁-C₆alkyl, —OCONH—C₁-C₆haloalkyl, —OCONH-phenyl or —OCONH-phenyl substituted by one to five R⁹, or by one of the following groups Z, with Z= 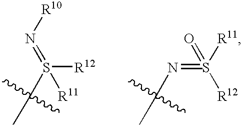
R¹⁰ is hydrogen, formyl, cyano, nitro, C₁-C₆alkylsulfonyl, C₁-C₁₀alkyl, C₁-C₁₀haloalkyl, C₁-C₁₀alkylcarbonyl, C₁-C₁₀haloalkylcarbonyl, C₁-C₁₀alkoxycarbonyl, and R¹¹ and R¹² are independently of each other C₁-C₁₀alkyl, C₁-C₁₀haloalkyl, C₁-C₁₀cyclo-alkyl, C₁-C₁₀cycloalkylalkyl, C₁-C₁₀alkoxyalkyl, or by —CONR⁷R⁸ wherein R⁷ and R³ are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R⁷ and R³ form a C₃-C₈alkylene group which optionally contains one oxygen or sulfur atom or one to two amino or C₁-C₆alkylamino groups;
R⁹ are independently from each other C₁-C₆haloalkyl, C₁-C₆alkoxycarbonyl, nitro, cyano, formyl, carboxyl or halogen;
and to N-oxides, salts and optical isomers of compounds of formula I.

2. A compound of formula I 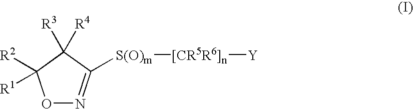
wherein
R¹ and R² are each independently of the other hydrogen, C₁-C₁₀alkyl, C₁-C₁₀haloalkyl, C₃-C₈cycloalkyl or C₃-C₈cycloalkyl-C₁-C₃alkyl, or
R¹ and R² together with the carbon atom to which they are bonded form a C₃-C₇ring, R³ is halogen, azide, cyano, —SCN, C₂-C₁₀alkynyl, C₂-C₁₀alkenyl, formyl, C₁-C₁₀alkoxy, C₁-C₁₀alkylsulfanyl, C₁-C₁₀haloalkoxy, C₁-C₁₀haloalkylsulfanyl,
R⁴ is hydrogen, C₁-C₁₀alkyl, C₁-C₁₀haloalkyl, C₃-C₈cycloalkyl-C₁-C₁₀alkyl, C₁-C₆alkoxy-C₁-C₁₀alkyl or C₃-C₈cycloalkyl, halogen, azide, cyano, —SCN, C₂-C₁₀alkynyl, C₂-C₁₀alkenyl, formyl, C₁-C₁₀alkoxy, C₁-C₁₀alkylsulfanyl, C₁-C₁₀haloalkoxy, C₁-C₁₀haloalkylsulfanyl,
or
R² with R⁴ and together with the carbon atoms to which they are bonded form a C₃-C₈ring;
R⁵ and R⁶ are each independently of the other hydrogen, cyano, C₁-C₆alkyl, C₁-C₆alkoxycarbonyl, halogen or C₁-C₆haloalkyl;
m is 0, 1 or 2;
n is 1, 2 or 3;
Y is phenyl, naphthyl or tetrahydronaphthyl, which is optionally substituted by one to three substituents independently selected from C₁-C₆alkyl, C₃-C₆cycloalkyl, C₁-C₆halo-alkyl, C₁-C₆hydroxyalkyl, C₁-C₆alkoxy-C₁-C₆alkyl, C₂-C₆alkenyl, C₂-C₆alkynyl, C₂-C₆halo-alkenyl, C₁-C₆alkylcarbonyl, C₁-C₆haloalkylcarbonyl, C₁-C₆alkoxycarbonyl, benzyloxy-carbonyl, nitro, cyano, formyl, carboxyl, halogen, azido, thiocyanato, tri(C₁-C₆alkyl)silyl, mercapto, phenylthio, phenylsulfinyl, —SF₅, C₁-C₆alkylthio, C₁-C₆haloalkylthio, C₁-C₆haloalkylsulfinyl, C₁-C₆haloalkylsulfonyl, C₁-C₆alkylsulfinyl, C₁-C₆alkylsulfonyl, benzylsulfonyl or benzylsulfonyl substituted by one to three R⁹, phenylsulfonyl or phenylsulfonyl substituted by one to three R⁹, hydroxyl, C₁-C₆alkoxy, C₃-C₆cycloalkyloxy wherein one of the CH₂ groups is optionally replaced by an oxygen atom, C₁-C₆haloalkoxy, C₂-C₆alkenyloxy, C₂-C₆alkynyloxy, C₁-C₆alkylsulfonyloxy, C₁-C₆halo-alkylsulfonyloxy, phenoxy or phenoxy substituted by one to three R⁹, benzyloxy or benzyloxy substituted by one to three R⁹, —CONH—SO₂—C₁-C₆alkyl, —CONH—SO₂—C₁-C₆haloalkyl, —NH—SO₂—C₁-C₆alkyl, —NH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —OCO—C₁-C₆alkyl, —OCO—C₁-C₆haloalkyl, —OCO-phenyl or —OCO-phenyl substituted by one to three R⁹, —OCONH—C₁-C₆alkyl, —OCONH—C₁-C₆haloalkyl, —OCONH-phenyl or —OCONH-phenyl substituted by one to three R⁹, or by one of the following groups Z, with Z= 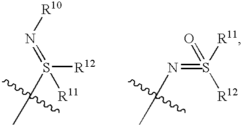
R¹⁰ is hydrogen, formyl, C₁-C₁₀alkyl, C₁-C₁₀haloalkyl, C₁-C₁₀alkylcarbonyl, C₁-C₁₀haloalkylcarbonyl, C₁-C₁₀alkoxycarbonyl, and
R¹¹ and R¹² are independently of each other C₁-C₁₀alkyl, C₁-C₁₀haloalkyl, C₁-C₁₀cycloalkyl, C₁-C₁₀cycloalkylalkyl, C₁-C₁₀alkoxyalkyl, or by —CONR⁷R⁸ wherein R⁷ and R³ are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R⁷ and R⁸ form a C₃-C₈alkylene group which optionally contains one oxygen or sulfur atom or one to two amino or C₁-C₆alkylamino groups, or Y is a 5- to 10-membered aromatic or non-aromatic heterocycle containing one to three nitrogen, oxygen or sulfur atoms, which is optionally benzo-fused, and which is optionally substituted by one to three substituents independently selected from C₁-C₆alkyl, C₃-C₆cycloalkyl, C₁-C₆haloalkyl, C₁-C₆hydroxyalkyl, C₁-C₆alkoxy-C₁-C₆alkyl, C₂-C₆alkenyl, C₂-C₆alkynyl, C₂-C₆haloalkenyl, C₁-C₆alkylcarbonyl, C₁-C₆halo-alkylcarbonyl, C₁-C₆alkoxycarbonyl, nitro, cyano, formyl, carboxyl, halogen, azido, thiocyanato, tri(C₁-C₆alkyl)silyl, mercapto, —SF₅, C₁-C₆alkylthio, C₁-C₆alkylsulfinyl, C₁-C₆alkylsulfonyl, C₁-C₆haloalkylthio, C₁-C₆haloalkylsulfinyl, C₁-C₆haloalkylsulfonyl, benzylsulfonyl or benzylsulfonyl substituted by one to three R⁹, phenylsulfonyl or phenylsulfonyl substituted by one to three R⁹, hydroxyl, C₁-C₆alkoxy, C₃-C₆cycloalkyloxy wherein one of the CH₂ groups is optionally replaced by an oxygen atom, C₁-C₆haloalkoxy, C₂-C₆alkenyloxy, C₂-C₆alkynyloxy, C₁-C₆alkylsulfonyloxy, C₁-C₆haloalkyl-sulfonyloxy, phenoxy or phenoxy substituted by one to three R⁹, benzyloxy or benzyloxy substituted by one to three R⁹, —CONH—SO₂—C₁-C₆alkyl, —CONH—SO₂—C₁-C₆haloalkyl, —NH—SO₂—C₁-C₆alkyl, —NH—SO₂—C₁-C₆haloalkyl, —NHCO—C₁-C₆alkyl, —NHCO—C₁-C₆haloalkyl, —NHCO₂—C₁-C₆alkyl, —NHCO₂—C₁-C₆haloalkyl, —OCO—C₁-C₆alkyl, —OCO—C₁-C₆haloalkyl, —OCO-phenyl or —OCO-phenyl substituted by one to three R⁹, —OCONH—C₁-C₆alkyl, —OCONH—C₁-C₆haloalkyl, —OCONH-phenyl or —OCONH-phenyl substituted by one to three R⁹, or by one of the following groups Z, with Z= 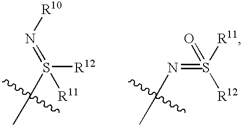
R¹⁰ is hydrogen, formyl, C₁-C₁₀alkyl, C₁-C₁₀haloalkyl, C₁-C₁₀alkylcarbonyl, C₁-C₁₀haloalkylcarbonyl, C₁-C₁₀alkoxycarbonyl, and
R¹¹ and R¹² are independently of each other C₁-C₁₀alkyl, C₁-C₁₀haloalkyl, C₁-C₁₀cycloalkyl, C₁-C₁₀cycloalkylalkyl, C₁-C₁₀alkoxyalkyl, or by —CONR⁷R⁸ wherein R⁷ and R³ are each independently of the other hydrogen, C₁-C₆alkyl, C₁-C₆haloalkyl, C₃-C₆cycloalkyl, phenyl or phenyl substituted by C₁-C₆haloalkyl, nitro, cyano or by halogen, or R⁷ and R³ together form a C₃-C₈alkylene group which optionally contains one oxygen or sulfur atom or one to two amino or C₁-C₆alkylamino groups;
R⁹ are independently from each other C₁-C₆haloalkyl, C₁-C₆alkoxycarbonyl, nitro, cyano, formyl, carboxyl or halogen;
and to N-oxides, salts and optical isomers of compounds of formula I.

3. A compound according to claim 1 in which R¹ and R² are independently C₁-C₆alkyl or C₁-C₆haloalkyl.

4. A compound according to claim 1 in which R¹ and R² are methyl.

5. A compound according to claim 1 in which R³ is halogen, azide or cyano.

6. A compound according to claim 1 in which R³ is fluoro or chloro.

7. A compound according to claim 1 in which R⁴ is hydrogen or halogen.

8. A compound according to claim 1 in which R⁴ is hydrogen, fluoro or chloro.

9. A compound according to claim 1 in which R⁵ is hydrogen, C₁-C₆alkyl or halogen.

10. A compound according to claim 1 in which R⁵ is hydrogen, methyl, fluoro or chloro.

11. A compound according to claim 1 in which R⁶ is hydrogen, methoxycarbonyl, C₁-C₆alkyl or halogen.

12. A compound according to claim 1 in which R⁶ is hydrogen, methyl, fluoro or chloro.

13. A compound according to claim 1 in which m is 1 or 2.

14. A compound according to claim 1 in which n is 1.

15. A compound according to claim 1 in which Y is an optionally substituted pyrazolyl, triazolyl, thiadiazolyl or triazolyl-N-oxide.

16. A compound according to claim 1 in which Y is an optionally substituted pyrazol-3-yl, pyrazol-4-yl, pyrazol-5-yl, 1,2,3-triazol-4-yl, 1,2,3-triazol-5-yl, 1,2,4-triazol-3-yl, 1,2,4-triazol-5-yl, 1,2,3-thiadiazol-5-yl or 1,2,3-triazol-4-yl-1-N-oxide.

17. A process for the preparation of a compound of formula V wherein R¹, R², R³ and R⁴ are as defined in claim 1 and X^{B} is a group selected from the group comprising halogen, alkylsulfinyl, arylsulfinyl, haloalkylsulfinyl, alkylsulfonyl, arylsulfonyl or haloalkylsulfonyl, wherein a compound of formula Va 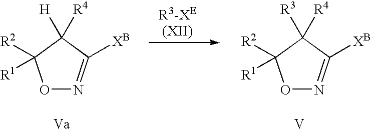
wherein R¹, R², R⁴ and X^{B} are defined as above is reacted with a base and a compound of formula XII, R³—X^{E}, wherein R³ is defined as in claim 1 and X^{E} is a suitable leaving group selected from the group comprising halide, perhaloalkyl, arylsulfonimide, imide, arylsulfonyl or tertiary amine in an inert solvent.

18. A process for the preparation of a compound of formula Vd wherein R¹, R², R³ and R⁴ are as defined in claim 1 and X^{H} is a group selected from the group comprising halogen, alkylsulfanyl, arylsulfanyl or haloalkylsulfanyl, wherein a compound of formula Ve, 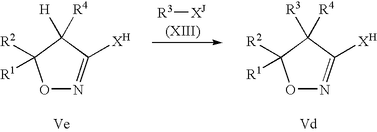
wherein R¹, R² and R⁴ are as defined in claim 1 and X^{H} is a group selected from the group comprising halogen, alkylsulfanyl, arylsulfanyl or haloalkylsulfanyl, is reacted with a compound of formula R³—X^{J}, wherein R³ is as defined in claim 1 and X^{G} is a functional group that may be cleaved to generate R³ as a radical in an inert solvent.

19. A process for the preparation of a compound of formula Vc wherein R¹, R², R³, R⁴ are as defined in claim 1 and X^{G} is a group selected from the group comprising alkylsulfanyl, arylsulfanyl or haloalkylsulfanyl, wherein a compound of formula Vf 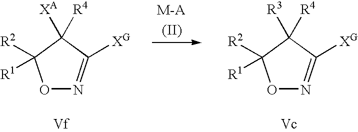
wherein R¹, R² and R⁴ are as defined in claim 1, X^{G} is a group selected from the group comprising alkylsulfanyl, arylsulfanyl or haloalkylsulfanyl, and X^{A} is a leaving group selected from the group comprising halide, alkylsulfonate, arylsulfonate or haloalkylsulfonate is reacted in an inert solvent with a suitable salt or a suitable organometal reagent of formula II.

20. A process for the preparation of a compound of formula XIV wherein R¹, R² and R³ are as described in claim 1 and R⁴ is hydrogen, wherein a compound of formula XV, 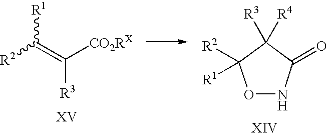
wherein R¹, R² and R³ are as described in claim 1 and R^{X} is an optionally substituted alkyl or an optionally substituted aryl is reacted with N-hydroxyurea or hydroxylamine in the presence of a suitable organic or inorganic base in an inert solvent.

21. A process for the preparation of a compound of formula I wherein R¹, R², R³, R⁴, R⁵, R⁶ and Y are as defined in claim 1, m is 0, and n is 1, wherein a compound of formula Ij, 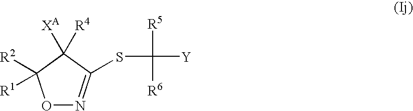
wherein R¹, R², R⁴, R⁵, R⁶ and Y are defined in claim 1 and X^{A} is a leaving group selected from the group comprising halide, an alkylsulfonate, an arylsulfonate or a haloalkylsulfonate is reacted with a suitable salt or a suitable organometal reagent of the formula II,
MA (II)
wherein M is an organic cation or an inorganic cation and A is an anion corresponding to R³ as defined in claim 1 such as halide, cyanide, azide or thiocyanate or an organic functionality such as an alkyl residue, or an alkenyl residue, or an alkynyl residue in an inert solvent.

22. A compound of formula V wherein R¹, R², R³ and R⁴ are as defined in claim 1 and X^{B} is group selected from the group comprising halogen, alkylsulfinyl, arylsulfinyl, haloalkylsulfinyl, alkylsulfonyl, arylsulfonyl or haloalkylsulfonyl, 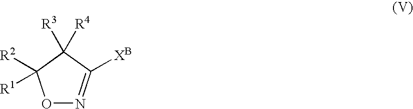
for use as an intermediate in the preparation of a compound of formula I, wherein R¹, R², R³, R⁴, R⁵, R⁶, m, n and Y are as defined in claim 1.

23. A compound of formula Vc wherein R¹, R², R³ and R⁴ are as defined in claim 1 and X^{G} is a selected from the group comprising alkylsulfanyl, arylsulfanyl or haloalkylsulfanyl 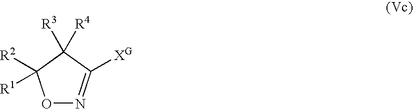
for use as an intermediate in the preparation of a compound of formula I, wherein R¹, R², R³, R⁴, R⁵, R⁶, m, n and Y are as defined in claim 1.

24. A herbicidal composition which comprises a herbicidally effective amount of a compound of formula I as defined in claim 1 in addition to formulation adjuvants.

25. A method of controlling plants which comprises applying to the plants or to the locus thereof a herbicidally effective amount of a compound of formula I as defined in claim 1.

26. A composition according to claim 24, which comprises a further herbicide in addition to the compound of formula I as defined in claim 1.

27. A composition according to claim 24, which comprises a safener in addition to the compound of formula I as defined in claim 1.
